SEQUENCE LISTING (127 sequences)

SEQ ID NO: 1 - DNA, 2535 bp, synthetic construct
  misc_feature 1..2535: note = hAce2-variant1-IgG4 nucleic acid sequence
  source 1..2535: mol_type = other DNA
atgtcaagctcttcctggctccttctcagccttgttgctgtaactgctgctcagtccaccattgaggaacaggccaagacatttttggacatgtttaaccacaaagccgaagacctgttctatcaaagttcacttgctgcttggaattataacaccaatattactgaagagaatgtccaaaacatgaataacgctggggacaaatggtctgcctttttaaaggaacagtccacatttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagtctaagtacggccctccttgtccttcctgccccgctcctgaatttctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtcgacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgccaagcagcatcgagaaaaccatcagcaaggccaagggccagcctagggaaccccaggtttacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggacagcgacggctcattcttcctgtactcccggctgaccgtggacaagtccagatggcaagagggcaacgtgttcagctgcagcgtgatgcacgaggccctgcacaatcactacacccagaagtccctgagtctgagcctgggcaagtga

SEQ ID NO: 2 - protein, 844 aa, synthetic construct
  REGION 1..844: note = hAce2-variant1-IgG4amino acid sequence
MSSSSWLLLSLVAVTAAQSTIEEQAKTFLDMFNHKAEDLFYQSSLAAWNYNTNITEENVQNMNNAGDKWSAFLKEQSTFAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - DNA, 2535 bp, synthetic construct
  misc_feature 1..2535: note = hAce2-variant2-IgG4 nucleic acid sequence
  source 1..2535: mol_type = other DNA
atgtcaagctcttcctggctccttctcagccttgttgctgtaactgctgctcagtccaccattgaggaacaggccaagacatttttggacatgtttaaccacaaagccgaagacctgttctatcaaagttcacttgctgcttggaattataacaccaatattactgaagagaatgtccaaaacatgaataacgctggggacaaatggtctgcctttttaaaggaacagtccacatttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagtctaagtacggccctccttgtccttcctgccccgctcctgaatttctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtcgacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgccaagcagcatcgagaaaaccatcagcaaggccaagggccagcctagggaaccccaggtttacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggacagcgacggctcattcttcctgtactcccggctgaccgtggacaagtccagatggcaagagggcaacgtgttcagctgcagcgtgatgcacgaggccctgcacaatcactacacccagaagtccctgagtctgagcctgggcaagtga

SEQ ID NO: 4 - protein, 844 aa, synthetic construct
  REGION 1..844: note = hAce2-variant2-IgG4amino acid sequence
MSSSSWLLLSLVAVTAAQSTIEEQAKTFLDMFNHKAEDLFYQSSLAAWNYNTNITEENVQNMNNAGDKWSAFLKEQSTFAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCLPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - DNA, 2535 bp, synthetic construct
  misc_feature 1..2535: note = hAce2-variant3-IgG4 nucleic acid sequence
  source 1..2535: mol_type = other DNA
atgtcaagctcttcctggctccttctcagccttgttgctgtaactgctgctcagtccaccattgaggaacaggccaagacatttttggacaggtttaaccacgtagccgaagacccgttctatcaaagttcacttgcttcttggaattataacaccaatatcactgaagagaatgcccaaaacatgaataatgctggggacaaatggtctgcctttttaaaggaacagtccacatttgcccaaatgtatccactacaagaaattcaggacctcacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagtctaagtacggccctccttgtccttcctgccccgctcctgaatttctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtcgacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgccaagcagcatcgagaaaaccatcagcaaggccaagggccagcctagggaaccccaggtttacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggacagcgacggctcattcttcctgtactcccggctgaccgtggacaagtccagatggcaagagggcaacgtgttcagctgcagcgtgatgcacgaggccctgcacaatcactacacccagaagtccctgagtctgagcctgggcaagtga

SEQ ID NO: 6 - protein, 844 aa, synthetic construct
  REGION 1..844: note = hAce2-variant3-IgG4amino acid sequence
MSSSSWLLLSLVAVTAAQSTIEEQAKTFLDRFNHVAEDPFYQSSLASWNYNTNITEENAQNMNNAGDKWSAFLKEQSTFAQMYPLQEIQDLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - DNA, 2535 bp, synthetic construct
  misc_feature 1..2535: note = hAce2-variant4-IgG4 nucleic acid sequence
  source 1..2535: mol_type = other DNA
atgtcaagctcttcctggctccttctcagccttgttgctgtaactgctgctcagtccaccattgaggaacaggccaagacatttttggacatgtttaaccacaaagccgaagacctgttctatcaaagttcacttgcttcttggaattataacaccaatattactgaagagaatgtccaaaacatgaataatgctggggacaaatggtctgcctttttaaaggaacagtccacaattgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagtctaagtacggccctccttgtccttcctgccccgctcctgaatttctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtcgacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgccaagcagcatcgagaaaaccatcagcaaggccaagggccagcctagggaaccccaggtttacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggacagcgacggctcattcttcctgtactcccggctgaccgtggacaagtccagatggcaagagggcaacgtgttcagctgcagcgtgatgcacgaggccctgcacaatcactacacccagaagtccctgagtctgagcctgggcaagtga

SEQ ID NO: 8 - protein, 844 aa, synthetic construct
  REGION 1..844: note = hAce2-variant4-IgG4amino acid sequence
MSSSSWLLLSLVAVTAAQSTIEEQAKTFLDMFNHKAEDLFYQSSLASWNYNTNITEENVQNMNNAGDKWSAFLKEQSTIAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - DNA, 1845 bp, synthetic construct
  misc_feature 1..1845: note = hAce2 variant 1 nucleic acid sequence
  source 1..1845: mol_type = other DNA
atgtcaagctcttcctggctccttctcagccttgttgctgtaactgctgctcagtccaccattgaggaacaggccaagacatttttggacatgtttaaccacaaagccgaagacctgttctatcaaagttcacttgctgcttggaattataacaccaatattactgaagagaatgtccaaaacatgaataacgctggggacaaatggtctgcctttttaaaggaacagtccacatttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagac

SEQ ID NO: 10 - protein, 615 aa, synthetic construct
  REGION 1..615: note = hAce2 variant 1 amino acid sequence
MSSSSWLLLSLVAVTAAQSTIEEQAKTFLDMFNHKAEDLFYQSSLAAWNYNTNITEENVQNMNNAGDKWSAFLKEQSTFAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 11 - DNA, 1845 bp, synthetic construct
  misc_feature 1..1845: note = hAce2 variant 2 nucleic acid sequence
  source 1..1845: mol_type = other DNA
atgtcaagctcttcctggctccttctcagccttgttgctgtaactgctgctcagtccaccattgaggaacaggccaagacatttttggacatgtttaaccacaaagccgaagacctgttctatcaaagttcacttgctgcttggaattataacaccaatattactgaagagaatgtccaaaacatgaataacgctggggacaaatggtctgcctttttaaaggaacagtccacatttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagac

SEQ ID NO: 12 - protein, 615 aa, synthetic construct
  REGION 1..615: note = hAce2 variant 2 amino acid sequence
MSSSSWLLLSLVAVTAAQSTIEEQAKTFLDMFNHKAEDLFYQSSLAAWNYNTNITEENVQNMNNAGDKWSAFLKEQSTFAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCLPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 13 - DNA, 1845 bp, synthetic construct
  misc_feature 1..1845: note = hAce2 variant 3 nucleic acid sequence
  source 1..1845: mol_type = other DNA
atgtcaagctcttcctggctccttctcagccttgttgctgtaactgctgctcagtccaccattgaggaacaggccaagacatttttggacaggtttaaccacgtagccgaagacccgttctatcaaagttcacttgcttcttggaattataacaccaatatcactgaagagaatgcccaaaacatgaataatgctggggacaaatggtctgcctttttaaaggaacagtccacatttgcccaaatgtatccactacaagaaattcaggacctcacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagac

SEQ ID NO: 14 - protein, 615 aa, synthetic construct
  REGION 1..615: note = hAce2 variant 3 amino acid sequence
MSSSSWLLLSLVAVTAAQSTIEEQAKTFLDRFNHVAEDPFYQSSLASWNYNTNITEENAQNMNNAGDKWSAFLKEQSTFAQMYPLQEIQDLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 15 - DNA, 1845 bp, synthetic construct
  misc_feature 1..1845: note = hAce2 variant 4 nucleic acid sequence
  source 1..1845: mol_type = other DNA
atgtcaagctcttcctggctccttctcagccttgttgctgtaactgctgctcagtccaccattgaggaacaggccaagacatttttggacatgtttaaccacaaagccgaagacctgttctatcaaagttcacttgcttcttggaattataacaccaatattactgaagagaatgtccaaaacatgaataatgctggggacaaatggtctgcctttttaaaggaacagtccacaattgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagac

SEQ ID NO: 16 - protein, 615 aa, synthetic construct
  REGION 1..615: note = hAce2 variant 4 amino acid sequence
MSSSSWLLLSLVAVTAAQSTIEEQAKTFLDMFNHKAEDLFYQSSLASWNYNTNITEENVQNMNNAGDKWSAFLKEQSTIAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 17 - DNA, 4884 bp, synthetic construct
  misc_feature 1..4884: note = plasmid comprising vector genome encoding hAce2(variant 1)-IgG4-FC
  repeat_region 1..130: note = 5'ITR
  regulatory 198..579: note = enhancer - CMV IE enhancer; regulatory_class = enhancer
  misc_feature 198..1928: note = CB7 hybrid promoter
  regulatory 582..863: note = promoter - CB promoter; regulatory_class = promoter
  misc_feature 956..1928: note = Intron - chicken beta actin intron (chimeric intron)
  misc_feature 1942..3786: note = hAce2 (variant 1) (GTP14)
  misc_feature 1942..1992: note = ACE2 signal peptide
  misc_feature 3787..4473: note = human IgG4 Fc
  regulatory 4540..4666: note = polyA_signal - rabbit globin polyA; regulatory_class = polyA_signal_sequence
  repeat_region 4755..4884: note = 3'ITR
  source 1..4884: mol_type = other DNA
  CDS 1942..4473: note = hAce2(Variant 1)(GTP14)-IgG4; protein_id = 18; translation = MSSSSWLLLSLVAVTAAQSTIEEQAKTFLDMFNHKAEDLFYQSS LAAWNYNTNITEENVQNMNNAGDKWSAFLKEQSTFAQMYPLQEIQNPTVKLQLQALQQ NGSSVLSEDKSKRLNTILNTMSTIYSTGKVCNPDNPQECLLLEPGLNEIMANSLDYNE RLWAWESWRSEVGKQLRPLYEEYVVLKNEMARANHYEDYGDYWRGDYEVNGVDGYDYS RGQLIEDVEHTFEEIKPLYEHLHAYVRAKLMNAYPSYISPIGCLPAHLLGDMWGRFWT NLYSLTVPFGQKPNIDVTDAMVDQAWDAQRIFKEAEKFFVSVGLPNMTQGFWEYSMLT DPGNVQKAVCHPTAWDLGKGDFRILMCTKVTMDDFLTAHHEMGHIQYDMAYAAQPFLL RNGANEGFHEAVGEIMSLSAATPKHLKSIGLLSPDFQEDNETEINFLLKQALTIVGTL PFTYMLEKWRWMVFKGEIPKDQWMKKWWEMKREIVGVVEPVPHDETYCDPASLFHVSN DYSFIRYYTRTLYQFQFQEALCQAAKHEGPLHKCDISNSTEAGQKLFNMLRLGKSEPW TLALENVVGAKNMNVRPLLNYFEPLFTWLKDQNKNSFVGWSTDWSPYADESKYGPPCP SCPAPEFLGGPSVFLFPPKPKDTLMISRTPEVTCVVVDVSQEDPEVQFNWYVDGVEVH NAKTKPREEQFNSTYRVVSVLTVLHQDWLNGKEYKCKVSNKGLPSSIEKTISKAKGQP REPQVYTLPPSQEEMTKNQVSLTCLVKGFYPSDIAVEWESNGQPENNYKTTPPVLDSD GSFFLYSRLTVDKSRWQEGNVFSCSVMHEALHNHYTQKSLSLSLGK
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgccaccatgtcaagctcttcctggctccttctcagccttgttgctgtaactgctgctcagtccaccattgaggaacaggccaagacatttttggacatgtttaaccacaaagccgaagacctgttctatcaaagttcacttgctgcttggaattataacaccaatattactgaagagaatgtccaaaacatgaataacgctggggacaaatggtctgcctttttaaaggaacagtccacatttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagtctaagtacggccctccttgtccttcctgccccgctcctgaatttctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtcgacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgccaagcagcatcgagaaaaccatcagcaaggccaagggccagcctagggaaccccaggtttacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggacagcgacggctcattcttcctgtactcccggctgaccgtggacaagtccagatggcaagagggcaacgtgttcagctgcagcgtgatgcacgaggccctgcacaatcactacacccagaagtccctgagtctgagcctgggcaagtgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 18 - protein, 844 aa, synthetic construct; identical to SEQ ID NO: 2
  REGION 1..844: note = Synthetic Construct

SEQ ID NO: 19 - DNA, 4884 bp, synthetic construct
  misc_feature 1..4884: note = plasmid comprising vector genome encoding hAce2(variant 2)-IgG4-FC
  repeat_region 1..130: note = 5'ITR
  regulatory 198..579: note = enhancer - CMV IE enhancer; regulatory_class = enhancer
  misc_feature 198..1928: note = CB7 hybrid promoter
  regulatory 582..863: note = promoter - CB promoter; regulatory_class = promoter
  misc_feature 956..1928: note = Intron - Chicken beta actin intron (chimeric intron)
  misc_feature 1942..3786: note = hAce(variant 2)(GTP14HL)
  misc_feature 1942..1992: note = ACE2 signal peptide
  misc_feature 3787..4473: note = human IgG4 FC
  regulatory 4540..4666: note = polyA_signal - Rabbit globin poly A; regulatory_class = polyA_signal_sequence
  repeat_region 4755..4884: note = 3'ITR
  source 1..4884: mol_type = other DNA
  CDS 1942..4473: note = hAce2(variant 2)(GTP14HL)-IgG4; protein_id = 20; translation = MSSSSWLLLSLVAVTAAQSTIEEQAKTFLDMFNHKAEDLFYQSS LAAWNYNTNITEENVQNMNNAGDKWSAFLKEQSTFAQMYPLQEIQNPTVKLQLQALQQ NGSSVLSEDKSKRLNTILNTMSTIYSTGKVCNPDNPQECLLLEPGLNEIMANSLDYNE RLWAWESWRSEVGKQLRPLYEEYVVLKNEMARANHYEDYGDYWRGDYEVNGVDGYDYS RGQLIEDVEHTFEEIKPLYEHLHAYVRAKLMNAYPSYISPIGCLPAHLLGDMWGRFWT NLYSLTVPFGQKPNIDVTDAMVDQAWDAQRIFKEAEKFFVSVGLPNMTQGFWEYSMLT DPGNVQKAVCLPTAWDLGKGDFRILMCTKVTMDDFLTAHHEMGHIQYDMAYAAQPFLL RNGANEGFHEAVGEIMSLSAATPKHLKSIGLLSPDFQEDNETEINFLLKQALTIVGTL PFTYMLEKWRWMVFKGEIPKDQWMKKWWEMKREIVGVVEPVPHDETYCDPASLFHVSN DYSFIRYYTRTLYQFQFQEALCQAAKHEGPLHKCDISNSTEAGQKLFNMLRLGKSEPW TLALENVVGAKNMNVRPLLNYFEPLFTWLKDQNKNSFVGWSTDWSPYADESKYGPPCP SCPAPEFLGGPSVFLFPPKPKDTLMISRTPEVTCVVVDVSQEDPEVQFNWYVDGVEVH NAKTKPREEQFNSTYRVVSVLTVLHQDWLNGKEYKCKVSNKGLPSSIEKTISKAKGQP REPQVYTLPPSQEEMTKNQVSLTCLVKGFYPSDIAVEWESNGQPENNYKTTPPVLDSD GSFFLYSRLTVDKSRWQEGNVFSCSVMHEALHNHYTQKSLSLSLGK
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgccaccatgtcaagctcttcctggctccttctcagccttgttgctgtaactgctgctcagtccaccattgaggaacaggccaagacatttttggacatgtttaaccacaaagccgaagacctgttctatcaaagttcacttgctgcttggaattataacaccaatattactgaagagaatgtccaaaacatgaataacgctggggacaaatggtctgcctttttaaaggaacagtccacatttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagtctaagtacggccctccttgtccttcctgccccgctcctgaatttctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtcgacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgccaagcagcatcgagaaaaccatcagcaaggccaagggccagcctagggaaccccaggtttacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggacagcgacggctcattcttcctgtactcccggctgaccgtggacaagtccagatggcaagagggcaacgtgttcagctgcagcgtgatgcacgaggccctgcacaatcactacacccagaagtccctgagtctgagcctgggcaagtgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 20 - protein, 844 aa, synthetic construct; identical to SEQ ID NO: 4
  REGION 1..844: note = Synthetic Construct

SEQ ID NO: 21 - DNA, 4884 bp, synthetic construct
  misc_feature 1..4884: note = plasmid comprising vector genome encoding hAce2(variant 3)-IgG4-FC
  repeat_region 1..130: note = 5'ITR
  regulatory 198..579: note = enhancer - CMV IE Enhancer; regulatory_class = enhancer
  misc_feature 198..1928: note = CB7 hybrid promoter
  regulatory 582..863: note = promoter - CB promoter; regulatory_class = promoter
  misc_feature 956..1928: note = Intron - Chicken beta actin intron (chimeric intron)
  misc_feature 1942..3786: note = hAce2 (Varinat 3) (Gen3-3)
  misc_feature 1942..1992: note = ACE2 signal peptide
  misc_feature 3787..4473: note = human IgG4 FC
  regulatory 4540..4666: note = polyA_signal - Rabbit Globin poly A; regulatory_class = polyA_signal_sequence
  repeat_region 4755..4884: note = 3'ITR
  source 1..4884: mol_type = other DNA
  CDS 1942..4473: note = hAce2 (variant 3) (Gen3-3) IgG4; protein_id = 22; translation = MSSSSWLLLSLVAVTAAQSTIEEQAKTFLDRFNHVAEDPFYQSS LASWNYNTNITEENAQNMNNAGDKWSAFLKEQSTFAQMYPLQEIQDLTVKLQLQALQQ NGSSVLSEDKSKRLNTILNTMSTIYSTGKVCNPDNPQECLLLEPGLNEIMANSLDYNE RLWAWESWRSEVGKQLRPLYEEYVVLKNEMARANHYEDYGDYWRGDYEVNGVDGYDYS RGQLIEDVEHTFEEIKPLYEHLHAYVRAKLMNAYPSYISPIGCLPAHLLGDMWGRFWT NLYSLTVPFGQKPNIDVTDAMVDQAWDAQRIFKEAEKFFVSVGLPNMTQGFWEYSMLT DPGNVQKAVCHPTAWDLGKGDFRILMCTKVTMDDFLTAHHEMGHIQYDMAYAAQPFLL RNGANEGFHEAVGEIMSLSAATPKHLKSIGLLSPDFQEDNETEINFLLKQALTIVGTL PFTYMLEKWRWMVFKGEIPKDQWMKKWWEMKREIVGVVEPVPHDETYCDPASLFHVSN DYSFIRYYTRTLYQFQFQEALCQAAKHEGPLHKCDISNSTEAGQKLFNMLRLGKSEPW TLALENVVGAKNMNVRPLLNYFEPLFTWLKDQNKNSFVGWSTDWSPYADESKYGPPCP SCPAPEFLGGPSVFLFPPKPKDTLMISRTPEVTCVVVDVSQEDPEVQFNWYVDGVEVH NAKTKPREEQFNSTYRVVSVLTVLHQDWLNGKEYKCKVSNKGLPSSIEKTISKAKGQP REPQVYTLPPSQEEMTKNQVSLTCLVKGFYPSDIAVEWESNGQPENNYKTTPPVLDSD GSFFLYSRLTVDKSRWQEGNVFSCSVMHEALHNHYTQKSLSLSLGK
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgccaccatgtcaagctcttcctggctccttctcagccttgttgctgtaactgctgctcagtccaccattgaggaacaggccaagacatttttggacaggtttaaccacgtagccgaagacccgttctatcaaagttcacttgcttcttggaattataacaccaatatcactgaagagaatgcccaaaacatgaataatgctggggacaaatggtctgcctttttaaaggaacagtccacatttgcccaaatgtatccactacaagaaattcaggacctcacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagtctaagtacggccctccttgtccttcctgccccgctcctgaatttctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtcgacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgccaagcagcatcgagaaaaccatcagcaaggccaagggccagcctagggaaccccaggtttacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggacagcgacggctcattcttcctgtactcccggctgaccgtggacaagtccagatggcaagagggcaacgtgttcagctgcagcgtgatgcacgaggccctgcacaatcactacacccagaagtccctgagtctgagcctgggcaagtgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 22 - protein, 844 aa, synthetic construct; identical to SEQ ID NO: 6
  REGION 1..844: note = Synthetic Construct

SEQ ID NO: 23 - DNA, 4884 bp, synthetic construct
  misc_feature 1..4884: note = plasmid comprising vector genome encoding hAce2(variant 4)-IgG4-FC
  repeat_region 1..130: note = 5'ITR
  regulatory 198..579: note = enhancer - CMV IE Enhancer; regulatory_class = enhancer
  misc_feature 198..1928: note = CB7 hybrid promoter
  regulatory 582..863: note = promoter - CB promoter; regulatory_class = promoter
  misc_feature 956..1928: note = Intron - Chicken beta actin intron (chimeric intron)
  misc_feature 1942..3786: note = hAce2 (variant 4) (GTP14-Gen3-5)
  misc_feature 1942..1992: note = ACE2 signal peptide
  misc_feature 3787..4473: note = Human IgG4 Fc
  regulatory 4540..4666: note = polyA_signal - Rabbit globin polyA; regulatory_class = polyA_signal_sequence
  repeat_region 4755..4884: note = 3'ITR
  source 1..4884: mol_type = other DNA
  CDS 1942..4473: note = hAce2(varinat 4) (GTP14 Gen3-5) IgG4; protein_id = 24; translation = MSSSSWLLLSLVAVTAAQSTIEEQAKTFLDMFNHKAEDLFYQSS LASWNYNTNITEENVQNMNNAGDKWSAFLKEQSTIAQMYPLQEIQNPTVKLQLQALQQ NGSSVLSEDKSKRLNTILNTMSTIYSTGKVCNPDNPQECLLLEPGLNEIMANSLDYNE RLWAWESWRSEVGKQLRPLYEEYVVLKNEMARANHYEDYGDYWRGDYEVNGVDGYDYS RGQLIEDVEHTFEEIKPLYEHLHAYVRAKLMNAYPSYISPIGCLPAHLLGDMWGRFWT NLYSLTVPFGQKPNIDVTDAMVDQAWDAQRIFKEAEKFFVSVGLPNMTQGFWEYSMLT DPGNVQKAVCHPTAWDLGKGDFRILMCTKVTMDDFLTAHHEMGHIQYDMAYAAQPFLL RNGANEGFHEAVGEIMSLSAATPKHLKSIGLLSPDFQEDNETEINFLLKQALTIVGTL PFTYMLEKWRWMVFKGEIPKDQWMKKWWEMKREIVGVVEPVPHDETYCDPASLFHVSN DYSFIRYYTRTLYQFQFQEALCQAAKHEGPLHKCDISNSTEAGQKLFNMLRLGKSEPW TLALENVVGAKNMNVRPLLNYFEPLFTWLKDQNKNSFVGWSTDWSPYADESKYGPPCP SCPAPEFLGGPSVFLFPPKPKDTLMISRTPEVTCVVVDVSQEDPEVQFNWYVDGVEVH NAKTKPREEQFNSTYRVVSVLTVLHQDWLNGKEYKCKVSNKGLPSSIEKTISKAKGQP REPQVYTLPPSQEEMTKNQVSLTCLVKGFYPSDIAVEWESNGQPENNYKTTPPVLDSD GSFFLYSRLTVDKSRWQEGNVFSCSVMHEALHNHYTQKSLSLSLGK
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgccaccatgtcaagctcttcctggctccttctcagccttgttgctgtaactgctgctcagtccaccattgaggaacaggccaagacatttttggacatgtttaaccacaaagccgaagacctgttctatcaaagttcacttgcttcttggaattataacaccaatattactgaagagaatgtccaaaacatgaataatgctggggacaaatggtctgcctttttaaaggaacagtccacaattgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagtctaagtacggccctccttgtccttcctgccccgctcctgaatttctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtcgacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgccaagcagcatcgagaaaaccatcagcaaggccaagggccagcctagggaaccccaggtttacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggacagcgacggctcattcttcctgtactcccggctgaccgtggacaagtccagatggcaagagggcaacgtgttcagctgcagcgtgatgcacgaggccctgcacaatcactacacccagaagtccctgagtctgagcctgggcaagtgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 24 - protein, 844 aa, synthetic construct; identical to SEQ ID NO: 8
  REGION 1..844: note = Synthetic Construct

SEQ ID NO: 25 - protein, 805 aa, synthetic construct [sequence omitted]

SEQ ID NO: 26 - DNA, 1794 bp, synthetic construct
  misc_feature 1..1794: note = hAce2 variant 1 nucleic acid sequence without signal/leader peptide
  source 1..1794: mol_type = other DNA
cagtccaccattgaggaacaggccaagacatttttggacatgtttaaccacaaagccgaagacctgttctatcaaagttcacttgctgcttggaattataacaccaatattactgaagagaatgtccaaaacatgaataacgctggggacaaatggtctgcctttttaaaggaacagtccacatttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagac

SEQ ID NO: 27 - protein, 598 aa, synthetic construct
  REGION 1..598: note = hAce2 variant 1 amino acid sequence without signal/leader peptide
QSTIEEQAKTFLDMFNHKAEDLFYQSSLAAWNYNTNITEENVQNMNNAGDKWSAFLKEQSTFAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 28 - DNA, 1794 bp, synthetic construct
  misc_feature 1..1794: note = hAce2 variant 2 nucleic acid sequence without signal/leader peptide
  source 1..1794: mol_type = other DNA
cagtccaccattgaggaacaggccaagacatttttggacatgtttaaccacaaagccgaagacctgttctatcaaagttcacttgctgcttggaattataacaccaatattactgaagagaatgtccaaaacatgaataacgctggggacaaatggtctgcctttttaaaggaacagtccacatttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagac

SEQ ID NO: 29 - protein, 598 aa, synthetic construct
  REGION 1..598: note = hAce2 variant 2 amino acid sequence without signal/leader peptide
QSTIEEQAKTFLDMFNHKAEDLFYQSSLAAWNYNTNITEENVQNMNNAGDKWSAFLKEQSTFAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCLPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 30 - DNA, 1794 bp, synthetic construct
  misc_feature 1..1794: note = hAce2 variant 3 nucleic acid sequence without signal/leader sequence
  source 1..1794: mol_type = other DNA
cagtccaccattgaggaacaggccaagacatttttggacaggtttaaccacgtagccgaagacccgttctatcaaagttcacttgcttcttggaattataacaccaatatcactgaagagaatgcccaaaacatgaataatgctggggacaaatggtctgcctttttaaaggaacagtccacatttgcccaaatgtatccactacaagaaattcaggacctcacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagac

SEQ ID NO: 31 - protein, 598 aa, synthetic construct
  REGION 1..598: note = hAce2 variant 3 amino acid sequence without signal/leader sequence
QSTIEEQAKTFLDRFNHVAEDPFYQSSLASWNYNTNITEENAQNMNNAGDKWSAFLKEQSTFAQMYPLQEIQDLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 32 - DNA, 1794 bp, synthetic construct
  misc_feature 1..1794: note = hAce2 variant 4 nucleic acid sequence without signal/leader peptide
  source 1..1794: mol_type = other DNA
cagtccaccattgaggaacaggccaagacatttttggacatgtttaaccacaaagccgaagacctgttctatcaaagttcacttgcttcttggaattataacaccaatattactgaagagaatgtccaaaacatgaataatgctggggacaaatggtctgcctttttaaaggaacagtccacaattgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagac

SEQ ID NO: 33 - protein, 598 aa, synthetic construct
  REGION 1..598: note = hAce2 variant 4 amino acid sequence without signal/leader peptide
QSTIEEQAKTFLDMFNHKAEDLFYQSSLASWNYNTNITEENVQNMNNAGDKWSAFLKEQSTIAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 34 - DNA, 2484 bp, synthetic construct
  misc_feature 1..2484: note = hAce2-variant1-IgG4 nucleic acid sequence without signal/leader peptide
  source 1..2484: mol_type = other DNA
cagtccaccattgaggaacaggccaagacatttttggacatgtttaaccacaaagccgaagacctgttctatcaaagttcacttgctgcttggaattataacaccaatattactgaagagaatgtccaaaacatgaataacgctggggacaaatggtctgcctttttaaaggaacagtccacatttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagtctaagtacggccctccttgtccttcctgccccgctcctgaatttctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtcgacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgccaagcagcatcgagaaaaccatcagcaaggccaagggccagcctagggaaccccaggtttacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggacagcgacggctcattcttcctgtactcccggctgaccgtggacaagtccagatggcaagagggcaacgtgttcagctgcagcgtgatgcacgaggccctgcacaatcactacacccagaagtccctgagtctgagcctgggcaagtga

SEQ ID NO: 35 - protein, 827 aa, synthetic construct
  REGION 1..827: note = hAce2-variant1-IgG4amino acid sequence without signal/leader peptide
QSTIEEQAKTFLDMFNHKAEDLFYQSSLAAWNYNTNITEENVQNMNNAGDKWSAFLKEQSTFAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - DNA, 2484 bp, synthetic construct
  misc_feature 1..2484: note = hAce2-variant2-IgG4 nucleic acid sequence without signal/leader peptide
  source 1..2484: mol_type = other DNA
cagtccaccattgaggaacaggccaagacatttttggacatgtttaaccacaaagccgaagacctgttctatcaaagttcacttgctgcttggaattataacaccaatattactgaagagaatgtccaaaacatgaataacgctggggacaaatggtctgcctttttaaaggaacagtccacatttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagtctaagtacggccctccttgtccttcctgccccgctcctgaatttctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtcgacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgccaagcagcatcgagaaaaccatcagcaaggccaagggccagcctagggaaccccaggtttacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggacagcgacggctcattcttcctgtactcccggctgaccgtggacaagtccagatggcaagagggcaacgtgttcagctgcagcgtgatgcacgaggccctgcacaatcactacacccagaagtccctgagtctgagcctgggcaagtga

SEQ ID NO: 37 - protein, 827 aa, synthetic construct
  REGION 1..827: note = hAce2-variant2-IgG4amino acid sequence without signal/leader peptide
QSTIEEQAKTFLDMFNHKAEDLFYQSSLAAWNYNTNITEENVQNMNNAGDKWSAFLKEQSTFAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCLPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - DNA, 2484 bp, synthetic construct
  misc_feature 1..2484: note = hAce2-variant3-IgG4 nucleic acid sequence without signal/leader peptide
  source 1..2484: mol_type = other DNA
cagtccaccattgaggaacaggccaagacatttttggacaggtttaaccacgtagccgaagacccgttctatcaaagttcacttgcttcttggaattataacaccaatatcactgaagagaatgcccaaaacatgaataatgctggggacaaatggtctgcctttttaaaggaacagtccacatttgcccaaatgtatccactacaagaaattcaggacctcacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagtctaagtacggccctccttgtccttcctgccccgctcctgaatttctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtcgacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgccaagcagcatcgagaaaaccatcagcaaggccaagggccagcctagggaaccccaggtttacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggacagcgacggctcattcttcctgtactcccggctgaccgtggacaagtccagatggcaagagggcaacgtgttcagctgcagcgtgatgcacgaggccctgcacaatcactacacccagaagtccctgagtctgagcctgggcaagtga

SEQ ID NO: 39 - protein, 827 aa, synthetic construct
  REGION 1..827: note = hAce2-variant3-IgG4amino acid sequence without signal/leader peptide
QSTIEEQAKTFLDRFNHVAEDPFYQSSLASWNYNTNITEENAQNMNNAGDKWSAFLKEQSTFAQMYPLQEIQDLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - DNA, 2484 bp, synthetic construct
  misc_feature 1..2484: note = hAce2-variant4-IgG4 nucleic acid sequence without signal/leader peptide
  source 1..2484: mol_type = other DNA
cagtccaccattgaggaacaggccaagacatttttggacatgtttaaccacaaagccgaagacctgttctatcaaagttcacttgcttcttggaattataacaccaatattactgaagagaatgtccaaaacatgaataatgctggggacaaatggtctgcctttttaaaggaacagtccacaattgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagtctaagtacggccctccttgtccttcctgccccgctcctgaatttctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtcgacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgccaagcagcatcgagaaaaccatcagcaaggccaagggccagcctagggaaccccaggtttacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggacagcgacggctcattcttcctgtactcccggctgaccgtggacaagtccagatggcaagagggcaacgtgttcagctgcagcgtgatgcacgaggccctgcacaatcactacacccagaagtccctgagtctgagcctgggcaagtga

SEQ ID NO: 41 - protein, 827 aa, synthetic construct
  REGION 1..827: note = hAce2-variant4-IgG4amino acid sequence without signal/leader peptide
QSTIEEQAKTFLDMFNHKAEDLFYQSSLASWNYNTNITEENVQNMNNAGDKWSAFLKEQSTIAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 788 aa, synthetic construct
  REGION 1..788: note = wild type hAce2 (based on isoform 1 amino acid sequence; NP_068576.1) without signal/leader peptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSF

SEQ ID NO: 43 - protein, 229 aa, synthetic construct
  REGION 1..229: note = SARS-CoV2 RBD (Wuhan) wild type
MYRMQLLSCIALSLALVTNSGSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSHHHHHH

SEQ ID NO: 44 - protein, 229 aa, synthetic construct
  REGION 1..229: note = SARS-CoV2 RBDSouth Africa Variant
MYRMQLLSCIALSLALVTNSGSPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSHHHHHH

SEQ ID NO: 45 - protein, 229 aa, synthetic construct
  REGION 1..229: note = SARS-CoV2 RBD439K 417V
MYRMQLLSCIALSLALVTNSGSPNITNLCPFGEVFNATRFASVYAWNRKRISNCVADYSVLYNSASFSTFKCYGVSPTKLNDLCFTNVYADSFVIRGDEVRQIAPGQTGVIADYNYKLPDDFTGCVIAWNSKNLDSKVGGNYNYLYRLFRKSNLKPFERDISTEIYQAGSTPCNGVEGFNCYFPLQSYGFQPTNGVGYQPYRVVVLSFELLHAPATVCGPKKSHHHHHH

SEQ ID NO: 46 - protein, 229 aa, synthetic construct
  REGION 1..229: note = SARS-CoV2 RBD UK 501Y
MYRMQLLSCIALSLALVTNSGSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSHHHHHH

SEQ ID NO: 47 - protein, 229 aa, synthetic construct
  REGION 1..229: note = SARS-CoV2 RBD 439K
MYRMQLLSCIALSLALVTNSGSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSKNLDSKVGGNYNYLYRLFRKSNLKPFERDISTEIYQAGSTPCNGVEGFNCYFPLQSYGFQPTNGVGYQPYRVVVLSFELLHAPATVCGPKKSHHHHHH

SEQ ID NO: 48 - protein, 229 aa, synthetic construct
  REGION 1..229: note = 453F
MYRMQLLSCIALSLALVTNSGSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLFRLFRKSNLKPFERDISTEIYQAGSTPCNGVEGFNCYFPLQSYGFQPTNGVGYQPYRVVVLSFELLHAPATVCGPKKSHHHHHH

SEQ ID NO: 49 - protein, 229 aa, synthetic construct
  REGION 1..229: note = SARS-CoV2 RBD 486L
MYRMQLLSCIALSLALVTNSGSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGLNCYFPLQSYGFQPTNGVGYQPYRVVVLSFELLHAPATVCGPKKSHHHHHH

SEQ ID NO: 50 - protein, 229 aa, synthetic construct
  REGION 1..229: note = 501T
MYRMQLLSCIALSLALVTNSGSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTTGVGYQPYRVVVLSFELLHAPATVCGPKKSHHHHHH

SEQ ID NO: 51 - protein, 228 aa, synthetic construct
  REGION 1..228: note = SARS CoV1 RBD
MYRMQLLSCIALSLALVTNSGSPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSHHHHHH

SEQ ID NO: 52 - protein, 615 aa, synthetic construct
  REGION 1..615: note = amino acid sequence of hAce mutant clone No. 44; 27A/43G/49D/79F/90D/330Y
MSSSSWLLLSLVAVTAAQSTIEEQAKAFLDKFNHEAEDLFYQGSLASWDYNTNITEENVQNMNNAGDKWSAFLKEQSTFAQMYPLQEIQDLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 53 - protein, 615 aa, synthetic construct
  REGION 1..615: note = amino acid sequence of hAce mutant clone No.12; 27A/49D/61D/75K/91P/330Y
MSSSSWLLLSLVAVTAAQSTIEEQAKAFLDKFNHEAEDLFYQSSLASWDYNTNITEENVQDMNNAGDKWSAFLKKQSTLAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 54 - protein, 615 aa, synthetic construct
  REGION 1..615: note = amino acid sequence of hAce mutant clone No.16; 27A/43G/61D/81R/91P/330Y
MSSSSWLLLSLVAVTAAQSTIEEQAKAFLDKFNHEAEDLFYQGSLASWNYNTNITEENVQDMNNAGDKWSAFLKEQSTLARMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 55 - protein, 615 aa, synthetic construct
  REGION 1..615: note = amino acid sequence of hAce mutant clone No.17; 27A/49D/61D/79F/90S/330Y
MSSSSWLLLSLVAVTAAQSTIEEQAKAFLDKFNHEAEDLFYQSSLASWDYNTNITEENVQDMNNAGDKWSAFLKEQSTFAQMYPLQEIQSLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 56 - protein, 615 aa, synthetic construct
  REGION 1..615: note = amino acid sequence of hAce mutant clone No.33; 27A/43G/49D/79F/91P/330Y
MSSSSWLLLSLVAVTAAQSTIEEQAKAFLDKFNHEAEDLFYQGSLASWDYNTNITEENVQNMNNAGDKWSAFLKEQSTFAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 57 - protein, 615 aa, synthetic construct
  REGION 1..615: note = amino acid sequence of hAce mutant clone No.24; 27A/43G/49D/61D/76R/330Y
MSSSSWLLLSLVAVTAAQSTIEEQAKAFLDKFNHEAEDLFYQGSLASWDYNTNITEENVQDMNNAGDKWSAFLKERSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 58 - protein, 615 aa, synthetic construct
  REGION 1..615: note = amino acid sequence of hAce mutant clone No.27; 43G/49D/79F/90D/330Y
MSSSSWLLLSLVAVTAAQSTIEEQAKTFLDKFNHEAEDLFYQGSLASWDYNTNITEENVQNMNNAGDKWSAFLKEQSTFAQMYPLQEIQDLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 59 - protein, 615 aa, synthetic construct
  REGION 1..615: note = amino acid sequence of hAce mutant clone No.43; 27A/43G/61D/75K/90D/330Y
MSSSSWLLLSLVAVTAAQSTIEEQAKAFLDKFNHEAEDLFYQGSLASWNYNTNITEENVQDMNNAGDKWSAFLKKQSTLAQMYPLQEIQDLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 60 - protein, 615 aa, synthetic construct
  REGION 1..615: note = amino acid sequence of hAce mutant clone No.35; 27A/43G/61D/79F/90S/330Y
MSSSSWLLLSLVAVTAAQSTIEEQAKAFLDKFNHEAEDLFYQGSLASWNYNTNITEENVQDMNNAGDKWSAFLKEQSTFAQMYPLQEIQSLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 61 - protein, 615 aa, synthetic construct
  REGION 1..615: note = amino acid sequence of hAce mutant clone No.39; 27A/49D/61D/81R/92A/330Y
MSSSSWLLLSLVAVTAAQSTIEEQAKAFLDKFNHEAEDLFYQSSLASWDYNTNITEENVQDMNNAGDKWSAFLKEQSTLARMYPLQEIQNLAVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 62 - protein, 598 aa, synthetic construct
  REGION 1..598: note = amino acid sequence of hAce mutant clone No.44; 27A/43G/49D/79F/90D/330Y; without leader sequence
QSTIEEQAKAFLDKFNHEAEDLFYQGSLASWDYNTNITEENVQNMNNAGDKWSAFLKEQSTFAQMYPLQEIQDLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 63 - protein, 598 aa, synthetic construct
  REGION 1..598: note = amino acid sequence of hAce mutant clone No.12; 27A/49D/61D/75K/91P/330Y; without leader sequence
QSTIEEQAKAFLDKFNHEAEDLFYQSSLASWDYNTNITEENVQDMNNAGDKWSAFLKKQSTLAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 64 - protein, 598 aa, synthetic construct
  REGION 1..598: note = amino acid sequence of hAce mutant clone No.16; 27A/43G/61D/81R/91P/330Y; without leader sequence
QSTIEEQAKAFLDKFNHEAEDLFYQGSLASWNYNTNITEENVQDMNNAGDKWSAFLKEQSTLARMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 65 - protein, 598 aa, synthetic construct
  REGION 1..598: note = amino acid sequence of hAce mutant clone No.17; 27A/49D/61D/79F/90S/330Y; without leader sequence
QSTIEEQAKAFLDKFNHEAEDLFYQSSLASWDYNTNITEENVQDMNNAGDKWSAFLKEQSTFAQMYPLQEIQSLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 66 - protein, 598 aa, synthetic construct
  REGION 1..598: note = amino acid sequence of hAce mutant clone No.33; 27A/43G/49D/79F/91P/330Y; without leader sequence
QSTIEEQAKAFLDKFNHEAEDLFYQGSLASWDYNTNITEENVQNMNNAGDKWSAFLKEQSTFAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 67 - protein, 598 aa, synthetic construct
  REGION 1..598: note = amino acid sequence of hAce mutant clone No.24; 27A/43G/49D/61D/76R/330Y; without leader sequence
QSTIEEQAKAFLDKFNHEAEDLFYQGSLASWDYNTNITEENVQDMNNAGDKWSAFLKERSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 68 - protein, 598 aa, synthetic construct
  REGION 1..598: note = amino acid sequence of hAce mutant clone No.27; 43G/49D/79F/90D/330Y; without leader sequence
QSTIEEQAKTFLDKFNHEAEDLFYQGSLASWDYNTNITEENVQNMNNAGDKWSAFLKEQSTFAQMYPLQEIQDLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 69 - protein, 598 aa, synthetic construct
  REGION 1..598: note = amino acid sequence of hAce mutant clone No.43; 27A/43G/61D/75K/90D/330Y; without leader sequence
QSTIEEQAKAFLDKFNHEAEDLFYQGSLASWNYNTNITEENVQDMNNAGDKWSAFLKKQSTLAQMYPLQEIQDLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 70 - protein, 598 aa, synthetic construct
  REGION 1..598: note = amino acid sequence of hAce mutant clone No.35; 27A/43G/61D/79F/90S/330Y; without leader sequence
QSTIEEQAKAFLDKFNHEAEDLFYQGSLASWNYNTNITEENVQDMNNAGDKWSAFLKEQSTFAQMYPLQEIQSLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 71 - protein, 598 aa, synthetic construct
  REGION 1..598: note = amino acid sequence of hAce mutant clone No.39; 27A/49D/61D/81R/92A/330Y; without leader sequence
QSTIEEQAKAFLDKFNHEAEDLFYQSSLASWDYNTNITEENVQDMNNAGDKWSAFLKEQSTLARMYPLQEIQNLAVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 72 - protein, 615 aa, synthetic construct
  REGION 1..615: note = hAce2 variant 5 amino acid sequence with signal/leader peptide
MSSSSWLLLSLVAVTAAQSTIEEQAKTFLDMFNHKAEDLFYRSSLASWNYNTNITEENVQNMNNAGDKWSAFLKEQSTLAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 73 - protein, 615 aa, synthetic construct
  REGION 1..615: note = hAce2 variant 6 amino acid sequence with signal/leader peptide
MSSSSWLLLSLVAVTAAQSTIEEQAKTFLDMFNHKAEDLFYQSSLASWNYNTNITEENVQNMNNAGDKWSAFLKEQSTFAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 74 - protein, 598 aa, synthetic construct
  REGION 1..598: note = hAce2 variant 5 amino acid sequence without signal/leader peptide
QSTIEEQAKTFLDMFNHKAEDLFYRSSLASWNYNTNITEENVQNMNNAGDKWSAFLKEQSTLAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 75 - protein, 615 aa, synthetic construct; identical to SEQ ID NO: 73
  REGION 1..615: note = hAce2 variant 6 amino acid sequence without signal/leader peptide

SEQ ID NO: 76 - protein, 17 aa, synthetic construct
  REGION 1..17: note = amino acid sequence of native signal/leader peptide
MSSSSWLLLSLVAVTAA

SEQ ID NO: 77 - protein, 229 aa, synthetic construct
  REGION 1..229: note = amino acid sequence of human IgG4 Fc domain
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 78 - protein, 348 aa, synthetic construct
  REGION 1..348: note = human IgM Fc domain
IAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 79 - protein, 347 aa, synthetic construct
  REGION 1..347: note = human IgM (without N-terminal "I") Fc domain
AELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 80 - protein, 615 aa, synthetic construct
  REGION 1..615: note = wild type hAce2 amino acids 1-615 (based on isoform 1 amino acid sequence; NP_068576.1)
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 81 - protein, 598 aa, synthetic construct
  REGION 1..598: note = wild type hAce2 amino acids 18-615 (based on isoform 1 amino acid sequence; NP_068576.1) without signal/leader peptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 82 - protein, 740 aa, synthetic construct
  REGION 1..740: note = wild type hAce2 amino acids 1-740 (based on isoform 1 amino acid sequence; NP_068576.1)
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 83 - protein, 723 aa, synthetic construct
  REGION 1..723: note = wild type hAce2 amino acids 18-740 (based on isoform 1 amino acid sequence; NP_068576.1) without signal/leader peptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 84 - protein, 12 aa, synthetic construct
  REGION 1..12: note = peptide for detection using Mass Spectrometry
ANHYEDYGDYWR

SEQ ID NO: 85 - DNA, 4893 bp, synthetic construct
  misc_feature 1..4893: note = vector genome encoding hAce2(variant 2)-IgG1-FC
  repeat_region 1..130: note = 5'ITR
  regulatory 198..579: note = enhancer - CMV IE promoter/enhancer; regulatory_class = enhancer
  misc_feature 198..1928: note = CB7 hybrid promoter
  regulatory 582..863: note = promoter - CB promoter; regulatory_class = promoter
  misc_feature 956..1928: note = Intron - chicken beta-actin intron (chimeric intron)
  misc_feature 1942..3786: note = hAce2HL
  misc_feature 1942..4482: note = hAce2HL
  misc_feature 1942..4482: note = hAce2(variant 2)(GTP14HL)-IgG1
  misc_feature 3787..4482: note = IgG1Fc
  regulatory 4549..4675: note = polyA_signal - Rabbit globin polyA; regulatory_class = polyA_signal_sequence
  repeat_region 4764..4893: note = 3' ITR
  source 1..4893: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgccaccatgtcaagctcttcctggctccttctcagccttgttgctgtaactgctgctcagtccaccattgaggaacaggccaagacatttttggacatgtttaaccacaaagccgaagacctgttctatcaaagttcacttgctgcttggaattataacaccaatattactgaagagaatgtccaaaacatgaataacgctggggacaaatggtctgcctttttaaaggaacagtccacatttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaacggccagcctgagaacaactacaagacaacccctcctgtgctggacagcgacggctcattcttcctgtacagcaagctgacagtggacaagtccaggtggcagcagggcaacgtgttcagctgcagcgtgatgcacgaggccctgcacaaccactacacccagaagtccctgagcctgtctcctggaaaatgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 86 - DNA, 4478 bp, synthetic construct
  misc_feature 1..4478: note = expression cassette encoding hAce2(variant 2)-IgG1-FC
  regulatory 1..382: note = enhancer - CMV IE promoter/enhancer; regulatory_class = enhancer
  misc_feature 1..1731: note = CB7 hybrid promoter
  regulatory 385..666: note = promoter - CB promoter; regulatory_class = promoter
  misc_feature 759..1731: note = Intron - chicken beta-actin intron (chimeric intron)
  misc_feature 1745..4285: note = hAce2(variant 2)(GTP14HL)-IgG1
  misc_feature 1745..3589: note = hAce2HL
  misc_feature 3590..4285: note = IgG1Fc
  regulatory 4352..4478: note = polyA_signal - Rabbit globin polyA; regulatory_class = polyA_signal_sequence
  source 1..4478: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgccaccatgtcaagctcttcctggctccttctcagccttgttgctgtaactgctgctcagtccaccattgaggaacaggccaagacatttttggacatgtttaaccacaaagccgaagacctgttctatcaaagttcacttgctgcttggaattataacaccaatattactgaagagaatgtccaaaacatgaataacgctggggacaaatggtctgcctttttaaaggaacagtccacatttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaacggccagcctgagaacaactacaagacaacccctcctgtgctggacagcgacggctcattcttcctgtacagcaagctgacagtggacaagtccaggtggcagcagggcaacgtgttcagctgcagcgtgatgcacgaggccctgcacaaccactacacccagaagtccctgagcctgtctcctggaaaatgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 87 - DNA, 4893 bp, synthetic construct
  misc_feature 1..4893: note = vector genome comprising hAce2MR27HL-IgG1Fc decoy
  repeat_region 1..130: note = 5"ITR
  regulatory 198..579: note = enhancer - CMV IE promoter/enhancer; regulatory_class = enhancer
  misc_feature 198..1928: note = CB7 hybrid promoter
  regulatory 582..863: note = promoter - CB promoter; regulatory_class = promoter
  misc_feature 956..1928: note = Intron - chicken beta actin intron (chimeric intron)
  misc_feature 1942..4488: note = MR27HLFc1 decoy
  misc_feature 1942..3786: note = Ace2
  misc_feature 3787..4482: note = IgGFc1
  regulatory 4549..4675: note = polyA_signal - Rabbit globin polyA; regulatory_class = polyA_signal_sequence
  repeat_region 4764..4893: note = 3'ITR
  source 1..4893: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgccaccatgtcaagctcttcctggctccttctcagccttgttgctgtaactgctgctcagtccaccattgaggaacaggccaagacatttttggacatgtttaaccacaaagccgaagacctgttctatcaaagttcacttgcttcttggaattacaacaccaatattactgaagagaatgtccaaaacatgaataatgctggggacaaatggtctgcctttttaaaggaacagtccacacttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaacggccagcctgagaacaactacaagacaacccctcctgtgctggacagcgacggctcattcttcctgtacagcaagctgacagtggacaagtccaggtggcagcagggcaacgtgttcagctgcagcgtgatgcacgaggccctgcacaaccactacacccagaagtccctgagcctgtctcctggaaaatgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 88 - DNA, 4478 bp, synthetic construct
  misc_feature 1..4478: note = expression cassette comprising hAce2MR27HL-IgG1Fc decoy
  regulatory 1..382: note = enhancer - CMV IE promoter/enhancer; regulatory_class = enhancer
  misc_feature 1..1731: note = CB7 hybrid promoter
  regulatory 385..666: note = promoter - CB promoter; regulatory_class = promoter
  misc_feature 759..1731: note = Intron - chicken beta actin intron (chimeric intron)
  misc_feature 1745..4285: note = ME27HLFc1 decoy
  misc_feature 1745..3589: note = Ace2
  misc_feature 3590..4285: note = IgG1Fc
  regulatory 4352..4478: note = polyA_signal - rabbit globin polyA; regulatory_class = polyA_signal_sequence
  source 1..4478: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgccaccatgtcaagctcttcctggctccttctcagccttgttgctgtaactgctgctcagtccaccattgaggaacaggccaagacatttttggacatgtttaaccacaaagccgaagacctgttctatcaaagttcacttgcttcttggaattacaacaccaatattactgaagagaatgtccaaaacatgaataatgctggggacaaatggtctgcctttttaaaggaacagtccacacttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaacggccagcctgagaacaactacaagacaacccctcctgtgctggacagcgacggctcattcttcctgtacagcaagctgacagtggacaagtccaggtggcagcagggcaacgtgttcagctgcagcgtgatgcacgaggccctgcacaaccactacacccagaagtccctgagcctgtctcctggaaaatgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 89 - DNA, 4884 bp, synthetic construct
  misc_feature 1..4884: note = vector genome comprising hAce2(variant 2)(GTP14HL)-IgG1Fc decoy revised linker
  repeat_region 1..130: note = 5'ITR
  regulatory 198..579: note = enhancer - CMV IE promoter/enhancer; regulatory_class = enhancer
  misc_feature 198..1928: note = CB7 hybrid promoter
  regulatory 582..863: note = promoter - CB promoter; regulatory_class = promoter
  misc_feature 956..1928: note = Intron - chicken beta actin intron (chimeric intron)
  misc_feature 1942..4473: note = hAce2(variant 2)(GTP14HL)-IgG1-revised linker
  misc_feature 1942..3786: note = Ace2
  misc_feature 3793..4473: note = IgG1Fc
  regulatory 4540..4666: note = polyA_signal - Rabbit globin polyA; regulatory_class = polyA_signal_sequence
  repeat_region 4755..4884: note = 3'ITR
  source 1..4884: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgccaccatgtcaagctcttcctggctccttctcagccttgttgctgtaactgctgctcagtccaccattgaggaacaggccaagacatttttggacatgtttaaccacaaagccgaagacctgttctatcaaagttcacttgctgcttggaattataacaccaatattactgaagagaatgtccaaaacatgaataacgctggggacaaatggtctgcctttttaaaggaacagtccacatttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggaagtgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaacggccagcctgagaacaactacaagacaacccctcctgtgctggacagcgacggctcattcttcctgtacagcaagctgacagtggacaagtccaggtggcagcagggcaacgtgttcagctgcagcgtgatgcacgaggccctgcacaaccactacacccagaagtccctgagcctgtctcctggaaaatgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 90 - DNA, 4469 bp, synthetic construct
  misc_feature 1..4469: note = expression cassette comprising hAce2(variant 2)(GTP14HL)-IgG1Fc decoy revised linker
  regulatory 1..382: note = enhancer - CMV IE promoter/enhancer; regulatory_class = enhancer
  misc_feature 1..1731: note = CB7 hybrid promoter
  regulatory 385..666: note = promoter - CB promoter; regulatory_class = promoter
  misc_feature 759..1731: note = Intron - chicken beta-actin promoter (chimeric intron)
  misc_feature 1745..4276: note = hAce2(variant 2)(GTP14HL)-IgG1 revised linker
  misc_feature 1745..3589: note = Ace2
  misc_feature 3596..4276: note = IgG1Fc
  regulatory 4343..4469: note = polyA_signal - Rabbit Globin polyA; regulatory_class = polyA_signal_sequence
  source 1..4469: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgccaccatgtcaagctcttcctggctccttctcagccttgttgctgtaactgctgctcagtccaccattgaggaacaggccaagacatttttggacatgtttaaccacaaagccgaagacctgttctatcaaagttcacttgctgcttggaattataacaccaatattactgaagagaatgtccaaaacatgaataacgctggggacaaatggtctgcctttttaaaggaacagtccacatttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggaagtgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaacggccagcctgagaacaactacaagacaacccctcctgtgctggacagcgacggctcattcttcctgtacagcaagctgacagtggacaagtccaggtggcagcagggcaacgtgttcagctgcagcgtgatgcacgaggccctgcacaaccactacacccagaagtccctgagcctgtctcctggaaaatgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 91 - DNA, 4884 bp, synthetic construct
  misc_feature 1..4884: note = vector genome comprising hAce2MR27HL-IgG1Fc decoy revised linker
  repeat_region 1..130: note = 5'ITR
  regulatory 198..579: note = enhancer - CMV IE promoter/enhancer; regulatory_class = enhancer
  misc_feature 198..1928: note = CB7 hybrid promoter
  regulatory 582..863: note = promoter - CB promoter; regulatory_class = promoter
  misc_feature 956..1928: note = Intron - chicken beta-actin intron (chimeric intron)
  misc_feature 1942..4479: note = MR27HL-Fc1 decoy revised linker
  misc_feature 1942..3786: note = Ace2
  misc_feature 3793..4473: note = IgG1Fc
  regulatory 4540..4666: note = polyA_signal - Rabbit globbin polyA; regulatory_class = polyA_signal_sequence
  repeat_region 4755..4884: note = 3'ITR
  source 1..4884: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgccaccatgtcaagctcttcctggctccttctcagccttgttgctgtaactgctgctcagtccaccattgaggaacaggccaagacatttttggacatgtttaaccacaaagccgaagacctgttctatcaaagttcacttgcttcttggaattacaacaccaatattactgaagagaatgtccaaaacatgaataatgctggggacaaatggtctgcctttttaaaggaacagtccacacttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggaagtgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaacggccagcctgagaacaactacaagacaacccctcctgtgctggacagcgacggctcattcttcctgtacagcaagctgacagtggacaagtccaggtggcagcagggcaacgtgttcagctgcagcgtgatgcacgaggccctgcacaaccactacacccagaagtccctgagcctgtctcctggaaaatgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 92 - DNA, 4469 bp, synthetic construct
  misc_feature 1..4469: note = expression cassette comprising hAce2MR27HL-IgG1Fc decoy revised linker
  regulatory 1..382: note = enhancer - CMV IE promoter/enhancer; regulatory_class = enhancer
  misc_feature 1..1731: note = CB7 hybrid promoter
  regulatory 385..666: note = promoter - CB promoter; regulatory_class = promoter
  misc_feature 759..1731: note = Intron - chicken beta actin intron (chimeric intron)
  misc_feature 1745..4282: note = MR27HL-IgG1Fc decoy revised linker
  misc_feature 1745..3589: note = Ace2
  misc_feature 3596..4276: note = IgG1Fc
  regulatory 4343..4469: note = polyA_signal - Rabbit globin polyA; regulatory_class = polyA_signal_sequence
  source 1..4469: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgccaccatgtcaagctcttcctggctccttctcagccttgttgctgtaactgctgctcagtccaccattgaggaacaggccaagacatttttggacatgtttaaccacaaagccgaagacctgttctatcaaagttcacttgcttcttggaattacaacaccaatattactgaagagaatgtccaaaacatgaataatgctggggacaaatggtctgcctttttaaaggaacagtccacacttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggaagtgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaacggccagcctgagaacaactacaagacaacccctcctgtgctggacagcgacggctcattcttcctgtacagcaagctgacagtggacaagtccaggtggcagcagggcaacgtgttcagctgcagcgtgatgcacgaggccctgcacaaccactacacccagaagtccctgagcctgtctcctggaaaatgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 93 - DNA, 2541 bp, synthetic construct
  misc_feature 1..2541: note = hAce2(variant 2)(GTP14HL)-IgG1Fc decoy
  misc_feature 1..1845: note = Ace2
  misc_feature 1..51: note = Ace2 signal peptide
  misc_feature 1846..2541: note = IgG1Fc
  source 1..2541: mol_type = other DNA
  CDS 1..2541: note = hAce2(variant 2)(GTP14HL)-IgG1Fc; protein_id = 94; translation = MSSSSWLLLSLVAVTAAQSTIEEQAKTFLDMFNHKAEDLFYQSS LAAWNYNTNITEENVQNMNNAGDKWSAFLKEQSTFAQMYPLQEIQNPTVKLQLQALQQ NGSSVLSEDKSKRLNTILNTMSTIYSTGKVCNPDNPQECLLLEPGLNEIMANSLDYNE RLWAWESWRSEVGKQLRPLYEEYVVLKNEMARANHYEDYGDYWRGDYEVNGVDGYDYS RGQLIEDVEHTFEEIKPLYEHLHAYVRAKLMNAYPSYISPIGCLPAHLLGDMWGRFWT NLYSLTVPFGQKPNIDVTDAMVDQAWDAQRIFKEAEKFFVSVGLPNMTQGFWEYSMLT DPGNVQKAVCLPTAWDLGKGDFRILMCTKVTMDDFLTAHHEMGHIQYDMAYAAQPFLL RNGANEGFHEAVGEIMSLSAATPKHLKSIGLLSPDFQEDNETEINFLLKQALTIVGTL PFTYMLEKWRWMVFKGEIPKDQWMKKWWEMKREIVGVVEPVPHDETYCDPASLFHVSN DYSFIRYYTRTLYQFQFQEALCQAAKHEGPLHKCDISNSTEAGQKLFNMLRLGKSEPW TLALENVVGAKNMNVRPLLNYFEPLFTWLKDQNKNSFVGWSTDWSPYADEPKSCDKTH TCPPCPAPELLGGPSVFLFPPKPKDTLMISRTPEVTCVVVDVSHEDPEVKFNWYVDGV EVHNAKTKPREEQYNSTYRVVSVLTVLHQDWLNGKEYKCKVSNKALPAPIEKTISKAK GQPREPQVYTLPPSRDELTKNQVSLTCLVKGFYPSDIAVEWESNGQPENNYKTTPPVL DSDGSFFLYSKLTVDKSRWQQGNVFSCSVMHEALHNHYTQKSLSLSPGK
atgtcaagctcttcctggctccttctcagccttgttgctgtaactgctgctcagtccaccattgaggaacaggccaagacatttttggacatgtttaaccacaaagccgaagacctgttctatcaaagttcacttgctgcttggaattataacaccaatattactgaagagaatgtccaaaacatgaataacgctggggacaaatggtctgcctttttaaaggaacagtccacatttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaacggccagcctgagaacaactacaagacaacccctcctgtgctggacagcgacggctcattcttcctgtacagcaagctgacagtggacaagtccaggtggcagcagggcaacgtgttcagctgcagcgtgatgcacgaggccctgcacaaccactacacccagaagtccctgagcctgtctcctggaaaa

SEQ ID NO: 94 - protein, 847 aa, synthetic construct
  REGION 1..847: note = Synthetic Construct
MSSSSWLLLSLVAVTAAQSTIEEQAKTFLDMFNHKAEDLFYQSSLAAWNYNTNITEENVQNMNNAGDKWSAFLKEQSTFAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCLPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - DNA, 2547 bp, synthetic construct
  misc_feature 1..2547: note = hAce2MR27HL-IgG1Fc decoy
  misc_feature 1..51: note = Ace2 signal peptide
  misc_feature 1..1845: note = Ace2
  misc_feature 1846..2541: note = IgG1Fc
  source 1..2547: mol_type = other DNA
  misc_feature 1..2547: note = MR27HLFc1 decoy
atgtcaagctcttcctggctccttctcagccttgttgctgtaactgctgctcagtccaccattgaggaacaggccaagacatttttggacatgtttaaccacaaagccgaagacctgttctatcaaagttcacttgcttcttggaattacaacaccaatattactgaagagaatgtccaaaacatgaataatgctggggacaaatggtctgcctttttaaaggaacagtccacacttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaacggccagcctgagaacaactacaagacaacccctcctgtgctggacagcgacggctcattcttcctgtacagcaagctgacagtggacaagtccaggtggcagcagggcaacgtgttcagctgcagcgtgatgcacgaggccctgcacaaccactacacccagaagtccctgagcctgtctcctggaaaatgatga

SEQ ID NO: 96 - protein, 847 aa, synthetic construct
  REGION 1..847: note = Synthetic Construct
MSSSSWLLLSLVAVTAAQSTIEEQAKTFLDMFNHKAEDLFYQSSLASWNYNTNITEENVQNMNNAGDKWSAFLKEQSTLAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCLPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - DNA, 2538 bp, synthetic construct
  misc_feature 1..2538: note = hAce2(variant 2)(GTP14HL)-IgG1Fc decoy revised linker
  misc_feature 1..1845: note = Ace2
  misc_feature 1852..2532: note = IgGFc1
  source 1..2538: mol_type = other DNA
  misc_feature 1..2538: note = hAce2(variant 2)(GTP14HL)-IgG1 decoy revised linker
atgtcaagctcttcctggctccttctcagccttgttgctgtaactgctgctcagtccaccattgaggaacaggccaagacatttttggacatgtttaaccacaaagccgaagacctgttctatcaaagttcacttgctgcttggaattataacaccaatattactgaagagaatgtccaaaacatgaataacgctggggacaaatggtctgcctttttaaaggaacagtccacatttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggaagtgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaacggccagcctgagaacaactacaagacaacccctcctgtgctggacagcgacggctcattcttcctgtacagcaagctgacagtggacaagtccaggtggcagcagggcaacgtgttcagctgcagcgtgatgcacgaggccctgcacaaccactacacccagaagtccctgagcctgtctcctggaaaatgatga

SEQ ID NO: 98 - protein, 844 aa, synthetic construct
  REGION 1..844: note = Synthetic Construct
MSSSSWLLLSLVAVTAAQSTIEEQAKTFLDMFNHKAEDLFYQSSLAAWNYNTNITEENVQNMNNAGDKWSAFLKEQSTFAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCLPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - DNA, 2538 bp, synthetic construct
  misc_feature 1..2538: note = hAce2MR27HL-IgG1Fc decoy revised linker
  misc_feature 1..51: note = Ace2 signal peptide
  misc_feature 1..1845: note = Ace2
  misc_feature 1852..2532: note = IgG1Fc
  source 1..2538: mol_type = other DNA
  misc_feature 1..2538: note = MR27HL-IgG1Fc decoy revised linker
atgtcaagctcttcctggctccttctcagccttgttgctgtaactgctgctcagtccaccattgaggaacaggccaagacatttttggacatgtttaaccacaaagccgaagacctgttctatcaaagttcacttgcttcttggaattacaacaccaatattactgaagagaatgtccaaaacatgaataatgctggggacaaatggtctgcctttttaaaggaacagtccacacttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggaagtgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaacggccagcctgagaacaactacaagacaacccctcctgtgctggacagcgacggctcattcttcctgtacagcaagctgacagtggacaagtccaggtggcagcagggcaacgtgttcagctgcagcgtgatgcacgaggccctgcacaaccactacacccagaagtccctgagcctgtctcctggaaaatgatga

SEQ ID NO: 100 - protein, 844 aa, synthetic construct
  REGION 1..844: note = Synthetic Construct
MSSSSWLLLSLVAVTAAQSTIEEQAKTFLDMFNHKAEDLFYQSSLASWNYNTNITEENVQNMNNAGDKWSAFLKEQSTLAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCLPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 5 aa, synthetic construct
  REGION 1..5: note = Linker
EPKSC

SEQ ID NO: 102 - DNA, 696 bp, synthetic construct
  misc_feature 1..696: note = nucleic acid sequence encoding IgG1 Fc
  source 1..696: mol_type = other DNA
  misc_feature 1..696: note = IgG1 Fc
gaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaacggccagcctgagaacaactacaagacaacccctcctgtgctggacagcgacggctcattcttcctgtacagcaagctgacagtggacaagtccaggtggcagcagggcaacgtgttcagctgcagcgtgatgcacgaggccctgcacaaccactacacccagaagtccctgagcctgtctcctggaaaa

SEQ ID NO: 103 - protein, 232 aa, synthetic construct
  REGION 1..232: note = Synthetic Construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - DNA, 1845 bp, synthetic construct
  misc_feature 1..1845: note = nucliec acid sequence encoding hAce2-MR27Variant
  source 1..1845: mol_type = other DNA
  CDS 1..1845: note = hAce2-MR27Variant; protein_id = 105; translation = MSSSSWLLLSLVAVTAAQSTIEEQAKTFLDMFNHKAEDLFYQSS LASWNYNTNITEENVQNMNNAGDKWSAFLKEQSTLAQMYPLQEIQNPTVKLQLQALQQ NGSSVLSEDKSKRLNTILNTMSTIYSTGKVCNPDNPQECLLLEPGLNEIMANSLDYNE RLWAWESWRSEVGKQLRPLYEEYVVLKNEMARANHYEDYGDYWRGDYEVNGVDGYDYS RGQLIEDVEHTFEEIKPLYEHLHAYVRAKLMNAYPSYISPIGCLPAHLLGDMWGRFWT NLYSLTVPFGQKPNIDVTDAMVDQAWDAQRIFKEAEKFFVSVGLPNMTQGFWEYSMLT DPGNVQKAVCLPTAWDLGKGDFRILMCTKVTMDDFLTAHHEMGHIQYDMAYAAQPFLL RNGANEGFHEAVGEIMSLSAATPKHLKSIGLLSPDFQEDNETEINFLLKQALTIVGTL PFTYMLEKWRWMVFKGEIPKDQWMKKWWEMKREIVGVVEPVPHDETYCDPASLFHVSN DYSFIRYYTRTLYQFQFQEALCQAAKHEGPLHKCDISNSTEAGQKLFNMLRLGKSEPW TLALENVVGAKNMNVRPLLNYFEPLFTWLKDQNKNSFVGWSTDWSPYAD
atgtcaagctcttcctggctccttctcagccttgttgctgtaactgctgctcagtccaccattgaggaacaggccaagacatttttggacatgtttaaccacaaagccgaagacctgttctatcaaagttcacttgcttcttggaattacaacaccaatattactgaagagaatgtccaaaacatgaataatgctggggacaaatggtctgcctttttaaaggaacagtccacacttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagac

SEQ ID NO: 105 - protein, 615 aa, synthetic construct
  REGION 1..615: note = Synthetic Construct
MSSSSWLLLSLVAVTAAQSTIEEQAKTFLDMFNHKAEDLFYQSSLASWNYNTNITEENVQNMNNAGDKWSAFLKEQSTLAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCLPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 106 - DNA, 1794 bp, synthetic construct
  misc_feature 1..1794: note = nucliec acid sequence encoding hAce2-MR27Variant (without leader)
  source 1..1794: mol_type = other DNA
  CDS 1..1794: note = hAce2-MR27Variant (without leader); protein_id = 107; translation = QSTIEEQAKTFLDMFNHKAEDLFYQSSLASWNYNTNITEENVQN MNNAGDKWSAFLKEQSTLAQMYPLQEIQNPTVKLQLQALQQNGSSVLSEDKSKRLNTI LNTMSTIYSTGKVCNPDNPQECLLLEPGLNEIMANSLDYNERLWAWESWRSEVGKQLR PLYEEYVVLKNEMARANHYEDYGDYWRGDYEVNGVDGYDYSRGQLIEDVEHTFEEIKP LYEHLHAYVRAKLMNAYPSYISPIGCLPAHLLGDMWGRFWTNLYSLTVPFGQKPNIDV TDAMVDQAWDAQRIFKEAEKFFVSVGLPNMTQGFWEYSMLTDPGNVQKAVCLPTAWDL GKGDFRILMCTKVTMDDFLTAHHEMGHIQYDMAYAAQPFLLRNGANEGFHEAVGEIMS LSAATPKHLKSIGLLSPDFQEDNETEINFLLKQALTIVGTLPFTYMLEKWRWMVFKGE IPKDQWMKKWWEMKREIVGVVEPVPHDETYCDPASLFHVSNDYSFIRYYTRTLYQFQF QEALCQAAKHEGPLHKCDISNSTEAGQKLFNMLRLGKSEPWTLALENVVGAKNMNVRP LLNYFEPLFTWLKDQNKNSFVGWSTDWSPYAD
cagtccaccattgaggaacaggccaagacatttttggacatgtttaaccacaaagccgaagacctgttctatcaaagttcacttgcttcttggaattacaacaccaatattactgaagagaatgtccaaaacatgaataatgctggggacaaatggtctgcctttttaaaggaacagtccacacttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagac

SEQ ID NO: 107 - protein, 598 aa, synthetic construct
  REGION 1..598: note = Synthetic Construct
QSTIEEQAKTFLDMFNHKAEDLFYQSSLASWNYNTNITEENVQNMNNAGDKWSAFLKEQSTLAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCLPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 108 - DNA, 2496 bp, synthetic construct
  misc_feature 1..2496: note = hAce2-Variant2-IgG1Fc decoy (without signal/leader peptide)
  misc_feature 1..1794: note = hAce2 Variant 2 (without signal/leader peptide)
  misc_feature 1795..1809: note = Linker
  misc_feature 1810..2490: note = IgG1 Fc
  source 1..2496: mol_type = other DNA
  misc_feature 1..2496: note = hAce2-Variant 2-IgG1-Fc (without signal/leader peptide)
cagtccaccattgaggaacaggccaagacatttttggacatgtttaaccacaaagccgaagacctgttctatcaaagttcacttgctgcttggaattataacaccaatattactgaagagaatgtccaaaacatgaataacgctggggacaaatggtctgcctttttaaaggaacagtccacatttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaacggccagcctgagaacaactacaagacaacccctcctgtgctggacagcgacggctcattcttcctgtacagcaagctgacagtggacaagtccaggtggcagcagggcaacgtgttcagctgcagcgtgatgcacgaggccctgcacaaccactacacccagaagtccctgagcctgtctcctggaaaatgatga

SEQ ID NO: 109 - protein, 830 aa, synthetic construct
  REGION 1..830: note = Synthetic Construct
QSTIEEQAKTFLDMFNHKAEDLFYQSSLAAWNYNTNITEENVQNMNNAGDKWSAFLKEQSTFAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCLPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - DNA, 2496 bp, synthetic construct
  misc_feature 1..2496: note = hAce2-MR27HL-Variant-IgG1Fc decoy (without signal/leader peptide)
  misc_feature 1..1794: note = hAce2-MR27HL-Variant
  misc_feature 1795..1809: note = linker
  misc_feature 1810..2490: note = IgG1 Fc
  source 1..2496: mol_type = other DNA
  misc_feature 1..2496: note = hAce2-MR27HL-Variant-IgG1-Fc
cagtccaccattgaggaacaggccaagacatttttggacatgtttaaccacaaagccgaagacctgttctatcaaagttcacttgcttcttggaattacaacaccaatattactgaagagaatgtccaaaacatgaataatgctggggacaaatggtctgcctttttaaaggaacagtccacacttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaacggccagcctgagaacaactacaagacaacccctcctgtgctggacagcgacggctcattcttcctgtacagcaagctgacagtggacaagtccaggtggcagcagggcaacgtgttcagctgcagcgtgatgcacgaggccctgcacaaccactacacccagaagtccctgagcctgtctcctggaaaatgatga

SEQ ID NO: 111 - protein, 830 aa, synthetic construct
  REGION 1..830: note = Synthetic Construct
QSTIEEQAKTFLDMFNHKAEDLFYQSSLASWNYNTNITEENVQNMNNAGDKWSAFLKEQSTLAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCLPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - DNA, 2487 bp, synthetic construct
  misc_feature 1..2487: note = hAce2-Variant2-IgG1Fc decoy (without signal/leader peptide; revised linker)
  misc_feature 1..1794: note = hAce2-Variant 2
  misc_feature 1795..1800: note = linker
  misc_feature 1801..2481: note = IgG1-Fc
  source 1..2487: mol_type = other DNA
  misc_feature 1..2487: note = hAce2-Variant2-IgG1-Fc (revised linker; without leader/signal peptide)
cagtccaccattgaggaacaggccaagacatttttggacatgtttaaccacaaagccgaagacctgttctatcaaagttcacttgctgcttggaattataacaccaatattactgaagagaatgtccaaaacatgaataacgctggggacaaatggtctgcctttttaaaggaacagtccacatttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggaagtgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaacggccagcctgagaacaactacaagacaacccctcctgtgctggacagcgacggctcattcttcctgtacagcaagctgacagtggacaagtccaggtggcagcagggcaacgtgttcagctgcagcgtgatgcacgaggccctgcacaaccactacacccagaagtccctgagcctgtctcctggaaaatgatga

SEQ ID NO: 113 - protein, 827 aa, synthetic construct
  REGION 1..827: note = Synthetic Construct
QSTIEEQAKTFLDMFNHKAEDLFYQSSLAAWNYNTNITEENVQNMNNAGDKWSAFLKEQSTFAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCLPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - DNA, 2487 bp, synthetic construct
  misc_feature 1..2487: note = hAce2-MR27HL-Variant-IgG1Fc decoy (without signal/leader peptide; revised linker)
  misc_feature 1..1794: note = hAce2-MR27 Variant
  misc_feature 1795..1800: note = linker
  misc_feature 1801..2481: note = IgG1 Fc
  source 1..2487: mol_type = other DNA
  misc_feature 1..2487: note = hAce2-MR27-Varisant-IgG1-Fc (without signal/leader peptide; revised linker)
cagtccaccattgaggaacaggccaagacatttttggacatgtttaaccacaaagccgaagacctgttctatcaaagttcacttgcttcttggaattacaacaccaatattactgaagagaatgtccaaaacatgaataatgctggggacaaatggtctgcctttttaaaggaacagtccacacttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacggaagtgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaacggccagcctgagaacaactacaagacaacccctcctgtgctggacagcgacggctcattcttcctgtacagcaagctgacagtggacaagtccaggtggcagcagggcaacgtgttcagctgcagcgtgatgcacgaggccctgcacaaccactacacccagaagtccctgagcctgtctcctggaaaatgatga

SEQ ID NO: 115 - protein, 827 aa, synthetic construct
  REGION 1..827: note = Synthetic Construct
QSTIEEQAKTFLDMFNHKAEDLFYQSSLASWNYNTNITEENVQNMNNAGDKWSAFLKEQSTLAQMYPLQEIQNPTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCLPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - DNA, 4469 bp, synthetic construct
  misc_feature 1..4469: note = expression cassette cb7-ci-hace2gtp14-fc4-rbg
  regulatory 1..382: note = enhancer - CMV IE enhancer; regulatory_class = enhancer
  misc_feature 1..1731: note = CB7 hybrid promoter
  regulatory 385..666: note = promoter - CB promoter; regulatory_class = promoter
  misc_feature 759..1731: note = Intron - chicken beta actin intron
  misc_feature 1745..1795: note = Ace2 signal peptide
  misc_feature 1745..3589: note = hAce2 (variant 1) (GTP14)
  misc_feature 1745..4276: note = hAce2(Variant 1)(GTP14)-IgG4
  misc_feature 3590..4276: note = human IgG4 Fc
  regulatory 4343..4469: note = polyA_signal - rabbit beta globin polyA; regulatory_class = polyA_signal_sequence
  source 1..4469: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgccaccatgtcaagctcttcctggctccttctcagccttgttgctgtaactgctgctcagtccaccattgaggaacaggccaagacatttttggacatgtttaaccacaaagccgaagacctgttctatcaaagttcacttgctgcttggaattataacaccaatattactgaagagaatgtccaaaacatgaataacgctggggacaaatggtctgcctttttaaaggaacagtccacatttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagtctaagtacggccctccttgtccttcctgccccgctcctgaatttctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtcgacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgccaagcagcatcgagaaaaccatcagcaaggccaagggccagcctagggaaccccaggtttacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggacagcgacggctcattcttcctgtactcccggctgaccgtggacaagtccagatggcaagagggcaacgtgttcagctgcagcgtgatgcacgaggccctgcacaatcactacacccagaagtccctgagtctgagcctgggcaagtgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 117 - DNA, 4469 bp, synthetic construct
  misc_feature 1..4469: note = expression cassette cb7-ci-hace2gtp14hl-fc4-rbg
  regulatory 1..382: note = enhancer - CMV IE Enhancer; regulatory_class = enhancer
  misc_feature 1..1731: note = CB7 hybrid promoter
  regulatory 385..666: note = promoter - CB promoter; regulatory_class = promoter
  misc_feature 1745..4276: note = hAce2(variant 2)(GTP14HL)-IgG4
  misc_feature 1745..1795: note = ACE2 signaling peptide
  misc_feature 1745..3589: note = hAce(variant 2)(GTP14HL)
  misc_feature 3590..4276: note = Human IgG4 Fc
  regulatory 4343..4469: note = polyA_signal - Rabbit beta globin polyA; regulatory_class = polyA_signal_sequence
  source 1..4469: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgccaccatgtcaagctcttcctggctccttctcagccttgttgctgtaactgctgctcagtccaccattgaggaacaggccaagacatttttggacatgtttaaccacaaagccgaagacctgttctatcaaagttcacttgctgcttggaattataacaccaatattactgaagagaatgtccaaaacatgaataacgctggggacaaatggtctgcctttttaaaggaacagtccacatttgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccttcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagtctaagtacggccctccttgtccttcctgccccgctcctgaatttctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtcgacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgccaagcagcatcgagaaaaccatcagcaaggccaagggccagcctagggaaccccaggtttacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggacagcgacggctcattcttcctgtactcccggctgaccgtggacaagtccagatggcaagagggcaacgtgttcagctgcagcgtgatgcacgaggccctgcacaatcactacacccagaagtccctgagtctgagcctgggcaagtgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 118 - DNA, 4469 bp, synthetic construct
  misc_feature 1..4469: note = expression cassette cb7-ci-hace2gen3-3-fc4-rbg
  regulatory 1..382: note = enhancer - CMV IE enhancer; regulatory_class = enhancer
  misc_feature 1..1731: note = CB7 promoter
  regulatory 385..666: note = promoter - CB promoter; regulatory_class = promoter
  misc_feature 759..1731: note = Intron - chicken beta actin intron
  misc_feature 1745..1795: note = ACE2 signal peptide
  misc_feature 1745..3589: note = hAce2 (Varinat 3) (Gen3-3)
  misc_feature 1745..4276: note = hAce2 (variant 3) (Gen3-3) IgG4
  misc_feature 3590..4276: note = Human IgG4 Fc
  regulatory 4343..4469: note = polyA_signal - rabit beta globin polyA; regulatory_class = polyA_signal_sequence
  source 1..4469: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgccaccatgtcaagctcttcctggctccttctcagccttgttgctgtaactgctgctcagtccaccattgaggaacaggccaagacatttttggacaggtttaaccacgtagccgaagacccgttctatcaaagttcacttgcttcttggaattataacaccaatatcactgaagagaatgcccaaaacatgaataatgctggggacaaatggtctgcctttttaaaggaacagtccacatttgcccaaatgtatccactacaagaaattcaggacctcacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagtctaagtacggccctccttgtccttcctgccccgctcctgaatttctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtcgacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgccaagcagcatcgagaaaaccatcagcaaggccaagggccagcctagggaaccccaggtttacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggacagcgacggctcattcttcctgtactcccggctgaccgtggacaagtccagatggcaagagggcaacgtgttcagctgcagcgtgatgcacgaggccctgcacaatcactacacccagaagtccctgagtctgagcctgggcaagtgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 119 - DNA, 4469 bp, synthetic construct
  misc_feature 1..4469: note = expression cassette cb7-ci-hace2gen3-5-fc4-rbg
  regulatory 1..382: note = enhancer - CMV IE enhancer; regulatory_class = enhancer
  misc_feature 1..1731: note = CB7 hybrid promoter
  regulatory 385..666: note = promoter - CB promoter; regulatory_class = promoter
  misc_feature 759..1731: note = Intron - chicken beta actin intron
  misc_feature 1745..1795: note = ACE2 signal peptide
  misc_feature 1745..3589: note = hAce2 (variant 4) (GTP14-Gen3-5)
  misc_feature 1745..4276: note = hAce2(varinat 4) (GTP14 Gen3-5) IgG4
  misc_feature 3590..4276: note = Human IgG4Fc
  regulatory 4343..4469: note = polyA_signal - Rabbit beta globin polyA; regulatory_class = polyA_signal_sequence
  source 1..4469: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcccgccaccatgtcaagctcttcctggctccttctcagccttgttgctgtaactgctgctcagtccaccattgaggaacaggccaagacatttttggacatgtttaaccacaaagccgaagacctgttctatcaaagttcacttgcttcttggaattataacaccaatattactgaagagaatgtccaaaacatgaataatgctggggacaaatggtctgcctttttaaaggaacagtccacaattgcccaaatgtatccactacaagaaattcagaatcccacagtcaagcttcagctgcaggctcttcagcaaaatgggtcttcagtgctctcagaagacaagagcaaacggttgaacacaattctaaatacaatgagcaccatctacagtactggaaaagtttgtaacccggataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcgagagcaaaccattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttcccttcggacagaaaccaaacatagatgttactgatgcaatggtggaccaggcctgggatgcacagagaatattcaaggaggccgagaagttctttgtatctgttggtcttcctaatatgactcaaggattctgggaatattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagtctaagtacggccctccttgtccttcctgccccgctcctgaatttctcggcggaccttccgtgttcctgtttcctccaaagcctaaggacaccctgatgatcagcagaacccctgaagtgacctgcgtggtggtggacgtgtcccaagaggatcctgaggtgcagttcaattggtacgtcgacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgccaagcagcatcgagaaaaccatcagcaaggccaagggccagcctagggaaccccaggtttacacactgcctccaagccaagaggaaatgaccaagaaccaggtgtccctgacctgcctggtcaagggcttctacccttccgatatcgccgtggaatgggagagcaacggccagcctgagaacaactacaagaccacacctcctgtgctggacagcgacggctcattcttcctgtactcccggctgaccgtggacaagtccagatggcaagagggcaacgtgttcagctgcagcgtgatgcacgaggccctgcacaatcactacacccagaagtccctgagtctgagcctgggcaagtgatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 120 - DNA, 382 bp, synthetic construct
  misc_feature 1..382: note = CMV IE Enhancer
  source 1..382: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 121 - DNA, 282 bp, synthetic construct
  misc_feature 1..282: note = CB promoter
  source 1..282: mol_type = other DNA
tggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 122 - DNA, 1735 bp, synthetic construct
  misc_feature 7..386: note = CMV IE enhancer
  source 1..1735: mol_type = other DNA
  misc_feature 388..665: note = CB promoter
  misc_feature 666..1735: note = chimeric intron comprising chicken beta actin intron
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 123 - DNA, 973 bp, synthetic construct
  misc_feature 1..973: note = chicken beta actin intron (chimeric intron)
  source 1..973: mol_type = other DNA
gtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 124 - DNA, 127 bp, synthetic construct
  misc_feature 1..127: note = rabbit beta globin polyA
  source 1..127: mol_type = other DNA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 125 - protein, 353 aa, synthetic construct
  REGION 1..353: note = IgA1 Fc region
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 126 - protein, 340 aa, synthetic construct
  REGION 1..340: note = IgA2 Fc region
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 127 - protein, 159 aa, synthetic construct
  REGION 1..159: note = J chain
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

(19) United States
(12) Patent Application Publication
(10) Pub. No.: US 2023/0338478 A1
(43) Pub. Date: Oct. 26, 2023

(54) COMPOSITIONS AND METHOD OF USE OF MUTANT ACE2 DECOY VARIANTS

(71) Applicant: The Trustees of the University of Pennsylvania, Blue Bell, PA (US)

(72) Inventors: Joshua Joyner Sims, Villanova, PA (US); James M. Wilson, Philadelphia, PA (US); Christian Hinderer, Baltimore, MD (US)

(21) Appl. No.: 18/304,143

(22) Filed: Apr. 20, 2023

Related U.S. Application Data
(63) Continuation-in-part of application No. PCT/US2022/014406, filed on Jan. 28, 2022.
(60) Provisional application No. 63/332,944, filed on Apr. 20, 2022.
(60) Provisional application No. 63/300,154, filed on Jan. 17, 2022.
(60) Provisional application No. 63/253,654, filed on Oct. 8, 2021.
(60) Provisional application No. 63/215,159, filed on Jun. 25, 2021.
(60) Provisional application No. 63/166,686, filed on Mar. 26, 2021.
(60) Provisional application No. 63/160,511, filed on Mar. 12, 2021.
(60) Provisional application No. 63/143,614, filed on Jan. 29, 2021.

(51) Int. Cl. A61K 48/00; C12N 15/86; A61P 31/14; A61K 38/48

(52) U.S. Cl. CPC A61K 38/4813; A61K 48/0041; A61P 31/14; C12N 15/86; C12Y 304/17023; C12N 2750/14143; C12N 2750/14171

(57) ABSTRACT

A mutant soluble human Ace2 (hAce2) protein useful in preventing infection with betacoronaviruses, including SARS-CoV2 is provided, as are compositions useful in treating disease associated with betacoronavirus, including, e.g, COVID-19. Also provided are compositions containing same formulated for intranasal and/or intrapulmonary delivery, methods of making same and assays. The use of the rAAV compositions for preventing symptoms of COVID-19 infection in humans is provided.

### CROSS-REFERENCE TO RELATED APPLICATIONS

This application claims the benefit under 35 USC 119(e) of U.S. Provisional Pat. Application No. 63/332,944, filed Apr. 20, 2022 and is a continuation-in-part of International Patent Application No. PCT/US2022/014406, filed Jan. 28, 2022, which claims the benefit of U.S. Provisional Pat. Application No. 63/300,154, filed Jan. 17, 2022, U.S. Provisional Pat. Application No. 63/253,654, filed Oct. 8, 2021, U.S. Provisional Pat. Application No. 63/215,159, filed Jun. 25, 2021, U.S. Provisional Pat. Application No. 63/166,686, filed Mar. 26, 2021, U.S. Provisional Pat. Application No. 63/160,511, filed Mar. 12, 2021, and U.S. Provisional Pat. Application No. 63/143,614, filed Jan. 29, 2021. These applications are incorporated by reference herein in their entireties.

### REFERENCE TO AN ELECTRONIC SEQUENCE LISTING

The contents of the electronic sequence listing (“UPN-22-10050USCIP.xml”; Size: 436,169 bytes; and Date of Creation: Apr. 19, 2023) is herein incorporated by reference in its entirety.

### BACKGROUND OF THE INVENTION

The coronavirus “SARS-CoV-2” outbreak that emerged in China and has rapidly spread across the world has been declared a public health emergency of international concern by the World Health Organization (WHO). Efforts are ongoing to discover effective therapeutic and prophylactic agents that will help to mitigate the spread of the disease.

AAVs are nonpathogenic parvoviruses that circulate broadly in humans and other species. Replacing all viral coding sequences with a gene of interest yields an AAV vector capable of efficient in vivo gene delivery without the insertional mutagenesis risks or robust inflammatory responses observed with lentiviral or adenoviral vectors. AAV vectors have demonstrated an acceptable safety profile in thousands of human subjects, and two products are now approved in the US with dozens more in late-stage clinical development. The stability of AAV vectors make them practical for widespread distribution as prophylactic vaccines.

During the 2003 SARS epidemic it was discovered that the receptor critical for SARS-CoV-1 entry into host cells is angiotensin-converting enzyme 2 (ACE2), which was subsequently shown to be shared with SARS CoV-2. The S1 domain of the viral spike protein of SARS CoV-2 binds to host cells via the ACE2 receptor with low nanomolar affinity. Soluble ACE2 has been administered intravenously to healthy volunteers and demonstrated an acceptable safety and immunogenicity profile. A pilot efficacy study with soluble human recombinant ACE2 is ongoing in patients with COVID-19 (Clinicaltrials.gov #NCT04287686).

Angiotensin-converting enzyme 2 (ACE2) is transmembrane glycoprotein with present increased expression in tissues of heart, kidneys, and testes (Kuba, K., et al., Pharmacol Ther, 2010, 128: 119-128). Targeting ACE2 has been used as therapeutic approach for hypertension and heart failure, and described to provide a protective effect in cardiovascular systems. Recent sequencing analysis studies have identified a large number of ACE2-expressing cells in the lung tissue, specifically the type I and II alveolar epithelial cells (Zhao,Y., et al., 2020, bioRxiv). This finding was consistent with the correlation of SARS-coronavirus (SARS-CoV) infection causing severe acute lung failure. The SARS-CoV protein spikes (containing “S” protein) contact ACE2 and uilize it to facilitate internalization, which thereby aids in infection (Kuba, K., et al., 2010, Pharmacol Ther, 128: 119-128). In ACE2 there is an HEXXH motif, containing two histidine in the active catalytic site, which is responsible for coordinating zinc ion, thereby facilitating the metabolism of circulating peptides.

There are a variety of needs in this area, including compositions and methods for preventing one or more COVID-19 symptoms and/or infection, and/or methods for reducing viral replication following infection.

### SUMMARY OF THE INVENTION

In one aspect provided herein is a recombinant AAV (rAAV) comprising an AAV capsid and a vector genome packaged therein, wherein the vector genome comprises a 5′ inverted terminal repeat (ITR), a nucleic acid sequence encoding at least one mutant hAce2 soluble decoy protein under the control of regulatory control sequences which direct expression of the hAce2 soluble decoy protein, and a 3′ ITR, wherein the mutant hAce2 soluble decoy protein comprises an amino acid sequence of: (a) SEQ ID NO: 12 (hAce2-Variant2) or an amino acid sequence at least 95% identical thereto, optionally fused to an immunoglobulin Fc region; (b) SEQ ID NO: 10 (hAce2-Variant1) or an amino acid sequence at least 95% identical thereto, optionally fused to an immunoglobulin Fc region; (c) SEQ ID NO: 14 (hAce2-Variant3) or an amino acid sequence at least 95% identical thereto, optionally fused to an immunoglobulin Fc region; (d) SEQ ID NO: 16 (hAce2-Variant4) or an amino acid sequence at least 95% identical thereto, optionally fused to an immunoglobulin Fc region; (e) SEQ ID NO: 72 (hAce2-Variant5) or an amino acid sequence at least 95% identical thereto, optionally fused to an immunoglobulin Fc region; or (f) SEQ ID NO: 73 (hAce2-Variant6) or an amino acid sequence at least 95% identical thereto, optionally fused to an immunoglobulin Fc region. In certain embodiments, the rAAV comprises nucleic acid sequence encoding the mutant hAce2 soluble decoy protein selected from: (a) SEQ ID NO: 11 or a sequence at least about 90% identical thereto encoding SEQ ID NO: 12 (hAce2-Variant2); (b) SEQ ID NO: 9 or a sequence at least about 90% identical thereto encoding SEQ ID NO: 10 (hAce2-Variant1); (c) SEQ ID NO: 13 or a sequence at least about 90% identical thereto encoding SEQ ID NO: 14 (hAce2-Variant3); (d) SEQ ID NO: 15 or a sequence at least about 90% identical thereto encoding SEQ ID NO: 16 (hAce2-Variant4); (e) SEQ ID NO: 1 or a sequence at least about 90% identical thereto encoding SEQ ID NO: 2 (hAce2-Variant1-IgG4 fusion); (f) SEQ ID NO: 3 or a sequence at least about 90% identical thereto encoding SEQ ID NO: 4 (hAce2-Variant2-IgG4 fusion); (g) SEQ ID NO: 5 or a sequence at least about 90% identical thereto encoding SEQ ID NO: 6 (hAce2-Variant3-IgG4 fusion); or (h) SEQ ID NO: 7 or a sequence at least about 90% identical thereto encoding SEQ ID NO: 8 (hAce2-Variant4-IgG4 fusion). In certain embodiments, the mutant hAce2 soluble decoy is a hAce2 soluble decoy fusion protein further comprising an immunoglobulin Fc region, optionally wherein the Fc is a human Fc, a human IgG1 Fc, a human IgG4 Fc or a human IgM Fc. In certain embodiments, the mutant hAce soluble decoy protein is (a) a protein comprising SEQ ID NO: 4 (a hAce2-Variant2-IgG4 fusion) or an amino acid sequence at least 95% identical thereto; (b) a protein comprising SEQ ID NO: 109 (hAce2-Variant2-IgG1 fusion) or an amino acid sequence at least 95% identical thereto; (c) a protein comprising SEQ ID NO: 113 (hAce2-Variant2-IgG1 fusion with “GS” linker) or an amino acid sequence at least 95% identical thereto; (d) a protein comprising SEQ ID NO: 111 (hAce2-MR27-Variant-IgG1 fusion) or an amino acid sequence at least 95% identical thereto; (e) a protein comprising SEQ ID NO: 115 (hAce2-MR27-Variant2-IgG1 fusion with GS linker) or an amino acid sequence at least 95% identical thereto; or (f) a protein comprising SEQ ID NO: 2 (hAce2-Variant1-IgG4 fusion) or an amino acid sequence at least 95% identical thereto; (g) a protein comprising SEQ ID NO: 6 (hAce2-Variant3-IgG4 fusion) or an amino acid sequence at least 95% identical thereto; (h) a protein comprising SEQ ID NO: 8 (hAce2-Variant4-IgG4 fusion) or an amino acid sequence at least 95% identical thereto; (i) a protein comprising SEQ ID NO: 72 and SEQ ID NO: 77 (hAce2-Variant4-IgG4 fusion) or an amino acid sequence at least 95% identical thereto; (j) a protein comprising SEQ ID NO: 73 and SEQ ID NO: 77 (hAce2-Variant4-IgG4 fusion), or an amino acid sequence at least 95% identical thereto; or (k) a protein comprising SEQ ID NO: 94, 96, 98, 100, or 105 or an amino acid sequence at least 95% identical thereto. In certain embodiments, the mutant hAce2 is fused to the Fc domain directly, or optionally fused via a flexible GSG linker, wherein the GSG linker is selected from 3 about to about 123 amino acids in total.

In certain embodiments, the rAAV comprises a vector genome comprising an expression cassette comprising regulatory control sequences comprising one or more of: a promoter, at least one enhancer, at least one intron, and a poly A signal, optionally wherein the regulatory control sequences comprise a CB7 hybrid promoter, a chicken beta actin intron, and a rabbit beta globin polyA. In certain embodiments, the rAAV comprises the vector genome comprising an expression cassette having nucleotide sequence of SEQ ID NO: 116, SEQ ID NO: 117, SEQ ID NO: 118, SEQ ID NO: 119, SEQ ID NO: 86; SEQ ID NO: 88; SEQ ID NO: 90, or SEQ ID NO: 92. In certain embodiments, the rAAV comprises the vector genome having the nucleic acid sequence of: SEQ ID NO: 17, SEQ ID NO: 19, SEQ ID NO: 21, SEQ ID NO: 23, SEQ ID NO: 85, SEQ ID NO: 87, SEQ ID NO: 89, or SEQ ID NO: 91.

In another aspect provided herein is a recombinant AAV (rAAV) comprising an AAV capsid and a vector genome packaged therein, wherein the vector genome comprises a 5′ inverted terminal repeat (ITR), a nucleic acid sequence encoding a fusion protein comprising a signal peptide, a mutant hAce2 soluble decoy protein, and an optional immunoglobulin Fc region, wherein the coding sequences for the fusion protein are under the control of regulatory control sequences which direct expression of the fusion protein, and a 3′ ITR, wherein the mutant hAce2 soluble decoy protein comprises a mutant amino acid in (a) R or M at residue 14 (K changed to R or M) and (b) V or K at residue (18) (E changed to V or K), and at least one further residue (c) P at residue (22) (L changed to P); (d) R at residue (25) (Q changes to R); (e) A at residue (30) (S changed to A); (f) A at residue (42) (V changed to A); (g) I or F at residue (62) (L changes to I or F); (h) D at residue (73) (N changed to D); (i) P at residue (74) (L changed to P); (j) Y at residue (313) (N changed to Y); and/or (k) H at residue (328) (H changed to L), wherein the mutant hAce2 soluble decoy protein amino acid position is based on SEQ ID NO: 81 or 83, or a hAce2 decoy protein at least 95% identical to SEQ ID NO: 81 or 83. In certain embodiments, a mutant hAce2 soluble decoy protein comprises an amino acid sequence selected from (i) the substitutions of (a), (b), and (j) or (ii) the substitutions of (a), (b), (g), and (j). In certain embodiments, the signal peptide is a human signal peptide, optionally wherein the signal peptide is the native hAce2 signal peptide.

In certain embodiments, the AAV capsid is an AAV9 capsid, a AAVhu68 capsid, an AAV5 capsid, an AAV6 capsid, an AAV6.2 capsid, or an AAVrh91 capsid.

In certain embodiments, a pharmaceutical composition is provided which comprises at least one recombinant AAV as described herein and one or more of any of: a pharmaceutically acceptable diluent, a suspending agent, a preservative, and/or a surfactant. In certain embodiments, the pharmaceutical composition is formulated for intranasal administration. In certain embodiments, the pharmaceutical composition is formulated for intrapulmonary administration. In certain embodiments, the pharmaceutical composition is formulated for intravenous administration. In certain embodiments, the pharmaceutical composition is formulated for intraperitoneal administration.

In another aspect, a method is provided for treating and/or preventing one or more symptoms of SARS-CoV2 by administering or co-administering a pharmaceutically effective amount of synthetic or recombinant hAce2 soluble decoy protein, an rAAV or a pharmaceutical composition as described herein, and combinations thereof. In certain embodiments, the symptoms are one or more of fever, cough, gastrointestinal distress, nausea, vomiting, diarrhea, eye pain, breathing difficulty, loss of taste, and/or loss of smell. In certain embodiments, the method comprises administering or co-administering the hAce2 soluble decoy protein, the rAAV or a pharmaceutical composition intranasally. In still other embodiments, the method comprises administering or co-administering the hAce2 soluble decoy protein, the rAAV or a pharmaceutical composition via inhalation. In other embodiments, the method comprises administering or co-administering the hAce2 soluble decoy protein, the rAAV or a pharmaceutical composition intravenously.

A mutant soluble human Ace2 (hAce2) protein useful in preventing infection with betacoronaviruses, including SARS-CoV2 is provided, as are compositions useful in treating disease associated with betacoronavirus-associated disease, including, e.g., COVID-19. Further provided herein are use of an rAAV in the manufacture (preparing) of a medicament for the prevention and/or treatment infection with a virus mediated by an Ace2 receptor, optionally wherein the virus is a SARS virus, or optionally wherein the virus is SARS-CoV2.

In another aspect, provided herein is a packaging host cell in culture or suspension comprising: (a) a nucleic acid molecule encoding a vector genome comprising a 5′ inverted terminal repeat, an expression cassette comprising a mutant soluble hAce2 fusion protein, and a 3′ inverted terminal repeat; (b) nucleic acid sequences encoding an rAAV capsid protein under control of sequences which regulate expression of the capsid protein in the packaging hose cell; and (c) helper sequences for replication and packaging of the vector genome into the rAAV capsid. In certain embodiments, an rAAV stock is produced from the packaging host cell described herein.

These and other advantages of the various embodiments described herein will be apparent from the following detailed description of the invention.

### BRIEF DESCRIPTION OF DRAWINGS

FIGS. 1A and 1B show a schematic representation of yeast-surface display technique. FIG. 1A shows a schematic expression cassette for use in a mutational library. FIG. 1B shows a schematic representation of a yeast-surface display used in the high throughput screening (a yeast display (YD) system with an HA antigen, wherein the antigen is a coronavirus spike protein. A large mutational library of Ace2 is generated using error prone PCR. Aga2 fusion directs the Ace2 variant to be exported to and ligated onto the exterior yeast cell wall, thus coating the exterior of the yeast cell that contains that variant’s gene. This provides the link between genotype and phenotype allowing a high-throughput screening required for protein engineering technique.

FIGS. 2A and 2B show dot plots of results from florescence-activated cell sorting (FACS) following multiple consecutive rounds of selection for receptor binding domain (RBD)-targeted binder. Each dot represents two fluorescent signals of a separate yeast cell in the analyzed (sorted) sample, wherein X-axis is a measure of Ace2 expression, and Y-axis is measure of RBD-binding. FIG. 2A shows results FACS run with goal to isolate the rare variants within the population with improved SARS-CoV-2 spike binding. FIG. 2B shows first round of selection with FACS for improved SARS-CoV-2 spike binding. FIG. 2C shows second round of selection with FACS for improved SARS-CoV-2 spike binding. FIG. 2D shows third round of selection with FACS for improved SARS-CoV-2 spike binding.

FIG. 3 shows the neutralization data as measured across the most improved variants identified from screen. These variants neutralized SARS-CoC2 activity in the range of 3-10 ng/mL (Generation 2).

FIGS. 4A to 4G show selection and characterization of best variant from digital recombinant variants screened over wild-type Ace2-IgG (Generation 3). The screening by flow cytometry, as in the primary round, was performed wherein the staining concentrations dropped to lower concentrations and the wash steps prolonged to promote selection of only the tightest RBD-binding clones. FIG. 4A shows a confirmation of binding of the yeast clones from the final rounds of sorting to SARS-CoV2 RBD by flow cytometry. FIG. 4B shows results of the neutralization assay with hAce2-Variant3-IgG4 purified decoy in full titration of SARS-CoV2 reporter virus. FIG. 4C shows results of the neutralization assay with hAce2-Variant3-IgG4 purified decoy in full titration of SARS-CoV1 reporter virus. FIG. 4D shows results of the neutralization assay with hAce2-Variant1-IgG4 purified decoy in full titration of SARS-CoV2 reporter virus. FIG. 4E shows results of the neutralization assay with hAce2-Variant1-IgG4 purified decoy in full titration of SARS-CoV1 reporter virus. FIG. 4F shows results of the neutralization assay with hAce2-Variant2-IgG4 purified decoy in full titration of SARS-CoV2 reporter virus. FIG. 4G shows results of the neutralization assay with hAce2-Variant2-IgG4 purified decoy in full titration of SARS-CoV1 reporter virus.

FIGS. 5A to 5C provide an alignment of wild-type (WT) human Ace2 (hAce2) soluble protein (SEQ ID NO: 25) and modified hAce2 soluble decoy proteins (SEQ ID NOs: 10 (hAce2-Variant1), 12 (hAce2-Variant2), 14 (hAce2-Variant 3), and 16(hAce2-Variant4)). FIG. 5A provides an alignment of amino acids 1 to 300 of wild-type and modified hAce2 soluble decoy protein. FIG. 5B provides an alignment of amino acids 301 to 540 of wild-type and modified hAce2 soluble decoy proteins. FIG. 5C provides an alignment of amino acids 541 to 600 of wild-type and modified hAce2 soluble decoy proteins.

FIGS. 6A and 6B show the measured binding affinity and fold change in binding affinity of modified hAce2-Variant1-IgG4 soluble decoy fusion protein and wild-type hAce2-IgG4 soluble decoy fusion protein for mutant RBD of SARS-CoV2. FIG. 6A shows the measured binding affinity of modified hAce2-Variant1-IgG4 soluble decoy fusion protein and wild-type hAce2-IgG4 soluble decoy fusion protein for mutant RBD of SARS-CoV2. FIG. 6B shows a fold change in binding affinity of modified hAce2-Variant1-IgG4 soluble decoy fusion protein and wild-type hAce2-IgG4 soluble decoy fusion protein for mutant RBD of SARS-CoV2 (affinity to mutant RBD of SARS-CoV2 over affinity to wild type RBD of SARS-CoV2).

FIG. 7 shows a graph comparison of mutant decoy affinity versus wild-type (WT) decoy affinity to various mutant RBD of SARS-CoV2.

FIGS. 8A and 8B show the alignment of wild-type (WT) CoV2 RBD, mutants of CoV2, and CoV1 (SEQ ID NOs: 43-51). FIG. 8A shows the alignment of amino acids 1 to 120. FIG. 8B shows the alignment of amino acids 121 to 229.

FIG. 9 shows an example AAV vector genome with engineered hAce2 decoy (hAce2-Variant1-IgG4). The construct contains Inverted Terminal Repeat (ITR) sequences for packaging, CMV IE enhancer with CB promoter (CB7 hybrid promoter), a Chicken Beta actin intron (chimeric intron), the engineered human Ace2 decoy gene fused to a human IgG4 Fc domain, and a Rabbit globin polyA terminator.

FIGS. 10A to 10C show analytics from mouse study comprising intranasal delivery of 1x10¹¹ Genome Copies (GC) of rAAVs encoding hAce2-Variant½-IgG4-Fc fusions of engineered decoys in either AAVhu68 or AAVrh91 capsids. BALF samples were collected at day 7 post-transduction. FIG. 10A shows a mass spectrometry with hAce2-IgG4 standards to determine the concentration of decoy in BALF from 5 animals in each dosing group. FIG. 10B shows results of CoV2 spike binding activity in the samples, wherein an immobilized recombinant CoV2 spike protein in an ELISA assay was with the human Fc tag on the decoy as the detection epitope. FIG. 10C shows results of a commercial CoV2 reporter virus used to detect neutralizing activity in the BALF samples. The dotted line represents complete neutralization in the assay as established with saturating concentrations of a neutralizing monoclonal antibody.

FIGS. 11A to 11F show analytics from NHP study, intranasal delivery of 5x10¹² GC of rAAVs encoding hAce2-Variant½-IgG4-Fc of mutant decoys. The capsids used were AAVhu68 and AAVrh91. This data is on NLF samples collected at day 7 post-transduction. FIG. 11A shows CoV2 spike binding activity in the 1x BALF samples (AAVrh91), wherein an immobilized recombinant CoV2 spike protein in an ELISA assay, with the human Fc tag on the decoy as the detection epitope. FIG. 11B shows CoV2 spike binding activity in the 10x BALF samples (AAVrh91), wherein an immobilized recombinant CoV2 spike protein in an ELISA assay, with the human Fc tag on the decoy as the detection epitope. FIG. 11C shows mass spectrometry with Ace2-IgG4 standards to determine the concentration of decoy in NLF 1X samples (AAVrh91) from 2 animals in each doing group. FIG. 11D shows mass spectrometry with Ace2-IgG4 standards to determine the concentration of decoy in NLF 10X samples (AAVrh91) from 2 animals in each doing group. FIG. 11E shows mass spectrometry with Ace2-IgG4 standards to determine the concentration of decoy in NLF 1X samples (AAVhu68) from 2 animals in each doing group. FIG. 11F shows mass spectrometry with Ace2-IgG4 standards to determine the concentration of decoy in NLF 10X samples (AAVhu68) from 2 animals in each doing group.

FIGS. 12A to 12D show analytics from an NHP study with intranasal delivery of 5x10¹² GC of rAAVs encoding hAce2-Variant½-IgG4-Fc of mutant decoys. The capsids used were AAVhu68 and AAVrh91. This data is on NLF samples collected at day 14 post-transduction. FIG. 12A shows CoV2 spike binding activity in the 1x BALF samples (AAVrh91), wherein an immobilized recombinant CoV2 spike protein in an ELISA assay, with the human Fc tag on the decoy as the detection epitope. FIG. 12B shows CoV2 spike binding activity in the 10x BALF samples (AAVrh91), wherein an immobilized recombinant CoV2 spike protein in an ELISA assay, with the human Fc tag on the decoy as the detection epitope. FIG. 12C shows mass spectrometry with Ace2-IgG4 standards to determine the concentration of decoy in NLF 1X samples (AAVrh91) from 2 animals in each doing group. FIG. 12D shows mass spectrometry with Ace2-IgG4 standards to determine the concentration of decoy in NLF 10X samples (AAVrh91) from 2 animals in each doing group.

FIGS. 13A to 13B shows neutralization data across various SARS-CoV-2 Variants as measured in an assay using a reporter virus. FIG. 13A shows inhibition of SARS-CoV-2 RBD-hAce2 interactions in presence engineered hAce2 soluble decoy, hAce2-Variant2-IgG4. FIG. 13B shows a plot of IC50 values for interaction inhibition between hAce2-Variant2-IgG4 and SARS-CoV2-RBD mutant variants, as measured in a neutralization assay.

FIG. 14 shows estimated IC50 of 6 “revertants” of hAce2-Variant1-IgG4 soluble decoy, wherein each “revertant” comprised of one amino acid substitution reverted from engineered back to wild type at the indicated positions (based on numbering of amino acid sequence of SEQ ID NO: 25).

FIGS. 15A to 15B show the effect of varying linker length on IC50 values of interaction between hAce2-Variant2-IgG4 soluble decoy and SARS-CoV2-RBD. FIG. 15A shows estimated IC50 values in comparison to varying “GSG” linker length, or varying decoy length (1-615 versus 1-740 amino acid of hAce2), which is linked at the amino terminus of IgG4 f the soluble decoy fusion protein. FIG. 15B shows a schematic representation of protein interaction structure between hAce2 and RBD of SARS-CoV2, showing amino acids 1-615 and 1-740, as they are mapped onto the structure of protein interaction.

FIGS. 16A to 16D show analytics from a mouse challenge study with intranasal delivery of 1 x 10¹¹ GC of engineered decoy in the absence or presence of 280 PFU SARS-CoV-2. 7 days prior to the delivery of SARS-CoV-2 or PBS, all groups were given either the AAV decoy or PBS. At Day 0 all groups were given either SARS-CoV-2 or PBS. At day 4 and 7 mice were euthanized for histopathology and viral PCR. FIG. 16A shows the change in body weight among the groups over a course of 5 days. FIG. 16B shows the change in body weight among the groups over a course of 8 days. FIG. 16C shows the concentration of AAV decoy in G4 and G5 at day 4 and 7, respectively. FIG. 16D shows inflammation scores from lung sections derived from the 5 groups at day 4 and 7.

FIGS. 17A to 17C shows analytics from a NHP study with intranasal delivery of 5x10¹² GC of rAAVs encoding hAce2-Variant2 (GTP14HL-IgG) of mutant decoys. The capsids used were AAVhu68 and AAVrh91. This data is on NLF samples collected at days 7, 14 and 28 post-transduction. FIG. 17A shows mass spectrometry to assess the change in concentration of the mutant decoys over a period of 28 days. FIG. 17B shows mass spectrometry of the concentration of AAVrh91 at 5x10¹² GC and 5x10¹¹ GC at day 14 post-transduction. FIG. 17C shows CoV-2 spike binding activity in NLF samples (AAVrh91), wherein an immobilized recombinant CoV2 spike protein in an ELISA assay, with the human Fc tag on the decoy as the detection epitope.

FIGS. 18A to 18F show ACE2 Decoy Receptor Engineering. FIG. 18A shows decoy affinity maturation and candidate selection process. FIG. 18B shows flow cytometry analysis of the naïve (dark gray) and sorted populations (light gray) from the secondary YD library. FIG. 18C shows NGS analysis of plasmid populations recovered from rounds of YD. FIG. 18D shows SPR binding analysis for CoV-2 RBD injected over surface-immobilized ACE2-wt. FIG. 18E shows SPR binding analysis for CoV-2 RBD injected over surface-immobilized hAce2-Variant2. FIG. 18F shows Wuhan CoV-2-Pseudotyped lentiviral reporter neutralization assay of ACE2-wt-Fc4 and hAce2-Variant2(CDY14HL)-Fc4. Data for at least three independent measurements are presented as average ± standard deviation.

FIGS. 19A to 19E show ACE2 Decoy Binding and Neutralization Across Diverse CoVs. FIG. 19A shows structural models (7DF4.pdb [Xu C, Wang Y, Liu C, Zhang C, Han W, Hong X, et al. Conformational dynamics of SARS-CoV-2 trimeric spike glycoprotein in complex with receptor ACE2 revealed by cryo-EM. Sci Adv. 2021;7(1). Epub 2020/12/06. doi: 10.1126/sciadv.abe5575. PubMed PMID: 33277323; PubMed Central PMCID: PMCPMC7775788.]) of Wuhan CoV-2 RBD (light) bound to human ACE2 (dark) RBD. The six RBD residues frequently mutated in emerging CoV-2 variants are shown. The Wuhan CoV-2 model with RBD residues highlighted at the ACE2 interface differs from CoV-1. FIG. 19B shows SPR measurements: ACE2-wt-Fc4 or hAce2-Variant2-Fc4 binding to various purified recombinant RBD proteins. FIG. 19C show Variant2 (CDY14HL)-Fc4 titrations using pseudotyped lentivirus reporters encoding CoV-2 spike proteins from four US-CDC Variants of Concern. RBD mutations are indicated in brackets, however in many cases other spike mutations exist that are not listed. FIG. 19D shows. Pseudotype titrations for six additional CoV-2 spike variants. FIG. 19E shows pseudotype titration for CoV-1 spike reporter virus. Reporter virus activity data are presented as mean ± standard error of the mean for at least three replicate titrations.

FIGS. 20A to 20J show protection in the human ACE2 transgenic mouse model. FIG. 20A shows BAL from vector-treated animals analyzed for decoy protein by MS. FIG. 20B shows BAL from vector-treated animals analyzed for SARS-CoV-2 spike ELISA. FIG. 20C shows BAL from vector-treated animals analyzed for neutralization of SARS-CoV-2 pseudotyped lentivirus. FIG. 20D shows challenge study design. FIG. 20E shows weight loss in the animals that were sustained for 7 days; one animal in the vehicle and vector treated groups required euthanasia. FIG. 20F shows MS assay of expression in ASF (corrected for BAL dilution). FIG. 20G shows pulmonary inflammation histopathology scores of tissues harvested at days 4 and 7. FIG. 20H shows viral RNA in BAL. FIG. 20I shows viral RNA in lung. FIG. 20J shows sub-genomic RNA in lung. Outliers are indicated with X.

FIGS. 21A to 21E show AAV-Delivered Decoy Expression in the Airway of NHP. FIG. 21A shows pooled capsid comparison using mRNA barcoding. mRNA barcode enrichment scores (average across 4 barcodes + SD) in (FIG. 21A nasopharynx) and (FIG. 21B) septum. FIG. 21C shows AAVrh91 transduction profile in airway barcode study. FIG. 21D shows MS assay of expression in ASF (corrected for BAL dilution) for NHPs (2/group) dosed with ACE2 decoy vector. FIG. 21E shows correlation between decoy protein by MS and spike binding by ELISA. Data includes d7 and d14 samples from (FIG. 21D) plus NLF of 3 naïve macaques. We excluded one naïve animal from ELISA analysis because of background binding presumably due to a prior coronavirus infection.

FIGS. 22A to 22E show characterization of initial ACE2 decoy construct. FIG. 22A shows results for construct expressed in HEK293 cells and detected in supernatant using a sandwich ELISA to ACE2 for constructs without an Fc domain. FIG. 22B shows result of an ELISA with SARS-CoV-2 spike protein as a capture antigen and an anti-human IgG polyclonal antibody used for detection of hAce2 with Fc fusion proteins expressed in HEK293 cells. FIG. 22C shows the purified ACE2-NN-Fc4 protein was titrated against Wuhan CoV2 pseudotyped lentivirus bearing a luciferase reporter. The IC50 was obtained from a fit of these data (15 µg/ml). FIG. 22D shows the candidate construct (ACE2-NN-Fc4) was packaged in an AAV vector (hu68 capsid) and administered IN to WT mice. Seven days after administration, BAL was collected for measurement of transgene expression using an ELISA with SARS-CoV-2 spike protein as a capture antigen to confirm that the decoy receptor expressed in vivo was functional. BAL from similar experiments was 6-fold diluted from the ASF as determined by comparison of BAL and serum urea. Thus, we determined that ASF concentrations of the decoy were likely below 2 ug/ml. FIG. 22E shows two NHPs (IDs 258 and 396) received 9 x 10¹²GC of an AAVhu68 vector expressing a soluble ACE2-NN-Fc fusion protein via the MAD. Nasal lavage samples were collected weekly after vector administration and concentrated 10-fold for analysis. The concentration of the decoy receptor in NLF was measured by MS. Urea measurements in similar experiments indicate that 10X nasal lavage is ~8-fold diluted from ASF. We therefore determined that ASF concentrations of the decoy were less than 100 ng/ml.

FIGS. 23A to 23D show design and selection of primary and secondary yeast display libraries. FIG. 23A shows the designed two primary yeast display libraries: 1) the whole ACE2 gene fragment was mutagenized (Whole) and 2) the mutagenesis was limited to only the first 96 amino acids (NC) to concentrate the mutagenesis on the region most likely to impact RBD binding. The regions shaded gray were subjected to error prone PCR to introduce mutations. FIG. 23B shows results of deep sequencing of yeast display plasmids extracted from the final round of sorting for the Whole and NC libraries. The fractional rate of mutation at each position in 18-615 of ACE2 is plotted. Improving mutations occurred mostly in the first 96 amino acids regardless of the input library. These include RBD contact residues, second-shell residues, and the distal consensus N-glycan site at position 90, an apparent negative regulator of RBD binding. Though C-terminal mutation load was generally associated with poor ACE2 expression (data not shown), several consensus C-terminal mutations emerged from the whole ACE2 primary sorts. These include a substitution to Y at position 330, which we identified in a clone with improved binding. FIG. 23C shows a detailed plot of the mutational frequencies in whole and NC library final round sorts for residues 18-100. The libraries yielded many of the same mutants this region with improved binding activity. FIG. 23D Schematic representation of secondary library design. We isolated 300 yeast colonies from the sorted primary (Whole and NC) libraries, analyzed them individually for RBD binding and ACE2 expression by flow cytometry, and selected 90 isolates with validated binding improvements. Next, we generated a secondary library by shuffling selected ACE2 genes using the staggered extension process (StEP) method. Given that most improving mutations were N-terminal, we shuffled only residues 18-103 of the input templates (orange shaded region in the schematic), matching these with a mixture of unmutated and N330Y C-terminal DNAs in a multi-fragment assembly yeast transformation.

FIG. 24A shows the parallel paths to the generation of affinity matured ACE2 decoy, selection and screening of primary display hits, and mutations from a secondary round of yeast display sorting. FIG. 24B shows estimate IC50 (ng/mL) for Expression titer relative to Ace2-WT-Fc4 (for primary (1°), secondary (2°) digital, and secondary (2°) molecular). FIG. 24C shows reporter virus activity for Ace-IgG variants GTP14-IgG (Variant1) and GTP14HL-IgG (Variant2)

FIGS. 25A and 25B show CoV2 challenge study in mice. FIG. 25A shows a challenge study in mice plotted as average weight loss (percentage) in males. FIG. 25B shows a challenge study in mice plotted as average weight loss (percentage) in females.

FIGS. 26A to 26I show AAV capsid selection for NHP IN delivery. FIG. 26A shows mRNA barcode enrichment in airway tissues of Maxillary sinus for a mixture of 9 barcoded serotypes delivered IN at 2.7 x 10¹¹ GC each. FIG. 26B shows mRNA barcode enrichment in airway tissues of Trachea prox. for a mixture of 9 barcoded serotypes delivered IN at 2.7 x 10¹¹ GC each. FIG. 26C shows mRNA barcode enrichment in airway tissues of trachea mid. for a mixture of 9 barcoded serotypes delivered IN at 2.7 x 10¹¹ GC each. FIG. 26D shows mRNA barcode enrichment in airway tissues of trachea dist. for a mixture of 9 barcoded serotypes delivered IN at 2.7 x 10¹¹ GC each. FIG. 26E shows mRNA barcode enrichment in airway tissues of mainstem bronchi for a mixture of 9 barcoded serotypes delivered IN at 2.7 x 10¹¹ GC each. FIG. 26F shows mRNA barcode enrichment in airway tissues of lung upper caudal for a mixture of 9 barcoded serotypes delivered IN at 2.7 x 10¹¹ GC each. FIG. 26G shows mRNA barcode enrichment in airway tissues of “lung lower L” for a mixture of 9 barcoded serotypes delivered IN at 2.7 x 10¹¹ GC each. We assigned four uniquely barcoded transgenes to each capsid at manufacture. Data show the enrichment score (tissue abundance in RT-PCR-NGS/ injection mixture abundance in PCR NGS) for all 4 barcodes per capsid with mean and SD. FIG. 26H shows the biodistribution of vector genomes in airway tissues (ethmoid, maxillary, cavity septum, nasopharynx) 28 days after dosing. FIG. 26I shows the biodistribution of vector genomes in various airway tissues including lower airway tissues at 28 days after dosing. Four NHP were IN dosed with AAVrh91 or AAVhu68 vectors encoding decoy transgenes at 5 x 10¹² GC. AAVrh91 achieved higher gene transfer in upper airway tissues, particularly in the maxillary sinuses and cavity septum. Gene transfer in lower airway tissues was more variable.

FIG. 27 shows neutralization data as measured across different sampled pools of the purified engineered hAce2 decoy Fc1 (IgG 1 Fc) fusion proteins, and compared with the engineered hAce2 decoy Fc4 (IgG4 Fc) fusion protein.

FIG. 28A shows a plot of neutralization data against Wuhan CoV2, as measured across different sampled pools of the purified engineered hAce2 decoy Fc1 (IgG 1 Fc) fusion proteins, and compared with the engineered hAce2 decoy Fc4 (IgG4 Fc) fusion protein.

FIG. 28B shows a plot of neutralization data against Delta CoV2 (variant), as measured across different sampled pools of the purified engineered hAce2 decoy Fc1 (IgG 1 Fc) fusion proteins, and compared with the engineered hAce2 decoy Fc4 (IgG4 Fc) fusion protein.

FIG. 29A shows concentration of the engineered hAce2 decoy Fc1 fusion protein, as measured with mass spectrometry in collected serum samples, post intraperitoneal administration of hAceMR27HL-Variant-IgG1 Fc decoy fusion at doses of 3 mg/kg, 10 mg/kg, and 30 mg/kg.

FIG. 29B shows concentration of the engineered hAce2 decoy Fc1 fusion protein, as measured with mass spectrometry in collected NJF samples, post intraperitoneal administration of hAceMR27-Variant-IgG1 Fc decoy fusion at doses of 3 mg/kg, 10 mg/kg, and 30 mg/kg.

FIG. 29C shows concentration of the engineered hAce2 decoy Fc1 fusion protein, as measured with mass spectrometry in collected serum samples, post intraperitoneal administration of hAceMR27HL-Variant-IgG1 Fc or GTP14HL-Fc1 decoy fusion from various pools of protein samples, when administered at doses of 3 mg/kg.

FIG. 29D shows concentration of the engineered hAce2 decoy Fc1 fusion protein, as measured with mass spectrometry in collected NLF samples, post intraperitoneal administration of hAceMR27HL-Variant-IgG1 Fc or GTP14HL-Fc1 decoy fusion from various pools of protein samples, when administered at doses of 3 mg/kg.

FIG. 30A shows decoy protein levels as determined by mass spectrometry analysis (ng/mL) post administration of either AAVhu68.GTP14HL or AAVrh91.GTP14HL.

FIG. 30B shows decoy protein levels as determined by mass spectrometry analysis (ng/mL) post administration of either AAVhu68.GTP14HL or AAVrh91.GTP14HL, and plotted as urea-corrected decoy protein concentration (ng/mL).

FIG. 31 shows exemplary pool hAce2 decoy protein expression levels (mg/L) in a 10 day process in a 10L Stirred Tank Controlled Bioreactor. These results show about 1.2 g/L of protein purified using a one or two step process with higher percent monomer purity and good recovery (~90%).

FIG. 32A shows serum decoy levels in NHP following administration by IV infusion of hAce2 decoy protein (MR27HL-Fc1) at a dose of 30 mg/kg (broken x axis for showing early timepoints (Hours)).

FIG. 32B shows potency of decoy in serum samples of NHP following administration by IV infusion of hAce2 decoy protein (MR27HL-Fc1) at a dose of 30 mg/kg, plotted as a reporter virus activity over decoy MS concentration (ng/ml) at 1 hour, 7 hours, and 24 hours post administration.

FIG. 33 shows viral neutralization assay using lentiviruses pseudotyped with the ancestral (Wuhan Hu1) or Omicron BA.1 and BA.2 variant spike protein.

FIG. 34 shows correlation of protein decoy expression in collected NLF following AAVrh91.hAce2GTP14HL-IgG4 administration at various doses (1.02 x 10¹¹, 3.40 x 10¹², and 1.02 x 10¹² GC).

FIG. 35 shows hAce2 decoy protein levels in NLF, plotted as ng/mL, measured in NHPs at day 30 and day 120 following AAVrh91.hAce2GTP14HL-IgG4 administration at various doses (1.02 x 10¹¹, 3.40 x 10¹², and 1.02 x 10¹² GC) in NHPs.

FIG. 36 shows Engineered ACE2 decoys bind diverse ACE2-dependent CoVs, plotted as decoy fluorescence for each specified CoV strain.

FIG. 37A shows a schematic overview of the fluorescence binding assay. Histograms are from a representative replicate of decoy binding to yeast-displayed Wuhan-Hu1 RBD. FIG. 37B shows representative decoy binding data for the RBD from the ancestral (Wuhan-Hu1) SARS-CoV-2 strain and both Omicron variants. The binding of the CDY14HL-Fc4 with RBD, was plotted as bound decoy fluorescence over decoy concentration in nM. RBDs from SARS-CoV-2 variants were individually expressed in budding yeast as fusion proteins that are trafficked to the external cell wall. Decoy or un-engineered ACE2 are incubated with the yeast, stained with antibodies, and the relative level of decoy binding is assessed by flow cytometry.

FIG. 38A shows phylogenetic tree of sarbecovirus RBDs created using the maximum likelihood method.

FIG. 38B shows a schematic overview of the fluorescence binding assay comprising budding yeast displaying RBD and ACE2 mFc.

FIG. 38C shows relative levels of decoy binding to diverse RBDs under several conditions as assessed by the yeast-display system. Similar to SARS-CoV2, all sarbecovirus RBDs retain >30% binding in the competition assay.

FIG. 38D shows yeast displayed RBDs used to test for decoy or wt-ACE2 binding at 100 nM.

FIG. 38E shows yeast displayed BtKY72 RBDs titrated with decoy and evaluated to determine the dissociation equilibrium constant.

FIG. 39A shows the complex [Xu C, Wang Y, Liu C, Zhang C, Han W, Hong X, et al. Conformational dynamics of SARS-CoV-2 trimeric spike glycoprotein in complex with receptor ACE2 revealed by cryo-EM. Sci Adv. 2021;7(1)] between the SARS-CoV-2 Wuhan-Hu1 RBD (yellow ribbons) and human ACE2 (blue ribbons).

FIG. 39B shows the Wuhan-Hu1 RBD from the ACE2 complex structure alone. Red spheres indicate the α-carbon of amino acid residues mutated in several pre-Omicron SARS-CoV-2 variant strains. Amino acid position numbers are show for each mutated residue.

FIG. 39C shows the Wuhan-Hu1 RBD from the ACE2 complex structure alone. Red spheres indicate the α-carbon of amino acid residues mutated around the interface with ACE2 in Omicron BA.1.

FIG. 39D shows the Wuhan-Hu1 RBD from the ACE2 complex structure alone. Red spheres indicate the α-carbon of amino acid residues mutated around the interface with ACE2 in Omicron.BA.2.

FIGS. 40A and 40B show Biolayer interferometry (BLI) kinetics for hAce2GTP14HL-Fc1 and SARS-CoV-2 Variants. FIG. 40A shows depiction of Biolayer interferometry assay format. hAce2GTP14HL-Fc1 was immobilized as ligand and SARS-CoV-2 variant RBD was used as analyte. FIG. 40B shows a representative fitted sensogram for hAce2GTP14HL-Fc1 and CoV-2 Wuhan-Hu1 spike RBD.

FIG. 41 shows plotted graph of evaluation of the relationship between decoy affinity and the amount of decoy retained in a competitive binding assay

### DETAILED DESCRIPTION OF THE INVENTION

A mutant soluble Ace2 protein is provided, as are vector systems for expressing the protein in vitro or in vivo depending on whether a gene therapy or protein-based therapeutic approach is desired, or a combination approach. Provided herein are soluble Ace2 decoys where are engineered to be retain inhibitory ability against SARS viruses and their variants which bind to human Ace2 receptors. In certain embodiments, the mutant soluble Ace2 proteins provided herein to function as a decoy receptor for viral pathogens using the Ace2 receptor, including SARS CoV-2, the virus which causes COVID-19 and SARS-CoV1. Furthermore, in certain embodiments, an AAV-expressed variant as provided herein Furthermore, (e.g., CDY14HL-Fc4) maintains tight binding or neutralizing activity for the distantly-related sarbecoviruses WIV1-CoV and SARS-CoV-1 despite being engineered for improved activity against SARS-CoV-2. This indicates that this illustrative decoy is useful to combat future pandemics from currently pre-emergent ACE2-dependent coronaviruses. Strong binding and neutralizing potency of an AAV-expressed ACE2 decoy against SARS-CoV-2 variants including Omicron BA.1 and Omicron BA.2, is illustrated in the examples. Tight decoy binding tracks with human ACE2 binding of viral spike receptor-binding domains across diverse clades of coronaviruses. Furthermore, in a coronavirus that cannot bind human ACE2, a variant that acquired human ACE2 binding is bound by an illustrative AAV-expressed decoy with nanomolar affinity.

In one aspect, a recombinant viral vector is replication-incompetent and comprises an exogenous viral genome which comprises coding sequences for at least one mutant hACE2 (or hAce2) soluble protein. In certain embodiments, the compositions and methods provided herein have advantages over broadly neutralizing antibodies (bNAbs), as viral escape mutants that evade neutralization by the soluble receptor lose infectivity due to reduced affinity for the endogenous receptor. In certain embodiments, the compositions and methods provide for use of a soluble decoy also avoids potential antibody-mediated enhancement of infection at non-neutralizing titers, which has been reported for SARS CoV-1 antibodies. In certain embodiments, the compositions provided herein are designed for intravenous delivery of a recombinant protein of an engineered soluble hACE2 decoy fusion protein. In certain embodiments, the compositions provided herein are designed for mucosal expression in order to provide lower systemic exposure than occurs following intravenous or intramuscular delivery, thereby further enhancing the safety of an AAV-mediated approach.

In certain embodiments, a protein is provided herein (e.g., hAce2-Variant2-Fc4 (CDY14HL-Fc4; GTP14HL-IgG4 or GTP14HL-Fc4)), which has high binding and neutralizing activity against a full range of SARS-CoV-2 variants. For example, as illustrated in the examples below, AAV-mediated expressed Fc-fused decoy, CDY14HL-Fc4 (hAce2-Variant2-IgG), contains six (6) amino acid substitutions that improve neutralization of CoV2 variants by 300-fold versus un-engineered ACE2 and contain an active site mutation that ablate its endogenous angiotensin-cleaving activity (see, e.g., Examples 1-4, and 7). Unexpectedly, this protein has also demonstrated equally potent binding and neutralization against other betacoronaviruses, including SARS-CoV-1, which was responsible for the 2003 SARS pandemic. This may be formulated for delivery in protein form or for expression in vivo from a suitable nucleic acid molecule (e.g., a viral vector such as AAV).

In another aspect, a mutant human Ace2 (hAce2) soluble decoy protein is provided which comprises a mutated amino acid sequence of SEQ ID NO: 81 or a hAce2 decoy protein at least 95% identical to SEQ ID NO: 81, wherein the mutant hAce2 soluble protein has one or more of the following amino acid residues, based on numbering of SEQ ID NO: 81, (a) R or M at residue 14 (K changed to R or M); (b) V or K at residue (18) (E changed to V or K); (c) P at residue (22) (L changed to P); (d) R at residue (25) (Q changes to R); (e) A at residue (30) (S changed to A); (f) A at residue (42) (V changed to A); (g) I or F at residue (62) (L changes to I or F); (h) D at residue (73) (N changed to D); (i) P at residue (74) (L changed to P); (j) Y at residue (313) (N changed to Y); and/or (k) H at residue (328) (H changed to L), or (1) a mutant hAce2 protein having one or more of the amino acid residues of (a) to (k) fused to a signal peptide and/or an exogenous polypeptide. In certain embodiments, a mutant human Ace2 (hAce2) soluble decoy protein, wherein the mutant hAce2 soluble protein has one or more of the following amino acid residues, based on numbering of SEQ ID NO: 81, (i) A at residue 10 (T changed to A), (ii) G at residue 26 (S changed to G), (ii) G or D at residue 32 (N changed to O or D), (iv) D at residue 44 (N changed to D), (v) K at residue 58 (E changed to K), (vi) R at residue 59 (Q changed to R), (vii) F at residue 62 (L changed to F), (vii) R at residue 64 (Q changed to R), (ix) D or S at residue 73 (N changed to D or S), (x) P at residue 74 (L changed to P), (xi) A at residue 75 (T changed to A), (xii) Y at residue 313 (N changed to Y), (xiii) H at residue (328) (H changed to L), or (xiv) a mutant hAce2 protein having one or more of the amino acid residues of (i) to (xiii) fused to a signal peptide and/or an exogenous polypeptide.

In certain embodiments, the mutant hAce2 soluble decoy protein comprises two or more of the substitutions of (a), (b), (g), (i) and (j). In certain embodiments, the mutant hAce2 soluble decoy protein comprises three or more of the substitutions of (a), (b), (g), (i) and (j), wherein (a) is a Met and/or (g) is a Phe. In certain embodiments, the mutant hAce2 soluble decoy protein comprises an amino acid sequence selected from: (a) all of the substitutions of (a), (b), and (j); (b) all of the substitutions of (a), (b), (g), and (j); (c) an amino acid sequence having the residues of (a), (b), (g), (i) and/or (j); (Variant 4); (d) an amino acid sequence having the residues of (a), (b), (e), (g), (i) and/or (j); (Variant 1); (e) an amino acid sequence having the residues of (a), (b), (e), (g), (j), (i) and/or (k); (Variant 2); (f) an amino acid sequence having the residues of (a), (b), (c), (f), (g), (h), and/or (j) (Variant 3); (g) amino acid sequence having the residues of (a), (b), (d), (i), and/or (j) (Variant 5); (h) amino acid sequence having the residues of (a), (b), (g), (i), and/or (j) (Variant 6). In certain embodiments, the mutant hAce2 soluble decoy protein comprises an amino acid sequence of SEQ ID NO: 29. In certain embodiments, the mutant hAce2 soluble decoy protein comprises an amino acid sequence of SEQ ID NO: 107.

A mutant hAce2 soluble decoy protein may be a fusion protein which comprises a mutated amino acid sequence of SEQ ID NO: 81 or a hAce2 decoy protein at least 95% identical to SEQ ID NO: 81 fused, directly or via a linker, to an immunoglobulin Fc region. In certain embodiments, the Fc region is a human IgG Fc or a human IgM Fc. In certain embodiments, the Fc region is a human IgG2 Fc or a human IgG4 Fc.

In certain embodiments, a mutant hAce2 soluble decoy fusion protein is (a) hAce2-Variant1-IgG4 fusion (SEQ ID NO: 35), (b) hAce2-Variant2-IgG4 fusion (SEQ ID NO: 37), (c) hAce2-Variant3-IgG4 fusion (SEQ ID NO: 39), (d) hAce2-Variant4-IgG4 fusion (SEQ ID NO: 41), (e) hAce2-Variant5-IgG4 fusion (SEQ ID NO: 74 linked to SEQ ID NO: 77), (f) hAce2-Variant6-IgG4 fusion (SEQ ID NO: 75 linked to SEQ ID NO: 77), or (g) or an amino acid sequence at least 95% identical to any of claims (a) to (f). In certain embodiments, a mutant hAce2 soluble decoy fusion protein is (a) hAce2-Variant2-IgG1 fusion (SEQ ID NO: 109), (b) hAce2-Variant2-IgG1 fusion with “GS” linker (SEQ ID NO: 113), (c) hAce2-MR27-Variant-IgG1 fusion (SEQ ID NO: 111), (d) hAce2-MR27-Variant2-IgG1 fusion with “GS” linker (SEQ ID NO: 115), or (e) an amino acid sequence at least 95% identical to any one of (a) to (d). Compositions and therapeutic regimens may contain a single mutant hAce2 soluble protein, fusion protein, or a mixture (cocktail) of these, optionally further in combination with other components.

In a further aspect, a nucleic acid molecule encoding a soluble Ace2 protein operably linked to regulatory sequences which direct expression of the protein thereof in a cell is provided, wherein the soluble Ace2 protein comprises: (a) a fusion protein comprising a signal peptide and the mutant Ace2 soluble protein; or a fusion protein comprising a signal peptide, the mutant Ace2 soluble decoy and an Fc tail. In certain embodiments, the vector (e.g., a plasmid) is used in production of a protein therapeutic. In such instances, the signal peptide may be a non-human or non-mammalian signal peptide suited for the production cell type. In certain other embodiments, the vector is selected for delivery to a patient. In such instances, the signal peptide is suitably a human signal peptide.

In another aspect, a vector having an expression cassette comprising a nucleic acid molecule is provided. In certain embodiments, the hAce2 soluble decoy transgene encoding the hAce2 soluble decoy protein is: (a) hAce2-Variant1 (SEQ ID NO: 27), (b) hAce2-Variant2 (SEQ ID NO: 29), (c) hAce2-Variant3 (SEQ ID NO: 31), (d) hAce2-Variant4 (SEQ ID NO: 33), (e) hAce2-Variant5 (SEQ ID NO: 74), (f) hAce2-Variant6 (SEQ ID NO: 75), (g) hAce2-Variant1-IgG4 fusion (SEQ ID NO: 35), (h) hAce2-Variant2-IgG4 fusion (SEQ ID NO: 37), (i) hAce2-Variant3-IgG4 fusion (SEQ ID NO: 39), (j) hAce2-Variant4-IgG4 fusion (SEQ ID NO: 41), (k) hAce2-Variant5-IgG4 fusion (SEQ ID NO: 74 linked to SEQ ID NO: 77), (1) hAce2-Variant6-IgG4 fusion (SEQ ID NO: 75 linked to SEQ ID NO: 77), or (m) or an amino acid sequence at least 95% identical to any of claims (a) to (1). In certain embodiments, the hAce2 soluble decoy transgene encoding the hAce2 soluble decoy protein is selected from SEQ ID NO: 93, 95, 97, and 99. In certain embodiments, the hAce2 soluble decoy transgene encoding the hAce2 soluble decoy protein is selected from SEQ ID NO: 108, 110, 112, and 114. In certain embodiments, the hAce2 soluble decoy transgene encoding the hAce2 soluble decoy protein has an amino acid sequence selected from SEQ ID NOs: 62 to 71. In certain embodiments, the vector is used for protein expression in a producer host cell (e.g., a plasmid), which may be a mammalian cell, a bacterial cell, a yeast cell, or an insect cell. The cells may be cell lines in cell suspension or another type of cell culture. In other embodiments, the vector is a viral vector for delivery of the transgene and expression of the hAce2 in vivo.

In certain embodiments, a viral vector is used for delivery of the mutant soluble Ace2-protein to the patient. Suitable viral vectors may include, e.g., adenoviruses, vaccinia, lentivirus, or parvoviruses. The vector genome comprises a nucleic acid sequence encoding a mutant hAce2 soluble decoy protein under the control of regulatory control sequences which direct expression of the hAce2 soluble decoy protein, wherein the mutant hAce2 soluble decoy protein comprises a signal peptide and a mutated amino acid sequence of SEQ ID NO: 81 or a hAce2 decoy protein at least 95% identical to SEQ ID NO: 81, wherein the mutant hAce2 soluble protein has one or more of the following amino acid residues, based on numbering of SEQ ID NO: 81, (a) R or M at residue 14 (K changed to R or M); (b) V or K at residue (18) (E changed to V or K); (c) P at residue (22) (L changed to P); (d) R at residue (25) (Q changes to R); (e) A at residue (30) (S changed to A); (f) A at residue (42) (V changed to A); (g) I or F at residue (62) (L changes to I or F); (h) D at residue (73) (N changed to D); (i) P at residue (74) (L changed to P); (j) Y at residue (313) (N changed to Y); and/or (k) H at residue (328) (H changed to L), or (1) a mutant hAce2 protein having one or more of the amino acid residues of (a) to (j) fused to an immunoglobulin Fc region. In certain embodiments, the mutant hAce2 protein comprises two or more of the substitutions of (a), (b), (f), (h) and (i). In other embodiments, the mutant hAce2 protein comprises three or more of the substitutions of (a), (b), (f), (h) and (i), wherein (a) is a Met and/or (f) is a Phe. In certain embodiments, the mutant hAce2 protein comprises an amino acid sequence selected from: (a) all of the substitutions of (a), (b), and (j); (b) all of the substitutions of (a), (b), (g), and (j); (c) an amino acid sequence having the residues of (a), (b), (g), (i) and/or (j); (Variant 4); (d) an amino acid sequence having the residues of (a), (b), (e), (g), (i) and/or (j); (Variant 1); (e) an amino acid sequence having the residues of (a), (b), (e), (g), (j), (i) and/or (k); (Variant 2); (f) an amino acid sequence having the residues of (a), (b), (c), (f), (g), (h), and/or (j) (Variant 3); (g) amino acid sequence having the residues of (a), (b), (d), (i), and/or (j) (Variant 5); (h) amino acid sequence having the residues of (a), (b), (g), (i), and/or (j) (Variant 6). In certain embodiments, the hAce2 soluble decoy is a hAce2 soluble decoy fusion protein further comprising an immunoglobulin Fc region, which may be a human IgG Fc or an IgM Fc. In certain embodiments, the signal peptide is a human signal peptide. In certain embodiments, the signal peptide is the native Ace2 signal. In certain embodiments, the transgene encodes a mutant soluble Ace2 protein which comprises an amino acid of: (a) hAce2-Variant1 (SEQ ID NO: 10), (b) hAce2-Variant2 (SEQ ID NO: 12), (c) hAce2-Variant3 (SEQ ID NO: 14), (d) hAce2-Variant4 (SEQ ID NO: 16), (e) hAce2-Variant5 (SEQ ID NO: 72), (f) hAce2-Variant6 (SEQ ID NO: 73), (g) hAce2-Variant1-IgG4 fusion (SEQ ID NO: 2), (h) hAce2-Variant2-IgG4 fusion (SEQ ID NO: 4), (i) hAce2-Variant3-IgG4 fusion (SEQ ID NO: 6), (j) hAce2-Variant4-IgG4 fusion (SEQ ID NO: 8), (k) hAce2-Variant4-IgG4 fusion (SEQ ID NO: 72 linked to SEQ ID NO: 77), (1) hAce2-Variant4-IgG4 fusion (SEQ ID NO: 73 linked to SEQ ID NO: 77), or (m) or an amino acid sequence at least 95% identical to any of claims (a) to (1). In certain embodiments, the nucleic acid sequence encoding the hAce2 soluble decoy protein is: (a) SEQ ID NO: 9 or a sequence at least about 90% identical thereto encoding hAce2-Variant1 (SEQ ID NO: 10), (b) SEQ ID NO: 11 or a sequence at least about 90% identical thereto encoding hAce2-Variant2 (SEQ ID NO: 12), (c) SEQ ID NO: 13 or a sequence at least about 90% identical thereto encoding hAce2-Variant3 (SEQ ID NO: 14), (d) SEQ ID NO: 15 or a sequence at least about 90% identical thereto encoding hAce2-Variant4 (SEQ ID NO: 16), (e) SEQ ID NO: 1 or a sequence at least about 90% identical thereto encoding hAce2-Variant1-IgG4 fusion (SEQ ID NO: 2), (f) SEQ ID NO: 3 or a sequence at least about 90% identical thereto encoding hAce2-Variant2-IgG4 fusion (SEQ ID NO: 4), (g) SEQ ID NO: 5 or a sequence at least about 90% identical thereto encoding hAce2-Variant3-IgG4 fusion (SEQ ID NO: 6), or (h) SEQ ID NO: 7 or a sequence at least about 90% identical thereto encoding hAce2-Variant4-IgG4 fusion (SEQ ID NO: 8). In certain embodiments, the capsid is an AAV capsid which transduce lung and/or epithelial cells. In other embodiments, the AAV capsid is an AAV9 capsid, a AAVhu68 capsid, an AAV5 capsid, an AAV6 capsid, an AAV6.2 capsid, or an AAVrh91 capsid.

In certain embodiments, a composition comprises one or more of a mutant hAce2 soluble decoy protein, a nucleic acid molecule, a vector, or an rAAV as described herein, optionally in combination with each other. In certain embodiments, the composition is formulated for aerosol administration. In certain embodiments, the composition is formulated for intravenous administration.

In other embodiments, a producer host cell comprising a nucleic acid molecule encoding the protein for expression in vitro and for purification for delivery of a mutant soluble Ace2-protein-based therapeutic. In certain embodiments, a composition comprising a population of mutant hAce2 soluble protein decoy produced using a producer host cell is provided. A composition comprising a population of mutant soluble Ace2-proteins may include up to 5% variation from the sequences provided herein in view of post-translational modifications such as, e.g., glycosylation, oxidation and deamidation. In certain embodiments, there is 0.5% to 5% variation, in other embodiments, there is about 1%, about 2%, about 3%, or about 4% variation. In other embodiments, no detectable variation is observed. Such post-translation modification may be detected by assessed by any suitable technique including, e.g., chromatographic and/or mass spectrometric analysis, or peptide mapping. These detection methods are not a limitation on the present invention.

In certain embodiments, a pharmaceutical composition is provided which comprises at least one mutant hAce2 soluble decoy protein as provided herein and one or more of any of: a pharmaceutically acceptable diluent, a suspending agent, a preservative, and/or a surfactant. In certain embodiments, a pharmaceutical composition is provided which comprises the nucleic acid molecule and one or more of any of: a pharmaceutically acceptable diluent, a suspending agent, a preservative, and/or a surfactant. In certain embodiments, the pharmaceutical composition is formulated for intranasal administration. In certain embodiments, the pharmaceutical composition is formulated for intrapulmonary administration. In certain embodiments, the pharmaceutical composition is formulated for intravenous administration.

### Novel Soluble ACE2 Viral Receptor

The novel soluble Ace2 proteins provided herein, whether delivered as protein therapeutics and/or via vector-mediated delivery, are believed to function as decoys for viruses which utilize an Ace 2 receptor as a mechanism for cellular update. Thus, the soluble Ace2 proteins significantly reduce and/or prevent viral attachment to the native Ace2 receptor in cells. Human respiratory coronaviruses have been associated with sudden acute respiratory syndrome (SARS), including SARS CoV-2 and CoV1, and are putatively associated with the common cold, non-A, B or C hepatitis. So called pre-emergent coronaviruses, such as WIV-CoV, are present in reservoir animal populations, use Ace2 as a receptor, and have a recognized potential to one day cross into humans and cause disease. Moreover, the coronavirus family, also includes a number of non-human viruses such as infectious bronchitis virus (poultry), porcine transmissible gastroenteric virus (pig), porcine hemagglutinatin encephalomyelitis virus (pig), feline infectious peritonitis virus (cat), feline enteric coronavirus (cat), canine coronavirus (dog). Still other infectious viruses utilizing the Ace2 receptor may be treated using the protein and viral vectors described herein. In certain embodiments, these proteins and vector constructs are useful in humans. Additionally or in alternative embodiments, these proteins and vector constructs are useful in non-human mammals, including, e.g., members of the dog and cat families.

Provided herein are novel sequences encoding soluble hACE2 proteins and fusion proteins thereof which comprise an Fc and/or a hinge region designed to increase half-life of the soluble proteins. The soluble hACE2 proteins comprise of a leader sequence, (with reference to the residue numbering of NCBI Reference ID: NP_068576; SEQ ID NO: 25) which comprises the extracellular/zinc binding domain (HEMGH), and a truncation in the C-terminus of the hACE2 protein, including the transmembrane domain (aa 741 to 760) and a C-terminal fragment (amino acids 761 to 805). In the embodiments described below, the protein comprises at least amino acid 18 to 615 of the hACE2 protein of SEQ ID NO: 25 (also referenced to as amino acids 1 to 588 of SEQ ID NO: 42, or amino acids of SEQ ID NO: 81). In certain embodiments, the protein comprises at least amino acid sequence of SEQ ID NO: 107. In other embodiments, optionally, the native leader signal (aa 1-17) may be present to provide a construct comprising amino acids 1 to 615 of the native sequence. In certain embodiments, the protein comprises at least amino acid 18 to 740 of the hACE2 protein of SEQ ID NO: 25 (also referenced to as amino acids 1 to 725 of SEQ ID NO: 42, or amino acids of SEQ ID NO: 83). In certain embodiments, the protein comprises at least amino acid 1 to 615 of the hACE2 protein of SEQ ID NO: 25 (also referenced to amino acids of SEQ ID NO: 80). In certain embodiments, the protein comprises at least amino acid 1 to 740 of the hACE2 protein of SEQ ID NO: 25 (also referenced to amino acids of SEQ ID NO: 82). In certain embodiments, the protein comprises at least amino acid sequence of SEQ ID NO: 105. In certain embodiments, an internal deletion mutant may be selected which deletes the transmembrane domain entirely or functionally (positions 741 to 760). In certain embodiments, the native zinc binding domain is retained. In other embodiments, this domain is rendered non-catalytic by mutating the histidine (H) residues. Such mutations may be at one or both of the H residues. A suitable mutation may be from H to asparagine (N). However, other suitable amino acids may be substituted in order to render this domain non-functional. For example, a suitable alternative mutation in a separate site may be H345L, which renders Ace2 inactive.

In some embodiments, modified hACE2 soluble decoy proteins and/or mutant hACE2 soluble decoy fusion proteins contain a signal sequence encoding a signal peptide which directs the expressed protein within the host cell, wherein the signal peptide (also may be referred to as a leader peptide) is at the amino terminus of the protein. Optionally, the signal sequence may encode a native hACE2 leader or may be from a source exogenous to the hACE2. For example, a sequence encoding a human interleukin-2 signal peptide, or a thrombin signal peptide may be selected. Optionally, another suitable signal peptide, e.g., human or a viral, may be selected. In certain embodiments, the signal peptide may be a non-human or non-mammalian signal peptide suited for the production cell type. In certain other embodiments, the signal peptide is suitably a human signal peptide. In certain embodiments, modified hACE2 soluble decoy proteins and/or modified hACE2 soluble decoy fusion proteins do not contain a signal sequence.

Optionally, a linking sequence may be present between a hACE2 soluble decoy proteins and the Fc region and/or hinge region. The linking sequence may be immediately adjacent to the C-terminus of a hACE2 protein and N-terminus of a second domain, being used to separate two, three or four of the domains or regions. Any suitable sequence of about 1 to 20 amino acids in length may be selected, but it is preferably 3 to 18 amino acids, or about 5 to about 12 amino acids in length. Multiple linking sequences may be present in a single encoded protein sequence, and these may be independently selected. Optionally, if more than one protein is encoded, the vector genome may be designed to contain an F2A or IRES between the coding sequences for the two proteins.

In certain embodiments, the proteins further comprise an Fc tail, i.e., hACE2 soluble decoy fusion proteins. Thus, in certain embodiments, the hACE2 soluble decoy proteins are linked to an Fc fragment of an antibody, preferably a human antibody, such as the Fc fragment of a human IgG antibody, e.g., an IgG1 (Fc1), IgG2 (Fc2), IgG3 (Fc3), or IgG4 (Fc4), IgA or a human IgM antibody. See also, U.S. Provisional Pat. Application No. 63/087,053, filed Oct. 2, 2020, U.S. Provisional Pat. Application No. 63/137,522, filed Jan. 14, 2021, and International Patent Application No. PCT/US2021/53169, filed Oct. 1, 2021 which are incorporated herein by reference in their entireties. The hACE2 soluble decoy protein may be genetically fused to an Fc fragment, either directly or using a linker. See also, U.S. Pat. Provisional Application No. 63/136,497, filed Jan. 12, 2021, and U.S. Provisional Pat. Application No. 63/137,519, filed Jan. 14, 2021, which are incorporated herein by reference. In certain embodiments, the binding molecules are linked to the Fc fragment by a linking sequence comprising from 1 to 100 amino acids, preferably from 1 to 60 amino acids, or from 10 to 60 amino acids. Examples of linkers include, but are not limited to, the linking sequences listed above. In certain embodiments, an immunoglobulin domain is genetically fused to the C-terminus of an Fc fragment. In further embodiments, an immunoglobulin domain or fusion protein is fused to both the N— and the C-terminus of an Fc fragment. See, e.g., WO 2016/124768, which is incorporated by reference herein.

In certain embodiments, the mutant hAce2 soluble decoy is connected to IgG4 Fc (also referred to as Fc4) domain via a flexible “GSG” linker, wherein the flexible “GSG” linker is selected from at least one or more repeats of “GSG”, yielding to total length of the linker of about 3 to about 125 amino acids. In certain embodiments, the mutant hAce2 soluble decoy is directly connected to IgG4 FC domain.

In certain embodiments, the mutant hAce2 soluble decoy is connected to IgG1 Fc (also referred to as Fc1 or IgG1) domain via a flexible “GSG” linker, wherein the flexible “GSG” linker is selected from at least one or more repeats of “GSG”, yielding to total length of the linker of about 3 to about 125 amino acids. In certain embodiments, the mutant hAce2 soluble decoy is directly connected to IgG1 FC domain. In certain embodiments, the mutant hAce2 soluble decoy is connected to IgG1 Fc (also referred to as Fc1 or IgG1) domain via a “GS” linker. In certain embodiments, the mutant hAce2 soluble decoy is connected to IgG1 Fc (also referred to as Fc1 or IgG1) domain via a “EPKSC” linker (SEQ ID NO: 101).

In certain embodiments, the mutant hAce2 soluble compromises amino acid sequence with length of 615 amino acids, based on the numbering of amino acids 1 to 615 of SEQ ID NO: 25 (or an amino acid sequence of SEQ ID NO: 80). In some embodiments, the mutant hAce2 soluble compromises amino acid sequence with length of 588 amino acids, based on the numbering of amino acids 18 to 615 SEQ ID NO: 25 (or an amino acid sequence of SEQ ID NO: 81). In some embodiments, the mutant hAce2 soluble decoy comprises amino acid sequence with length 740 amino acids, based on amino acids 1 to 740 of SEQ ID NO SEQ ID NO: 25 (or an amino acid sequence of SEQ ID NO: 82). In some embodiments, the mutant hAce2 soluble decoy comprises amino acid sequence with length of 723 amino acids, based in the numbering or amino acids 18 to 740 of SEQ ID NO: 25 (or an amino acid sequence of SEQ ID NO: 83). In certain embodiments, the mutant hAce2 soluble decoy with length of 615 amino acids is directly connected to IgG4 Fc domain. In certain embodiments, the mutant hAce2 soluble decoy with length of 615 amino acids is connected to IgG4 Fc domain via a flexible linker, as described herein. In certain embodiments, the mutant hAce2 soluble decoy with length of 588 amino acids is directly connected to IgG4 Fc domain. In certain embodiments, the mutant hAce2 soluble decoy with length of 588 amino acids is connected to IgG4 Fc domain via a flexible linker as described herein. In certain embodiments, the mutant hAce2 soluble decoy with length of 740 amino acids is directly connected to IgG4 Fc domain. In certain embodiments, the mutant hAce2 soluble decoy with length of 723 amino acids is directly connected to IgG4 Fc domain.

In certain embodiments, the mutant hAce2 soluble decoy with length of 615 amino acids is directly connected to IgG1 Fc domain. In certain embodiments, the mutant hAce2 soluble decoy with length of 615 amino acids is connected to IgG1 Fc domain via a flexible linker, as described herein. In certain embodiments, the mutant hAce2 soluble decoy with length of 588 amino acids is directly connected to IgG1 Fc domain. In certain embodiments, the mutant hAce2 soluble decoy with length of 588 amino acids is connected to IgG1 Fc domain via a flexible linker as described herein. In certain embodiments, the mutant hAce2 soluble decoy with length of 740 amino acids is directly connected to IgG1 Fc domain. In certain embodiments, the mutant hAce2 soluble decoy with length of 723 amino acids is directly connected to IgG1 Fc domain.

Provided herein are compositions which are useful for intravenous delivery of a mutant hACE2 soluble decoy and/or hAce2 soluble decoy fusion proteins for therapeutic or prophylactic purpose. In certain embodiments, the mutant hAce2 may be linked via a hinge region to an Fc1 or Fc4 domain encoding an hAce2_Fc1 or hAce2_Fc4 (hAce2-IgG1, hAce2-IgG4 or hAce2-Variant1/2/3/4-IgG) soluble decoy fusion protein (hAce2 fusion). In certain embodiments, the mutant hAce2 may be linked to an IgM domain. In certain embodiments, the mutant hAce2 may be linked to an IgA domain. In some embodiments, the mutant hAce2 contains two inactivating mutations H374N and H378N. In other embodiments, the mutant hAce2 may comprise at least one inactivating mutation of H345L. In certain embodiments, the mutant hAce2 soluble decoy comprise one or more of the mutations: R or M at residue 14 (K changed to R or M), V or K at residue (18) (E changed to V or K), P at residue (22) (L changed to P), R at residue 25 (Q changed to A), A at residue (30) (S changed to A), A at residue (42) (V changed to A), I or F at residue (62) (L changes to I or F), D at residue (73) (N changed to D), P at residue (74) (L changed to P), Y at residue (313) (N changed to Y), and/or H at residue (328) (H changed to L), wherein the numbering is based on SEQ ID NO: 81. In certain embodiments, the mutant hAce2 soluble decoy comprise one or more of the mutations: A at residue 10 (T changed to A), G at residue 26 (S changed to G), G or D at residue 32 (N changed to O or d), D at residue 44 (N changed to D), K at residue 58 (E changed to K), R at residue 59 (Q changed to R), F at residue 62 (L changed to F), R at residue 64 (Q changed to R), D or S at residue 73 (N changed to D or S), P at residue 74 (L changed to P), A at residue 75 (T changed to A), Y at residue 313 (N changed to Y), and/or H at residue (328) (H changed to L), wherein the numbering is based on SEQ ID NO: 81. In certain embodiments, the mutated hAce2 soluble decoy comprise at least one or more of above-mentioned mutations and is linked to an IgG1, an IgG4, IgA or an IgM Fc domain encoding mutant hAce2 soluble decoy fusion protein.

Provided herein also are composition which are useful for intracellular delivery and expression of a soluble form of the mutated soluble ACE2 viral constructs (hAce2) for therapeutic or prophylactic purpose. In certain embodiments, the modified hAce2 may be linked via a hinge region to an Fc1 or Fc4 domain encoding an hAce2_Fc1 or hAce2_Fc4 fusion protein (hAce2 fusion). In certain embodiments, hAce2 contains two inactivating mutations H374N and H378N encoding hAce2_NN. In certain embodiments, the mutated hAce2_NN is linked to a IgG1, IgG4, IgA or IgM Fc domain. In certain embodiments, the mutated hAce2 soluble decoy used for intracellular delivery comprises at least one or more mutations as described herein.

In certain embodiments, the mutant hAce2 soluble decoy and/or the mutant hAce2 soluble decoy fusion protein, as described above, can be engineered into a suitable expression cassette in which the coding sequence for the amino acid sequence is operably linked to a regulatory sequence which direct expression thereof. The expression cassette may optionally contain varied leader sequences preceding a mature protein or protein fragment. In certain embodiments, hAce2 DNA sequence contains a native leader sequence encoding a native signal peptide. In certain embodiments, hAce2 DNA sequence contains an interleukin-2 (IL-2) encoding a IL-2 signal peptide. In certain embodiments, hAce2 DNA sequence contains a thrombin leader sequence (Trb) encoding a Trb signal peptide.

In certain embodiments, the mutant hAce2 soluble decoy fusion protein comprises a native signal peptide, wherein the native signal peptide comprises amino acid residues of 1 to 17 of SEQ ID NO: 25 (or amino acid sequence of SEQ ID NO: SEQ ID NO: 76), the hAce2 soluble decoy comprises amino acid residues of 18 to 615 of SEQ ID NO: 25 (or an amino acid sequence of SEQ ID NO: 81), comprising one or more of mutations as described herein, and an Fc domain (i.e., IgG, IgA, or IgM). In certain embodiments, the nucleic acid sequence encoding the mutant hAce2 soluble decoy fusion protein comprising a native signal peptide is SEQ ID NO: 1 or a sequence at least about 85% identical thereto and encoding an amino acid sequence of SEQ ID NO: 2 (hAce2-Variant1-IgG4). In certain embodiments, the nucleic acid sequence encoding the mutant hAce2 soluble decoy fusion protein comprising a native signal peptide is SEQ ID NO: 3 or a sequence at least about 85% identical thereto and encoding an amino acid sequence of SEQ ID NO: 4 (hAce2-Variant2-IgG4). In certain embodiments, the nucleic acid sequence encoding the mutant hAce2 soluble decoy fusion protein comprising a native signal peptide is SEQ ID NO: 5 or a sequence at least about 85% identical thereto and encoding an amino acid sequence of SEQ ID NO: 6 (hAce2-Variant3-IgG4). In certain embodiments, the nucleic acid sequence encoding the mutant hAce2 soluble decoy fusion protein comprising a native signal peptide is SEQ ID NO: 7 or a sequence at least about 85% identical thereto and encoding an amino acid sequence of SEQ ID NO: 8 (hAce2-Variant4-IgG4). In certain embodiments, the nucleic acid sequence encoding the mutant hAce2 soluble decoy fusion protein comprising a native signal peptide is SEQ ID NO: 93 or a sequence at least about 85% identical thereto and encoding an amino acid sequence of SEQ ID NO: 94 (hAce2-Variant2-IgG1). In certain embodiments, the nucleic acid sequence encoding the mutant hAce2 soluble decoy fusion protein comprising a native signal peptide is SEQ ID NO: 97 or a sequence at least about 85% identical thereto and encoding an amino acid sequence of SEQ ID NO: 98 (hAce2-Variant2-IgG1 with “GS” linker). In certain embodiments, the nucleic acid sequence encoding the mutant hAce2 soluble decoy fusion protein comprising a native signal peptide is SEQ ID NO: 95 or a sequence at least about 85% identical thereto and encoding an amino acid sequence of SEQ ID NO: 96 (hAce2-MR27HL-Variant-IgG1). In certain embodiments, the nucleic acid sequence encoding the mutant hAce2 soluble decoy fusion protein comprising a native signal peptide is SEQ ID NO: 99 or a sequence at least about 85% identical thereto and encoding an amino acid sequence of SEQ ID NO: 100 (hAce2-MR27HL-Variant-IgG1 with “GS” linker).

In certain embodiments, the mutant hAce2 soluble decoy protein comprises a native signal peptide, wherein the native signal peptide comprises amino acid residues of 1 to 17 of SEQ ID NO: 25 (or an amino acid sequence of SEQ ID NO: 76), and the hAce2 soluble decoy comprises amino acid residues of 18 to 615 of SEQ ID NO: 25 (or an amino acid sequence of SEQ ID NO: 81), comprising one or more of listed mutations as described herein. In certain embodiments, the nucleic acid sequence encoding mutant hAce2 soluble decoy protein comprising a native signal peptide is SEQ ID NO: 9 or a sequence at least about 85% identical thereto and encoding an amino acid sequence of SEQ ID NO: 10 (hAce2-Variant1). In certain embodiments, the nucleic acid sequence encoding mutant hAce2 soluble decoy protein comprising a native signal peptide is SEQ ID NO: 11 or a sequence at least about 85% identical thereto and encoding an amino acid sequence of SEQ ID NO: 12 (hAce2-Variant2). In certain embodiments, the nucleic acid sequence encoding mutant hAce2 soluble decoy protein comprising a native signal peptide is SEQ ID NO: 13 or a sequence at least about 85% identical thereto and encoding an amino acid sequence of SEQ ID NO: 14 (hAce2-Variant3). In certain embodiments, the nucleic acid sequence encoding mutant hAce2 soluble decoy protein comprising a native signal peptide is SEQ ID NO: 15 or a sequence at least about 85% identical thereto and encoding an amino acid sequence of SEQ ID NO: 16 (hAce2-Variant4). In certain embodiments, the nucleic acid sequence encoding mutant hAce2 soluble decoy protein comprising a native signal peptide is SEQ ID NO: 104 or a sequence at least about 85% identical thereto and encoding an amino acid sequence of SEQ ID NO: 105 (hAce2-MR27HL-Variant).

In certain embodiments, the mutant hAce2 soluble decoy fusion protein comprises amino acid residues of 18 to 615 of SEQ ID NO: 25 (or an amino acid sequence of SEQ ID NO: 81), comprising one or more of mutations as described herein, and an Fc domain (i.e., IgG, IgA or IgM). In certain embodiments, the nucleic acid sequence encoding the mutant hAce2 soluble decoy fusion protein is SEQ ID NO: 34 or a sequence at least about 85% identical thereto and encoding an amino acid sequence of SEQ ID NO: 35 (hAce2-Variant1-IgG4). In certain embodiments, the nucleic acid sequence encoding the mutant hAce2 soluble decoy fusion protein is SEQ ID NO: 36 or a sequence at least about 85% identical thereto and encoding an amino acid sequence of SEQ ID NO: 37 (hAce2-Variant2-IgG4). In certain embodiments, the nucleic acid sequence encoding the mutant hAce2 soluble decoy fusion protein is SEQ ID NO: 38 or a sequence at least about 85% identical thereto and encoding an amino acid sequence of SEQ ID NO: 39 (hAce2-Variant3-IgG4). In certain embodiments, the nucleic acid sequence encoding the mutant hAce2 soluble decoy fusion protein is SEQ ID NO: 40 or a sequence at least about 85% identical thereto and encoding an amino acid sequence of SEQ ID NO: 41 (hAce2-Variant4-IgG4). In certain embodiments, the nucleic acid sequence encoding the mutant hAce2 soluble decoy fusion protein is SEQ ID NO: 108 or a sequence at least about 85% identical thereto and encoding an amino acid sequence of SEQ ID NO: 109 (hAce2-Variant2-IgG1). In certain embodiments, the nucleic acid sequence encoding the mutant hAce2 soluble decoy fusion protein is SEQ ID NO: 110 or a sequence at least about 85% identical thereto and encoding an amino acid sequence of SEQ ID NO: 111 (hAce2-MR27HL-Variant-IgG1). In certain embodiments, the nucleic acid sequence encoding the mutant hAce2 soluble decoy fusion protein is SEQ ID NO: 112 or a sequence at least about 85% identical thereto and encoding an amino acid sequence of SEQ ID NO: 113 (hAce2-Variant2-IgG1 comprising “GS” linker). In certain embodiments, the nucleic acid sequence encoding the mutant hAce2 soluble decoy fusion protein is SEQ ID NO: 114 or a sequence at least about 85% identical thereto and encoding an amino acid sequence of SEQ ID NO: 115 (hAce2-MR27HL-Variant-IgG1 comprising “GS” linker).

In certain embodiments, the mutant hAce2 soluble decoy protein comprises amino acid residues of 18 to 615 of SEQ ID NO: 25 (or an amino acid sequence of SEQ ID NO: 81), comprising one or more of listed mutations as described herein. In certain embodiments, the nucleic acid sequence encoding mutant hAce2 soluble decoy protein comprising a native signal peptide is SEQ ID NO: 27 or a sequence at least about 85% identical thereto and encoding an amino acid sequence of SEQ ID NO: 28 (hAce2-Variant1). In certain embodiments, the nucleic acid sequence encoding mutant hAce2 soluble decoy protein comprising a native signal peptide is SEQ ID NO: 29 or a sequence at least about 85% identical thereto and encoding an amino acid sequence of SEQ ID NO: 30 (hAce2-Variant2). In certain embodiments, the nucleic acid sequence encoding mutant hAce2 soluble decoy protein comprising a native signal peptide is SEQ ID NO: 31 or a sequence at least about 85% identical thereto and encoding an amino acid sequence of SEQ ID NO: 32 (hAce2-Variant3). In certain embodiments, the nucleic acid sequence encoding mutant hAce2 soluble decoy protein comprising a native signal peptide is SEQ ID NO: 33 or a sequence at least about 85% identical thereto and encoding an amino acid sequence of SEQ ID NO: 34 (hAce2-Variant4). In certain embodiments, the nucleic acid sequence encoding mutant hAce2 soluble decoy protein comprising a native signal peptide is SEQ ID NO: 106 or a sequence at least about 85% identical thereto and encoding an amino acid sequence of SEQ ID NO: 107 (hAce2-MR27-Variant).

In certain embodiments, the mutant hAce2 soluble decoy fusion protein comprises an engineered hAce2 decoy protein connected via a linker to a IgG1 Fc domain. In certain embodiments, the hAce2 soluble decoy fusion protein is selected from: (a) hAce2-Variant2-IgG1 fusion (aa 18 to 847 of SEQ ID NO: 94 or an amino acid sequence of SEQ ID NO: 109), (b) hAce2-Variant2-IgG1 fusion with “GS” linker (aa 18 to 844 of SEQ ID NO: 98 or an amino acid sequence of SEQ ID NO: 113), (c) hAce2-MR27-Variant-IgG1 fusion (aa 18 to 847 of SEQ ID NO: 96 or an amino acid sequence of SEQ ID NO: 111), (d) hAce2-MR27-Variant2-IgG1 fusion with “GS” linker (aa 18 to 844 of SEQ ID NO: 100 or an amino acid sequence of SEQ ID NO: 115), or (e) an amino acid sequence at least 95% identical to any one of (a) to (d).

In certain embodiments, the mutant hAce2 soluble decoy protein comprises amino acid residues of 18 to 615 of SEQ ID NO: 25 (or SEQ ID NO: 81), comprising one or more of listed mutations as described in Table 1 below. In some embodiments, the mutant hAce2 soluble decoy protein comprises amino acid residues of 1 to 615 of SEQ ID NO: 25 (or an amino acid sequence of SEQ ID NO: 80), comprising one or more of listed mutations as described in Table 1 below.

TABLE 1

| Clone/ Variant # | Genotype^{∗} | SEQ ID NOs (with signal/leader peptide) | SEQ ID NOs (without signal/leader peptide) |
|---|---|---|---|
| 44 | 27A/43G/49D/79F/90D/330Y | 52 | 62 |
| 12 | 27A/49D/61D/75K/91P/330Y | 53 | 63 |
| 16 | 27A/43G/61D/81R/91P/330Y | 54 | 64 |
| 17 | 27A/49D/61D/79F/90S/330Y | 55 | 65 |
| 33 | 27A/43G/49D/79F/91P/330Y | 56 | 66 |
| 24 | 27A/43G/49D/61D/76R/330Y | 57 | 67 |
| 27 | 43G/49D/79F/90D/330Y | 58 | 68 |
| 43 | 27A/43G/61D/75K/90D/330Y | 59 | 69 |
| 35 | 27A/43G/61D/79F/90S/330Y | 60 | 70 |
| 39 | 27A/49D/61D/81R/92A/330Y | 61 | 71 |

^{∗}Amino Acid (AA) position numbering is based on SEQ ID NO: 25.

In certain embodiments, an amino acid sequence for the mutant hAce2 soluble decoy (with signal or leader peptide) is selected from SEQ ID NO: 52 to 61. In certain embodiments, an amino acid sequence for the mutant hAce2 soluble decoy (without signal or leader peptide) is selected from SEQ ID NO: 62 to 71. In certain embodiments, an amino acid sequence for the mutant hAce2 soluble decoy (with signal or leader peptide) is selected from SEQ ID NO: 10, 12, 14, 16, 72 or 73. In certain embodiments, an amino acid sequence for the mutant hAce2 soluble decoy (without signal or leader peptide) is selected from SEQ ID NO: 27, 29, 31, 33, 74, or 75. In certain embodiments, the mutant hAce2 soluble decoy (with signal or leader peptide) is linked to the IgG1, IgG4, IgA, or IgM Fc domain. In certain embodiments, the mutant hAce2 soluble decoy (without signal or leader peptide) is linked to the IgG1, IgG4, IgA or IgM Fc domain.

In certain embodiments, hAce2-Variant1 soluble decoy protein has an amino acid sequence of SEQ ID NO: 10, wherein a soluble decoy fusion protein comprising a native signal peptide (aa 1 to 17 of SEQ ID NO: 10 or an amino acid sequence of SEQ ID NO: 76), and a soluble hAce2 protein fragment, i.e., soluble decoy (aa 18 to 615 of SEQ ID NO: 10 or an amino acid sequence of SEQ ID NO: 27), or a sequence at least about 95% identical thereto. In certain embodiments, hAce2-Variant2 soluble decoy protein has an amino acid sequence of SEQ ID NO: 12, wherein a soluble decoy fusion protein comprising a native signal peptide (aa 1 to 17 of SEQ ID NO: 12 or an amino acid sequence of SEQ ID NO: 76), and a soluble hAce2 protein fragment, i.e., soluble decoy (aa 18 to 615 of SEQ ID NO: 12 or an amino acid sequence of SEQ ID NO: 29), or a sequence at least about 95% identical thereto. In certain embodiments, hAce2-Variant3 soluble decoy protein has an amino acid sequence of SEQ ID NO: 14, wherein a soluble decoy fusion protein comprising a native signal peptide (aa 1 to 17 of SEQ ID NO: 14 or an amino acid sequence of SEQ ID NO: 76), and a soluble hAce2 protein fragment, i.e., soluble decoy (aa 18 to 615 of SEQ ID NO: 14 or an amino acid sequence of SEQ ID NO: 31), or a sequence at least about 95% identical thereto. In certain embodiments, hAce2-Variant4 soluble decoy protein has an amino acid sequence of SEQ ID NO: 16, wherein a soluble decoy fusion protein comprising a native signal peptide (aa 1 to 17 of SEQ ID NO: 16 or an amino acid sequence of SEQ ID NO: 76), and a soluble hAce2 protein fragment, i.e., soluble decoy (aa 18 to 615 of SEQ ID NO: 16 or an amino acid sequence of SEQ ID NO: 33), or a sequence at least about 95% identical thereto. In certain embodiments, hAce2-Variant5 soluble decoy protein has an amino acid sequence of SEQ ID NO: 72, wherein a soluble decoy fusion protein comprising a native signal peptide (aa 1 to 17 of SEQ ID NO: 72 or an amino acid sequence of SEQ ID NO: 76), and a soluble hAce2 protein fragment, i.e., soluble decoy (aa 18 to 615 of SEQ ID NO: 72 or an amino acid sequence of SEQ ID NO: 74), or a sequence at least about 95% identical thereto. In certain embodiments, hAce2-Variant6 soluble decoy protein has an amino acid sequence of SEQ ID NO: 73, wherein a soluble decoy fusion protein comprising a native signal peptide (aa 1 to 17 of SEQ ID NO: 73 or an amino acid sequence of SEQ ID NO: 76), and a soluble hAce2 protein fragment, i.e., soluble decoy (aa 18 to 615 of SEQ ID NO: 73 or an amino acid sequence of SEQ ID NO: 75), or a sequence at least about 95% identical thereto. In certain embodiments, hAce2-MR27-Variant soluble decoy protein has an amino acid sequence of SEQ ID NO: 105, wherein a soluble decoy fusion protein comprising a native signal peptide (aa 1 to 17 of SEQ ID NO: 105 or an amino acid sequence of SEQ ID NO: 76), and a soluble hAce2 protein fragment, i.e., soluble decoy (aa 18 to 615 of SEQ ID NO: 105 or an amino acid sequence of SEQ ID NO: 107), or a sequence at least about 95% identical thereto.

In certain embodiments, hAce2-Variant1-IgG4 soluble decoy fusion protein has an amino acid sequence of SEQ ID NO: 2, wherein a soluble decoy fusion protein comprising a native signal peptide (aa 1 to 17 of SEQ ID NO: 2 or an amino acid sequence of SEQ ID NO: 76), a soluble hAce2 protein fragment, i.e., soluble decoy (aa 18 to 615 of SEQ ID NO: 2 or an amino acid sequence of SEQ ID NO: 35), and a human Fc immunoglobulin fragment IgG4 (aa 616 to 844 of SEQ ID NO: 2 or an amino acid sequence of SEQ ID NO: 77), or a sequence at least about 95% identical thereto. In certain embodiments, hAce2-Variant2-IgG4 soluble decoy fusion protein has an amino acid sequence of SEQ ID NO: 4, wherein a soluble decoy fusion protein comprising a native signal peptide (aa 1 to 17 of SEQ ID NO: 4 or an amino acid sequence of SEQ ID NO: 76), a soluble hAce2 protein fragment, i.e., soluble decoy (aa 18 to 615 of SEQ ID NO: 4 or an amino acid sequence of SEQ ID NO: 37), and a human Fc immunoglobulin fragment IgG4 (aa 616 to 844 of SEQ ID NO: 4 or an amino acid sequence of SEQ ID NO: 77), or a sequence at least about 95% identical thereto. In certain embodiments, hAce2-Variant3-IgG4 soluble decoy fusion protein has an amino acid sequence of SEQ ID NO: 6, wherein a soluble decoy fusion protein comprising a native signal peptide (aa 1 to 17 of SEQ ID NO: 6 or an amino acid sequence of SEQ ID NO: 76), a soluble hAce2 protein fragment, i.e., soluble decoy (aa 18 to 615 of SEQ ID NO: 6 or an amino acid sequence of SEQ ID NO: 39), and a human Fc immunoglobulin fragment IgG4 (aa 616 to 844 of SEQ ID NO: 6 or an amino acid sequence of SEQ ID NO: 77), or a sequence at least about 95% identical thereto. In certain embodiments, hAce2-Variant4-IgG4 soluble decoy fusion protein has an amino acid sequence of SEQ ID NO: 8, wherein a soluble decoy fusion protein comprising a native signal peptide (aa 1 to 17 of SEQ ID NO: 8 or an amino acid sequence of SEQ ID NO: 76), a soluble hAce2 protein fragment, i.e., soluble decoy (aa 18 to 615 of SEQ ID NO: 8 or an amino acid sequence of SEQ ID NO: 41), and a human Fc immunoglobulin fragment IgG4 (aa 616 to 844 of SEQ ID NO: 8 or an amino acid sequence of SEQ ID NO: 77), or a sequence at least about 95% identical thereto. In certain embodiments, hAce2-Variant5-IgG4 soluble decoy fusion protein has an amino acid sequence, wherein a soluble decoy fusion protein a soluble hAce2 protein fragment, i.e., soluble decoy is an amino acid sequence of SEQ ID NO: 72), and is linked to a human Fc immunoglobulin fragment IgG4 (SEQ ID NO: 77), or a sequence at least about 95% identical thereto. In certain embodiments, hAce2-Variant6-IgG4 soluble decoy fusion protein has an amino acid sequence, wherein a soluble decoy fusion protein a soluble hAce2 protein fragment, i.e., soluble decoy is an amino acid sequence of SEQ ID NO: 73), and is linked to a human Fc immunoglobulin fragment IgG4 (SEQ ID NO: 77), or a sequence at least about 95% identical thereto. In certain embodiments, a soluble decoy fusion protein comprises a soluble hAce2 protein fragment selected from hAce2-Variant1, 2, 3, 4, 5, 6, or MR27HL, wherein the soluble decoy is further linked to a human Fc immunoglobulin fragment IgG1 (SEQ ID NO: 103).

In certain embodiments, a hAce2-Variant2-IgG1 soluble decoy fusion protein has an amino acid sequence of SEQ ID NO: 94, wherein a soluble decoy fusion protein comprising a native signal peptide (aa 1 to 17 of SEQ ID NO: 94 or an amino acid sequence of SEQ ID NO: 76), a soluble hAce2 protein fragment, i.e., soluble decoy (aa 18 to 615 of SEQ ID NO: 94 or an amino acid sequence of SEQ ID NO: 29), a linker “EPKSC” (aa 616 to 620 of SEQ ID NO: 94 or an amino acid sequence if SEQ ID NO: 101), and a human Fc immunoglobulin fragment IgG1 (aa 621 to 847 of SEQ ID NO: 94 or an amino acid sequence of SEQ ID NO: 103), or a sequence at least about 95% identical thereto.

In certain embodiments, a hAce2-Variant2-IgG1 soluble decoy fusion protein has an amino acid sequence of SEQ ID NO: 98, wherein a soluble decoy fusion protein comprising a native signal peptide (aa 1 to 17 of SEQ ID NO: 98 or an amino acid sequence of SEQ ID NO: 76), a soluble hAce2 protein fragment, i.e., soluble decoy (aa 18 to 615 of SEQ ID NO: 98 or an amino acid sequence of SEQ ID NO: 29), a linker “GS” (aa 616 to 617 of SEQ ID NO: 98), and a human Fc immunoglobulin fragment IgG1 (aa 618 to 844 of SEQ ID NO: 98 or an amino acid sequence of SEQ ID NO: 103), or a sequence at least about 95% identical thereto.

In certain embodiments, a hAce2-MR27HL-Variant-IgG1 soluble decoy fusion protein has an amino acid sequence of SEQ ID NO: 96, wherein a soluble decoy fusion protein comprising a native signal peptide (aa 1 to 17 of SEQ ID NO: 96 or an amino acid sequence of SEQ ID NO: 76), a soluble hAce2 protein fragment, i.e., soluble decoy (aa 18 to 615 of SEQ ID NO: 96 or an amino acid sequence of SEQ ID NO: 107), a linker “EPKSC” (aa 616 to 620 of SEQ ID NO: 96 or an amino acid sequence of SEQ ID NO: 101), and a human Fc immunoglobulin fragment IgG1 (aa 619 to 844 of SEQ ID NO: 96 or an amino acid sequence of SEQ ID NO: 103), or a sequence at least about 95% identical thereto.

In certain embodiments, a hAce2-MR27HL-Variant-IgG1 soluble decoy fusion protein has an amino acid sequence of SEQ ID NO: 100, wherein a soluble decoy fusion protein comprising a native signal peptide (aa 1 to 17 of SEQ ID NO: 100 or an amino acid sequence of SEQ ID NO: 76), a soluble hAce2 protein fragment, i.e., soluble decoy (aa 18 to 615 of SEQ ID NO: 100 or an amino acid sequence of SEQ ID NO: 107), a linker “GS” (aa 616 to 617 of SEQ ID NO: 100), and a human Fc immunoglobulin fragment IgG1 (aa 619 to 844 of SEQ ID NO: 100 or an amino acid sequence of SEQ ID NO: 103), or a sequence at least about 95% identical thereto.

In certain embodiments, a hAce2-Variant1 soluble decoy protein has an amino acid sequence of SEQ ID NO: 27, or a sequence at least about 95% identical thereto. In certain embodiments, hAce2-Variant2 soluble decoy protein has an amino acid sequence of SEQ ID NO: 29, or a sequence at least about 95% identical thereto. In certain embodiments, hAce2-Variant3 soluble decoy protein has an amino acid sequence of SEQ ID NO:31, or a sequence at least about 95% identical thereto. In certain embodiments, hAce2-Variant4 soluble decoy protein has an amino acid sequence of SEQ ID NO: 33, or a sequence at least about 95% identical thereto. In certain embodiments, a hAce2-MR27HL-Variant soluble decoy protein has an amino acid sequence of SEQ ID NO: 107, or a sequence at least about 95% identical thereto.

In certain embodiments, a hAce2-Variant1-IgG4 soluble decoy fusion protein has an amino acid sequence of SEQ ID NO: 35, wherein a soluble decoy fusion protein a soluble hAce2 protein fragment, i.e., soluble decoy (aa 1 to 598 of SEQ ID NO: 35 or an amino acid sequence of SEQ ID NO: 27), and a human Fc immunoglobulin fragment IgG4 (aa 599 to 827 of SEQ ID NO: 35 or an amino acid sequence of SEQ ID NO: 77), or a sequence at least about 95% identical thereto. In certain embodiments, a hAce2-Variant2-IgG4 soluble decoy fusion protein has an amino acid sequence of SEQ ID NO: 37, wherein a soluble decoy fusion protein a soluble hAce2 protein fragment, i.e., soluble decoy (aa 1 to 598 of SEQ ID NO: 37 or an amino acid sequence of SEQ ID NO: 29), and a human Fc immunoglobulin fragment IgG4 (aa 599 to 827 of SEQ ID NO: 37 or an amino acid sequence of SEQ ID NO: 77), or a sequence at least about 95% identical thereto. In certain embodiments, a hAce2-Variant3-IgG4 soluble decoy fusion protein has an amino acid sequence of SEQ ID NO: 39, wherein a soluble decoy fusion protein a soluble hAce2 protein fragment, i.e., soluble decoy (aa 1 to 598 of SEQ ID NO: 39 or an amino acid sequence of SEQ ID NO: 31), and a human Fc immunoglobulin fragment IgG4 (aa 599 to 827 of SEQ ID NO: 39 or an amino acid sequence of SEQ ID NO: 77), or a sequence at least about 95% identical thereto. In certain embodiments, a hAce2-Variant4-IgG4 soluble decoy fusion protein has an amino acid sequence of SEQ ID NO: 41, wherein a soluble decoy fusion protein a soluble hAce2 protein fragment, i.e., soluble decoy (aa 1 to 598 of SEQ ID NO: 41 or an amino acid sequence of SEQ ID NO: 33), and a human Fc immunoglobulin fragment IgG4 (aa 599 to 827 of SEQ ID NO: 41 or an amino acid sequence of SEQ ID NO: 77), or a sequence at least about 95% identical thereto. In certain embodiments, a hAce2-Variant5-IgG4 soluble decoy fusion protein has an amino acid sequence, wherein a soluble decoy fusion protein a soluble hAce2 protein fragment, i.e., soluble decoy is an amino acid sequence of SEQ ID NO: 74), and is linked to a human Fc immunoglobulin fragment IgG4 (SEQ ID NO: 77), or a sequence at least about 95% identical thereto. In certain embodiments, a hAce2-Variant6-IgG4 soluble decoy fusion protein has an amino acid sequence, wherein a soluble decoy fusion protein a soluble hAce2 protein fragment, i.e., soluble decoy comprises an amino acid sequence of SEQ ID NO: 75), and is linked to a human Fc immunoglobulin fragment IgG4 (SEQ ID NO: 77), or a sequence at least about 95% identical thereto.

In certain embodiments, a hAce2-Variant2-IgG1 soluble decoy fusion protein has an amino acid sequence, wherein a soluble decoy fusion protein a soluble hAce2 protein fragment, i.e., soluble decoy comprises an amino acid sequence of SEQ ID NO: 29), and is linked to a human Fc immunoglobulin fragment IgG1 (SEQ ID NO: 103), or a sequence at least about 95% identical thereto. In certain embodiments, a hAce2-MR27HL-Variant-IgG1 soluble decoy fusion protein has an amino acid sequence, wherein a soluble decoy fusion protein a soluble hAce2 protein fragment, i.e., soluble decoy comprises an amino acid sequence of SEQ ID NO: 107), and is linked to a human Fc immunoglobulin fragment IgG1 (SEQ ID NO: 103), or a sequence at least about 95% identical thereto.

In certain embodiments, an expression cassette or a vector genome comprises at least one mutated soluble hAce2 decoy protein (mutated hAce2 soluble decoy and/or mutated hAce2 soluble decoy fusion proteins) is provided herein. In certain embodiments, an expression cassette or a vector genome comprises a single mutated soluble hAce2 decoy (mutated hAce2 soluble decoy and/or mutated hAce2 soluble decoy fusion proteins) protein as described herein and a second coding sequence encoding a different gene product.

Examples of suitable expression vectors include, without limitation, plasmids and viral vectors such as herpes viruses, retroviruses, vaccinia viruses, attenuated vaccinia viruses, canary pox viruses, adenoviruses, parvoviruses (e.g., bocavirus or adeno-associated viruses, lentiviruses and herpes viruses, among others. In certain embodiments, the vectors are selected for delivery of an expression cassette encoding the protein to a patient and in vivo expression of the protein. In other embodiments, a selected protein(s) (e.g., MR27HL fusion proteins) is suited for expression in vitro and delivery to a patient as a protein-based therapeutic.

### Recombinant Protein Production

In certain embodiments, a nucleic acid molecule (e.g., a plasmid) is used for in vitro expression of a recombinant protein to produce a recombinant fusion protein therapeutic. In such instances, the signal peptide may be a non-human or non-mammalian signal peptide suited for the production cell type. In certain other embodiments, the vector is selected for expression in a human cell. In such instances, the signal peptide is suitably a human signal peptide. In certain embodiments, the vector comprises CMV (cytomegalovirus), EF1 (elongation factor-1 alpha), or EEF2 (Eukaryotic Translation Elongation Factor 2) promoter. In certain embodiments, the protein production is with co-transfection of glycosylation enzymes to promote sialyation of decoy proteins produced.

The transgene and other nucleic acids may be contained within a production vector. For production of a protein-based therapeutic, a vector may include, but is not limited to, any plasmid, phagemid, F-factor, virus, cosmid, or phage in double or single stranded linear or circular form which may or may not be self-transmissible or mobilizable, or non-therapeutic because it is designed for in vitro protein production and may contain elements not suitable for delivery to a patient. The production vector can also transform a prokaryotic or eukaryotic host either by integration into the cellular genome or exist extra-chromosomally (e.g., autonomous replicating plasmid with an origin of replication).

In vitro expression systems that may be used for small- or large-scale production of the hAce2 proteins, polypeptides or peptide fragments of the invention include, but are not limited to, cells or microorganisms that are transformed with a recombinant nucleic acid construct that contains a nucleic acid segment of the invention. Examples of recombinant nucleic acid constructs may include bacteriophage DNA, plasmid DNA, cosmid DNA, or viral expression vectors. Examples of cells and microorganisms that may be transformed include bacteria (for example, E. coli or B. subtilis); yeast (for example, Saccharomyces and Pichia); insect cell systems (for example, baculovirus); plant cell systems; or mammalian cell systems (for example, COS, Chinese Hamster Ovary (CHO), BHK, 293, VERO, HeLa, MDCK, W138, and NIH 3T3 cells). Also useful as host cells are primary or secondary cells obtained directly from a mammal that are transfected with a plasmid vector or infected with a viral vector. Synthetic methods may also be used to produce polypeptides and peptide fragments of the invention. Such methods are known and have been reported. Merrifield, Science, 85:2149 (1963).

In certain embodiments, the production cell lune is banked, thawed, and cultured in chemically defined media without any components of animal origin. In certain embodiments, the production cell line is a suspension cell line, wherein the cells are grown in suspension mode. In certain embodiments, the protein production comprises process in a single use stir tank-controlled bioreactor in fed batch mode, wherein the protein is secreted into the cell culture and separated from cell components as a part of harvest. In certain embodiments, the harvest is performed using continuous centrifugation followed by depth filtration. In certain embodiments, the harvest is performed with depth filtration. In certain embodiments, the filtered product is further treated with stability enhancing components. In certain embodiments, the filtered product is further concentrated and buffer-exchanged using tangential flow filtration.

In certain embodiments, the recombinant protein is produced using a production cell line as described herein, wherein the production process comprises a chromatography step comprising binding and elution steps; a neutralization step; a filtration step using 0.2-micron depth filter; a further filtration step using virus removal nanofilter; and a further filtration step (e.g., using a 0.2 micron filter). This may optionally further comprise at least one or more further chromatography steps; and/or solvent detergent viral inactivation step before or after chromatography steps. In certain embodiments, the at least one or more chromatography steps are selected from ion exchange membrane chromatography, an anion exchange resin chromatography, a cation exchange resin chromatography, a hydrophobic interaction chromatography (HIC) resin chromatography, a mixed mode of anion and HIC chromatography, or a mixed mode of cation and HIC resin column chromatography. The protein product is then formulated into the final formulation buffer by initial concentration to a target concentration, a 10x buffer exchange, followed by a final concentration and filter flush. The protein product is examined for bioburden, endotoxins and related contaminants (i.e., host cell proteins), residual DNA, and/or residual affinity leached ligand throughout protein production process and in the final formulation. In certain embodiments, the protein product comprises a final spike of formulation component and filtration step to produce the bulk drug (protein) substance.

In certain embodiments, the formulation buffer for the protein drug product is a buffered saline. One suitable buffered saline is a phosphate buffer saline with 0.001% poloxamer 188 (137 mM Sodium Chloride, 2.7 mM Potassium Chloride, 10 mM Sodium Phosphate Dibasic, 1.8 mM Potassium Phosphate Monobasic, 0.001% Poloxamer 188, pH 7.3-7.5). Other suitable buffers may be selected. In certain embodiments, the final protein product is stored in -80°. In certain embodiments, the final protein product is formulated in a suitable buffer for stability and stored, e.g., at about 2° C. to about 8° C. In certain embodiments, the final protein product is formulated in a buffer suitable for lyophilization, which is further suitable for storage at room temperature.

In certain embodiments, modified hAce2 soluble decoy protein and/or hAce2 soluble decoy fusion protein as described herein may comprise any suitable number of additional amino acid residues, i.e., at least one additional amino acid residue. The additional amino acid residues may be added in order to improve or simplify production, purification and/or detection of a protein. For example, additional amino acid residue may include an addition of a cysteine residue at either the amino or carboxy terminus of a protein, which may provide a “tag” for purification or detection of the protein, i.e., a His6 tag, a c-myc tag, a FLAG tag for interaction with antibodies specific to the tag or immobilized metal affinity chromatography (IMAC) in the case of the hexa-histidine tag.

In certain embodiments, the nucleic acid molecule selected for in vitro production (e.g., a production vector) comprises a transgene encoding a modified hAce2 soluble decoy protein and/or hAce2 soluble decoy fusion protein as described herein under regulatory control of sequences designed to optimize in vitro production levels. In certain embodiments, the transgene comprises a nucleic acid sequence of SEQ ID NO: 1 or a sequence at least about 95% identical thereto, encoding an amino acid sequence of SEQ ID NO: 2. In certain embodiments, the transgene comprises a nucleic acid sequence of SEQ ID NO: 3 or a sequence at least about 95% identical thereto, encoding an amino acid sequence of SEQ ID NO: 4. In certain embodiments, the transgene comprises a nucleic acid sequence of SEQ ID NO: 95 or a sequence at least about 95% identical thereto, encoding an amino acid sequence of SEQ ID NO: 6 In certain embodiments, the transgene comprises a nucleic acid sequence of SEQ ID NO: 7 or a sequence at least about 95% identical thereto, encoding an amino acid sequence of SEQ ID NO: 8. In certain embodiments, the transgene comprises a nucleic acid sequence of SEQ ID NO: 93 or a sequence at least about 95% identical thereto, encoding an amino acid sequence of SEQ ID NO: 94. In certain embodiments, the transgene comprises a nucleic acid sequence of SEQ ID NO: 95 or a sequence at least about 95% identical thereto, encoding an amino acid sequence of SEQ ID NO: 96. In certain embodiments, the transgene comprises a nucleic acid sequence of SEQ ID NO: 97 or a sequence at least about 95% identical thereto, encoding an amino acid sequence of SEQ ID NO: 98. In certain embodiments, the transgene comprises a nucleic acid sequence of SEQ ID NO: 99 or a sequence at least about 95% identical thereto, encoding an amino acid sequence of SEQ ID NO: 100. The examples below demonstrate that the protein hAce2-MR27HL-Variant-IgG1 (amino acid sequence of SEQ ID NOs: 96 and 100 (comprising leader sequence); SEQ ID NOs: 111 and 115) showed increased expression in vitro while maintaining similar or same potency against SARS-CoV2 as in comparison to hAce2-Variant2-IgG1. Thus, the hAce2-MR27HL fusion proteins are believed to be particularly well suited for in vitro production and delivery as a protein-based therapeutic.

### Vector-Mediated Gene Delivery

In certain embodiments, vectors are particularly well suited for prophylactic expression of a soluble protein(s) provided herein. Optionally, such vectors may be used in combination with protein therapeutics which confer therapeutic effect or passive immunization. Examples of suitable expression vectors include, without limitation, plasmids and viral vectors such as herpes viruses, retroviruses, vaccinia viruses, attenuated vaccinia viruses, canary pox viruses, adenoviruses, parvoviruses (e.g., bocavirus or adeno-associated viruses, lentiviruses and herpes viruses, among others.

### Capsids

In certain embodiments, a parvovirus capsid is used to generate a recombinant vector suitable for delivery to a patient. In certain embodiments, the parvovirus capsid is selected from adeno-associated viruses which target nasal epithelial cells, nasopharynx cells, lung cells, or another target tissue which expresses the soluble protein. For example, capsids from Clade F AAV such as AAVhu68 or AAV9 may be selected. Methods of generating vectors having the AAV9 capsid or AAVhu68 capsid, and/or chimeric capsids derived from AAV9 have been described. See, e.g., US 7,906,111, which is incorporated by reference herein. Other AAV serotypes which transduce nasal cells or another suitable target (e.g., muscle or lung) may be selected as sources for capsids of AAV viral vectors including, e.g., AAV1, AAV2, AAV3, AAV4, AAV5, AAV6, AAV6.2, AAV7, AAV8, AAV9, AAVhu68, AAVrh91, rh10, AAVrh64R1, AAVrh64R2, rh8 (See, e.g., U.S. Published Pat. Application No. 2007-0036760-A1; U.S. Published Pat. Application No. 2009-0197338-A1; and EP 1310571). See, e.g., U.S. 7,906,111, which is incorporated by reference herein. See also, WO 2003/042397 (AAV7 and other simian AAV), U.S. Pat. 7790449 and U.S. Pat. 7282199 (AAV8), WO 2005/033321 (AAV9), and WO 2006/110689, or yet to be discovered, or a recombinant AAV based thereon, may be used as a source for the AAV capsid. See, e.g., WO 2020/223232 A1 (AAV rh90), WO 2020/223231 A1 (AAV rh91), and WO 2020/223236 A1 (AAV rh92, AAV rh93, AAVrh91.93), which are incorporated herein by reference in its entirety. In certain embodiments, a modified rAAV capsid is selected, wherein the capsid comprises at least one exogenous peptide which is a targeting motif (i.e., nasal epithelial cells, nasopharynx cells, and/or lung cells). See also, U.S. Provisional Pat. Application No. 63/119,863, filed Dec. 1, 2020, and International Patent Application No. PCT/US21/61312, filed Dec. 2, 2021, which are incorporated herein by reference. These documents also describe other AAV which may be selected for generating AAV and are incorporated by reference. In some embodiments, an AAV capsid (cap) for use in the viral vector can be generated by mutagenesis (i.e., by insertions, deletions, or substitutions) of one of the aforementioned AAV caps or its encoding nucleic acid. In some embodiments, the AAV capsid is chimeric, comprising domains from two or three or four or more of the aforementioned AAV capsid proteins. In some embodiments, the AAV capsid is a mosaic of Vpl, Vp2, and Vp3 monomers from two or three different AAVs or recombinant AAVs. In some embodiments, an rAAV composition comprises more than one of the aforementioned caps.

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp 1 amino acid sequence. The Neighbor-Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades. See, e.g., G Gao, et al, J Virol, 2004 Jun; 78(12): 6381-6388, which identifies Clades A, B, C, D, E and F, and provides nucleic acid sequences of novel AAV, GenBank Accession Numbers AY530553 to AY530629. See, also, WO 2005/033321.

As used herein, an “AAV9 capsid” is a self-assembled AAV capsid composed of multiple AAV9 vp proteins. The AAV9 vp proteins are typically expressed as alternative splice variants encoded by a nucleic acid sequence which encodes the vp1 amino acid sequence of GenBank accession: AAS99264. These splice variants result in proteins of different length. In certain embodiments, “AAV9 capsid” includes an AAV having an amino acid sequence which is 99% identical to AAS99264 or 99% identical thereto. See, also US 7,906,111 and WO 2005/033321. As used herein “AAV9 variants” include those described in, e.g., WO2016/049230, US 8,927,514, US 2015/0344911, and US 8,734,809.

Also provided herein are the use of novel capsids based on the AAV9 insertional library provided in the examples below.

A rAAVhu68 is composed of an AAVhu68 capsid and a vector genome. An AAVhu68 capsid is an assembly of a heterogenous population of vp1, a heterogenous population of vp2, and a heterogenous population of vp3 proteins. As used herein when used to refer to vp capsid proteins, the term “heterogenous” or any grammatical variation thereof, refers to a population consisting of elements that are not the same, for example, having vp1, vp2 or vp3 monomers (proteins) with different modified amino acid sequences. See, also, PCT/US2018/019992, WO 2018/160582, entitled “Adeno-Associated Virus (AAV) Clade F Vector and Uses Therefor”, and which are incorporated herein by reference in its entirety. In certain embodiments, the AAVhu68 capsid coding sequence is a coding sequence which is useful in manufacturing of recombinant AAV (rAAV) for generating higher yields of recombinant AAV having AAVhu68 capsids. See also, U.S. Provisional Pat. Application No. 63/093,275, filed Oct. 18, 2020, and International Patent Application No. PCTPCT/US2021/055436, filed Oct. 18, 2021, which are incorporated herein by reference in their entireties.

For use in producing an AAV viral vector (e.g., a recombinant (r) AAV), the expression cassettes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art.

Methods of preparing AAV-based vectors (e.g., having an AAV9 or another AAV capsid) are known. See, e.g., U.S. Published Pat. Application No. 2007/0036760 (Feb. 15, 2007), which is incorporated by reference herein. The invention is not limited to the use of AAV9 or other clade F AAV amino acid sequences, but encompasses peptides and/or proteins containing the terminal β-galactose binding generated by other methods known in the art, including, e.g., by chemical synthesis, by other synthetic techniques, or by other methods. The sequences of any of the AAV capsids provided herein can be readily generated using a variety of techniques. Suitable production techniques are well known to those of skill in the art. See, e.g., Sambrook et al, Molecular Cloning: A Laboratory Manual, Cold Spring Harbor Press (Cold Spring Harbor, NY). Alternatively, peptides can also be synthesized by the well-known solid phase peptide synthesis methods (Merrifield, (1962) J. Am. Chem. Soc., 85:2149; Stewart and Young, Solid Phase Peptide Synthesis (Freeman, San Francisco, 1969) pp. 27-62). These methods may involve, e.g., culturing a host cell which contains a nucleic acid sequence encoding an AAV capsid; a functional rep gene; a minigene composed of, at a minimum, AAV inverted terminal repeats (ITRs) and a transgene; and sufficient helper functions to permit packaging of the minigene into the AAV capsid protein. These and other suitable production methods are within the knowledge of those of skill in the art and are not a limitation of the present invention.

The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

These rAAVs are particularly well suited to gene delivery for therapeutic purposes and for preventing infection. Further, the compositions of the invention may also be used for production of a desired gene product in vitro. For in vitro production, a desired product (e.g., a protein) may be obtained from a desired culture following transfection of host cells with a rAAV containing the molecule encoding the desired product and culturing the cell culture under conditions which permit expression. The expressed product may then be purified and isolated, as desired. Suitable techniques for transfection, cell culturing, purification, and isolation are known to those of skill in the art. Methods for generating and isolating AAVs suitable for use as vectors are known in the art. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety. For packaging a transgene into virions, the ITRs are the only AAV components required in cis in the same construct as the nucleic acid molecule containing the expression cassettes. The cap and rep genes can be supplied in trans.

In one embodiment, the expression cassettes described herein are engineered into a genetic element (e.g., a shuttle plasmid) which transfers the immunoglobulin construct sequences carried thereon into a packaging host cell for production a viral vector. In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. Alternatively, the expression cassettes may be used to generate a viral vector other than AAV, or for production of mixtures of antibodies in vitro. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, NY (2012).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant AAV described herein may be generated using techniques which are known. See, e.g., WO 2003/042397; WO 2005/033321, WO 2006/110689; US 7588772 B2. Such a method involves culturing a host cell which contains a nucleic acid sequence encoding an AAV capsid; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, cells are manufactured in a suitable cell culture (e.g., HEK 293 cells). Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, which is incorporated herein by reference in its entirety. Methods of making and using these and other AAV production systems are also described in the following U.S. patents, the contents of each of which is incorporated herein by reference in its entirety: 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. In certain embodiments, the methods of making and using AAV production systems includes that of which uses pseudorabies viruses (rPRV) described in U.S. Pat. Application 63/016,894 filed on Apr. 28, 2020, incorporated herein by reference.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A two-step affinity chromatography purification at high salt concentration followed anion exchange resin chromatography are used to purify the vector drug product and to remove empty capsids. These methods are described in more detail in International Patent Application No. PCT/US2016/065970, filed Dec. 9, 2016, entitled “Scalable Purification Method for AAV9”, which is incorporated by reference. Purification methods for AAV8, International Patent Application No. PCT/US2016/065976, filed Dec. 9, 2016, and rh10, International Patent Application No. PCT/US16/66013, filed Dec. 9, 2016, entitled “Scalable Purification Method for AAVrh10”, also filed Dec. 11, 2015, and for AAV1, International Patent Application No. PCT/US2016/065974, filed Dec. 9, 2016 for “Scalable Purification Method for AAV1”, filed Dec. 11, 2015, are all incorporated by reference herein.

To calculate empty and full particle content, vp3 band volumes for a selected sample (e.g., in examples herein an iodixanol gradient-purified preparation where number of GC = number of particles) are plotted against GC particles loaded. The resulting linear equation (y = mx+c) is used to calculate the number of particles in the band volumes of the test article peaks. The number of particles (pt) per 20 µL loaded is then multiplied by 50 to give particles (pt) /mL. Pt/mL divided by GC/mL gives the ratio of particles to genome copies (pt/GC). Pt/mL-GC/mL gives empty pt/mL. Empty pt/mL divided by pt/mL and x 100 gives the percentage of empty particles.

Generally, methods for assaying for empty capsids and AAV vector particles with packaged genomes have been known in the art. See, e.g., Grimm et al., Gene Therapy (1999) 6:1322-1330; and Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer’s instructions or other suitable staining method, i.e., SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

Additionally, another example of measuring empty to full particle ratio is also known in the art. Sedimentation velocity, as measured in an analytical ultracentrifuge (AUC) can detect aggregates, other minor components as well as providing good quantitation of relative amounts of different particle species based upon their different sedimentation coefficients. This is an absolute method based on fundamental units of length and time, requiring no standard molecules as references. Vector samples are loaded into cells with 2-channel charcoal-epon centerpieces with 12 mm optical path length. The supplied dilution buffer is loaded into the reference channel of each cell. The loaded cells are then placed into an AN-60Ti analytical rotor and loaded into a Beckman-Coulter ProteomeLab XL-I analytical ultracentrifuge equipped with both absorbance and RI detectors. After full temperature equilibration at 20° C. the rotor is brought to the final run speed of 12,000 rpm. A₂₈₀ scans are recorded approximately every 3 minutes for ~5.5 hours (110 total scans for each sample). The raw data is analyzed using the c(s) method and implemented in the analysis program SEDFIT. The resultant size distributions are graphed and the peaks integrated. The percentage values associated with each peak represent the peak area fraction of the total area under all peaks and are based upon the raw data generated at 280 nm; many labs use these values to calculate empty: full particle ratios. However, because empty and full particles have different extinction coefficients at this wavelength, the raw data can be adjusted accordingly. The ratio of the empty particle and full monomer peak values both before and after extinction coefficient-adjustment is used to determine the empty-full particle ratio.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay. Quantification also can be done using ViroCyt or flow cytometry.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 Apr;25(2):115-25. doi: 10.1089/hgtb.2013.131. Epub 2014 Feb 14.

### Compositions and Uses

Provided herein are compositions containing at least one mutant hAce2 soluble decoy protein and an optional carrier, excipient and/or preservative. In certain embodiments, a composition may contain at least one mutant hAce2 soluble decoy fusion protein and an optional carrier, excipient and/or preservative. In certain embodiments, the composition may comprise at least a second, different hAce2 soluble decoy and/or hAce2 soluble decoy fusion protein. For example, the composition may comprise of hAce2-Variant1-IgG soluble decoy fusion protein and hAce2-Variant4-IgG soluble decoy fusion protein.

Provided herein are also compositions containing at least one rAAV stock (e.g., an rAAV9.hACE2 stock) and an optional carrier, excipient and/or preservative. An rAAV stock refers to a plurality of rAAV vectors which are the same, e.g., such as in the amounts described below in the discussion of concentrations and dosage units.

As used herein, the term “AAV.hAce2” refers to an rAAV having a coding sequence for either wild-type or mutated hAce2, where indicated and as defined herein, which has packaged therein a vector genome encoding the hACE2 soluble protein. The term “AAV.hACE2-VariantX-IgG ” refers to a rAAV having a coding sequence for mutated hACE2 soluble decoy fusion as defined herein, wherein X stands for 1, 2, 3, or 4, which has packaged therein a vector genome encoding the hACE2 soluble protein and an IgG Fc portion, designed to expression and assemble as a fusion protein in a target cell. As used herein, the term “AAV9.hACE2-VariantX-IgG ” refers to a rAAV having an AAV9 capsid as defined herein which has packaged therein the vector genome, wherein X stands for 1,2,3,4,5 or 6.

In certain embodiments, the rAAV stock comprise a vector genome selected from: SEQ ID NO: 17 (hAce2-Variant1-IgG4), SEQ ID NO: 19 (hAce2-Variant2-IgG4), SEQ ID NO: 21 (hAce2-Variant3-IgG4), SEQ ID NO: 23 (hAce2-Variant4-IgG4), SEQ ID NO: 85 (hAce2-Variant2-1gG1), SEQ ID NO: 87 (hAce2-MR27-Variant-IgG1), SEQ ID NO: 89 (hAce2-Variant2-IgG1 comprising “GS” linker between hAce2 and IgG1 Fc domain), and/or SEQ ID NO: 91 (hAce2-MR27-Variant-IgG1 comprising “GS” linker). In certain embodiments, the rAAV stock comprise an expression cassette selected from: nt 198 to 4666 of SEQ ID NO: 17 or SEQ ID NO: 116 (hAce2-Variant1-IgG4), nt 198 to 4666 of SEQ ID NO: 19 or SEQ ID NO: 117 (hAce2-Variant2-IgG4), nt 198 to 4666 of SEQ ID NO: 21 or SEQ ID NO: 118(hAce2-Variant3-IgG4), nt 198 to 4666 of SEQ ID NO: 23 or SEQ ID NO: 119 (hAce2-Variant4-IgG4), SEQ ID NO: 86 (hAce2-Variant2-IgG1), SEQ ID NO: 88 (hAce2-MR27-Variant-IgG1), SEQ ID NO: 90 (hAce2-Variant2-IgG1 comprising “GS” linker between hAce2 and IgG1 Fc domain), and/or SEQ ID NO: 92 (hAce2-MR27-Variant-IgG1 comprising “GS” linker).

In certain embodiments, the rAAV stock comprise a vector genome comprising a nucleic acid sequence encoding for an hAce2 soluble decoy with an amino acid sequence selected from SEQ ID NOs: 10, 12, 14, 16, 72 or 73, wherein the hAce2 soluble decoy is further linked to an Fc domain selected from IgG1, IgG4, IgA or IgM. In certain embodiments, the rAAV stock comprise a vector genome comprising a nucleic acid sequence encoding for an hAce2 soluble decoy with an amino acid sequence selected from SEQ ID NOs: 52 to 61, wherein the hAce2 soluble decoy is further linked to an Fc domain selected from IgG1, IgG4, IgA or IgM.

In certain embodiments, a composition may contain at least a second, different rAAV stock. This second vector stock may vary from the first by having a different AAV capsid and/or a different vector genome. In certain embodiments, a composition as described herein may contain a different vector expressing an expression cassette as described herein, or another active component (e.g., an antibody construct, another biologic, and/or a small molecule drug).

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like.

The term “soluble” as used herein refers to the ability of a polypeptide to be solvated in an aqueous solution. For example, a soluble peptide can be mixed with an aqueous medium such that at least a detectable portion of the peptide is present in the aqueous medium. The peptide may be detected through use of common techniques, such as absorbance of light, fluorescence, the ability to bind dyes, the ability to reduce silver ions, and the like.

A pharmaceutical composition comprising the mutant hACE2 protein(s) or a mutant hACE2-encoding nucleic acid may be made. Such compositions may include pharmaceutically suitable salts thereof, plus buffers, tonicity components or pharmaceutically suitable vehicles. Pharmaceutical vehicle substances are used to improve the tolerability of the composition and allow a better solubility as well as better bioavailability of the active substances. Examples here include emulsifiers, thickeners, redox components, starch, alcohol solutions, polyethylene glycol, or lipids. Other carriers may include additives used in tablets, granules and capsules, etc. Typically, such carriers contain excipients such as starch, milk, sugar, certain types of clay, gelatin, stearic acid or salts thereof, magnesium or calcium stearate, talc, vegetable fats or oils, gum, glycols or other known excipients. Such carriers may also include flavor and color additives or other ingredients. Compositions comprising such carriers are formulated by well-known conventional methods. The selection of a suitable pharmaceutical vehicle depends to a great extent on how the substance is administered. Liquid or solid vehicles may be used for oral administration, but for injections the final composition must be a liquid.

The medication to be used may comprises buffer substances or tonic substances. By means of buffers, the pH of the medication can be adjusted to physiological conditions and furthermore fluctuations in pH can be diminished and/or buffered. One example of this is a phosphate buffer. Tonic substances are used to adjust the osmolarity and may include ionic substances, for example, inorganic salts such as NaCl or nonionic salts such as glycerol or carbohydrates. The composition to be used according to the present invention is preferably prepared to be suitable for systemic, topical, oral, or intranasal administration. These forms of administration of the medication according to the present invention allow a rapid and uncomplicated uptake. For oral administration, for example, solid and/or liquid medications may be taken directly or dissolved and/or diluted. The medication to be used according to the invention is preferably prepared for intravenous, intra-arterial, intramuscular, intravascular, intraperitoneal or subcutaneous administration. For example, injections or transfusions are suitable for this purpose. Administration directly into the bloodstream has the advantage that the active ingredients of the medication are distributed throughout the entire body and rapidly reach the target tissue.

The dosage of the compositions and/or the pharmaceutical composition comprising modified hAce2 soluble decoy and/or hAce2 soluble decoy fusion protein of the invention depends on factors including the route of administration, and physical characteristics of subject in need, e.g., age, weight, general health, of the subject. For example, the amount of an hAce2 soluble decoy and/or hAce2 soluble decoy fusion protein in a single dose may be in an amount that effectively prevents, ameliorate symptoms, or treats the SARS-CoV (or other virus mediated by binding the Ace2 receptor) without inducing significant toxicity. A pharmaceutical composition of the invention may include a dosage of an hAce2 soluble decoy and/or hAce2 soluble decoy fusion protein, as described herein, ranging from 0.01 to 500 mg/kg (e.g., 0.01, 0.1, 0.2, 0.3, 0.4, 0.5, 1, 2, 3, 4, 5, 10, 15, 20, 25, 30, 35, 40, 45, 50, 100, 150, 200, 250, 300, 350, 400, 450, or 500 mg/kg) and, at least about 1 to at least about 100 mg/kg and, at least about 1 to at least about 50 mg/kg. The dosage may be adapted in accordance to the extent of the SARS-CoV (or other virus mediated by binding the Ace2 receptor) infection and according to different parameters of the subject. The pharmaceutical compositions are administered in a manner compatible with the dosage formulation and in such amount as is therapeutically effective to result in an amelioration of the symptoms, treatment and/or prevention of a viral infection mediated by the Ace2 receptor, e.g., SARS-CoV1 or SARS-CoV2, among others identified herein. The pharmaceutical compositions are administered in a variety of dosages, e.g., intravenous dosage, intranasal dosage, intramuscular dosage and oral dosage.

In certain embodiments, one or more mutant soluble human Ace2 (hAce2) proteins provided herein are useful in treating and, in certain embodiments preventing, infection with betacoronaviruses, including SARS-CoV2 is provided, as are compositions useful in treating disease associated with betacoronavirus-associated disease, including, e.g., SARS-associated infection and COVID-19.

In certain embodiments, compositions comprising one or more hAce2 soluble decoy proteins and/or hAce2 soluble decoy fusion proteins may be dosed at 1 mg/kg to 100 mg/kg, or doses therebetween, e.g., 1 mg/kg to 50 mg/kg. In certain embodiments, the composition may be administered to a subject in a need thereof, at least one or more times, daily for a pre-determined time period, weekly, monthly, biannually, annually, or as necessary. In certain embodiments, the compositions are delivered intravenously.

In certain embodiments, effectiveness of hAce2 soluble decoy and/or hAce2 soluble decoy fusion protein may be used in determining the effective dose. In certain embodiments IC50 is used to measure the effectiveness of hAce2 soluble decoy and/or hAce2 soluble decoy fusion protein. “IC50” is the half maximal inhibitory concentration, which is a measure of the effectiveness of a substance for inhibiting a specific quantifiable biological or biochemical function. This quantitative measure indicates how much of a particular substance is needed to inhibit a specific biological function by 50% and is commonly used in the art. In certain embodiments, the IC50 of hAce2 soluble decoy and/or hAce2 soluble decoy fusion protein is measured as capable of blocking binding ACE2 binding to RBD. In certain embodiments, the IC50 is at least about 3 ng/mL to at least about 10 ng/mL. In certain embodiments, the IC50 for mutant hAce2 soluble decoy fusion protein is at least about 10 times lower, at least about 100 times lower, at least about 300 times lower, and/or at least about 1000 times lower as compared wild-type hAce2 soluble decoy fusion protein.

In certain embodiments, a pharmaceutical composition is provided which comprises at least one recombinant AAV as described herein and one or more of any of: a pharmaceutically acceptable diluent, a suspending agent, a preservative, and/or a surfactant. In certain embodiments, the pharmaceutical composition is formulated for intranasal administration. In certain embodiments, the pharmaceutical composition is formulated for intrapulmonary administration. In certain embodiments, the pharmaceutical composition is formulated for intravenous administration. In certain embodiments, the pharmaceutical composition is formulated for intraperitoneal administration.

In another aspect, a method is provided for treating and/or preventing one or more symptoms of SARS-CoV2 by administering or co-administering a pharmaceutically effective amount of synthetic or recombinant hAce2 soluble decoy protein, an rAAV or a pharmaceutical composition as described herein, and combinations thereof. In certain embodiments, the symptoms are one or more of fever, cough, gastrointestinal distress, nausea, vomiting, diarrhea, eye pain, breathing difficulty, loss of taste, and/or loss of smell. In certain embodiments, the method comprises administering or co-administering the hAce2 soluble decoy protein, the rAAV or a pharmaceutical composition intranasally. In still other embodiments, the method comprises administering or co-administering the hAce2 soluble decoy protein, the rAAV or a pharmaceutical composition via inhalation. In other embodiments, the method comprises administering or co-administering the hAce2 soluble decoy protein, the rAAV or a pharmaceutical composition intravenously.

Methods suitable for assessing antibodies that bind to ACE2 extracellular domain include 20 those of Enzyme Linked Immunosorbent Assay (ELISA). Specifically, NOVUS NBP2 human ACE-2 ELISA Chemiluminescent Kit, which can specifically detect human ACE2 in various samples such as serum, plasma and other biological fluids (available from the following website: novusbio.com/products/ace-2-elisa-kit_nbp2-66387#datasheet). The kit is utilized according to manufacturing instruction. In some embodiments of the claimed, the kit is used to immobilize the 25 hAce2, and hAce2_NN for SARS-CoV-2 S-protein binding assay. For hAce2-fuson and hAce2_NN-fuison protein binding with S-protein of SARS-CoV-2, anti-human IgG-HRP linked detection antibody was used.

In some embodiments, assessing levels of SARS-CoV neutralization activity is performed using the adopted SARS-CoV-2 S-protein binding assay, as indicated above. A pseudotyped Human Immunodeficiency Viral (HIV) vector with the S-protein of SARSCoV-2 and expressing eGFP was used in evaluating in vitro neutralization activity of the constructs (Kobinger, G.P., et al., 2007, Hum Gene Ther, 18(5): 413-422). Vero E 6 cells (ATCC CRL1586) is used for viral replication of S-protein pseudotyped HIV vector. Ace2 is expressed in stably transduced HEK293 cells (selected for expression with Zeocin).

In certain embodiments, hACE2 soluble decoy and/or hAce2 soluble decoy fusion proteins are tested for neutralizing activity using a pseudotyped retroviral transduction inhibition assay. A lentiviral vector is generated carrying a GFP-reporter gene and pseudotyped with a SARS-CoV-2 spike protein with a heterologous C-terminus for optimal incorporation into a lentiviral vector. The pseudotyped vector is used to transduce target HEK 293 cells that carry a human ACE2 transgene. Neutralizing activity of candidate soluble ACE2 proteins and ACE2-Fc fusion proteins are evaluated by pre-incubating the purified protein with the lentiviral vector before it is applied to the ACE2-expressing target cells. Neutralization are quantified based on the concentration of the protein that yields a 50% reduction in GFP expression in target cells relative to cells treated with the lentiviral vector alone.

In certain embodiments, K_{D} value is used to measure the effectiveness of hAce2 soluble decoy and/or hAce2 soluble decoy fusion protein, wherein K_{D} value reflects the interaction between the hAce2 soluble decoy and/or hAce2 soluble decoy fusion protein, and RBD of SARS-CoV. In certain embodiments, K_{D} value is at least about 40 pM to at least about 300 pM.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL® HS 15 (Macrogol-15 Hydroxystearate), LABRASOL® (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits x 100 give the approximate molecular mass of the polyoxypropylene core, and the last digit x 10 gives the percentage polyoxyethylene content. In one embodiment Poloxamer 188 is selected. The surfactant may be present in an amount up to about 0.0005% to about 0.001% of the suspension.

In one embodiment, the formulation buffer is phosphate-buffered saline (PBS) with total salt concentration of 200 mM, 0.001% (w/v) pluronic F68 (Final Formulation Buffer, FFB). In another embodiment, the formulation buffer is phosphate buffer saline with 0.001% poloxamer 188 (137 mM Sodium Chloride, 2.7 mM Potassium Chloride, 10 mM Sodium Phosphate Dibasic, 1.8 mM Potassium Phosphate Monobasic, 0.001% Poloxamer 188, pH 7.3-7.5).

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. In certain embodiments, the vectors are formulated for delivery via intranasal delivery devices for targeted delivery to nasal and/or nasopharynx epithelial cells. In certain embodiments, vectors are formulated for aerosol delivery devices, e.g., via a nebulizer or through other suitable devices. Other conventional and pharmaceutically acceptable routes of administration include, but are not limited to, direct delivery to a desired organ (e.g., lung), oral, inhalation, intrathecal, intratracheal, intraarterial, intraocular, intravenous, intramuscular, subcutaneous, intradermal, and other parenteral routes of administration. In one embodiment, the vector is administered intranasally using intranasal mucosal atomization device (LMA® MAD Nasal™- MAD110). In another embodiment the vector is administered intrapulmonary in nebulized form using Vibrating Mesh Nebulizer (Aerogen® Solo) or MADgic™ Laryngeal Mucosal Atomizer. In certain embodiments, Ace2 decoys are administered via inhalation. Routes of administration may be combined, if desired. Routes of administration and utilization of which for delivering rAAV vectors are also described in the following published U.S. Pat. Applications, the contents of each of which is incorporated herein by reference in its entirety: US 2018/0155412A1, US 2018/0243416A1, US 2014/0031418 A1, and US 2019/0216841A1.

Dosages of the viral vector will depend primarily on factors such as the condition being treated, the age, weight and health of the patient, and may thus vary among patients. For example, a therapeutically effective human dosage of the viral vector is generally in the range of from about 25 to about 1000 microliters to about 5 mL of aqueous suspending liquid containing doses of from about 10⁹ to 4x10¹⁴ GC of AAV vector. The dosage will be adjusted to balance the therapeutic benefit against any side effects and such dosages may vary depending upon the therapeutic application for which the recombinant vector is employed. The levels of expression of the transgene can be monitored to determine the frequency of dosage resulting in viral vectors, preferably AAV vectors containing the minigene. Optionally, dosage regimens similar to those described for therapeutic purposes may be utilized for immunization using the compositions of the invention.

The replication-defective virus compositions can be formulated in dosage units to contain an amount of replication-defective virus that is in the range of about 10⁹ GC to about 10¹⁶ GC (to treat an average subject of 70 kg in body weight) including all integers or fractional amounts within the range, and preferably 10¹² GC to 10¹⁴ GC for a human patient. In one embodiment, the compositions are formulated to contain at least 10⁹, 2x10⁹, 3x10⁹, 4x10⁹, 5x10⁹, 6x10⁹, 7x10⁹, 8x10⁹, or 9x10⁹ GC per dose including all integers or fractional amounts within the range. In another embodiment, the compositions are formulated to contain at least 10¹⁰, 2x10¹⁰, 3x10¹⁰, 4x10¹⁰, 5x10¹⁰, 6x10¹⁰, 7x10¹⁰, 8x10¹⁰, or 9x10¹⁰ GC per dose including all integers or fractional amounts within the range. In another embodiment, the compositions are formulated to contain at least 10¹¹, 2x10¹¹, 3x10¹¹, 4x10¹¹, 5x10¹¹, 6x10¹¹, 7x10¹¹, 8x10¹¹, or 9x10¹¹ GC per dose including all integers or fractional amounts within the range. In another embodiment, the compositions are formulated to contain at least 10¹², 2x10¹², 3x10¹², 4x10¹², 5x10¹², 6x10¹², 7x10¹², 8x10¹², or 9x10¹² GC per dose including all integers or fractional amounts within the range. In another embodiment, the compositions are formulated to contain at least 10¹³, 2x10¹³, 3x10¹³, 4x10¹³, 5x10¹³, 6x10¹³, 7x10¹³, 8x10¹³, or 9x10¹³ GC per dose including all integers or fractional amounts within the range. In another embodiment, the compositions are formulated to contain at least 10¹⁴, 2x10¹⁴, 3x10¹⁴, 4x10¹⁴, 5x10¹⁴, 6x10¹⁴, 7x10¹⁴, 8x10¹⁴, or 9x10¹⁴ GC per dose including all integers or fractional amounts within the range. In another embodiment, the compositions are formulated to contain at least 10¹⁵, 2x10¹⁵, 3x10¹⁵, 4x10¹⁵, 5x10¹⁵, 6x10¹⁵, 7x10¹⁵, 8x10¹⁵, or 9x10¹⁵ GC per dose including all integers or fractional amounts within the range. In one embodiment, for human application the dose can range from 10¹⁰ to about 10¹² GC per dose including all integers or fractional amounts within the range. In one embodiment, for human application the dose can range from 10⁹ to about 7x10¹³ GC per dose including all integers or fractional amounts within the range. In one embodiment, for human application the dose ranges from 6.25x10¹² GC to 5.00x10¹³ GC. In a further embodiment, the dose is about 6.25x10¹² GC, about 1.25x10¹³ GC, about 2.50x10¹³ GC, or about 5.00x10¹³ GC. In a further embodiment, the dose is about 1.02x10¹¹ GC, about 3.40x10¹¹ GC, or about 1.02x10¹² GC. In certain embodiments, the dose is about 1.5 x 10¹¹ GC, about 1.5 x 10¹² GC, or about 1.5 x 10¹³ GC. In certain embodiments, the dose is about 5 x 10¹¹ GC, about 5 x 10¹² GC, or about 5 x 10¹³ GC. In certain embodiment, the dose is divided into one half thereof equally and administered to each nostril. In certain embodiments, for human application the dose ranges from 6.25x10¹² GC to 5.00x10¹³ GC administered as two aliquots of 0.2 ml per nostril for a total volume delivered in each subject of 0.8 ml.

These above doses may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method. In one embodiment, the volume of carrier, excipient or buffer is at least about 25 µL. In one embodiment, the volume is about 50 µL. In another embodiment, the volume is about 75 µL. In another embodiment, the volume is about 100 µL. In another embodiment, the volume is about 125 µL. In another embodiment, the volume is about 150 µL. In another embodiment, the volume is about 175 µL. In yet another embodiment, the volume is about 200 µL. In another embodiment, the volume is about 225 µL. In yet another embodiment, the volume is about 250 µL. In yet another embodiment, the volume is about 275 µL. In yet another embodiment, the volume is about 300 µL. In yet another embodiment, the volume is about 325 µL. In another embodiment, the volume is about 350 µL. In another embodiment, the volume is about 375 µL. In another embodiment, the volume is about 400 µL. In another embodiment, the volume is about 450 µL. In another embodiment, the volume is about 500 µL. In another embodiment, the volume is about 550 µL. In another embodiment, the volume is about 600 µL. In another embodiment, the volume is about 650 µL. In another embodiment, the volume is about 700 µL. In another embodiment, the volume is between about 700 and 1000 µL.

In certain embodiments, the recombinant vectors may be dosed intranasally by using two sprays to each nostril. In one embodiment, the two sprays are administered by alternating to each nostril, e.g., left nostril spray, right nostril spray, then left nostril spray, right nostril spray. In certain embodiments, there may be a delay between alternating sprays. For example, each nostril may receive multiple sprays which are separated by an interval of about 10 to 60 seconds, or 20 to 40 seconds, or about 30 seconds, to a few minutes, or longer. Such sprays may deliver, e.g., about 150 µL to 300 µL, or about 250 µL in each spray, to achieve a total volume dosed of about 200 µL to about 600 µL, 400 µL to 700 µL, or 450 µL to 1000 µL.

In certain embodiment, the recombinant AAV vector may be dosed intranasally to achieve a concentration of 5-20 ng/ml of the expression product of the transgene as measured in a nasal wash solution post-dosing, e.g., one week to four weeks, or about two weeks after administration of the vector. Methods of acquiring the nasal wash solution in the subjected as well as methods of quantification of the expression product of the transgene are conventional.

For other routes of administration, e.g., intravenous or intramuscular, dose levels would be higher than for intranasal delivery. For example, such suspensions may be volumes doses of about 1 mL to about 25 mL, with doses of up to about 2.5 x 10¹⁵GC.

In certain embodiments, the intranasal delivery device provides a spay atomizer which delivers a mist of particles having an average size range of about 30 microns to about 100 microns in size. In certain embodiments, the average size range is about 10 microns to about 50 microns. Suitable devices have been described in the literature and some are commercially available, e.g., the LMA MAD NASAL™ (Teleflex Medical; Ireland); Teleflex VaxINator™ (Teleflex Medical; Ireland); Controlled Particle Dispersion® (CPD) from Kurve Technologies. See, also, PG Djupesland, Drug Deliv and Transl. Res (2013) 3: 42-62. In certain embodiments, the particle size and volume of delivery is controlled in order to preferentially target nasal epithelial cells and minimize targeting to the lung. In other embodiments, the mist of particles is about 0.1 micron to about 20 microns, or less, in order to deliver to lung cells. Such smaller particle sizes may minimize retention in the nasal epithelium.

One device mists particles at an average diameter of about 16 microns to about 22 microns. The mist may be delivered directly to the tracheobronchial tree inserted through the suction channel of a 3.5-mm flexible fiberoptic bronchoscope (Olympus, Melville, NY). Other suitable delivery devices may include a laryngo-tracheal mucosal atomizer, which provides for administration across the upper airway past the vocal cords. It fits through vocal cords and down a laryngeal mask or into nasal cavity. The droplets are atomized at an average diameter of about 30 microns to about 100 microns. A standard device has a tip diameter of about 0.18 in (4.6 mm), a length of about 4.5-8.5 inches, and is inserted through the suction channel and advanced approximately 3 mm beyond the distal tip of the scope. Doses may be administered is 10 aliquots (approximately 150 µl each) of 99 control with saline or 99 rAAV.hACE2 sprayed into right and left main stem bronchi.

In one embodiment, a frozen composition is provided which contains an rAAV in a buffer solution as described herein, in frozen form. Optionally, one or more surfactants (e.g., Pluronic F68), stabilizers or preservatives is present in this composition. Suitably, for use, a composition is thawed and titrated to the desired dose with a suitable diluent, e.g., sterile saline or a buffered saline.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine -glycine pairs. Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, 97%, 99%, up to about 100% deamidated at a referenced amino acid position, as compared to the predicted amino acid sequence at the reference amino acid position. Such percentages may be determined using 2D-gel, mass spectrometry techniques, or other suitable techniques.

In certain embodiments, composition neutralizes more than one subtype of the target airborne pathogen. In certain embodiments, the target airborne pathogen is a virus which binds to the Ace2 receptor. In certain embodiments, the virus is selected from SARS-CoV2, SARS-CoV1, or mutated SARS-CoV2. In some embodiments, the composition treats infection with mutated SARS-CoV2 variants, e.g., EU, UK, South Africa), mink, and further variants including, e.g., Delta, Delta plus, Gamma, Epsioon, Kappa, Ioata, Zeta, omnicron, or related betacoronaviruses, e.g., CoV1. See also, Ku, Z., et al., 2021, Molecular determinants and mechanisms for antibody cocktail preventing SARS-CoV2 escape, Nature Communications, 12(469), pub Jan. 20, 2021; Greaney, A. J., et al., Comprehensive mapping of mutations to the SARS-CoV-2 receptor-binding domain that affect recognition by polyclonal human serum antibodies, 2021, bioRXiv, 2020.12.31, pub Jan. 4, 2021; Xie, X., et al., Neutralization of N501Y mutant SARS-CoV-2 by BNT162b2 vaccine-elicited sera, bioRXiv, 2021.01.07, pub Jan. 7, 2021; van Dorp, L., et al., Recurrent mutations in SARS-CoV-2 genomes isolated from mink point to rapid host-adaptation, bioRXiv, 2020.11.16, pub Nov. 16, 2020). In certain embodiments, the composition neutralizes mutated SARS-CoV2, wherein SARS-CoV2 comprises one or more variants, such as listed above. In certain embodiments, the composition is useful for binding RBD of human ACE2 of betacoronaviruses, including both SARS-COV1 and SARS-CoV2 and having neutralizing activity for both.

In certain embodiments, compositions are provided herein which comprise the hAce2 fusion proteins in a non-viral delivery system and one or more suspending agents, carriers, excipients, preservatives, or the like. Excipients and carriers that can be used to prepare parenteral formulations comprising the proteins include, without limitation, aqueous solvents such as water, saline, physiological saline, buffered saline (e.g., phosphate-buffered saline), balanced salt solutions [e.g., Ringer’s BSS] and aqueous dextrose solutions), isotonic/iso-osmotic agents (e.g., salts [e.g., NaCl, KCI and CaC12] and sugars [e.g., sucrose]), buffering agents and pH adjusters (e.g., sodium dihydrogen phosphate [monobasic sodium phosphate]/disodium hydrogen phosphate [dibasic sodium phosphate], citric acid/sodium citrate and L-histidine/L-histidine HCl), non-solvents, or combinations of one or more aqueous and one or more non-aqueous solvents, and emulsifiers (e.g., non-ionic surfactants such as polysorbates [e.g., polysorbate 20 and 80] and poloxamers [e.g., poloxamer 188]). In certain embodiments, the proteins may be delivered in a lipid particle, nanoparticle, in a liposome, micelle, an implant, hydrogel, or other suitable non-viral delivery or carrier system.

Suitable doses may include, e.g., 1 µg up to 100 mg/dose, but may vary based on the delivery system and route of administration. For example, intranasal delivery systems may delivery about 10 µg/ml to about 1 mg/ml, or about 100 µg/ml to about 1 to 10 mg/ml per dose. Higher doses may be delivered intravenously, or via other delivery routes. Dosing may be over period of 1 to 3 days, or daily over a week, two weeks, three weeks, 4 weeks, 6 weeks, 2 months, 3 months, or longer. The doses may be higher in the beginning and taper. Optionally, doses may be delivered multiple times a day and/or days may be skipped between doses.

In certain embodiments, the rAAV encoding mutated hAce2 soluble decoy and/or mutated hAce2 soluble decoy fusion protein may be administered intravenously to achieve expression levels sufficient for treatment of coronaviruses which utilize and/or rely on ACE2 receptor for intracellular entry and infection. In certain embodiments, the coronavirus is SARS-CoV1. In certain embodiments, the coronavirus is SARS-CoV2. In certain embodiments, the coronavirus is a naturally mutated SARS-CoV2.

In certain embodiments, the hAce2 soluble decoy and/or hAce2 soluble decoy fusion protein may be administered intravenously at an effective dose for treatment of betacoronaviruses which utilize and/or rely on ACE2 receptor for intracellular entry and infection. In certain embodiments, the coronavirus is SARS-CoV1. In certain embodiments, the coronavirus is SARS-CoV2. In certain embodiments, the coronavirus is mutated SARS-CoV2. In certain embodiments, the hAce2 soluble decoy and/or hAce2 soluble decoy fusion protein may be administered intravenously at an effective dose for treatment in a subject in need thereof, wherein the subject had been diagnosed with COVID-19 (SARS-CoV2, including SARS-CoV2 mutants). In certain embodiments, the mutant hAce2 soluble decoy fusion protein is administered in a composition comprising a mixture of the mutant hAce2 soluble decoy proteins, e.g.,hAce2-Variant2-Fc and hAce2-MMR27-Fc.

In certain embodiments, rAAV encoding mutated hAce2 soluble decoy fusion protein (hAce2-IgG) may be administered intranasally to achieve expression levels sufficient for COVID-19 prophylaxis and prevention in un-infected subjects in the need thereof. In certain embodiments, rAAV encoding mutated hAce2 soluble decoy fusion protein may be administered intrapulmonary to achieve expression levels sufficient for COVID-19 treatment in infected subjects in need thereof. In certain embodiments, rAAV encoding mutated hAce2 soluble decoy fusion protein may be administered intravenously. In certain embodiments, rAAV encoding mutated hAce2 soluble decoy fusion protein may be administered intramuscularly. Optionally, a co-therapy may be selected either prior to, simultaneously with, or following administration of a rAAV provided herein.

In certain embodiment, a co-therapy may involve co-administration of a combination of two or more different populations of rAAV expressing an hACE2 soluble protein as described herein. In certain embodiments, a co-therapy may involve co-administration of a combination of different populations of rAAV expressing two or more of mutated hAce2 soluble decoy fusion proteins, i.e., hAce2- Variant1-IgG, hAce2-Variant2-IgG, hAce2-Variant3-IgG, hAce2-Variant4-IgG, hAce2-MR27HL-Variant-IgG, wherein the IgG is IgG1 Fc or IgG4 Fc. In certain embodiments, the co-therapy may involve co-administration of a combination of rAAV expressing an hAce2 soluble protein as described herein and an rAAV expressing an hAce2 soluble protein as described in US Provisional Pat. Application No. 63/002,100, filed Mar. 3, 2020; US Provisional Pat. Application No. 63/027,731, filed May 20, 2020; and US Provisional Pat. Application No. 63/069,651, filed Aug. 24, 2020, which are incorporated herein by reference. In certain embodiments, a co-therapy may involve co-administration of a combination of two or more of mutated hAce2 soluble decoy fusion proteins, i.e., hAce2-Variant1-IgG, hAce2-Variant2-IgG, hAce2-Variant3-IgG, hAce2-Variant4-IgG, hAce2-Variant5-IgG, hAce2-Variant6-IgG, hAce2-MR27HL-Variant-IgG. In certain embodiments, the co-therapy may involve co-administration of a combination of rAAV expressing an hAce2 soluble protein as described herein a mutated hAce2 soluble decoy fusion proteins as described herein.

Additionally or alternatively, a different drug or biological may be co-administered to a subject for treatment of one or more symptoms of COVID-19. Such a co-therapeutic may be, e.g., remdesivir (GS-5734), Sarilumab, chloroquine or hydroxychloroquine, hyperimmune plasma, DAS181 (e.g., Nebulized DAS181 9 mg/day (4.5 mg bid/day) for 10 days), recombinant human angiotensin-converting enzyme 2 (rhACE2), Sildenafil, Tocilizumab, tetrandrine 60 m QD for one week (take 6 days, stop using for 1 day), methylprednisolone, camostat mesylate (a Serine protease inhibitor that blocks TMPRSS-2 mediated cell entry of SARS-CoV-2), bevacizumab, danoprevirr combined with ritonavir, baricitinib, hydroxychloroquine with azithromycin, chloroquine or a pharmaceutically acceptable salt thereof, and azithromycin, lopinavir/ritonavir; eculizumab (Soliris), IFN-α2β, Darunavir and Cobicistat, Anti-CD147 Humanized Meplazumab, Ribavirin and IFN-beta.

Such co-therapies may be delivered by the same route of delivery as a rAAV.hACE2, or may be delivered by other routes of delivery. In certain embodiments, a co-therapy may involve co-administration of a combination of rAAV-hAce2 and at least one or more of mutated hAce2 soluble decoy fusion proteins, i.e., hAce2-Variant1-IgG, hAce2-Variant2-IgG, hAce2-Variant3-IgG, hAce2-Variant4-IgG, hAce2-Variant5-IgG, hAce2-Variant6-IgG, hAce2-MR27HL-Variant-IgG intravenously. In certain embodiments, a co-therapy may involve co-administration via a combination of routes intravenously, intramuscularly, intrapulmonary and/or intranasally. In certain embodiments, the rAAV-hAce2 may encode for wild type hAce2 or comprise “NN” mutation as described herein, and co-administered with and at least one or more of mutated hAce2 soluble decoy fusion proteins, i.e., hAce2-Variant1-IgG, hAce2-Variant2-IgG, hAce2-Variant3-IgG, hAce2-Variant4-IgG hAce2-Variant5-IgG, hAce2-Variant6-IgG, hAce2-MR27HL-Variant-IgG intravenously. In certain embodiments, the rAAV-hAce2 may encode for mutated hAce2 as described herein, and co-administered with and at least one or more of mutated hAce2 soluble decoy fusion proteins, i.e., hAce2-Variant1-IgG, hAce2-Variant2-IgG, hAce2-Variant3-IgG, hAce2-Variant4-IgG, hAce2-Variant5-IgG, hAce2-Variant6-IgG, hAce2-MR27HL-Variant-IgG intravenously. In certain embodiments, the rAAV-Ace2 may be administered intranasally, wherein a co-therapy of and at least one or more of mutated hAce2 soluble decoy fusion proteins, i.e., hAce2-Variant1-IgG, hAce2-Variant2-IgG, hAce2-Variant3-IgG, hAce2-Variant4-IgG, hAce2-Variant5-IgG, hAce2-Variant6-IgG, hAce2-MR27HL-Variant-IgG may be administered intravenously.

A “synthetic protein” or “recombinant protein” refers to protein, which has been expressed and purified from a producer host cell. Wherein the producer host cell, comprising a vector encoding for soluble Ace2 proteins as described herein, is cultured under conditions suitable for leading to expression of protein. In certain embodiments, a mutant soluble Ace2 proteins (hAce2 soluble decoy or hAce2 soluble decoy fusion proteins) may comprise a population of mutant soluble Ace2-proteins, which may include up to 5% variation from the sequences provided herein in view of post-translational modifications such as, e.g., glycosylation, oxidation and deamidation. In certain embodiments, there is 0.5% to 5% variation, in other embodiments, there is about 1%, about 2%, about 3%, or about 4% variation. In other embodiments, no detectable variation is observed. Such post-translation modification may be detected by assessed by any suitable technique including, e.g., chromatographic and/or mass spectrometric analysis, or peptide mapping. These detection methods are not a limitation on the present invention.

A “post-translational modification” may encompass any one of or combination of modification (s), including covalent modification, which a protein undergoes after translation is complete and after being released from the ribosome or on the nascent polypeptide co-translationally. Post-translational modification includes but is not limited to phosphorylation, myristylation, ubiquitination, glycosylation, coenzyme attachment, methylation and acetylation. Post-translational modification can modulate or influence the activity of a protein, its intracellular or extracellular destination, its stability or half-life, and/or its recognition by ligands, receptors or other proteins. Post-translational modification can occur in cell organelles, in the nucleus or cytoplasm or extracellularly.

An antibody “Fc region” and or “Fc domain” refers to the crystallizable fragment which is the region of an antibody which interacts with the cell surface receptors (Fc receptors). In one embodiment, the Fc region is a human IgG4 Fc (amino acid sequence of SEQ ID NO: 77). In one embodiment, the Fc region is a human IgG1 Fc (amino acid sequence of SEQ ID NO: 103). In one embodiment, the Fc region is a human IgA1 Fc (UniProt amino acid sequence of SEQ ID NO: 125). In one embodiment, the Fc region is a human IgA2 Fc (amino acid sequence of SEQ ID NO: 126). In one embodiment, the Fc region is a human IgM Fc (amino acid sequence of SEQ ID NO: 78). In one embodiment, the Fc region is a modified human IgM Fc without N-terminal “I” (amino acid sequence of SEQ ID NO: 79). See, also, US Provisional Pat. Application No. 63/087,053, filed Oct. 2, 2020. In one embodiment, the Fc region is an engineered Fc fragment.

An antibody “hinge region” is a flexible amino acid portion of the heavy chains of IgG and IgA immunoglobulin classes, which links these two chains by disulfide bonds.

An “immunoglobulin molecule” is a protein containing the immunologically-active portions of an immunoglobulin heavy chain and immunoglobulin light chain covalently coupled together and capable of specifically combining with antigen. The immunoglobulin molecules described herein as IgM. However, they may be used in regimens including immunoglobulins of other types, e.g., IgG, IgE, IgM, IgD, IgA and IgY, classes (e.g., IgG1, IgG2, IgG3, IgG4, IgA1 and IgA2) or subclasses. The terms “antibody” and “immunoglobulin” may be used interchangeably herein.

A “polypeptide J chain” is referred to a joining chain, which is a polypeptide comprising in IgM antibodies, wherein the polypeptide J chain regulates the antibody formation for isotype IgA or IgM. In some embodiments, the J chain is of sequence identified by sequence with NCBI accession Gene ID: 3512 (SEQ ID NO: 127). Optionally, this chain may not be expressed when the antibody is delivered via a viral vector and expressed in vivo.

A “hexavalent structure” is referred to an Fc region of the fusion protein which comprises six monomers of IgM Fc region.

A “pentavalent structure” is referred to an Fc region of the fusion protein which comprises five monomers of IgM Fc region and a joining polypeptide J chain.

An “immunoglobulin heavy chain” is a polypeptide that contains at least a portion of the antigen binding domain of an immunoglobulin and at least a portion of a variable region of an immunoglobulin heavy chain or at least a portion of a constant region of an immunoglobulin heavy chain. Thus, the immunoglobulin derived heavy chain has significant regions of amino acid sequence homology with a member of the immunoglobulin gene superfamily. For example, the heavy chain in a Fab fragment is an immunoglobulin-derived heavy chain.

An “immunoglobulin light chain” is a polypeptide that contains at least a portion of the antigen binding domain of an immunoglobulin and at least a portion of the variable region or at least a portion of a constant region of an immunoglobulin light chain. Thus, the immunoglobulin-derived light chain has significant regions of amino acid homology with a member of the immunoglobulin gene superfamily.

As used herein, the term “NAb titer” a measurement of how much neutralizing antibody (e.g., anti-AAV NAb) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009, 199 (3): p. 381-390, which is incorporated by reference herein.

Methods suitable for assessing antibodies that bind to ACE2 extracellular domain include those of Enzyme Linked Immunosorbent Assay (ELISA). Specifically, NOVUS NBP2 human ACE-2 ELISA Chemiluminescent Kit, which can specifically detect human ACE2 in various samples such as serum, plasma and other biological fluids (novusbio.com/products/ace-2-elisa-kit_nbp2-66387#datasheet). The kit is utilized according to manufacturing instruction.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

As used herein, the term “operably linked” refers to both expression control sequences that are contiguous with the gene of interest and expression control sequences that act in trans or at a distance to control the gene of interest.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless” - containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

A “recombinant AAV” or “rAAV” is a nuclease-resistant viral particle containing two elements, an AAV capsid and a vector genome containing at least non-AAV coding sequences packaged within the AAV capsid. In certain embodiments, the capsid contains about 60 proteins composed of vp1 proteins, vp2 proteins, and vp3 proteins, which self-assemble to form the capsid. Unless otherwise specified, “recombinant AAV” or “rAAV” may be used interchangeably with the phrase “rAAV vector”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid. The term “nuclease-resistant” indicates that the AAV capsid has assembled around the expression cassette which is designed to deliver a transgene to a host cell and protects these packaged genomic sequences from degradation (digestion) during nuclease incubation steps designed to remove contaminating nucleic acids which may be present from the production process.

As used herein, a “stock” of rAAV refers to a population of rAAV. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected.

In certain embodiments, a “vector genome” refers to the nucleic acid sequence packaged inside a parvovirus (e.g., rAAV) capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs, e.g., scAAV ITRs, may be used. Further, the vector genome contains regulatory sequences which direct expression of the gene products. Suitable components of a vector genome are discussed in more detail herein. In certain embodiments, the vector genome is an expression cassette having inverted terminal repeat (ITR) sequences necessary for packaging the vector genome into the AAV capsid at the extreme 5′ and 3′ end and containing therebetween an hAce2 decoy as described herein operably linked to sequences which direct expression thereof.

In certain embodiments, non-viral particles used in manufacture of a rAAV, will be referred to as vectors (e.g., production vectors). In certain embodiments, these production vectors are plasmids, but the use of other suitable genetic elements is contemplated. Such production plasmids may encode sequences expressed during rAAV production, e.g., AAV capsid or rep proteins required for production of a rAAV, which are not packaged into the rAAV. Alternatively, such a production plasmid may carry the vector genome which is packaged into the rAAV.

As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. As used herein, “operably linked” sequences include both regulatory sequences that are contiguous or non-contiguous with the nucleic acid

sequence and regulatory sequences that act in trans or cis nucleic acid sequence. Such regulatory sequences typically include, e.g., one or more of a promoter, an enhancer, an intron, a Kozak sequence, a polyadenylation sequence, and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (3′ UTR) comprising a polyadenylation site, among other elements. In certain embodiments, the regulatory sequences are operably linked to the nucleic acid sequence of a gene product, wherein the regulatory sequences are separated from nucleic acid sequence of a gene product by an intervening nucleic acid sequences, i.e., 5′-untranslated regions (5′UTR). In certain embodiments, the expression cassette comprises nucleic acid sequence of one or more of gene products. In some embodiments, the expression cassette can be a monocistronic or a bicistronic expression cassette. In other embodiments, the term “transgene” refers to one or more DNA sequences from an exogenous source which are inserted into a target cell. Typically, such an expression cassette can be used for generating a viral vector and contains the coding sequence for the gene product described herein flanked by packaging signals of the viral genome and other expression control sequences such as those described herein. In certain embodiments, a vector genome may contain two or more expression cassettes.

The term “translation” in the context of the present specification relates to a process at the ribosome, wherein an mRNA strand controls the assembly of an amino acid sequence to generate a protein or a peptide.

The term “expression” is used herein in its broadest meaning and comprises the production of RNA or of RNA and protein. Expression may be transient or may be stable.

The term “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence identity in at least about 95 to 99% of the aligned sequences. Preferably, the homology is over full-length sequence, or an open reading frame thereof, or another suitable fragment which is at least 15 nucleotides in length. Examples of suitable fragments are described herein.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g., of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length and may be up to about 700 amino acids. Examples of suitable fragments are described herein.

The term “substantial homology” or “substantial similarity,” when referring to amino acids or fragments thereof, indicates that, when optimally aligned with appropriate amino acid insertions or deletions with another amino acid (or its complementary strand), there is amino acid sequence identity in at least about 95 to 99% of the aligned sequences. Preferably, the homology is over full-length sequence, or a protein thereof, e.g., an immunoglobulin region or domain, an AAV cap protein, or a fragment thereof which is at least 8 amino acids, or more desirably, at least 15 amino acids in length. Examples of suitable fragments are described herein.

By the term “highly conserved” is meant at least 80% identity, preferably at least 90% identity, and more preferably, over 97% identity. Identity is readily determined by one of skill in the art by resort to algorithms and computer programs known by those of skill in the art.

Generally, when referring to “identity”, “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Examples of such programs include, “Clustal Omega”, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

As used herein, an “effective amount” may refer to the amount of the rAAV composition which delivers and expresses in the target cells an amount of decoy receptor sufficient to reduce or prevent SARS-CoV2 infection and/or one or more symptoms thereof. Additionally, an “effective amount” may refer to the amount of recombinant protein composition which delivers an amount of decoy receptor sufficient to reduce or prevent SARS-CoV2 infection and/or one or more symptoms thereof. An effective amount may be determined based on an animal model, rather than a human patient. Examples of a suitable murine model are described herein.

As used herein, the term “target tissue” refers to a tissue, an organ or a cell type, that an embodiment, a regimen or a composition as described herein targets. In one embodiment, the target tissue is a respiratory organ or a respiratory tissue. In an alternative or additional embodiment, the target tissue is lung. In an alternative or additional embodiment, the target tissue is nose, e.g., nasal epithelial. In an alternative or additional embodiment, the target tissue is nasopharynx. In an alternative or additional embodiment, the target tissue is respiratory epithelium. In an alternative or additional embodiment, the target tissue is nasal airway epithelium. In an alternative or additional embodiment, the target tissue is nasal cells. In an alternative or additional embodiment, the target tissue is nasopharynx cells. In an alternative or additional embodiment, the target tissue is nasal epithelial cells, which may be ciliated nasal epithelial cells, columnar epithelial cells, goblet cells (which secrete mucous onto the surface of the nasal cavity which is composed of the ciliated epithelial cells) and stratified squamous nasal epithelial cells which line the surface of the nasopharynx. In an alternative or additional embodiment, the target tissue is lung epithelial cells. In still other embodiments, the target tissue is muscle, e.g., skeletal muscle.

As described above, the term “about” when used to modify a numerical value means a variation of ±10%, (±10%, e.g., ±1, ±2, ±3, ±4, ±5, ±6, ±7, ±8, ±9, ±10, or values therebetween) from the reference given, unless otherwise specified.

In certain instances, the term “E+#” or the term “e+#” is used to reference an exponent. For example, “5E10” or “5e10” is 5 x 10¹⁰. These terms may be used interchangeably.

As used throughout this specification and the claims, the terms “comprise” and “contain” and its variants including, “comprises”, “comprising”, “contains” and “containing”, among other variants, is inclusive of other components, elements, integers, steps and the like. The term “consists of” or “consisting of” are exclusive of other components, elements, integers, steps and the like.

It is to be noted that the term “a” or “an”, refers to one or more, for example, “an enhancer”, is understood to represent one or more enhancer(s). As such, the terms “a” (or “an”), “one or more,” and “at least one” is used interchangeably herein.

With regard to the description of these inventions, it is intended that each of the compositions herein described, is useful, in another embodiment, in the methods of the invention. In addition, it is also intended that each of the compositions herein described as useful in the methods, is, in another embodiment, itself an embodiment of the invention.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### EXAMPLES

The following examples are illustrative only and are not a limitation on the invention described herein.

As the COVID-19 pandemic worsened, variants of SARS-CoV-2 emerged and were characterized by increased binding to the angiotensin-converting enzyme 2 (ACE2) receptor as well as enhanced pathogenicity and transmissibility. These variants can circumvent pre-existing immunity to COVID-19 infections, which suggests that first-generation vaccines and monoclonal antibodies may now be less effective. We describe an approach for preventing clinical sequalae and the spread of SARS-CoV-2 variants by expressing an affinity-matured decoy of the ACE2 receptor in the proximal airways following intranasal delivery of an adeno-associated viral (AAV) vector. We show that the ACE2 decoy is not only highly active against all relevant CoV-2 variants, but also against other CoV members of the Clade 1 betacoronavirus family. Intranasal delivery of an AAV expressing the affinity-matured ACE2 decoy significantly diminished clinical and pathologic consequences of a SARS-CoV-2 challenge in a mouse model. Moreover, we achieved therapeutic levels of decoy expression at the surface of proximal airways when we delivered the decoy intranasally to nonhuman primates. This approach could help combat COVID-19 surges caused by SARS-CoV-2 variants and may serve as a countermeasure in future pandemics caused by pre-emergent ACE2-dependent CoVs poised for zoonosis.

We set out to address these issues by developing a gene-therapy-based, passive prophylaxis to SARS-CoV-2 infection using an antiviral decoy transgene that may be especially resistant to viral evolution. ACE2 decoys-soluble form of angiotensin-converting enzyme 2 -are considered a protein therapeutic in the treatment of COVID-19 patients [Chan KK, et al, An engineered decoy receptor for SARS-CoV-2 broadly binds protein S sequence variants. Sci Adv. 2021;7(8). Epub 2021/02/19. doi: 10.1126/sciadv.abf1738. PubMed PMID: 33597251; PubMed Central PMCID: PMCPMC7888922; Glasgow A, et al. Engineered ACE2 receptor traps potently neutralize SARS-CoV-2. Proc Natl Acad Sci U S A. 2020;117(45):28046-55. Epub 2020/10/24. doi: 10.1073/pnas.2016093117. PubMed PMID: 33093202; PubMed Central PMCID: PMCPMC7668070]. We isolated an affinity-matured ACE2 decoy that broadly neutralizes SARS-CoV-2 variants and demonstrate its potential for preventing COVID-19 when expressed from an adeno-associated virus (AAV) following intranasal (IN) delivery. We have reported previously on the effectiveness of IN AAV to express antibodies that broadly neutralize pandemic strains of influenza. We report a novel AAV capsid with superior gene transfer in the primate proximal airway and demonstrate therapeutic levels of active protein expression following AAV-decoy treatment. We also discuss the applications of the IN-AAV decoy approach for current and future coronavirus pandemics.

### Example 1. Engineering an ACE2 Decoy Variant With Enhanced Activity for Intranasal Gene Therapy to Prevent Infection by SARS-CoV-2 Variants

### ACE2 Decoy Affinity Maturation of ACE2 Decoy Enhances Neutralization 100-fold of SARS-CoV-2 300-fold

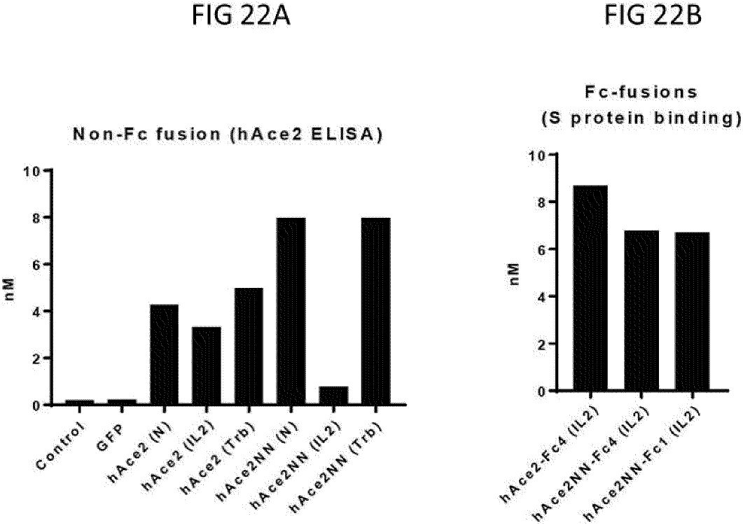

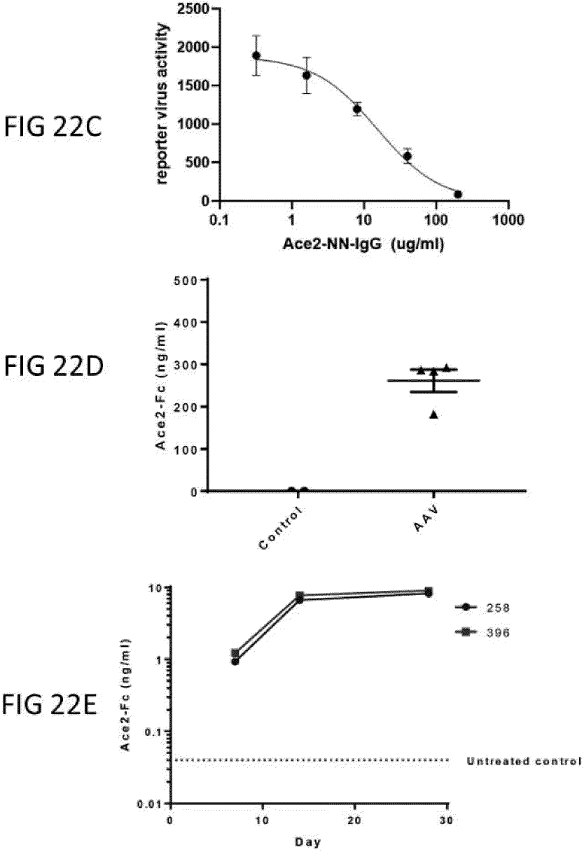

We initially constructed a decoy receptor by fusing a human ACE2 fragment to the human IgG4 Fc domain (IgG4 and Fc4 are used interchangeably throughout to indicate an Fc domain of IgG4 in various hAce2 decoy constructs). We cloned this first-generation decoy into AAV and delivered it as a nasal spray into nonhuman primates (NHPs). Although we detected decoy expression in nasal lavage fluid (NLF), the decoy was not produced at levels sufficient to overcome the low neutralizing potency of this protein (FIGS. 22A to 22 E). FIGS. 22A to 22E shows characterization of initial ACE2 decoy construct. FIG. 22A shows results for construct expressed in HEK293 cells and detected in supernatant using a sandwich ELISA to ACE2 for constructs without an Fc domain. FIG. 22B shows result of an ELISA with SARS-CoV-2 spike protein as a capture antigen and an anti-human IgG polyclonal antibody used for detection of hAce2 with Fc fusion proteins expressed in HEK293 cells. FIG. 22C shows the purified ACE2-NN-Fc4 protein was titrated against Wuhan CoV2 pseudotyped lentivirus bearing a luciferase reporter. The IC50 was obtained from a fit of these data (15 µg/ml). FIG. 22D shows the candidate construct (ACE2-NN-Fc4) was packaged in an AAV vector (hu68 capsid) and administered IN to WT mice. Seven days after administration, BAL was collected for measurement of transgene expression using an ELISA with SARS-CoV-2 spike protein as a capture antigen to confirm that the decoy receptor expressed in vivo was functional. BAL from similar experiments was 6-fold diluted from the ASF as determined by comparison of BAL and serum urea. Thus, we determined that ASF concentrations of the decoy were likely below 2 µg/ml. FIG. 22E shows two NHPs (IDs 258 and 396) received 9 x 10¹²GC of an AAVhu68 vector expressing a soluble ACE2-NN-Fc fusion protein via the MAD . Nasal lavage samples were collected weekly after vector administration and concentrated 10-fold for analysis. The concentration of the decoy receptor in NLF was measured by MS. Urea measurements in similar experiments indicate that 10X nasal lavage is ~8-fold diluted from ASF. We therefore determined that ASF concentrations of the decoy were less than 100 ng/ml. We therefore set out to affinity-mature the ACE2 protein sequence.

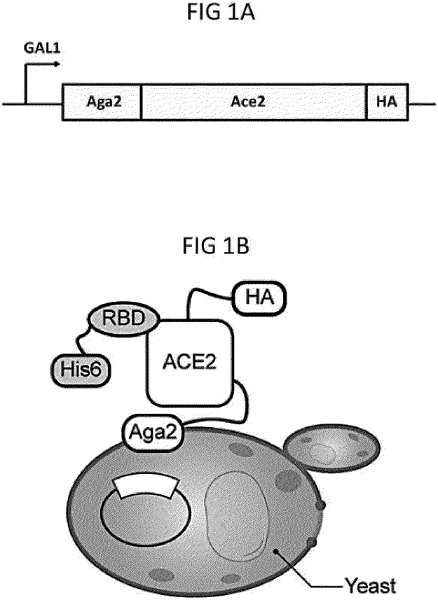

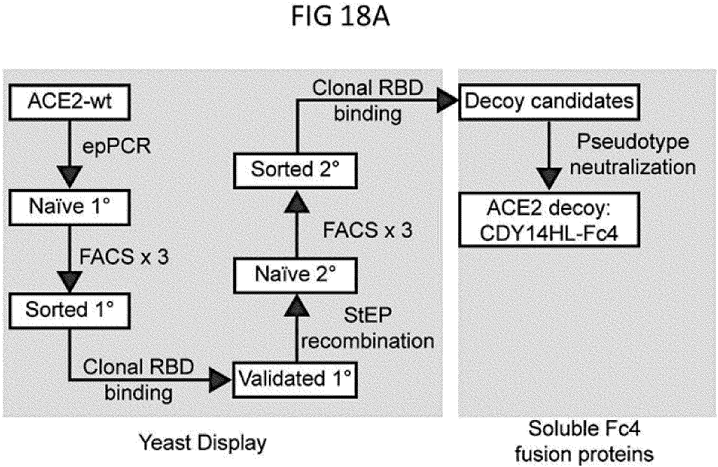

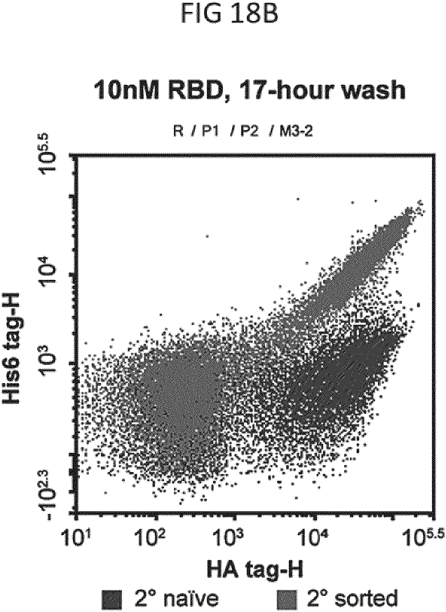

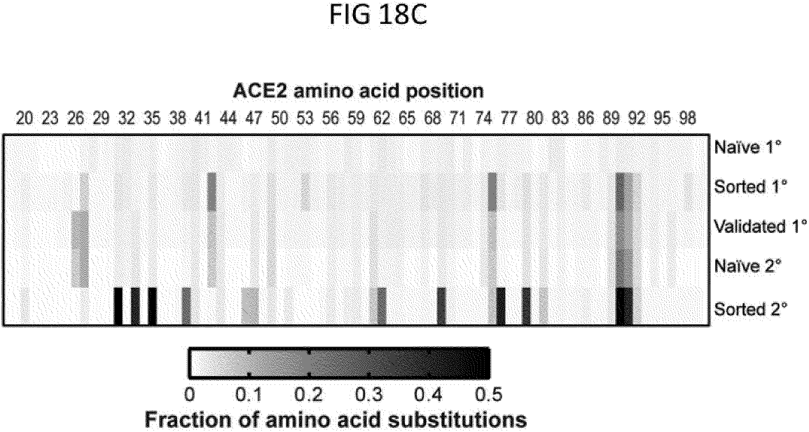

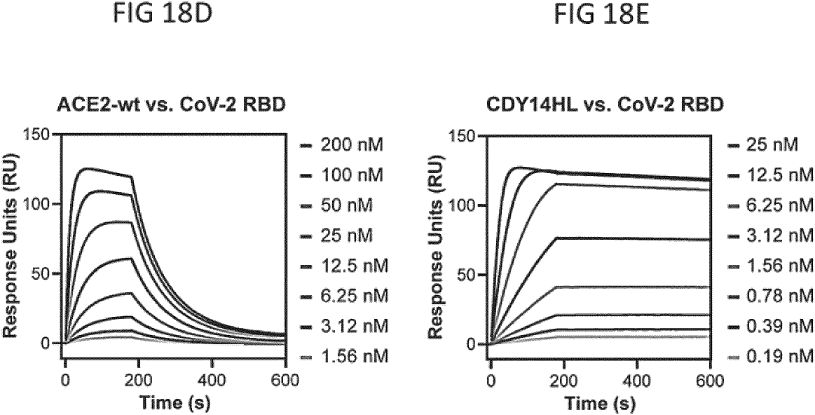

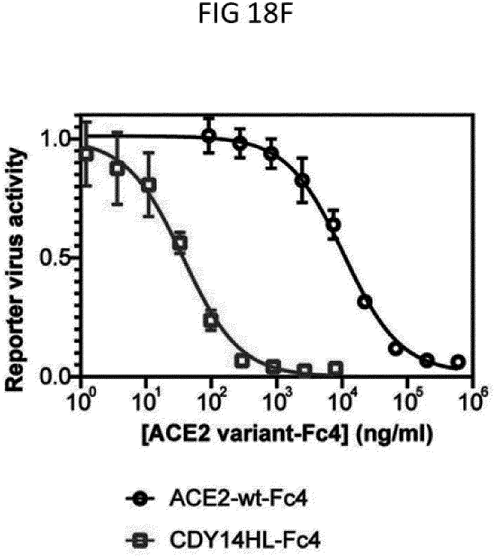

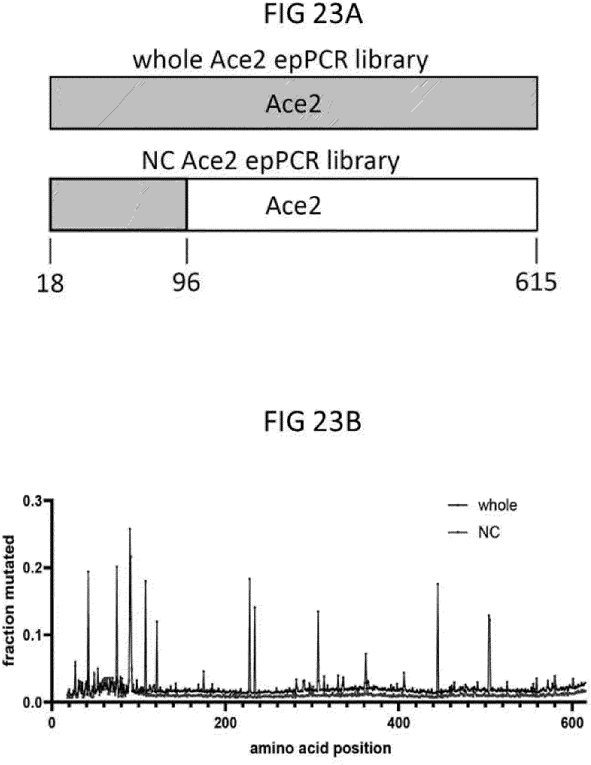

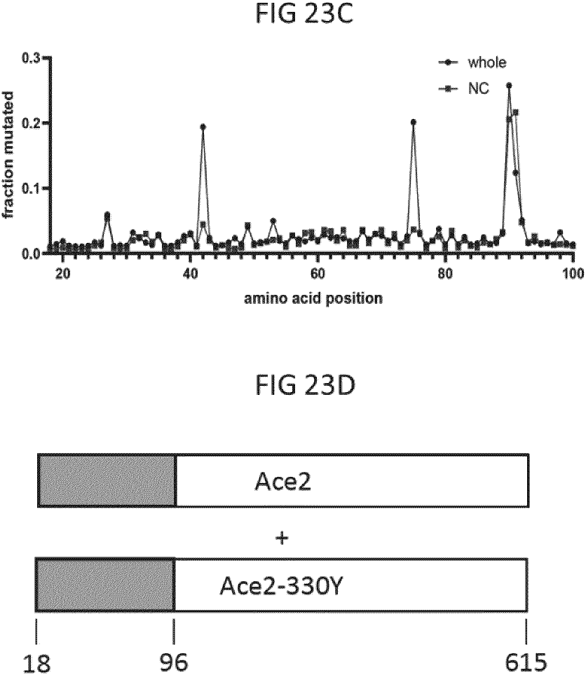

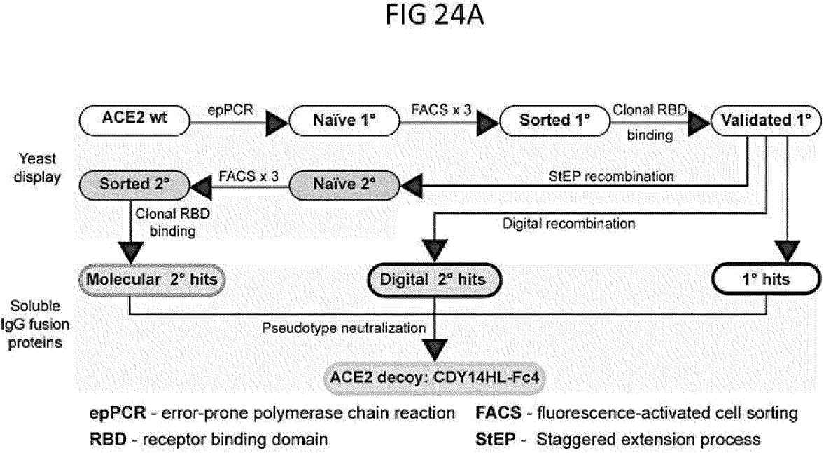

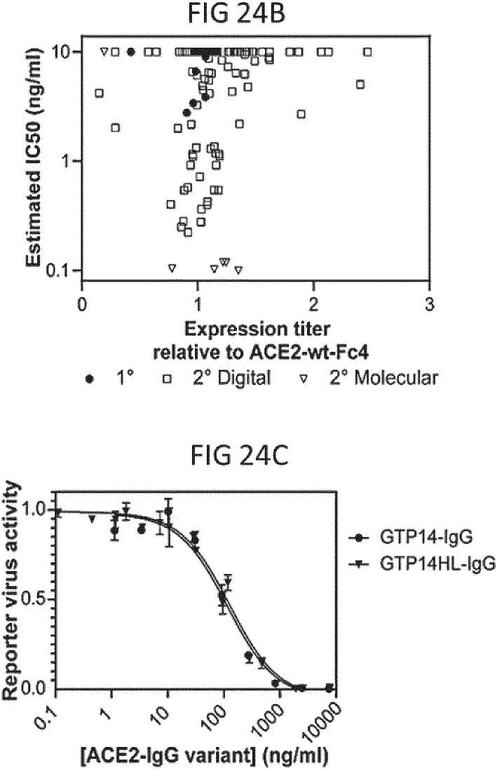

We generated diverse (>10⁸ transformants) ACE2 variant libraries in a yeast-display format⁷ using error-prone polymerase chain reaction (PCR; FIG. 1B and FIGS. 23A to 23D). We screened the primary libraries in rounds of fluorescence-activated cell sorting (FACS; FIG. 18B). We selected populations with better binding to SARS-CoV-2 receptor binding domain (RBD) and tracked library convergence with deep sequencing (FIGS. 18B and 18C). Frequently observed mutations from our primary library sorts overlap partially with mutations reported by others including substitutions at T27 and N90 glycan disruption (FIGS. 23A to 23D). Validated clones from the sorted primary libraries (FIGS. 24A to 24C) seeded a secondary library formed by mutagenic recombination [Aguinaldo AM, Arnold FH. Staggered extension process (StEP) in vitro recombination. Methods Mol Biol. 2003;231:105-10. Epub 2003/06/26. doi: 10.1385/1-59259-395-X:105. PubMed PMID: 12824608; Eckert-Boulet N, et al, Optimization of ordered plasmid assembly by gap repair in Saccharomyces cerevisiae. Yeast. 2012;29(8):323-34. Epub 2012/07/19. doi: 10.1002/yea.2912. PubMed PMID: 22806834], which we screened using stringent off-rate sorting [Boder ET, Midelfort KS, Wittrup KD. Directed evolution of antibody fragments with monovalent femtomolar antigen-binding affinity. Proc Natl Acad Sci U S A. 2000;97(20):10701-5. Epub 2000/09/14. doi: 10.1073/pnas.170297297. PubMed PMID: 10984501; PubMed Central PMCID: PMCPMC27086.] (FIG. 18B).

We expressed ACE2 variants from several stages of the yeast-display screening as soluble IgG4 Fc fusions, evaluated expression titers, and predicted IC₅₀ for SARS-CoV-2 neutralization using reporter virus (FIGS. 24A to 24C). The most potent neutralizing variants converged upon similar substitutions at five positions: 31, 35, 79, 330, and N90 glycan disruption (top 5 accumulated mutations in hAce2 decoy (from molecular library) are shown in a table immediately below).

| | | | | | | | | | | |
|---|---|---|---|---|---|---|---|---|---|---|
| AA position | 31 | 35 | 39 | 42 | 47 | 59 | 79 | 90 | 91 | 330 |
| ACE2 | K | E | L | Q | S | V | L | N | L | N |
| CDY01 | M | K | | R | | | | | P | Y |
| CDY03 | R | V | P | | | A | F | D | | Y |
| CDY05 | M | K | | | | | I | | P | Y |
| CDY09 | M | K | | | | | F | | P | Y |
| CDY14 | M | K | | | A | | F | | P | Y |

Additionally, shown in a table immediately below are the mutations from digital library.

| | | | | | | | | | | | | | | | | | |
|---|---|---|---|---|---|---|---|---|---|---|---|---|---|---|---|---|---|
| AA position | 27 | 31 | 35 | 39 | 42 | 43 | 47 | 49 | 59 | 61 | 75 | 76 | 79 | 81 | 90 | 91 | 330 |
| ACE2 | T | K | E | L | Q | S | S | N | V | N | E | Q | L | Q | N | L | N |
| CDY12 | A | | | | | | | D | | D | K | | | | | P | Y |
| CDY16 | A | | | | | G | | | | D | | | | R | | P | Y |
| CDY17 | A | | | | | | | D | | D | | | F | | S | | Y |
| CDY23 | A | | | | | G | | D | | | | | F | | | P | Y |
| CDY24 | A | | | | | G | | D | | D | | R | | | | | Y |

After further characterization, we selected hAce2-Variant1-Fc4 (CDY14-FC4) as the most improved ACE2 decoy variant. To avoid off-target effects in vivo, we ablated ACE2 enzyme activity by introducing H345L at no cost to potency [Guy, J. L., Jackson, R. M., Jensen, H. A., Hooper, N. M. & Turner, A. J. Identification of critical active-site residues in angiotensin-converting enzyme-2 (ACE2) by site-directed mutagenesis. FEBS J 272, 3512-3520, doi:10.1111/j.1742-4658.2005.04756.x25 (2005)]. By surface plasmon resonance (SPR) the active site-null hAce2-Variant2-Fc4 (CDY14HL- Fc4) bound SARS-CoV-2 RBD with 1,000-fold improved affinity (29 nM for wtACE2 vs. 31 pM for hAce2-Variant2-Fc4; see FIG. 18D and FIG. 18E). hAce2-Variant2-Fc4 neutralized Wuhan-Hu-1 SARS-CoV-2 reporter nearly 300-fold better than the un-engineered ACE2 decoy (IC₅₀ 127 ng/ml for hAce2- hAce2-Variant2-Fc4 (CDY14HL- Fc4) vs. 11 µg/ml for ACE2-wt-Fc4; see FIG. 18F).

Table: Potency of CDY14HL-Fc4 in the neutralization of lentiviruses pseudotyped with CoV spike variants.

| | | | | |
|---|---|---|---|---|
| 1^{st} Seq Location/ Common Name | PANGO lineage (other designation) | RBD mutations | 614 | IC50 mean ± SEM (ng/ml) |
| Wuhan | A.1 (Nextstrain 19B) | - | D | 37 ± 6 |
| 614G | B.1 | - | G | 22 ± 3 |
| Scotland/EU | B.1.141 | N439K | G | 15 ± 3 |
| UK | B.1.1.7 | N501Y | G | 29 ± 4 |
| South Africa | B.1.351 | K417N, E484K, N501Y | G | 28 ± 5 |
| Brazil | P.1 | K417T, E484K, N501Y | G | 16 ± 5 |
| Ohio-501Y | (COH.20G/501Y) | 501Y | G | 22 ± 3 |
| Ohio-677H | (COH.20G/677H) | - | G | 28 ± 4 |
| California | B.1.427/B.1.429 | L452R | G | 23 ± 6 |
| Australia/Europe | (20A.EU2) | S477N | G | 36 ± 5 |
| New York | B.1.526 | E484K | G | 53 ± 17 |
| CoV1-Urbani | N/A | N/A | N/A | 18 ± 5 |

### ACE2 Decoy Is Effective Against SARS-CoV-2 Variants and SARS-CoV-1

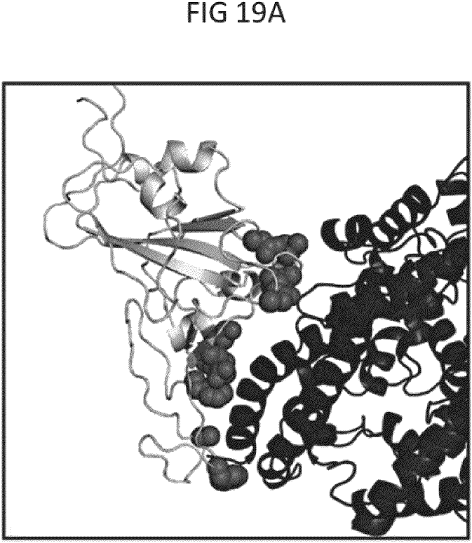

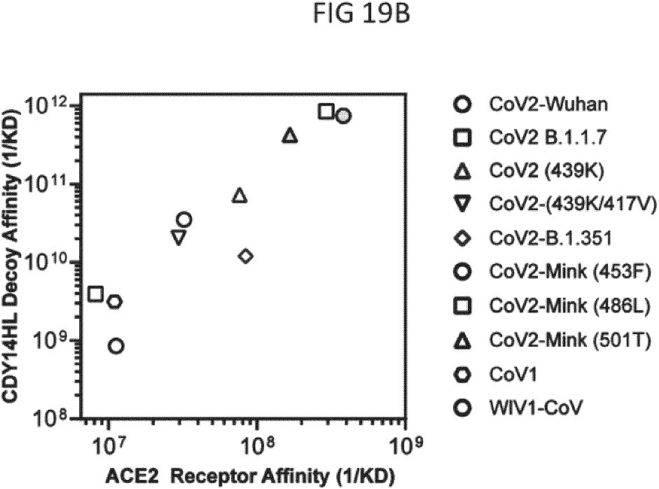

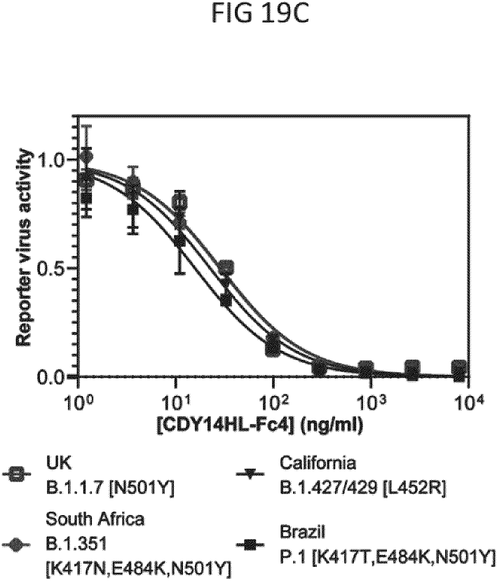

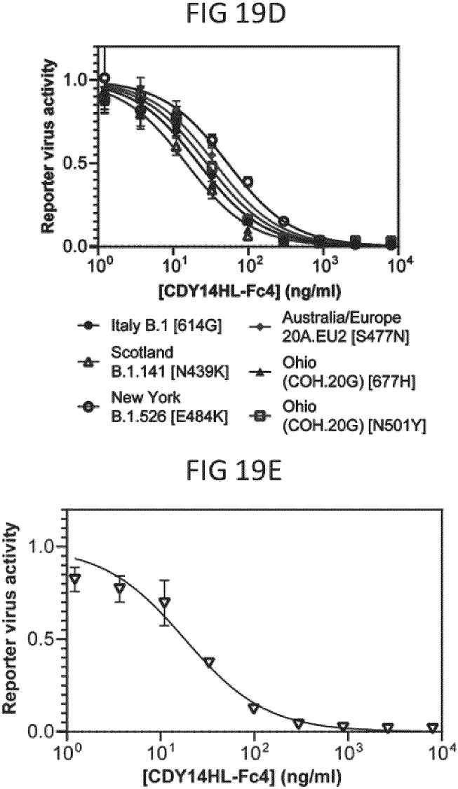

Unlike antibodies, decoy inhibitors may achieve broad neutralization and escape mutant resistance; changes that reduce decoy binding would also decrease ACE2 receptor binding, thus reducing viral fitness. Escape mutations at the immunodominant ACE2 binding site of the RBD is of major concern for emerging SARS-CoV-2 variants [Starr, T. N. et al. Prospective mapping of viral mutations that escape antibodies used to treat COVID-19. Science 371, 850-854, doi:10.1126/science.abf9302 (2021)]. RBDs from more distant ACE2-dependent CoVs also differ substantially from the original SARS-CoV-2 at the ACE2 interface (FIG. 19A). By the same logic, decoys should maintain neutralizing potency even as viruses evolve tighter binding to their receptor to achieve more efficient infection. Mutations at six sites at the RBD:ACE2 interface have arisen in multiple independent SARS-CoV-2 lineages (FIG. 19A). To varying degrees, mutations at these sites have been reported to confer enhanced ACE2 binding, transmissibility, virulence, and escape from first-generation monoclonal antibody therapeutics or even active vaccines. To assess the potential of our decoy for broad neutralization, we measured ACE2-Fc4 (a surrogate for the native receptor) and hAce2-Variant2-Fc4 (CDY14HL- Fc4) (the therapeutic decoy) affinities across a diverse panel of CoV RBDs using SPR. We chose RBDs that contain mutation combinations observed at these six sites (FIG. 19A), as well as other strains that have been reported. These include strains under positive selection in the course of the 2020 pandemic (e.g., 439 K from B.1.141, first isolated in Scotland [Thomson EC, et al. Circulating SARS-CoV-2 spike N439K variants maintain fitness while evading antibody-mediated immunity. Cell. 2021;184(5):1171-87 e20. Epub 2021/02/24. doi: 10.1016/j.cell.2021.01.037. PubMed PMID: 33621484; PubMed Central PMCID: PMCPMC7843029]; 501Y from B.1.1.7, first isolated in the UK; 417N/484K/501Y from B1.351, first isolated in the Republic of South Africa; 452R/484Q from B.1.617, first isolated in India), and mink-adapted isolates [Oude Munnink BB, et al. Transmission of SARS-CoV-2 on mink farms between humans and mink and back to humans. Science. 2021;371(6525):172-7. Epub 2020/11/12. doi: 10.1126/science.abe5901. PubMed PMID: 33172935; PubMed Central PMCID: PMCPMC7857398]. Several emerging SARS-CoV-2 variants with improved affinity for ACE2-Fc4 (501Y, 453F, and 501T) also bind CDY14HL-Fc4 more tightly (FIG. 19B and table immediately below), while 417N/484K/501Y, 439 K, and 439 K/417V, and 452R/484Q only modestly alter binding to ACE2-Fc4 or CDY14HL-Fc4. While 486L reduces affinity for CDY14HL-Fc4, it does so for ACE2-Fc4 proportionally.

| Binding Sample | ka (⅟Ms) | Kd (⅟s) | K_{D} (M) | Rₘₐₓ (RU) |
|---|---|---|---|---|
| WT Ace2 vs. WT SARS-CoV-2 RBD | 5.05 x 10⁵ | 0.01441 | 2.86 x 10⁻⁸ | 140.7 |
| WT Ace2 vs. WT SARS-CoV-1 RBD | 4.26 x 10⁵ | 3.94 x 10-² | 9.24 x 10⁻⁸ | 142.6 |
| WT Ace2 vs. UK 501Y RBD | 6.26 x 10⁵ | 2.07 x 10⁻³ | 3.30 x 10⁻⁹ | 150.5 |
| WT Ace2 vs. RSA 417N 484 K 501Y RBD | 4.39 x 10⁵ | 5.37 x 10⁻³ | 1.22 x 10⁻⁸ | 148.7 |
| CDY14-HL vs. WT SARS-CoV-2 RBD | 4.62 x 10⁶ | 1.42 x 10⁻⁴ | 3.08 x10⁻¹¹ | 125.3 |
| CDY14-HL vs. WT SARS-CoV-1 RBD | 2.94 x 10⁶ | 9.21 x 10⁻⁴ | 3.13 x 10⁻¹⁰ | 134.9 |
| CDY14-HL vs. UK 501Y RBD | 5.36 x 10⁶ | 5.57 x 10⁻⁶ | 1.04 x10⁻¹² | 149.7 |
| CDY14-HL vs. RSA 417N 484 K 501Y RBD | 1.85 x 10⁶ | 1.56 x 10⁻⁴ | 8.42 x 10⁻¹¹ | 123.1 |

Table immediately above shows example SPR data for RBD binding assay. Raw data (colored lines) and fits (black lines) for immobilized ACE2-wt-Fc4 on the SPR chip surface binding injected RBDs (concentrations listed). Parameters of the fits, including the dissociation equilibrium constant (KD) are listed below each panel. These data contributed to FIG. 19B and19. B. Raw data (colored lines) and fits (black lines) for immobilized hAce2-Variant2-Fc4 (CDY14HL-Fc4) on the SPR chip surface binding injected RBDs.

Next, we examined RBDs from more distantly related CoVs. Comparative structural analysis indicates that SARS-CoV-1 and SARS-CoV2 differ at 11 of the 19 interfacial residues critical for ACE2-binding (FIG. 19B). A similar degree of divergence at the ACE2 binding site exists for the pre-emergent bat coronavirus, WIV1-CoV. Remarkably, the decoy binds both of these distantly related CoV domains with higher affinity than the endogenous ACE2 receptor (FIG. 19C). The tight coupling of decoy and receptor affinities seen in the panel of CoV-2 variants is extended across the clade of ACE2-dependent betacoronaviruses and underscores the potential for broad neutralization by CDY14HL-Fc4.

Next, we compared decoy neutralization across diverse SARS-CoVs using pseudotyped lentivirus reporters. We observed potent neutralization of pseudotypes for the 4 strains currently considered Variants of Concern by the United States Centers for Disease Control (FIG. 19D). The neutralization IC50s are lower for these strains than for the Wuhan pseudotype, reaching 16 ng/ml for the P.1 reporter (Table above).

This increased potency may be explained by a combination of effects, including the RBD-exposing 614G mutation that occurs in the later lineages (FIG. 19C and Table above), and the decoy affinity-enhancement associated with some RBD substitutions contained within these strains (FIG. 19B). We measured neutralizing potencies for 6 additional pseudotypes encoding spike variants that have emerged on different continents and at different stages of the COVID-19 pandemic (FIGS. 19D and 19E). Most are neutralized with IC50 values under 30 ng/ml (Table above). Remarkably, selecting for increased binding to the SARS-CoV-2 RBD resulted in very potent neutralization of the phylogenetically distinct SARS-CoV-1 reporter virus (IC50 = 18 ng/ml, FIG. 19E and Table above). Taken together with the binding survey, these data indicate that structural features of the ACE2 interface have been retained through the stages of directed evolution. Moreover, the data predict that CDY14HL-Fc4 could protect against current, emerging, and future pandemic ACE2-dependent CoVs.

### ACE2 Decoy Diminishes SARS-CoV-2 Sequelae in Transgenic Mice

We considered SARS-CoV-2 challenge studies in hamsters, macaques, and the hACE2 transgenic (TG) mice to evaluate the in vivo efficacy of an AAV vector expressing hAce2-Variant2-Fc4 (CDY14HL-Fc4). In all models, achieving evidence of viral replication in vivo requires virus doses that far exceed those required for human transmission. Furthermore, the clinical and pathologic sequalae of SARS-CoV-2 exposure is attenuated in these species compared to severely affected humans. The most significant limitation, however, is that all the challenge models require direct pathogen delivery to the lung in order to demonstrate pathology, which does not simulate the mechanism of the AAV decoy product, which focuses on localized expression in the proximal airway following intranasal delivery to reduce SARS-CoV-2 infection and its consequences. We therefore selected the hACE2 TG mouse model for three reasons: 1) we can characterize disease by measuring viral loads, clinical sequalae, and histopathology; 2) we can use an IN route of administration as we would in humans, realizing this deposits vector in the proximal and distal airways of the mouse, while IN delivery in humans is restricted to the proximal airway; and 3) we can leverage the extensive experience of murine models in de-risking human studies of AAV gene transfer.

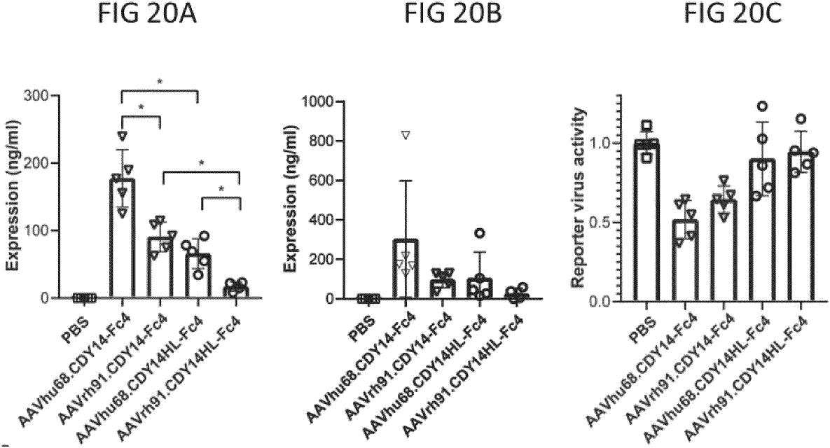

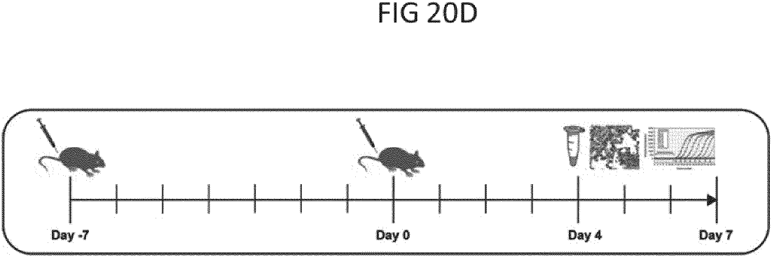

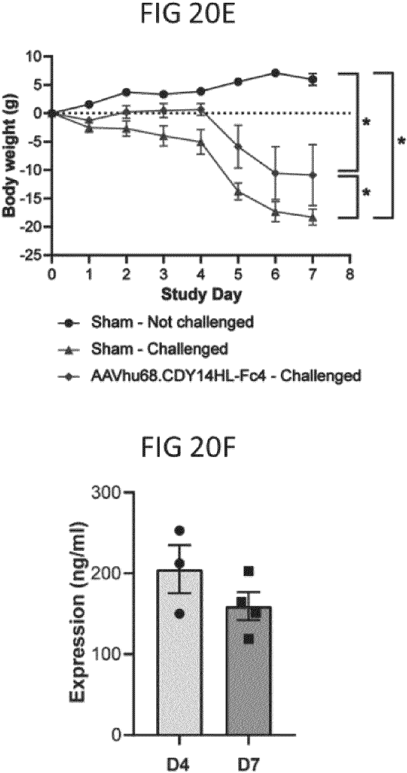

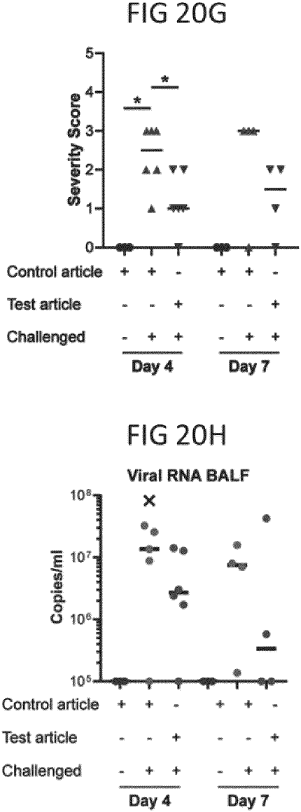

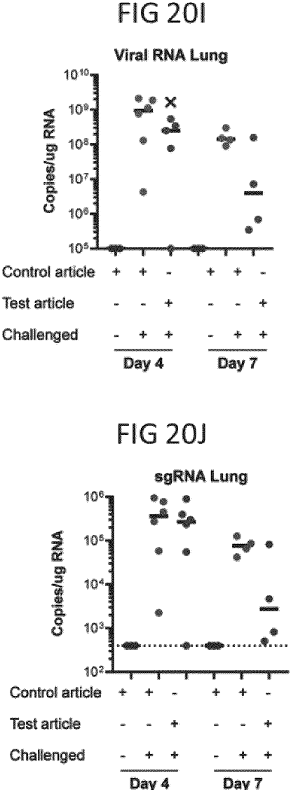

We conducted pilot studies in wild-type mice to determine which decoy protein (hAce2-Variant1-Fc4 (CDY14-Fc4) vs. hAce2-Variant2-Fc4 (CDY14HL- Fc4)) and capsid (clade F AAVhu68 vs. clade A AAVrh91) maximized expression following in vivo gene delivery. We administered 10¹¹ GC of vector and recovered broncho- alveolar lavage samples (BAL) 7 days later to evaluate ACE2 decoy protein expression and activity (FIGS. 20A to 20C). Based on mass spectrometry (MS) protein measurements, the AAVhu68 capsid was more efficient than the AAVrh91 capsid in transducing mouse lung. The HL mutation modestly reduced expression (p<0.007). Importantly, we found a direct correlation between decoy expression levels and the ability to bind to SARS-CoV-2 spike protein and neutralize a SARS-CoV-2 pseudotype, demonstrating function of the decoy expressed from airway tissues (FIGS. 20A to 20C). We selected hAce2-Variant2-Fc4 (CDY14HL- Fc4) as the clinical candidate transgene and the AAVhu68 capsid for the mouse challenge studies (FIG. 20D).

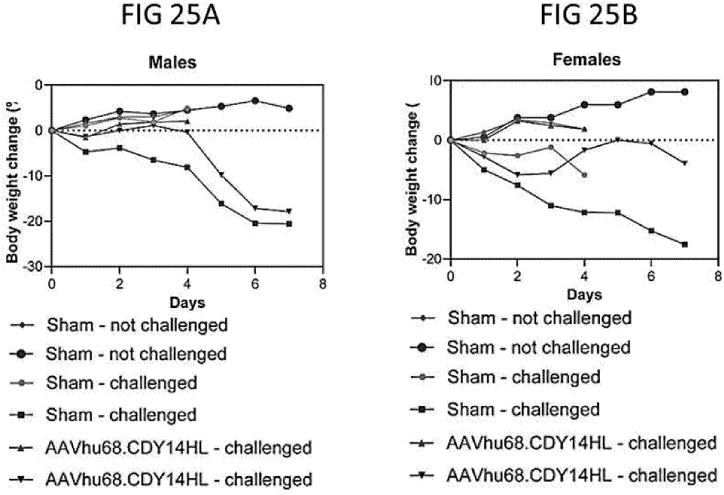

We intranasally (IN) delivered AAVhu68-hAce2-Variant2-Fc4 (AAVhu68-CDY14HL- Fc4) or vehicle to hACE2-TG mice. Seven days later, animals were challenged with SARS-CoV-2 (280 pfu), followed clinically (observation and daily weights), and necropsied on days 4 and 7 after challenge for tissue and BAL analysis (FIG. 20D). Expression of hAce2-Variant2-Fc4 (CDY14HL-Fc4) in BAL normalized for dilution was in the range of the IC50 measured in vitro and in the pilot studies (FIG. 20F). Sham-treated SARS-CoV-2 challenged animals demonstrated statistically significant weight loss as has been described by others [Winkler, E. S. et al. SARS-CoV-2 infection of human ACE2-transgenic mice causes severe lung inflammation and impaired function. Nature Immunology 21, 1327-1335, doi:10.1038/s41590- 020-0778-2 (2020); Zheng, J. et al. COVID-19 treatments and pathogenesis including anosmia in K18-hACE2 mice. Nature 589, 603-607, doi:10.1038/s41586-020-2943-z (2021); Yinda, C. K. et al. K18-hACE2 mice develop respiratory disease resembling severe COVID-19. PLOS Pathogens 17, e1009195, doi:10.1371/journal.ppat.1009195 (2021)]. We observed significantly less weight loss amongst vector-treated animals (which we followed for 7 days) compared to untreated animals (observed on days 4 and 7; p<0.05, linear mixed effect modeling). The vector-treated animals also significantly differed from the untreated, unchallenged animals (FIG. 20E). Interestingly, the clinical outcome of the treatment was better among females than males, although we noted significant variations within the treated group (FIGS. 25A and 25B).

Histopathology of the lungs from vehicle treated animals challenged with SARS-CoV-2 revealed findings similar to that previously described in this model [Yinda CK, et al. K18-hACE2 mice develop respiratory disease resembling severe COVID-19. PLOS Pathogens. 2021;17(1):e1009195. doi: 10.1371/journal.ppat. 1009195]. As expected, tissues from animals not challenged with SARS-CoV-2 demonstrated no histopathology. Samples from days 4 and 7 showed reduced lung pathology in AAVhu68-hAce2-Variant2-Fc4 (AAVhu68-CDY14HL- Fc4) treated animals vs. the vehicle-treated animals; the day-4 samples achieved statistical significance (p<0.05; Wilcoxon Rank Sum Test). (FIG. 20G). Compared to vehicle-treated animals, viral RNA in BAL and lung homogenate was diminished at day 4 and 7 in AAVhu68-hAce2-Variant2-Fc4 (AAVhu68.CDY14HL-Fc4) treated animals (FIGS. 20H and 20I). The greatest reductions were at day 7 for both BAL (26-fold) and lung tissue (35- fold). Impact of the AAVhu68-hCAe2-Variant2-Fc4 (AAVhu68.CDY14HL-Fc4) on SARS-CoV-2 replication, as determined by median sgRNA levels, was greatest at day 7 (27-fold reduction, FIG. 20J). Although there was substantial inter-animal variation, ⅖ animals in the treated groups showed nearly complete abrogation of viral replication and little weight loss by day 7.

### AAV Delivery Yields Therapeutic ACE2 Decoy Levels in Nonhuman Primates

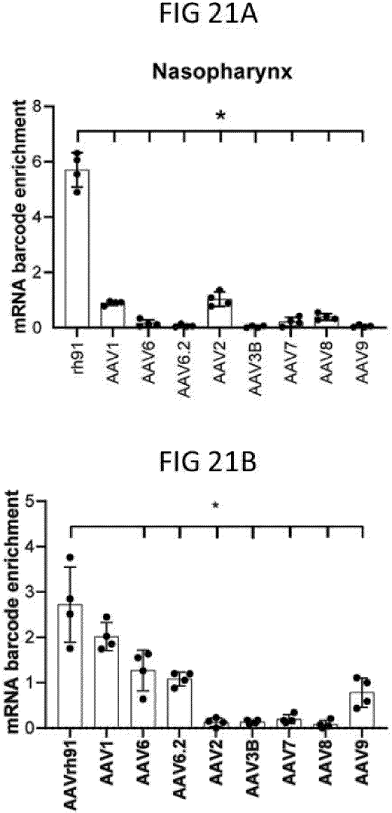

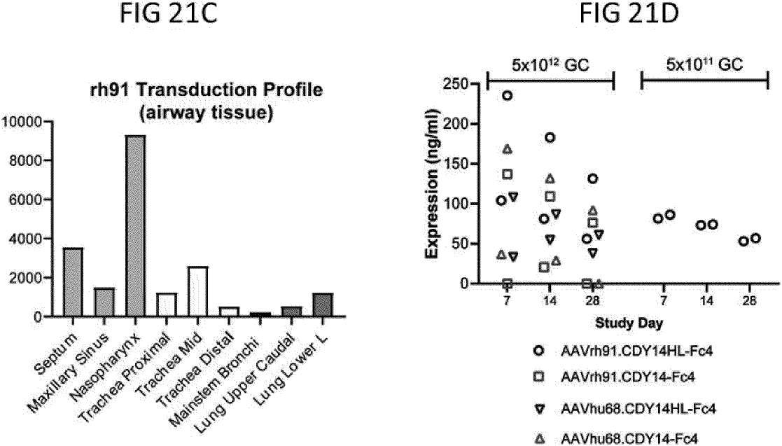

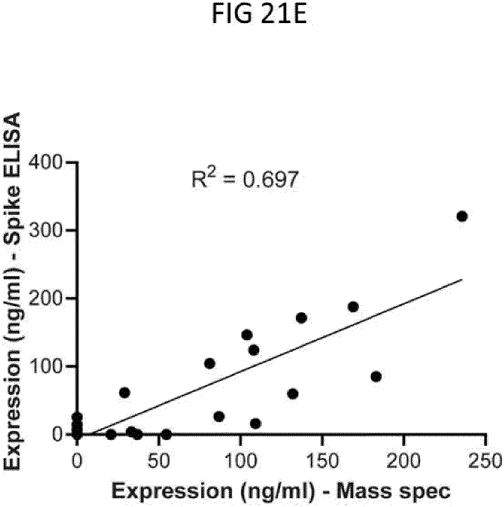

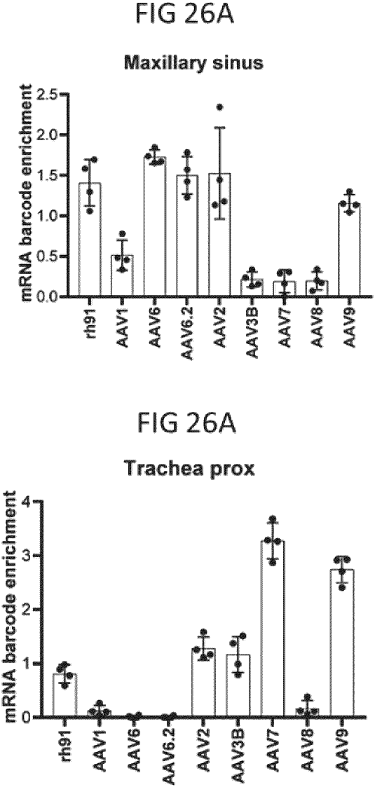

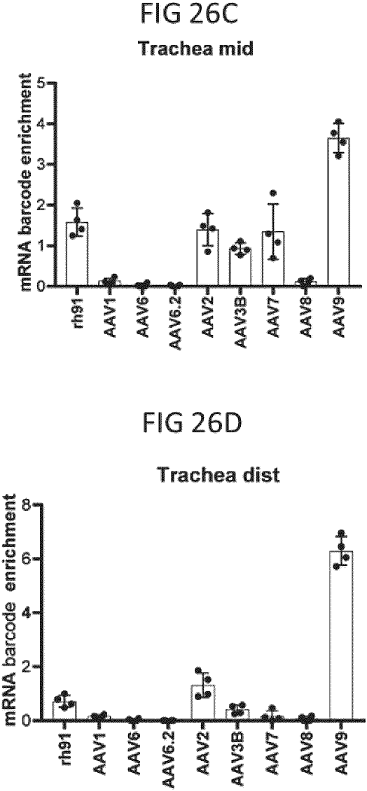

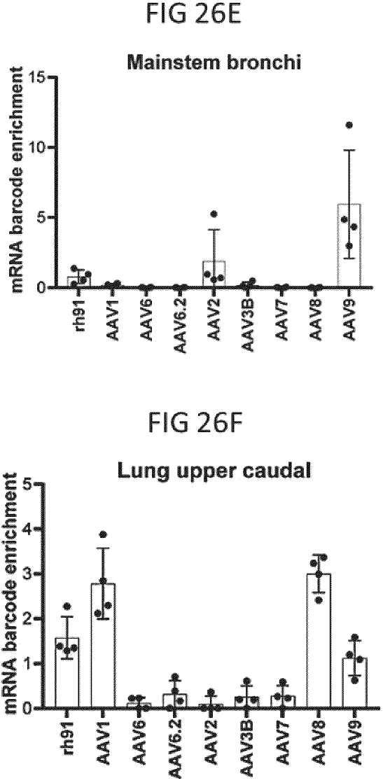

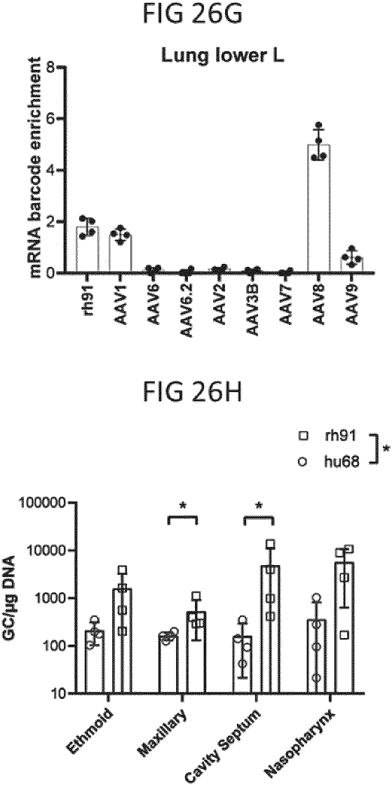

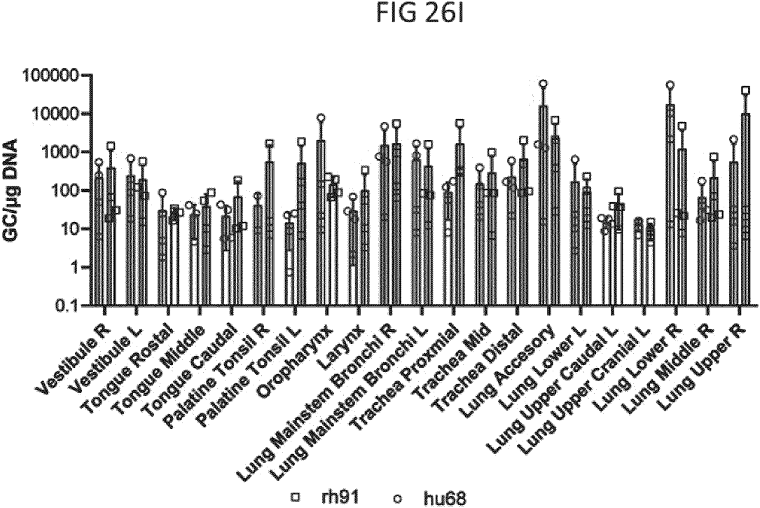

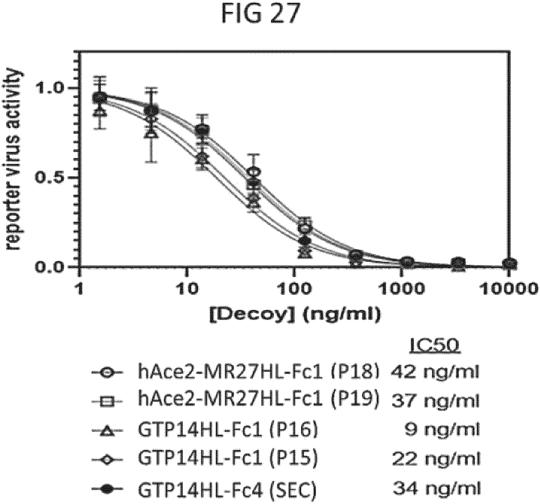

Next, we determined which AAV capsid is most efficient at transducing cells of the nonhuman primate (NHP) proximal airways—the desired cellular targets for COVID-19 prophylaxis following nasal delivery of vector. We administered vector using a previously approved intranasal mucosal atomization device (MAD Nasal™), which comprises an atomizing tip with a soft conical nostril seal fit on a standard syringe). A mixture of 9 AAV serotypes with uniquely barcoded transgenes were administered via the MAD Nasal^{Tm} to an NHP. Tissues were harvested 14 days later for evaluation of relative transgene expression using the mRNA bar-coding technique [Zheng J, Wong, et al. COVID-19 treatments and pathogenesis including anosmia in K18-hACE2 mice. Nature. 2021;589(7843):603-7. doi: 10.1038/s41586-020-2943-z.]. The novel Clade A capsid (AAVrh91) we isolated from macaque liver performed best in the nasopharynx and septum (FIGS. 21A and 21B) with low but detectable expression levels in large airways and distal lung (FIGS. 26A to 26I). Clade E and F capsids performed better than AAVrh91 in some non-target tissues such as distal lung (FIGS. 26A to 26I). The profile of expression from AAVrh91 illustrates relative distribution of transgene expression with proximal airway structures>intra-pulmonary conducting airway>distal lung (FIG. 21C).

To determine the candidate for clinical evaluation, we conducted a final NHP study where groups of 2 animals were administered 5x10¹² GC of vectors that differed with respect to capsid (AAVhu68 vs. AAVrh91) and transgene cassettes (hAce2-Variant1-Fc4 (CDY14-Fc4) vs. hAce2-Variant2-Fc4 (CDY14HL-Fc4)). NLFs were harvested on days 7, 14, and 28, and animals were necropsied on day 28 for biodistribution. Analysis of pulmonary tissues from day 28 revealed broad distribution throughout the proximal and distal airway, with AAVrh91 demonstrating superior gene transfer to proximal airway structures, as suggested by the barcode study (FIGS. 26A to 26I). We estimated decoy protein concentrations in the air-surface liquid (ASF) based on dilution-adjusted MS measurements of NLF (FIG. 21C). The effective concentrations at the ASF were in the range that demonstrated neutralization in the in vitro assay. Expression was slightly higher with AAVrh91 vs. AAVhu68, and hAce2-Variant1-Fc4 (CDY14-Fc4) vs. hAce2-Variant2-Fc4 (CDY14HL-Fc4). A subset of samples evaluated for binding to the spike protein of SARS-CoV-2 showed a good correlation with decoy protein as measured by MS. This indicates that the decoy protein produced in vivo in proximal airways is indeed functional (FIG. 21E).

Based on these data, we selected a candidate for subsequent clinical evaluation AAVrh91-hAce2-Variant2-Fc4. This candidate utilizes AAVrh91 as the capsid because of its transduction profile and hAce2-Variant2-Fc4 (CDY14HL-Fc4) as the transgene because it retained broad and potent neutralizing activity in the setting of an ACE2-disabling mutation. In preparation for IND-enabling studies, we administered clinical candidate at a 10-fold lower dose to two additional NHPs (FIG. 21D). Impressive levels of decoy protein were present in nasal ASF with concentrations only slightly reduced in comparison to those achieved with the higher dose.

### Discussion

The rapid emergence of more dangerous and transmissible variants of SARS-CoV-2 in this pandemic is troubling, but not unexpected. The immunological pressures on the virus during natural infections, following antibody therapies, and active vaccines have promoted the emergence of variants [Yinda CK, et al. K18-hACE2 mice develop respiratory disease resembling severe COVID-19. PLOS Pathogens. 2021;17(1):e1009195. doi: 10.1371/journal.ppat.1009195]. It appears that SARS-CoV-2 improved fitness through mutations that both increased affinity for ACE2 and decreased neutralization by antibodies elicited to precursor strains of the virus [Yinda CK, et al. 2021, cited above; Adachi K, et al, Drawing a high-resolution functional map of adeno-associated virus capsid by massively parallel sequencing. Nat Commun. 2014;5:3075. Epub 2014/01/18. doi: 10.1038/ncomms4075. PubMed PMID: 24435020; PubMed Central PMCID: PMCPMC3941020; Ozono S, et al. SARS-CoV-2 D614G spike mutation increases entry efficiency with enhanced ACE2-binding affinity. Nat Commun. 2021;12(1):848. Epub 2021/02/10. doi: 10.1038/s41467-021-21118-2. PubMed PMID: 33558493; PubMed Central PMCID: PMCPMC7870668]. The density of ACE2 in the nose and airway has been linked to pathogenicity and transmissibility of SARS-CoV-2 [Zhou D, Dejnirattisai W, Supasa P, Liu C, Mentzer AJ, Ginn HM, et al. Evidence of escape of SARS-CoV-2 variant B.1.351 from natural and vaccine-induced sera. Cell. 2021. Epub 2021/03/18. doi: 10.1016/j.cell.2021.02.037. PubMed PMID: 33730597; PubMed Central PMCID: PMCPMC7901269]. The lower levels of ACE2 in the proximal airways of children may be responsible for the lower infection rates and milder symptoms in this group [Bunyavanich, S., Do, A. & Vicencio, A. Nasal Gene Expression of Angiotensin-Converting Enzyme 2 in Children and Adults. JAMA 323, 2427-2429, doi:10.1001/jama.2020.8707 (2020)]. It has been proposed that SARS-CoV-2 variants achieve greater infection and transmission through increased affinity [Adachi, et al, cited above; Bunyavanich S, et al, Nasal Gene Expression of Angiotensin-Converting Enzyme 2 in Children and Adults. JAMA. 2020;323(23):2427-9. Epub 2020/05/21. doi: 10.1001/jama.2020.8707. PubMed PMID: 32432657; PubMed Central PMCID: PMCPMC7240631]; here we confirm increased affinity for ACE2 in SARS-CoV-2 strains under positive selection during 2020.

We have generated a decoy with high binding and neutralizing activity against a full range of SARS-CoV-2 variants, including B1.1.7 and B1.351, which emerged from the UK and Republic of South Africa, respectively. However, we were surprised to see equally potent binding and neutralization against other betacoronaviruses, including SARS-CoV-1, which was responsible for the 2003 SARS pandemic. The presence of several second-shell mutations in the affinity-matured decoy may contribute to this breadth since the majority of the ACE2 contact surface was preserved (FIGS. 24A to 24C). The engineered decoy may be the Achilles’ heel of any ACE2-dependent CoV whose primary driver of fitness - higher binding to its receptor - should further enhance the potency of the ACE2 decoy.

We focused on IN delivery of AAV to express hAce2-Variant2-Fc4 (CDY14HL-Fc4) to prevent COVID-19. We used the previously described hACE2-TG mouse challenge model to demonstrate efficacy of the decoy in vivo. Treated animals lost less weight, showed reduced lung pathology, and showed less replication of the challenge virus. Our results are consistent with the use of this model to evaluate convalescent plasma [Yurkovetskiy L, et al. Structural and Functional Analysis of the D614G SARS-CoV-2 Spike Protein Variant. Cell. 2020;183(3):739-51 e8. Epub 2020/09/30. doi: 10.1016/j.cell.2020.09.032. PubMed PMID: 32991842; PubMed Central PMCID: PMCPMC7492024], protease inhibitors [Ramanathan M, et al., SARS-CoV-2 B.1.1.7 and B.1.351 Spike variants bind human ACE2 with increased affinity. bioRxiv. 2021. Epub 2021/03/04. doi: 10.1101/2021.02.22.432359. PubMed PMID: 33655251; PubMed Central PMCID: PMCPMC7924271], and monoclonal antibodies [Joaquín Cáceres C, et al. Efficacy of GC-376 against SARS-CoV-2 virus infection in the K18 hACE2 transgenic mouse model. bioRxiv. 2021:2021.01.27.428428. doi: 10.1101/2021.01.27.428428], where weight loss, pulmonary pathology, and viral load were decreased, but not completely abrogated [Yurkovetskiy L, 2020, cited above]. We believe that the mouse challenge model underestimates the potential efficacy of IN AAV-hAce2-Variant2-Fc4 (AAV-CDY14HL-Fc4). The dose of SARS-CoV-2 that results in human infection is likely much lower, and therefore, easier to neutralize than the inoculating dose used in the mouse challenge model (2.5x10⁶ particles or 280 PFU). We used a novel AAV Clade A capsid called AAVrh91 to maximize transduction in the proximal airways of NHPs. At a relatively low dose (5x10¹¹ GC), we achieved levels of hAce2-Variant2-Fc4 (CDY14HL-Fc4) in the ASF that should be sufficient to neutralize SARS- CoV-2 variants. Based on our previous studies—using AAV to deliver broadly neutralizing antibodies against influenza-we believe expression should be durable for at least six months and can be effectively readministered [Glasgow A, et al. Engineered ACE2 receptor traps potently neutralize SARS-CoV-2. Proc Natl Acad Sci U S A. 2020;117(45):28046-55. Epub 2020/10/24. doi: 10.1073/pnas.2016093117. PubMed PMID: 33093202; PubMed Central PMCID: PMCPMC7668070; Adam VS, et al., Adeno-associated virus 9-mediated airway expression of antibody protects old and immunodeficient mice against influenza virus. Clin Vaccine Immunol. 2014;21(11):1528-33. Epub 2014/09/12. doi: 10.1128/CVI.00572-14. PubMed PMID: 25209558; PubMed Central PMCID: PMCPMC4248762; Laursen NS, et al. Universal protection against influenza infection by a multidomain antibody to influenza hemagglutinin. Science. 2018;362(6414):598-602. Epub 2018/11/06. doi: 10.1126/science.aaq0620. PubMed PMID: 30385580; PubMed Central PMCID: PMCPMC6241527; Limberis MP, et al. Intranasal antibody gene transfer in mice and ferrets elicits broad protection against pandemic influenza. Sci Transl Med. 2013;5(187):187ra72. Epub 2013/05/31. doi: 10.1126/scitranslmed.3006299. PubMed PMID: 23720583; PubMed Central PMCID: PMCPMC45965].

The emergence of three lethal and highly contagious CoV outbreaks in two decades - SARS in 2003, MERS in 2012, and COVID-19 in 2019 - suggests that CoVs will remain a threat to global health. Surveillance of potential zoonotic sources of these CoVs, such as bats, revealed reservoirs of related viruses capable of evolution and cross-species transmission [Latinne A, et al. Origin and cross-species transmission of bat coronaviruses in China. Nat Commun. 2020;11(1):4235. Epub 2020/08/28. doi: 10.1038/s41467-020-17687-3. PubMed PMID: 32843626; PubMed Central PMCID: PMCPMC7447761]. One possible therapeutic application of hAce2-Variant2-Fc4 (CDY14HL-Fc4) is in the prevention and treatment of future outbreaks caused by new CoVs that utilize ACE2 as a receptor. AAVrh91-hAce2-Variant2-Fc4 could be rapidly deployed from stockpiles to contain the initial outbreak and the hAce2-Variant2-Fc4 (CDY14HL-Fc4) protein can be leveraged to improve outcomes in those who are infected. The hAce2-Variant2-Fc4 (CDY14HL-Fc4) products may be useful in the current COVID-19 pandemic if SARS-CoV-2 variants confound current treatment and prevention strategies. An immediate application could be in immune-suppressed individuals who do not respond to traditional vaccines, develop chronic infection with SARS-CoV-2, and may be reservoirs for new variants [Kemp SA, et al. SARS-CoV-2 evolution during treatment of chronic infection. Nature. 2021. Epub 2021/02/06. doi: 10.1038/s41586-021-03291-y. PubMed PMID: 33545711]. The advantage of vector-expressed decoy in preventing COVID-19 infections in immune-suppressed individuals is that this therapy does not rely on the recipient’s adaptive immune system to be effective.

### Example 2. ACE2 Variant IgG Fc4 Fusions and RBDs

### Methods.

### A. Yeast Display Library Construction, Outgrowth, Induction, Staining, and Sorting.

Yeast display: We generated mutagenized ACE2 gene fragments by error prone PCR using the Diversify PCR Random Mutagenesis Kit (TakaraBio) at multiple mutation levels, mixing the PCR products. We used Gap-repair cloning and high-efficiency LiAc transformation [Gietz RD, Schiestl RH. Large-scale high-efficiency yeast transformation using the LiAc/SS carrier DNA/PEG method. Nat Protoc. 2007;2(1):38-41. Epub 2007/04/03. doi: 10.1038/nprot.2007.15. PubMed PMID: 17401336] to assemble the ACE2 gene fragments into a centromeric plasmid. The plasmid contained an upstream Aga2 gene fragment, a downstream HA epitope tag with flexible GSG linkers, and was driven by an inducible GAL1 promoter, and contained a low-copy centromeric origin, similar to pTCON2 [Chao G, Lau WL, Hackel BJ, Sazinsky SL, Lippow SM, Wittrup KD. Isolating and engineering human antibodies using yeast surface display. Nat Protoc. 2006;1(2):755-68. Epub 2007/04/05. doi: 10.1038/nprot.2006.94. PubMed PMID: 17406305]. Following transformation or sorting rounds, we passaged the libraries at 10X diversity 3 times in SD-trp before inducing in log phase for 24 hrs at 30° C. in SG-CAA [Gietz, cited above]. For FACS, we stained yeast with recombinant CoV2 RBD-His6 (Genscript) at diminishing concentrations through the rounds (5 nM, 1 nM, 0.1 nM +/- extended washes of up to 17 hrs for off-rate sorting). We followed up with Mouse anti His6 (Genscript), rabbit anti-HA-PE (Cell Signaling Technology), and goat anti mouse-488 secondary antibody (ThermoFisher) all in phosphate buffered saline (PBS) with 0.1% BSA. Libraries were sorted and analyzed for RBD binding and ACE2 expression at the Penn Flow Core on BD Influx and BD FACSAria II instruments. We extracted plasmid from clones or pools of clones using the Yeast Plasmid MiniPrep Kit (Zymo) and transformed this into bacteria for amplification.

### B. Next-Generation Sequencing and Analysis

We performed 2x250 paired-end Illumina sequencing on randomly sheared and size-selected ACE2 amplicons from the yeast display rounds. After removing adapters and low-quality reads, we mapped clean reads no shorter than 200 bp to the WT ACE2 nucleotide sequences using NovoAlign (v.4.03.01). We translated in-frame sequences with mapping quality scores no lower than 30 and without indels into amino acid sequences and compared to the WT ACE2 protein sequence. We tallied non-synonymous changes for each codon across the ACE2 sequence (18- 615). We calculated mutation rates at each codon as follows: (sum of non-synonymous AAs)/(sum of all AAs).

### C. Expression of ACE2 Variant IgG Fc4 Fusions and RBDs

Further, we sub-cloned candidate ACE2 synthetic DNA or decoy sequences from the yeast display format into pCDNA3.1, using the endogenous ACE2 signal peptide and appending a human IgG4 Fc domain (residues 99 to 327 from Uniprot reference sequence P01861; SEQ ID NO: 77) and a C-terminal His6 tag. To generate protein for screening we transiently transfected HEK293 cells with plasmid DNA in six-well plates using PEI and collected and clarified supernatant 72 hours later. We quantified expression using the IgG4 Human ELISA kit (Invitrogen BMS2095) with IgG4 standards provided in the kit. For hAce2-Variant1-Fc4 (CDY14-Fc4) and hAce2-Variant2-Fc4 (CDY14HL-Fc4), we produced the protein in a similar manner but purified it on protein A sepharose followed by size-exclusion chromatography on Superose 6 resin (Cytiva) to remove inactive aggregates. We determined the concentration using the predicted extinction coefficient at 280 nm (1 g/l = 1.995). Some neutralization measurements (figure not shown) used crude decoy protein tittered using and human IgG4 ELISA kit; this typically yielded higher IC50s. We cloned the synthetic sequences (IDT gBlocks) of RBD [Spike amino acids 330-530 (CoV2), 317-516 (CoV1), and 318-517 (WIV1-CoV)] into pCDNA3.1 between an IL2 signal peptide plus Gly-Ser and a C-terminal His6 tag. We transfected RBD plasmids into HEK293 cells using PEI and collected supernatants 72 hrs later for clarification, concentration, and purification on Ni- NTA resin, followed by dialysis into PBS. We confirmed purity using Coomassie-stained SDS- PAGE analysis. We determined concentrations of the RBD using predicted extinction coefficient at 280 nm.

### D. RBD Binding With SPR

We performed SPR binding analysis using a Biacore T200 instrument (GE Healthcare) at room temperature in HBS-EP(+) buffer (10 mM HEPES pH 7.4, 150 mM NaCl, 3 mM EDTA, and 0.05% P20 surfactant, Cat# BR100669, Cytiva) using a protein A/G derivatized sensor chip (Cat# SCBS PAGHC30M, XanTec Bioanalytics). We injected WT ACE2-hFc4 or hAce2-Variant1-Fc4 (CDY14-hFc4) diluted to 60 nM in HBS-EP(+) at a flow rate of 10 µL/min for 3 min to capture ~1,000 response units (RU) on the sensor surface in each cycle. We measured binding of various SARS-CoV RBD proteins to this surface at concentrations ranging from 200 nM to 0.195 nM. RBD binding was measured at a flow rate of 30 µL/min, with a 3 min association time and a 15 min dissociation time. We performed regeneration between binding cycles using 10 mM glycine pH 1.5 injected at a flow rate of 60 µL/min for 1 min. KD values were determined for each interaction using kinetics parameter fitting in the Biacore T200 Evaluation software. We used a global 1:1 binding model and did not adjust for refractive index shift. Data presented are the average of two or more replicates were measured for each RBD domain tested.

### E. CoV Pseudotyped Lentiviral Neutralization Assay

We obtained non-replicating lentivirus pseudotyped with CoV spike proteins from Integral Molecular [catalog numbers 701, 702, 704, 706, 707, 708, 710, 712, 713, 711, 726, and Urbani-Cov1]. The reporter virus particles encoded a Renilla luciferase reporter gene. We set up neutralization reactions with 100 ul of inhibitor diluted in full serum media and 10 ul of reporter virus. After 1 hour at 37° C., we added 20,000 cells/well in 50 ul of a HEK 293T cell line overexpressing ACE2 (Integral Molecular) and incubated the cells for 48 hours. We measured reporter virus transduction activity on a luminometer (BioTek) using the Renilla Glo Kit (Promega) following manufacturer’s instructions. For higher throughput screens of neutralizing potency, we used crude expression supernatant (described above) in the neutralization assay at 1 or 2 dilutions (typically 10-or 100-fold). We transformed the luciferase reading to an estimated potency (EP) using the following formula: EP = (L*[decoy])/(1-L), where L is the fractional luciferase level as compared to a mock sample (no inhibitor), and [decoy] is the concentration of the decoy in the neutralization well. This was sufficient to rank clones without performing a full titration.

### F. AAV Vector Production

The University of Pennsylvania Vector Core produced recombinant AAV vectors as previously described [Lock M, et al. Rapid, simple, and versatile manufacturing of recombinant adeno-associated viral vectors at scale. Hum Gene Ther. 2010;21(10):1259-71. Epub 2010/05/26. doi: 10.1089/hum.2010.055. PubMed PMID: 20497038; PubMed Central PMCID: PMCPMC2957274; Lock M, Alvira MR, Chen SJ, Wilson JM. Absolute determination of single-stranded and self-complementary adeno-associated viral vector genome titers by droplet digital PCR. Hum Gene Ther Methods. 2014;25(2):115-25. Epub 2013/12/18. doi: 10.1089/hgtb.2013.131. PubMed PMID: 24328707; PubMed Central PMCID: PMCPMC3991984]. Briefly, we grew HEK293 cells in Cellstack vessels (Corning), co-transfected them with a mixture of vector genome plasmid (containing ITRs, Promoter, intron, Ace2-Fc decoy sequence, terminator), trans plasmid containing AAV2 rep and cap (hu68 or rh91) genes, and adenovirus helper plasmid. We used PEI as the transfection reagent. Five days post transfection, the supernatant was harvested, clarified, concentrated, purified on iodixanol gradient, and further buffer exchanged into PBS.

### G. Decoy Quantification by Mass Spectrometry Standards

Soluble hACE2Fc (produced in-house) was spiked at different levels (0.5-500 ng/mL) into PBS or NLF acquired from a naïve rhesus macaque. Samples were denatured and reduced at 90° C. for 10 minutes in the presence of 10 mM dithiothreitol (DTT) and 2 M Guanadinium-HC1 (Gnd-HCl). We cooled the samples to room temperature, then alkylated samples with 30 mM iodoacetamide (IAM) at room temperature for 30 minutes in the dark. The alkylation reaction was quenched by adding 1µL DTT. We added 20 mM ammonium bicarbonate to the denatured protein solution, pH 7.5-8 at a volume to dilute the final Gnd-HC1 concentration to 200 mM. Trypsin solution was added at ~4 ng of trypsin per sample ratio and incubated at 37° C. overnight. After digestion, formic acid was added to a final of 0.5% to quench digestion reaction.

### Lc-ms/ms

We performed online chromatography with an Acclaim PepMap column (15 cm long, 300-µm inner diameter) and a Thermo UltiMate 3000 RSLC system (Thermo Fisher Scientific) coupled to a Q Exactive HF with a NanoFlex source (Thermo Fisher Scientific). During online analysis, the column temperature was regulated to a temperature of 35° C. Peptides were separated with a gradient of mobile phase A (MilliQ water with 0.1% formic acid) and mobile phase B (acetonitrile with 0.1% formic acid). We ran the gradient from 4% B to 6% B over 15 min, then to 10% B for 25 min (40 minutes total), then to 30% B for 46 min (86 minutes total). Samples were loaded directly to the column. The column size was 75 cm x 15 um I.D. and was packed with 2-micron C18 media (Acclaim PepMap). Due to the loading, lead-in, and washing steps, the total time for an LC-MS/MS run was about 2 hours.

We acquired MS data using a data-dependent top-20 method for the Q Exactive HF; we dynamically chose the most abundant not-yet-sequenced precursor ions from the survey scans (200-2000 m/z). Sequencing was performed via higher energy collisional dissociation fragmentation with a target value of 1e5 ions, determined with predictive automatic gain control. We performed an isolation of precursors with a window of 4 m/z. Survey scans were acquired at a resolution of 120,000 at m/z 200. Resolution for HCD spectra was set to 30,000 at m/z 200 with a maximum ion injection time of 50 ms and a normalized collision energy of 30. We set the S- lens RF level at 50, which gave optimal transmission of the m/z region occupied by the peptides from our digest. We excluded precursor ions with single, unassigned, or six and higher charge states from fragmentation selection.

### Data Processing

We used BioPharma Finder 1.0 software (Thermo Fischer Scientific) to analyze all data. For peptide mapping, we used a single-entry protein FASTA database to perform searches. The mass area of the target peptide was plotted against the spike concentration to complete a standard curve.

### Selection of Target Peptide

Based on initial in silico studies, we selected four peptides as possible sequence-specific matches for targeted quantification. We evaluated sensitivity performance for quantification of the four peptide targets in the NLF background matrix. Following blank injections to establish system cleanliness, replicate injections (n = 3) were made at all levels, from 0.5 ng/mL to 500 ng/mL. Three of the peptides were detected with ANHYEDYGDYWR (SEQ ID NO: 84) providing the greatest response across the whole range. We determined retention time (RT) reproducibility across all samples (n = 24) and determined peak area reproducibility and quantification accuracy for each level. Excellent linearity was observed for the levels tested with typical R2 > 0.94 for ANHYEDYGDYWR (SEQ ID NO: 84). For ANHYEDYGDYWR (SEQ ID NO: 84), we observed excellent precision and accuracy at all levels, with all replicates within 10% CV. For test articles, 1x or 10x NLF and/or bronchoalveolar lavage fluid (BAL) is treated as previously described without any dilution or protein precipitation. The mass area of target peptide in test articles was compared to the linear calibration generated for the spiked material to determine the level of decoy present in the test article.

### H. ASF Dilutions From Serum and Lavage Urea

We used the urea concentrations in BAL or NLF and in serum collected at the same time to determine the dilution that the lavage introduced to the ASF [Kaulbach HC, et al, Estimation of nasal epithelial lining fluid using urea as a marker. J Allergy Clin Immunol. 1993;92(3):457-65. Epub 1993/09/01. doi: 10.1016/0091-6749(93)90125-y. PubMed PMID: 8360397]. We quantified urea in mouse BAL and serum, and in NHP NLF using the Urea Assay Kit (Abcam). We obtained serum urea concentrations from NHP from the blood urea nitrogen as part of standard bloodwork lab panels (Antech).

### I. Spike Binding ELISA

SARS-CoV-2 Spike Protein RBD (Sinobio #40592-V08H) was immobilized on a 96-well plate (0.25 ug/mL in PBS, 100 ul/well) at 4° C. overnight. Plates were then washed 5x with PBS/0.05% Tween and blocked with PBS/1.0% BSA for 1 hour with shaking. Samples (2x dilution in PBS/2.0% BSA) and standards (soluble hACE2-Fc at starting concentration 100 ng/ml, 12-point, 1:2 serial dilution, plus a 0.0 ng/ml blank, in PBS/1.0% BSA) were added at 100 ul/well in duplicate and incubated for 2 hours at room temperature with shaking. Wells were washed as described and biotin conjugated goat anti-human IgG (Jackson AffiniPure #109-065-098; 1:30,000 or Southern Biotech 2049-08; 1:1,000) in PBS/1.0% BSA detection antibody was added to the wells at 100 ul/well and incubated for 2 hours at room temperature with shaking. Wells were washed as described, followed by the addition of 100 ul/well Streptavidin-HRP (Abcam #ab7403; 1:30,000) in PBS/1.0% BSA for 30 minutes with shaking. Wells were washed as described and incubated in 100 ul/well TMB substrate (Seracare #5120-0076) in the dark at room temperature with shaking until reaction was stopped with 100ul/well TMB Stop Solution (Seracare #5150-0021). Absorbances were read at 450 nm using a Spectramax M3 plate reader. We exported and analyzed the data in GraphPad Prism Version 9.0.2. All raw data was blank subtracted. We plotted a standard curve of soluble hACE2-Fc, and the X-axis (concentration) was log10 transformed. We performed a 4-parameter nonlinear regression upon the transformed standard curve, and interpolated sample concentrations.

### J. Determination of Matrix Interference in BAL and NLF Samples

Soluble hACE2Fc was spiked into NLF (0.0, 0.5, 2.0, and 10.0 ng/ml) acquired from a naïve rhesus macaque on the same plate with a standard curve (soluble hACE2Fc starting concentration 100 ng/ml, 12-point, 1:2 serial dilution, plus a 0.0 ng/ml blank) in PBS/1.0% BSA. We performed the spike binding assay and data analysis as described above.

### K. Expression Study in Mice

All animal procedures were performed in accordance with protocols approved by the Institutional Animal Care and Use Committee of the University of Pennsylvania. C57BL/6J mice were purchased from The Jackson Laboratory. Anesthetized mice received an IN (intranasal) administration of 10¹¹ GC of AAVhu68-hAce2-Variant1-Fc4 (AAVhu68.CDY14-Fc4), AAVrh91-hAce2-Variant1-Fc4 (AAVrh91.CDY14-Fc4), AAVhu68-hAce2-Variant2-Fc4 (AAVhu68.CDY14HL-Fc4), or AAVrh91-hAce2-Variant2-Fc4 (AAVrh91.CDY14HL-Fc) in a volume of 50 µL or the same volume of vehicle control (PBS) on day 0. On day 7, mice were euthanized, and BAL was collected (1 ml of PBS administered intratracheally).

### L. hACE2 TG Mouse Study

To evaluate the prophylactic efficacy potential of AAV expressing hACE2 receptor decoys against SARS-CoV-2, BIOQUAL, Inc. (Rockville, MD) conducted a challenge study using hACE2 TG mice (Stock No: 034860, The Jackson Laboratory). Mice were administered with either vehicle or 10¹¹ GC of AAVhu68-hAce2-Variant2-Fc4 (AAVhu68.CDY14HL-Fc4) IN on day -7 as described above. On day 0, mice were administered with mock or the SARS-CoV-2 challenge (50 µl of 2.8x10² pfu of SARS-CoV-2, USA_WA1/2020 isolate [NR-52281, BEI Resources]). Mice were euthanized on either day 4 or 7 via cervical dislocation. BAL was collected as described above and aliquoted for viral load assays into Trizol LS (Thermo Fisher Scientific, Waltham, MA) or heat inactivated (60° C. for 30 minutes) for decoy protein expression. The lung was collected and split for histopathology into 10% neutral buffered formalin or snap frozen for viral load analysis. RNA extraction for RT-qPCR, the quantitative RT-PCR assay for SARS-CoV-2 RNA, and subgenomic RNA were performed as described [Baum A, Ajithdoss D, Copin R, Zhou A, Lanza K, Negron N, et al. REGN-COV2 antibodies prevent and treat SARS-CoV-2 infection in rhesus macaques and hamsters. Science. 2020;370(6520):1110-5. Epub 2020/10/11. doi: 10.1126/science.abe2402. PubMed PMID: 33037066; PubMed Central PMCID: PMCPMC7857396].

### M. Histopathology of Collected Organs

The organs collected at necropsy were trimmed and routinely processed for hematoxylin and eosin (H&E) staining. Slides were blindly evaluated by a blinded pathologist using a severity score of 0 (no lesions observed), 1 (minimal), 2 (mild), 3 (moderate), 4 (marked) and 5 (severe) for each finding.

### N. Intranasal Capsid Comparison by AAV Barcoding

We generated a set of custom barcoded plasmids using degenerate nucleotides that anneal immediately downstream of the stop codon in a GFP reporter construct that contains the AAV2 ITRs. We produced barcoded AAV vectors for each serotype in the study separately by transfecting HEK293 cells as described [Lock M, et al. Rapid, simple, and versatile manufacturing of recombinant adeno-associated viral vectors at scale. Hum Gene Ther. 2010;21(10):1259-71. Epub 2010/05/26. doi: 10.1089/hum.2010.055. PubMed PMID: 20497038; PubMed Central PMCID: PMCPMC2957274], replacing the typical single ITR-containing plasmid in the transfection mix with an equimolar mixture of 4 uniquely barcoded reporter constructs. We pooled the individual vector preps on an equimolar basis using their digital droplet PCR titers. We determined the absolute barcode distribution in the AAV pool by deep sequencing; we extracted AAV genomes from the pool and performed linear-range PCR using primers that flank the barcode region to generate an amplicon for paired-end Illumina sequencing.

### O. NHP Studies

Rhesus and cynomolgus macaques were obtained from Primgen (PreLabs). NHP studies were conducted at the University of Pennsylvania or Children’s Hospital of Philadelphia within facilities that are United States Department of Agriculture-registered, Association for Assessment and Accreditation of Laboratory Animal Care-accredited, and Public Health Service- assured. For the barcode study, 4x10¹² GC of the pool AAV preps was delivered IN in a total volume of 0.28 ml to an adult male rhesus macaque using the MAD Nasal™ device. After 14 days, we collected airway tissues at necropsy, and extracted total RNA using Trizol Reagent (Thermo Fisher). We generated cDNAs using Superscript III reverse transcriptase (ThermoFisher) and an oligo dT primer. We used the cDNAs to prepare barcode amplicons for Illumina sequencing as described above. We extracted the relative barcode abundances in input (AAV mixture) and output (tissue cDNAs) from Illumina data. The ratio of output to input relative abundances (“mRNA barcode enrichment”, FIGS. 21A and 21B) for each barcode in each tissue is proportional to the relative efficiency of the capsid linked to that barcode in that tissue. Agreement among the 4 barcodes assigned to each capsid allows us to assess assay noise, and detect rare, tissue-specific effects of the barcode itself on transcript stability (none detected). For each tissue, we quantified the total capsid- derived transcript per ug of total RNA using qPCR with a primer/probe set common to all the barcoded reporters. Using the total transcript counts and capsid-specific barcode distribution from NGS of the cDNA barcode amplicon allowed us to quantify absolute transduction from each capsid in a particular tissue (FIG. 21C).

For the decoy expression in NHPs, cynomolgus macaques (n=2/vector) were administered IN with 5x10¹² GC of AAVhu68-hAce2-Variant1-Fc4 (AAVhu68.CDY14), AAVrh91-hAce2-Variant1-Fc4 (AAVrh91.CDY14), AAVhu68-hAce2-Variant2-Fc4 (AAVhu68.CDY14HL), or AAVrh91-hAce2-Variant 2-Fc4 (AAVrh91.CDY14HL) as described above. An additional two NHPs were administered with 5x10¹¹ GC of AAVrh91-hAce2-Variant 2-Fc4 (AAVrh91.CDY14HL). All NHPs were negative for pre-existing neutralizing antibody titers to the administered AAV capsid prior to study initiation (Immunology Core at the Gene Therapy Program). Animals were monitored throughout the in-life phase for complete blood counts, clinical chemistries, and coagulation panels by Antech Diagnostics (Lake Success, NY). On days 7, 14, and 28 NLF was collected (animals placed in ventral recumbency with head tilted to the right, up to 5 mL of PBS delivered in 1 mL aliquots, and fluid collected via gravity). Animals were necropsied on day 28 and a full histopathological evaluation was performed.

### P. Ethics Statement for Study Conducted at BIOQUAL (hACE2 TG Mouse Challenge Study)

This research was conducted under BIOQUAL Institute Institutional Animal Care and Use Committee (IACUC) approved protocol number 21-005, in compliance with the Animal Welfare Act and other federal statutes, and regulations relating to animals and experiments involving animals. BIOQUAL is accredited by the Association for Assessment and Accreditation of Laboratory Animal Care International and adheres to principles stated in the Guide for the Care and Use of Laboratory Animals, National Research Council. Animals were monitored twice daily for clinical signs (specifically ruffled fur, heavy breathing, lethargy) and weighed daily.

### Q. Statistical Analysis Performed

Statistical analyses performed using R (version 4.0.0). Statistical tests described in figure legends.

### R. Data Availability Statement

All datasets presented in this study are included in the article/Supplementary Material.

### Example 3. Engineering an ACE2 Decoy Variant With Enhanced Activity

This example describes identifying ACE2 variants of Example 1 with increased affinity for the SARS-CoV-2 spike protein, as well as increased stability and expression yield.

Yeast display is especially appropriate for ACE2 decoy engineering for several reasons. First, yeast display uses conserved eukaryotic protein secretion pathways in the budding yeast Saccharomyces cerevisiae to fold and export protein variants to the yeast cell surface. For this reason, large, disulfide-linked extracellular proteins (such as ACE2) are well tolerated as compared to alternative display technologies like phage display. This system also applies selection pressure on the folding and secretion of protein variants that is relevant to the ultimate application in human gene therapy. Secondly, large libraries (>10⁹ variants) of decoy variants can be generated and selected rapidly, allowing the discovery of a small number of rare decoy mutations that improve both target-binding affinity and decoy expression.

We used a standard molecular biology technique, i.e., error prone PCR, to generate large mutational libraries of the ACE2 decoy receptor in a plasmid format that fuses the decoy to Aga2, a yeast cell-surface protein (FIGS. 1A and 1B). More than 10 million clones were generated. Next, we transformed this plasmid library into yeast such that each yeast cell receives only one ACE2 variant fusion. The Aga2 fusion directs the ACE2 variant to be exported to and ligated onto the exterior yeast cell wall. Thus, an ACE2 variant coats the exterior of the yeast cell that contains that variant’s gene -this provides the essential link between genotype and phenotype that is required for pooled, high-throughput protein engineering techniques.

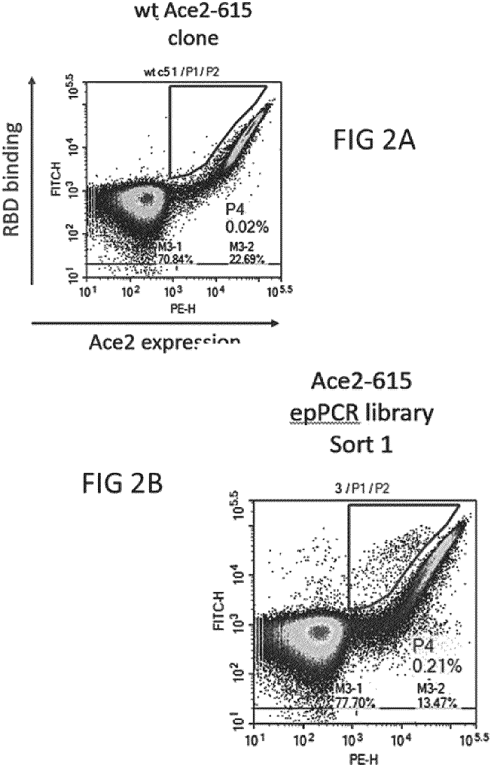

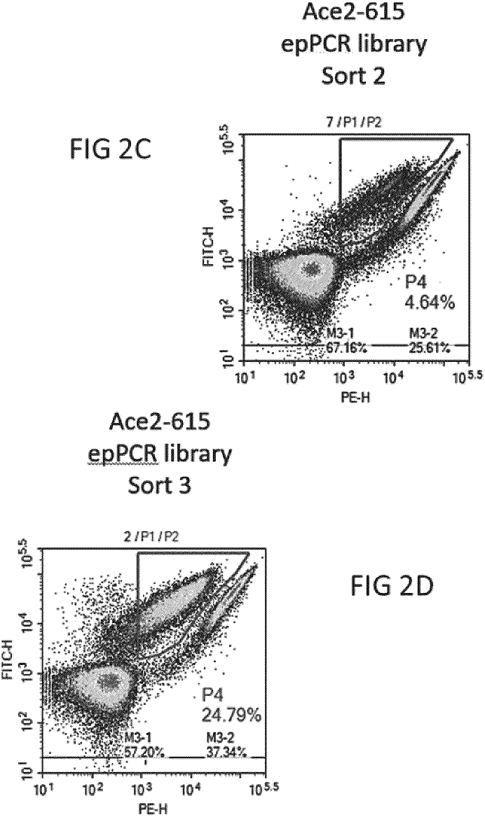

We screened through rounds of flow cytometry sorting, using low level of fluorescent RBD as the target binder. Briefly, to select for rare variants with improved target binding, we incubated the ACE2 decoy yeast display library with a fluorescently labeled SARS-CoV-2 spike protein at a concentration at or below the equilibrium binding constant of the wild-type ACE2:spike interaction (low nM initially). ACE2 variants bind the fluorescent spike protein in proportion to their affinity. Next, we used fluorescence-assisted cell sorting (FACS) to isolate the rare variants within the population with improved SARS-CoV-2 spike binding (FIGS. 2A-2D). In practice, purifying these rare variants from the population typically takes several rounds of selection. We put FACS-purified yeast directly back into culture for expansion, incubation with target, and sorting in the next round (FIGS. 2B-2D).

After 3 rounds of sorting, we isolated Ace2 clones, validated their capacity to bind higher levels of RBD in the yeast display format, and sequenced the hAce2 genes (summarized in FIG. 18C). Briefly, we identified improved variants by sequencing plasmids extracted from the rounds of yeast display selection. Because FACS selection is multi-dimensional, we simultaneously stained the yeast library with a distinct fluorophore marker for ACE2 expression level (FIGS. 2A-2D) - in this way, at each round we only selected ACE2 variants with improved expression yield, as well as improved spike-binding affinity. We identified hotspot-residues where mutational frequencies were elevated in clones with high binding activity. These were further grouped into six structurally close groups (summarized in FIG. 18C).

Next, we digitally recombined primary hit mutations to give the best chance for potency improvements. The goal was to identify sets of mutations that would likely work well together. These include mutations that are structurally distant, i.e., not touching each other, and are limited to second shell mutations, in order to limit escape mutant concerns. A collection of 100 digital recombinants were designed that incorporated 4-7 mutations across the identified hotspot zones.

The improving mutations at each hotspot were allowed to vary in relationship to their observed frequency in the sequencing data set. The frequency for wild type (WT) was set at 10% at each position. Yields included 4, 5, and 6 mutation recombinants with highest likelihood of super-decoy activity. We cloned 50 of these digital recombinants with and without a mutation at a 7^{th} spot (amino acid 330, based on hAce2 amino acid numbering, SEQ ID NO: 25). About 100 digital recombinants are expressed and screened.

Next, 25 variants from the primary screen and 100 variants from digital recombinants were cloned into mammalian expression plasmid in an IgG Fc fusion format. These 125 proteins were expressed and assayed for SARS-Cov2 neutralization using a pseudotyped lentiviral reporter assay system.

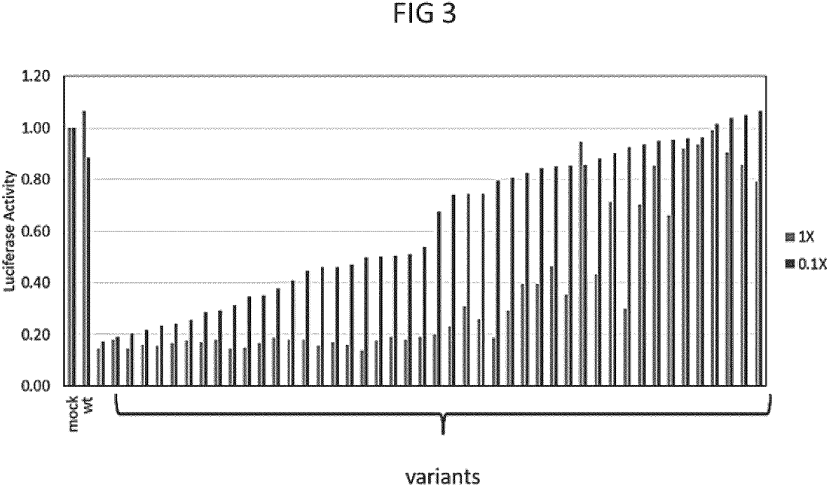

The neutralization data was used to rank the variants by the highest potency for SARS-CoV2 neutralization (FIG. 3). The results of the neutralization activity assay showed that best digital recombinant variants have 5 to 6 point mutations. Results of estimated IC50 for the best digital recombinant variants are summarized in Table 2 below.

TABLE 2

| Clone # | IC50 (ng/ml) | Titer (Frac. of WT) | Genotype | SEQ ID NOs |
|---|---|---|---|---|
| 44 | 350 | 0.77 | 27A/43G/49D/79F/90D/330Y | 52 |

| | | | | |
|---|---|---|---|---|
| 12 | 380 | 0.92 | 27A/49D/61D/75K/91P/330Y | 53 |
| 16 | 430 | 0.86 | 27A/43G/61D/81R/91P/330Y | 54 |
| 17 | 500 | 1.03 | 27A/49D/61D/79F/90S/330Y | 55 |
| 33 | 700 | 1.08 | 27A/43G/49D/79F/91P/330Y | 56 |
| 24 | 750 | 0.87 | 27A/43G/49D/61D/76R/330Y | 57 |
| 27 | 800 | 1.03 | 43G/49D/79F/90D/330Y | 58 |
| 43 | 800 | 1.14 | 27A/43G/61D/75K/90D/330Y | 59 |
| 35 | 850 | 1.08 | 27A/43G/61D/79F/90S/330Y | 60 |
| 39 | 1100 | 1.18 | 27A/49D/61D/81R/92A/330Y | 61 |

^{∗}Amino Acid (AA) position numbering is based on SEQ ID NO: 25 (with signal/leader peptide).

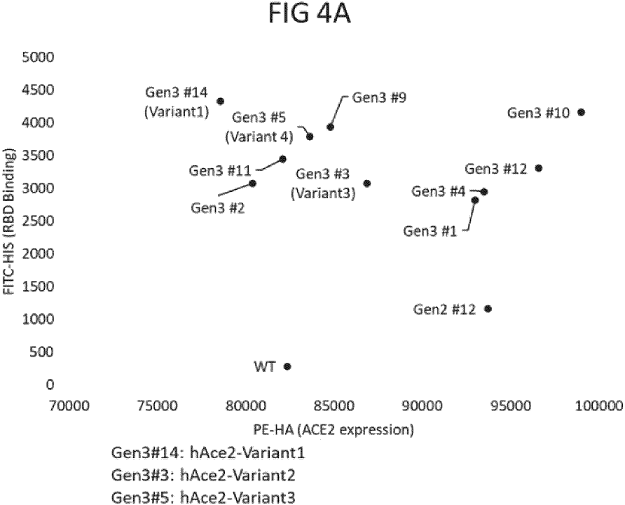

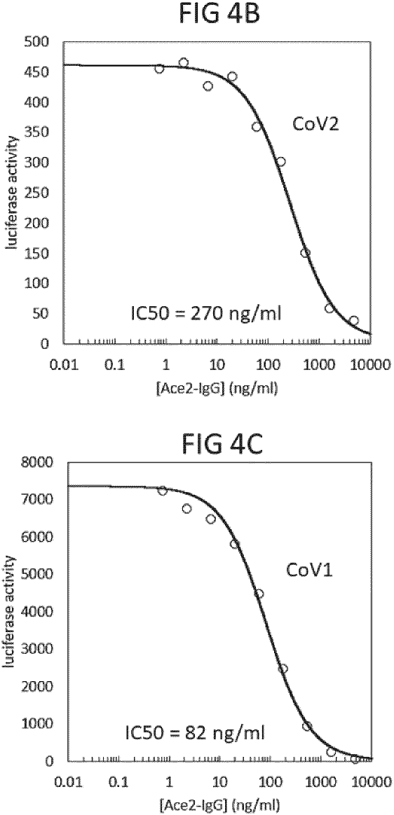

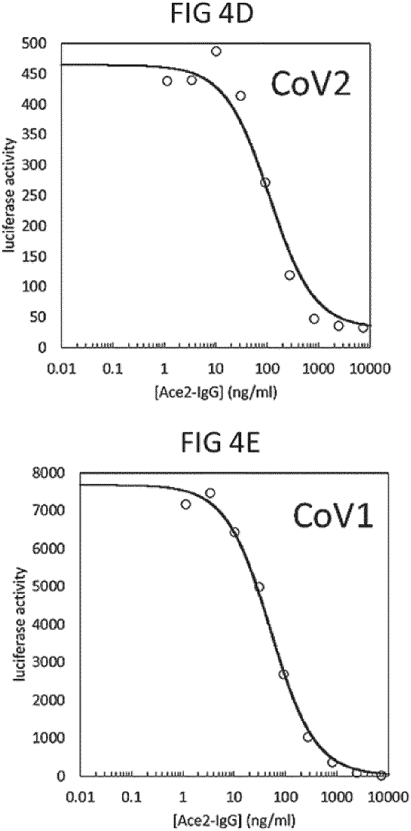

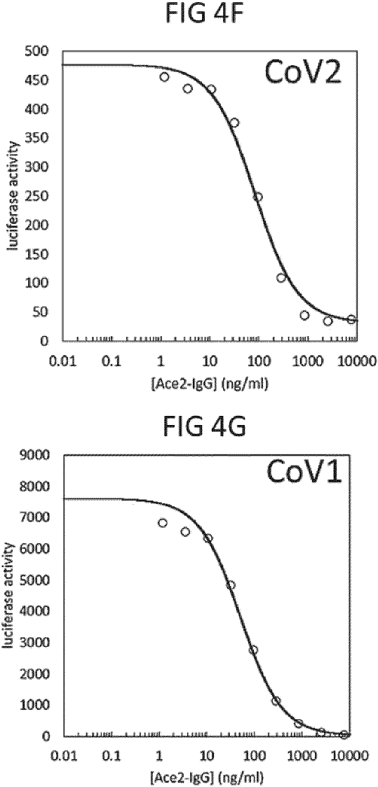

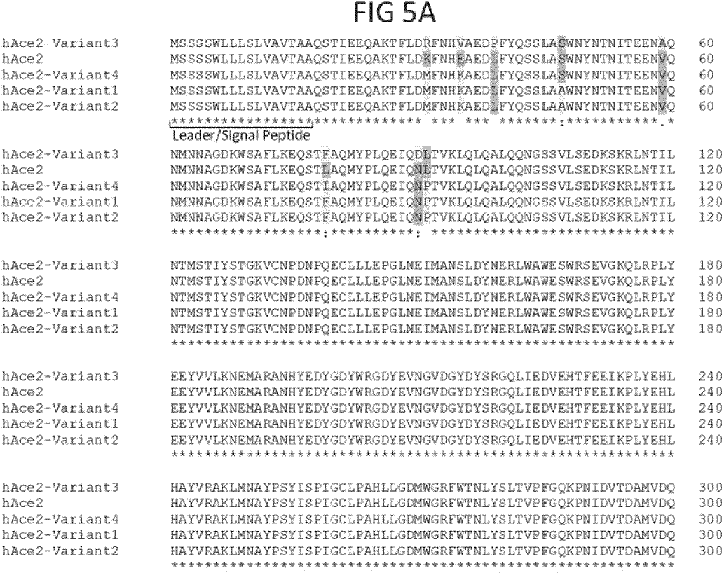

Next, we carried out molecular recombination using plasmid pools recovered from the initial yeast display screening. We used the Staggered Extension Process (StEP) to promote strand switching during PCR and effectively mix the pool of primary genotypes collected in the initial yeast display screening. The StEP library was then transformed into yeast, and screened by flow cytometry as in the primary round, with the staining concentrations dropped to lower concentrations and the wash steps were prolonged to promote selection of only the tightest RBD-binding clones (data not shown). We isolated yeast clones from the final rounds of sorting, confirmed their binding to SARS-CoV2 RBD by flow cytometry (FIG. 4A), and sequenced the best performing yeast clones. We next subcloned the best RBD binders to mammalian expression plasmids as IgG4 fusions. We produced Ace2 variant-Fc fusion proteins and screened them in a neutralization assay, selecting the top 5 performing clones as candidates (Table 3).

TABLE 3

| AA position | SEQ ID NOs | 31 (17) | 35 (18) | 39 (22) | 42 (25) | 47 (30) | 59 (42) | 79 (62) | 90 (73) | 91 (74) | 330 (313) | IC50 (µg/mL) |
|---|---|---|---|---|---|---|---|---|---|---|---|---|
| hAce2-Variant 5-IgG4 | 72 (74) | M | K | | R | | | | | P | Y | 0.135 |
| hAce2-Variant 3-IgG4 | 14 (31) | R | V | P | | | A | F | D | | Y | 0.303 |
| hAce2-Variant 4-IgG4 | 16 (33) | M | K | | | | | I | | P | Y | 0.239 |
| hAce2-Variant 6-IgG4 | 73 (75) | M | K | | | | | F | | P | Y | 0.143 |
| hAce2-Variant 1-IgG4 | 10 (27) | M | K | | | A | | F | | P | Y | 0.090 |

^{∗}Amino Acid (AA) position numbering is based on SEQ ID NO: 25, which reproduces the human Ace2 isoform 1 protein of NCBI Reference NP_068576.1; AA position numbering as indicated in parenthesis is based on SEQ ID NO: 81 (without signal/leader peptide); SEQ ID NO in parenthesis is without signal/leader peptide.

Next, we expressed and purified 2 of these candidate decoy-Fc fusions (hAce2-Variant 3-IgG4 and hAce2-Variant1-IgG4) with and without an active site mutation (H345L). The candidate decoy hAce2-Variant2-IgG4 is identical to the amino acid sequence to that of hAce2-Variant1IgG4, and further comprising active site mutation (H345L). We characterized the active site intact version of hAce2-Variant3-IgG4 in full titrations against SARS-CoV2 (FIG. 4B) and SARS CoV1 (FIG. 4C) in a reporter virus neutralization assay. We also characterized the active site intact version of hAce2-Variant1-IgG4 in full titrations against SARS-CoV2 (FIG. 4D) and SARS CoV1 (FIG. 4E) in a reporter virus neutralization assay. Additionally, we characterized the active site intact version of hAce2-Variant2-IgG4 in full titrations against SARS-CoV2 (FIG. 4B) and SARS CoV1 (FIG. 4C) in a reporter virus neutralization assay.

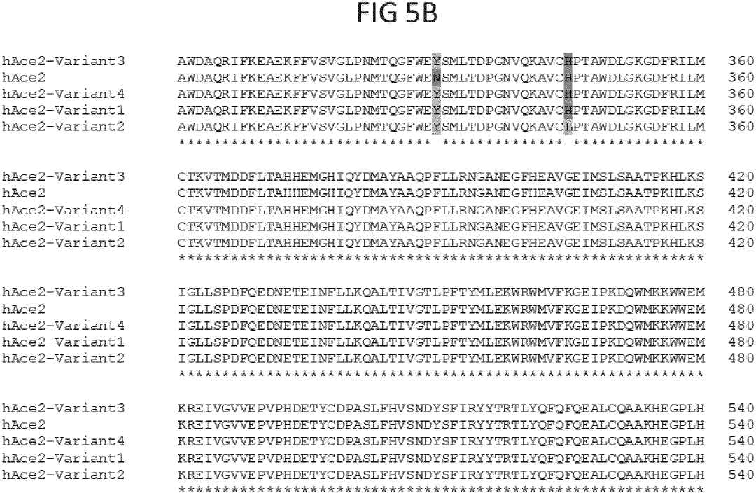

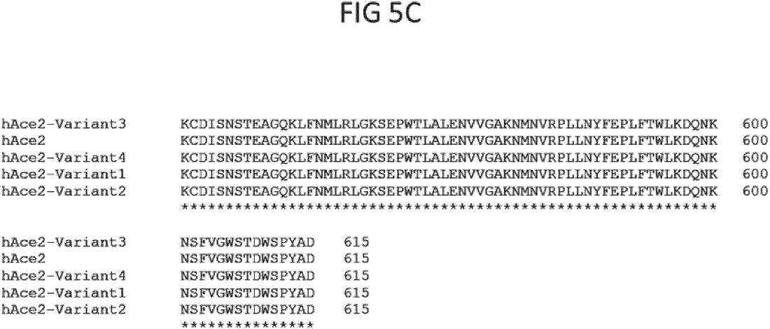

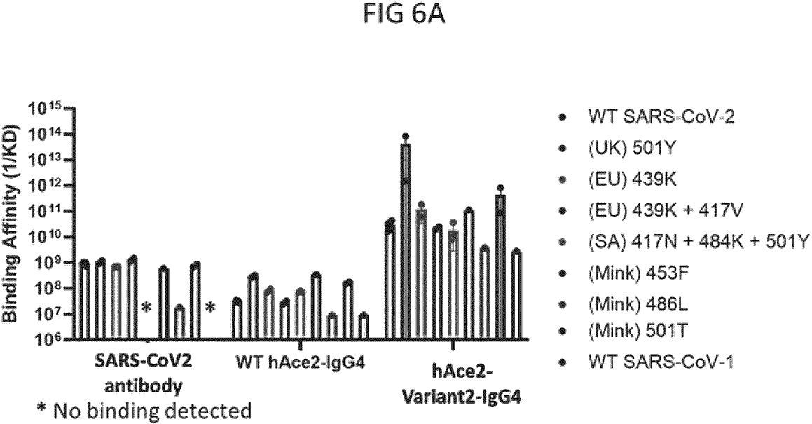

The alignment of wild-type (WT) human Ace2 (hAce2) soluble protein fragment (amino acids 1 to 615 of SEQ ID NO: 25) and mutant hAce2 soluble decoy proteins (SEQ ID NOs: 10 (hAce2-Variant1), 12 (hAce2-Variant2), 14 (hAce2-Variant 3), and 16 (hAce2-Variant4)) are shown in FIGS. 5A to 5C.

Next, we constructed variations around hAce2-Variant2-IgG4 soluble decoy fusion, in which each of the 6 hyperactive mutations was reverted to the WT amino acid. All of these reversions lost neutralizing activity. Therefore, each of the mutations provide contribution, i.e., increased affinity, at each site. FIG. 14 shows estimated IC50 of 6 “revertants” of hAce2-Variant1-IgG4 soluble decoy, wherein each “revertant” comprised of one amino acid substitution reverted from engineered back to wild type at the indicated positions (based on numbering of amino acid sequence of SEQ ID NO: 25). The measurement of the “estimated IC50” was performed from a single point neutralization measurement by using the formula IC50 = ([decoy concentration]* [F])/(1-[F]). F is the fractional activity of the luciferase reporter for the sample, that is, the level of luciferase activity as compared to a negative control (fully active) and a positive control (fully inhibited), ranging from 0-1. The [decoy concentration] is the concentration of the decoy in the measurement well. This estimation is only performed with the specific rang of F value, optimally wherein F is between 0.2 and 0.8.

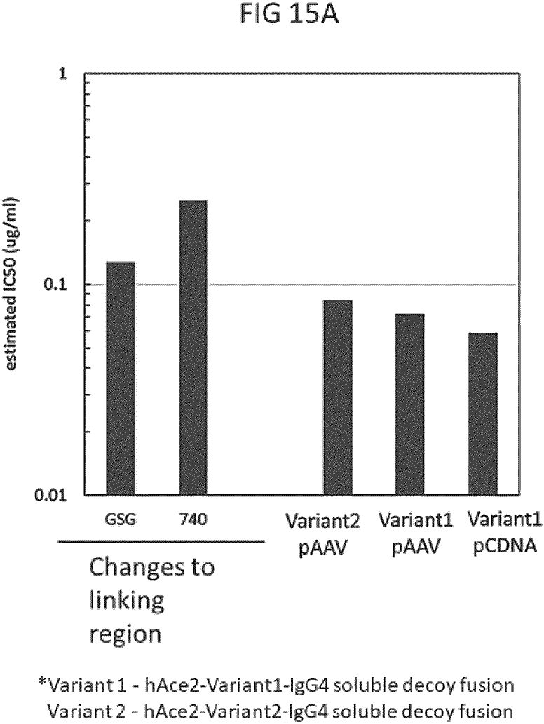

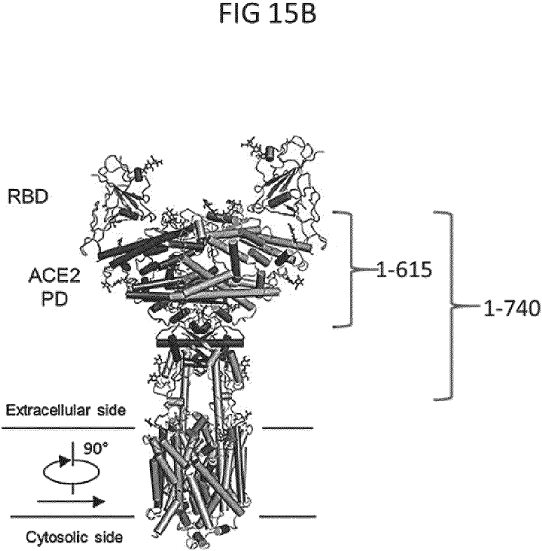

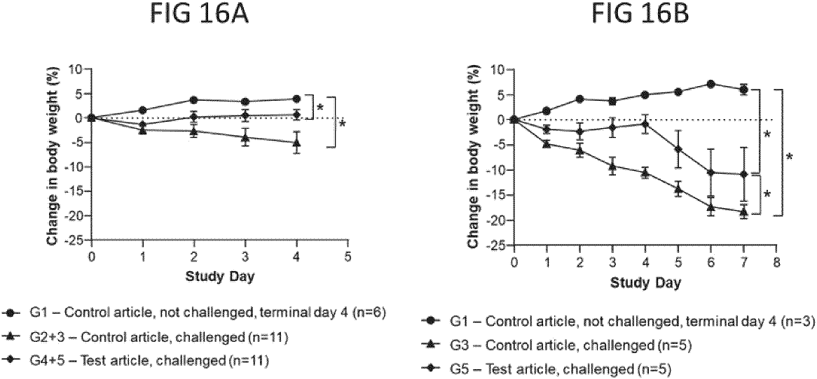

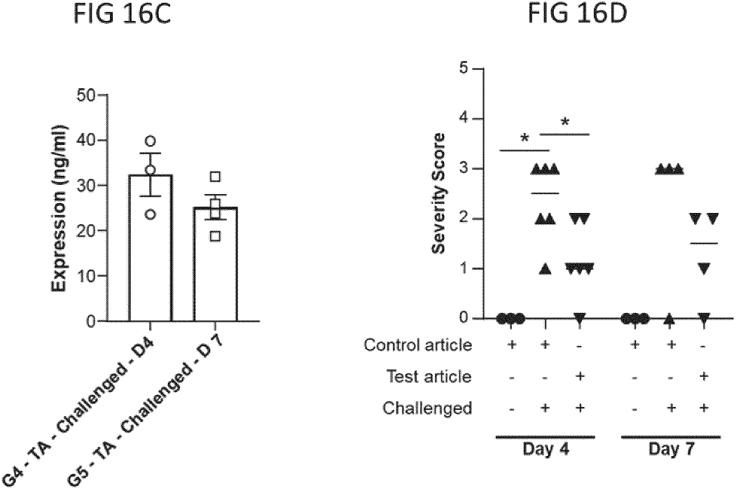

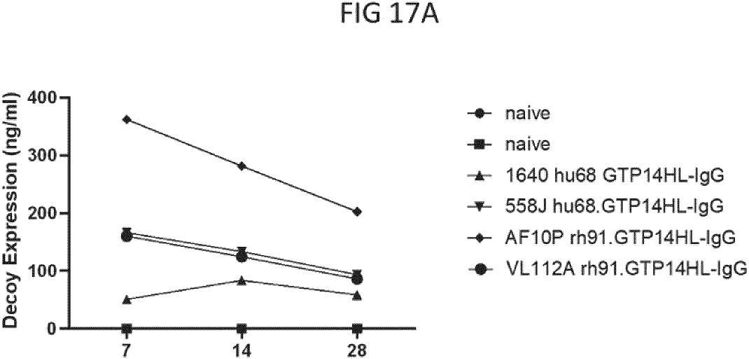

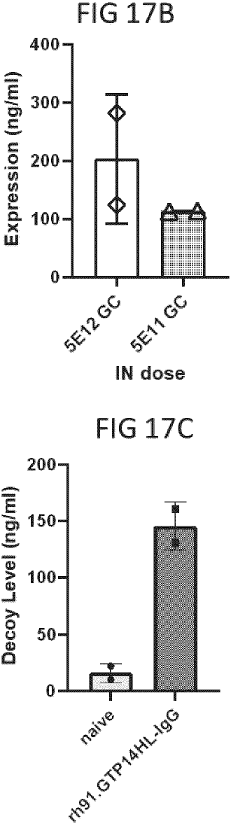

Additionally, we constructed variations with extending the hAce2 sequence from 615 to 740 or adding a GSG linker between Ace2- amino acid 615 and the IgG4 domain (FIGS. 15A and 15B). Addition of flexible GSG linker or extension to 740 amino acids of hAce2 did not improved the neutralization activity in comparison to the original constructs of Variant1 and Variant 2. FIG. 15A shows estimated IC50 values in comparison to varying “GSG” linker length, or varying decoy length (1-615 versus 1-740 amino acid of hAce2), which is linked at the amino terminus of IgG4 f the soluble decoy fusion protein. FIG. 15B shows a schematic representation of protein interaction structure between hAce2 and RBD of SARS-CoV2, showing amino acids 1-615 and 1-740, as they are mapped onto the structure of protein interaction.

### Example 4. Therapeutic Affinity of hAce2 Soluble Decoy Fusion Protein to Receptor Binding Domain (RBD) Variant of SARS-CoV2 of Example 1

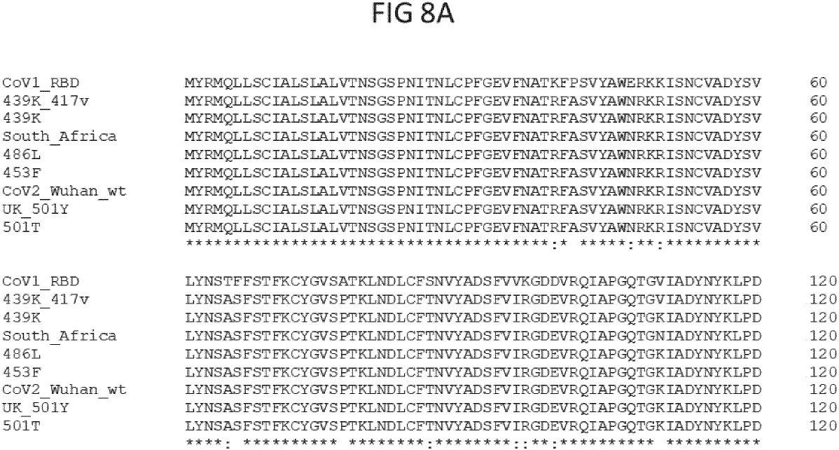

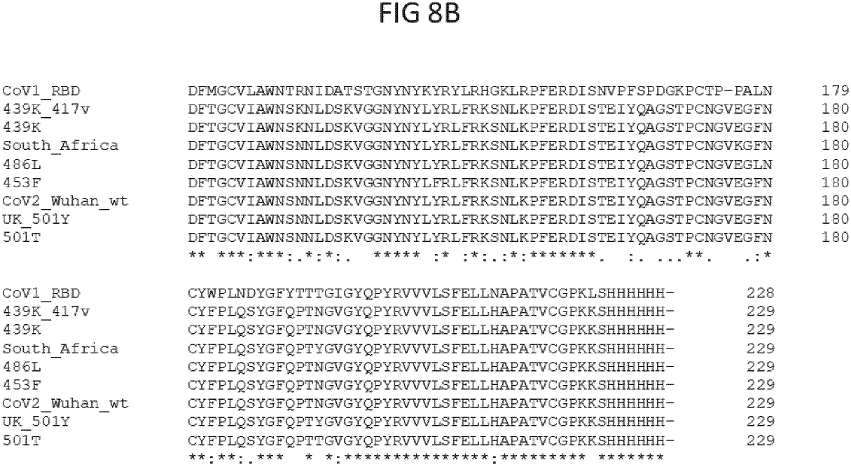

There have been reports of multiple emerging SARS-CoV2 variants that may evade antibodies. See also, Ku, Z., et al., 2021, Molecular determinants and mechanisms for antibody cocktail preventing SARS-CoV2 escape, Nature Communications, 12(469), pub Jan. 20, 2021; Greaney, A. J., et al., Comprehensive mapping of mutations to the SARS-CoV-2 receptor-binding domain that affect recognition by polyclonal human serum antibodies, 2021, bioRXiv, 2020.12.31, pub Jan. 4, 2021; Xie, X., et al., Neutralization of N501Y mutant SARS-CoV-2 by BNT162b2 vaccine-elicited sera, bioRXiv, 2021.01.07, pub Jan. 7, 2021; van Dorp, L., et al., Recurrent mutations in SARS-CoV-2 genomes isolated from mink point to rapid host-adaptation, bioRXiv, 2020.11.16, pub Nov. 16, 2020. The SARS-CoV2 RBD variants comprise of amino acid mutations at: 439K, 417V (EU), 501Y (UK), 417N, 484K, 501Y (South Africa), 501T (mink), 486L (mink), 453F (mink), and 439R, 455Y, 486L, 493N, 498Y, 501T (CoV1). The alignment of wild-type (WT) CoV2 RBD, mutants of CoV2, and CoV1 (SEQ ID NOs: 43-51) are shown in FIGS. 8A to 8B.

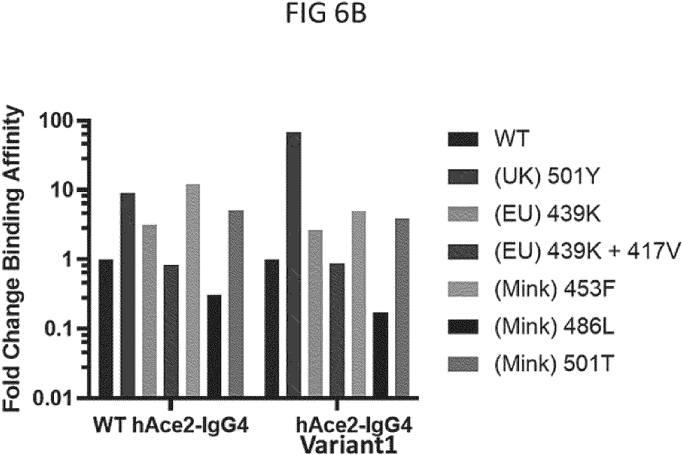

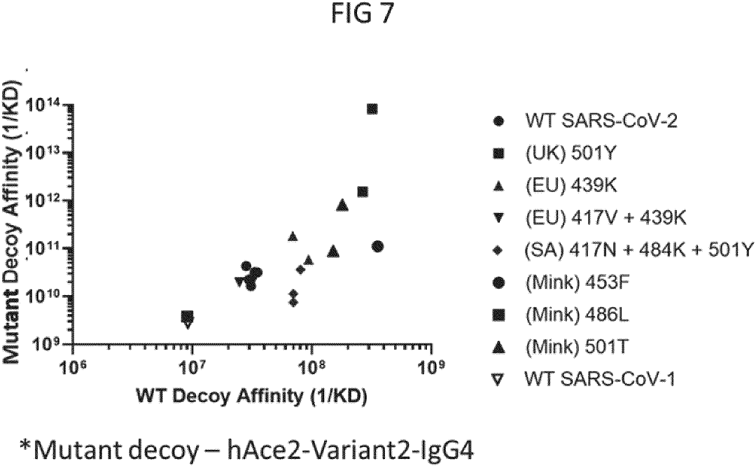

We measured the binding affinity of modified hAce2-Variant2-IgG4 soluble decoy fusion protein for SARS-CoV2 RBD mutants and compared results to the wild-type hAce2-IgG4 soluble decoy fusion protein and SARS2-CoV-antibody binding affinity to RBD mutants (FIG. 8A). FIG. 8B show the measured fold change in binding affinity of modified hAce2-Variant1-IgG4 soluble decoy fusion protein and wild-type hAce2-IgG4 soluble decoy fusion protein for mutant RBD of SARS-CoV2 (affinity to mutant RBD of SARS-CoV2 over affinity to wild type RBD of SARS-CoV2). FIG. 7 shows a graph comparison of mutant decoy affinity (hAce2-Variant2-IgG4) versus wild-type (WT) decoy affinity to various mutant RBD of SARS-CoV2.

We measured binding affinity of the soluble decoys using Surface Plasmon Resonance (SPR). The interaction kinetics and binding affinity of the modified hAce2-Variant1-IgG4 soluble decoy fusion protein were examined to the wild-type (K_{D} = 44.5 pM) and to the mutant (486L) RBD of SARS-CoV2 (K_{D} = 263 pM). The measurements of binding affinity of the modified hAce2-Variant1-IgG4 soluble decoy fusion protein to the wild-type RBD of SARS-CoV2 were: kₒₙ= 5.72 x 10⁶ M⁻¹ s⁻ ¹; k_{off}= 2.54 x 10⁻⁴ s⁻¹; K_{D} = 44.5 pM; Rₘₐₓ = 58.14 RU, as measured at SARS-CoV2 RBD concentrations of 12.5 nM, 6.25 nM, 3.13 nM, 1.53 nM, 781 pM, 391 pM, 195 pM. The measurements of binding affinity of the modified hAce2-Variant1-IgG4 soluble decoy fusion protein to the mutant (486L) RBD of SARS-CoV2 were: kₒₙ = 2.64 x 10⁶ M⁻¹ s⁻¹; k_{off} = 6.93 x 10⁻⁴ s⁻¹; K_{D} = 263 pM; Rₘₐₓ = 46.99 RU, as measured at SARS-CoV2 RBD concentrations of 12.5 nM, 6.25 nM, 3.13 nM, 1.53 nM, 781 pM, 391 pM, 195 pM. The measurements of binding affinity of the modified hAce2-Variant1-IgG4 soluble decoy fusion protein to the mutant South Africa (SA; 417N, 484 K, 501Y) RBD of SARS-CoV2 were: kₒₙ = 1.27 x 10⁶ M⁻¹ s⁻¹; k_{off} = 1.68 x 10⁻⁴ s⁻¹; K_{D} = 132 pM; Rₘₐₓ = 47.62 RU, as measured at SARS-CoV2 RBD concentrations of 12.5 nM, 6.25 nM, 3.13 nM, 1.53 nM, 781 pM, 391 pM, 195 pM. This binding analysis was performed using SPR binding analysis using a Biacore T200 instrument (GE Healthcare) at room temperature in HBS-EP(+) buffer (10 mM HEPES pH 7.4, 150 mM NaCl, 3 mM EDTA, and 0.05% P20 surfactant, Cat# BR100669, Cytiva) using a protein A/G derivatized sensor chip (Cat# SCBS PAGHC30M, XanTec Bioanalytics). We injected modified hAce2-Variant1-IgG4 or wild-type hAce2-IgG4 diluted to 60 nM in HBS-EP(+) at a flow rate of 10 µL/min for 3 min to capture ~800 response units (RU) on the sensor surface in each cycle. We measured SARS-CoV2 wild-type and mutant RBD binding to this surface at concentrations ranging from 200 nM to 195 pM. We collected binding data at a flow rate of 30 uL/min using a 3 min association time and 15 min dissociation time. We performed regeneration between binding cycles using 10 mM glycine pH 1.5 injected at a flow rate of 60 µL/min for 1 min. All kinetic fitting was performed using a global 1:1 binding model.

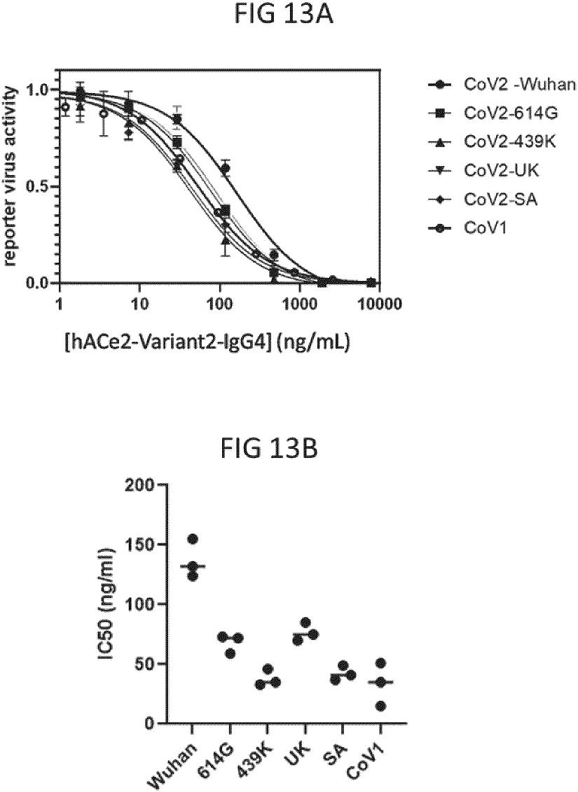

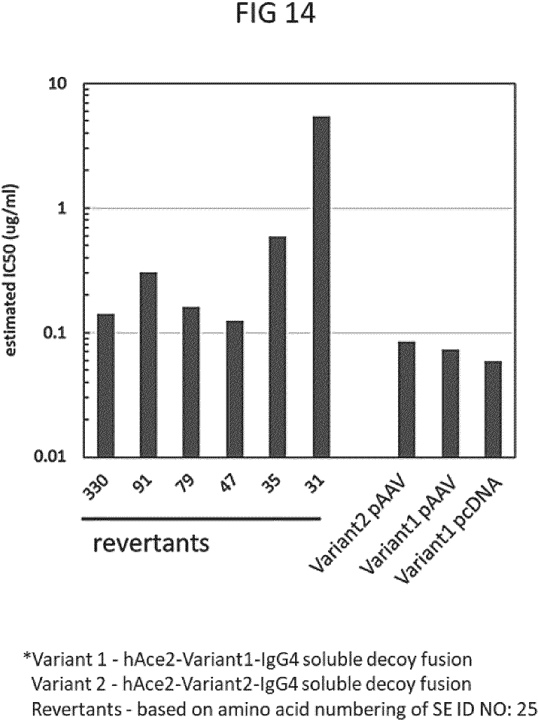

Next, we measured the neutralization activity of modified hAce2-Variant2-IgG4 soluble decoy fusion protein for SARS-CoV2 RBD mutants in a reporter virus assay (FIGS. 13A-13B). FIG. 13A shows inhibition of SARS-CoV-2 RBD-hAce2 interactions in presence engineered hAce2 soluble decoy, hAce2-Variant2-IgG4. FIG. FIG. 13B shows a plot of IC50 values for interaction inhibition between hAce2-Variant2-IgG4 and SARS-CoV2-RBD mutant variants, as measured in neutralization assay. Additional results for hAce2-Variant-2-IgG4 are provided in the following table.

| | hAce2-Variant2-IgG IC50 (ng/ml) |
|---|---|
| CoV2-Wuhan | 127 |
| CoV2-614G | 77 |
| CoV2-439K | 42 |
| CoV2 B1.1.7 | 93 |
| CoV2 B1.351 | 45 |
| CoV1 | 53 |

### Example 5. rAAV-mediated Expression of Engineered Ace2 Decoys of Example 1

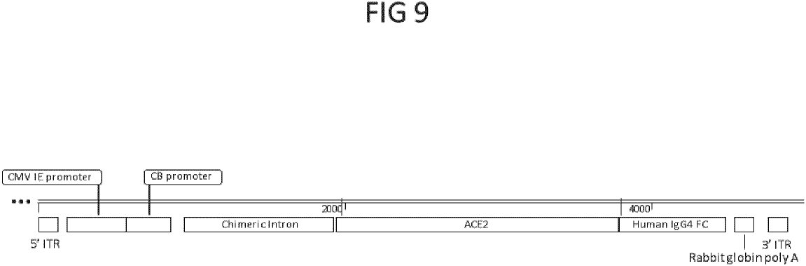

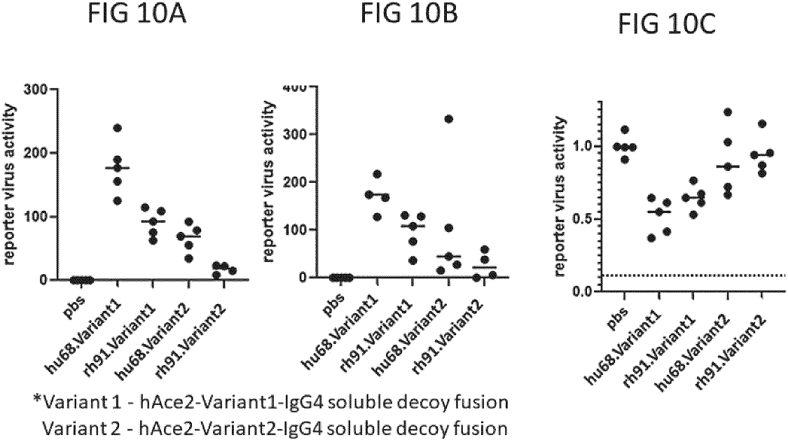

We cloned the engineered hAce2 decoys (hAce2-Variant1-IgG4 (Variant1), hAce2-Variant2-IgG4 (Variant2)) into AAV packaging plasmids comprising a vector genome (FIG. 9) and manufactured rAAVhu68 or AAVrh91 using conventional triple transduction techniques, i.e., co-expression of the plasmid encoding the vector genome to be packaged, a plasmid expressing the rep and the AAV capsid coding sequences (AAVhu68 or AAVrh91, respectively) and a plasmid having helper functions. This allowed us to test the activity of various decoys expressed from the airway tissues of animal models. We tested the expression of the engineered decoys in mice, with dosing at 1x10¹¹ genome copies (GC) of AAV intranasally, and collected bronchoaveolar lavage fluid (BALF) at necropsy on day 7 post transduction. The capsids used were hu68 and rh91. The engineered decoys were hAce2-Variant1-IgG4-Fc and hAce2-Variant2-IgG4-Fc, the latter of which is a variant with the H345L mutation which inactivates the Ace2 enzymatic activity. The data in FIGS. 10A to 10C is on BALF samples collected at day 7 post-transduction. Analysis of the BALF samples indicates that rAAVs encoding the engineered decoy-Fc fusions when dosed intranasally are capable of generating concentrations of active decoy protein in the air-surface liquid (ASL) of mice (FIG. 10A). Decoy recovered from the mouse ASL is capable of binding the CoV2 spike protein and neutralizing CoV2 reporter virus (FIGS. 10B and 10C). It is important to note that the BALF fluid is collected by introducing a volume of PBS to the lungs of the mice, and then recovering the buffer solution with a syringe. Thus, the BALF represents a significant dilution of the ASL with the PBS buffer. We expect concentrations of the active decoy in the actual ASL to be significantly higher than in the BALF.

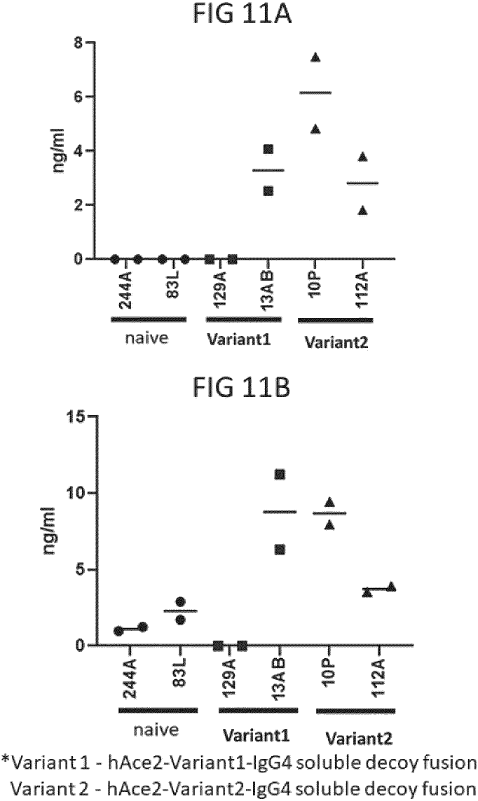

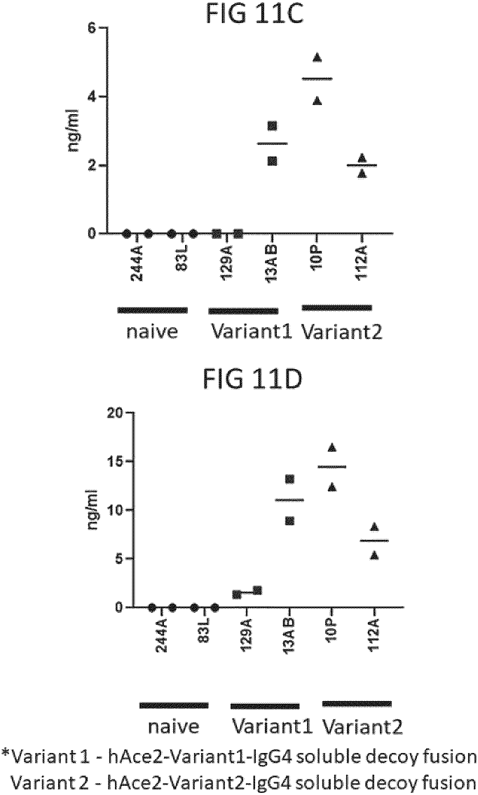

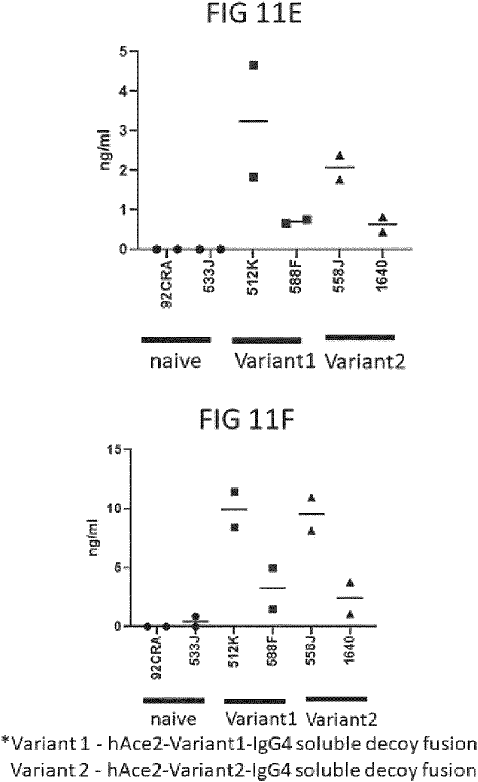

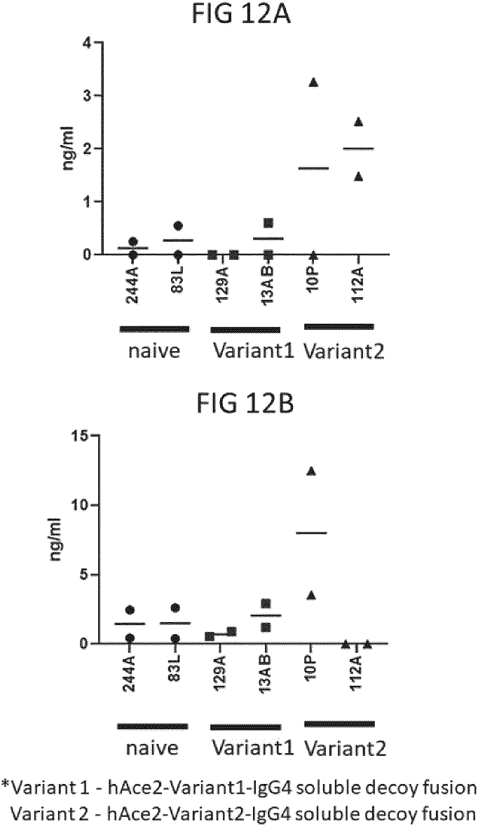

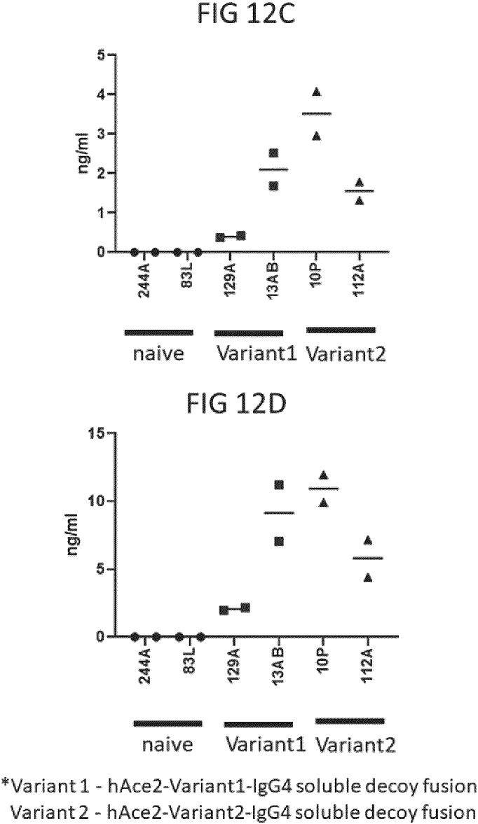

Next, we delivered the engineered Ace2 decoy AAVs intranasally to non-human primates (NHP) at 5x10¹² genome copies per animal. At day 7 (FIGS. 11A to 11F) and day 14 (FIGS. 12A to 12D) post-transduction we collected nasal lavage fluid (NLF) from sedated NHP in treated or control groups. Analysis of the NLF samples indicates that rAAVs encoding the engineered decoy-Fc fusions when dosed intranasally are capable of generating concentrations of active decoy protein in the air-surface liquid (ASL) of the NHP nose. Decoy recovered from the NHP nose ASL is capable of binding the CoV2 spike protein. It is important to note that the NLF fluid is collected by introducing a volume of PBS to the nasal passages of the sedated NHP, and then recovering the buffer solution. Thus, the NLF represents a significant dilution of the nose ASL with the PBS buffer. We expect concentrations of the active decoy in the actual NHP nose ASL to be significantly higher than in the NLF.

FIGS. 11A to 11B show analytics from NHP study, intranasal delivery of 5x10¹² GC of rAAVs encoding hAce2-Variant½-IgG4-Fc of mutant decoys. The capsids used were AAVhu68 and AAVrh91. The engineered decoys were hAce2-Variant1-IgG4 (Variant1) and hAce2-Variant2-IgG4 (Variant2), the latter of which is a variant with the H345L mutation which inactivates the Ace2 enzymatic activity. FIG. 11A shows CoV2 spike binding activity in the 1x BALF samples (AAVrh91), wherein an immobilized recombinant CoV2 spike protein in an ELISA assay, with the human Fc tag on the decoy as the detection epitope. FIG. 11B shows CoV2 spike binding activity in the 10x BALF samples (AAVrh91), wherein an immobilized recombinant CoV2 spike protein in an ELISA assay, with the human Fc tag on the decoy as the detection epitope. FIG. 11C shows mass spectrometry with Ace2-IgG4 standards to determine the concentration of decoy in NLF 1X samples (AAVrh91) from 2 animals in each doing group. FIG. 11D shows mass spectrometry with Ace2-IgG4 standards to determine the concentration of decoy in NLF 10X samples (AAVrh91) from 2 animals in each doing group. FIG. 11E shows mass spectrometry with Ace2-IgG4 standards to determine the concentration of decoy in NLF 1X samples (AAVhu68) from 2 animals in each doing group. FIG. 11F shows mass spectrometry with Ace2-IgG4 standards to determine the concentration of decoy in NLF 10X samples (AAVhu68) from 2 animals in each doing group.

FIGS. 12A to 12D show analytics from NHP study, intranasal delivery of 5x10¹² GC of rAAVs encoding hAce2-Variant½-IgG4-Fc of mutant decoys. The capsids used were AAVhu68 and AAVrh91. This data is on NLF samples collected at day 14 post-transduction. FIG. 12A shows CoV2 spike binding activity in the 1x BALF samples (AAVrh91), wherein an immobilized recombinant CoV2 spike protein in an ELISA assay, with the human Fc tag on the decoy as the detection epitope. FIG. 12B shows CoV2 spike binding activity in the 10x BALF samples (AAVrh91), wherein an immobilized recombinant CoV2 spike protein in an ELISA assay, with the human Fc tag on the decoy as the detection epitope. FIG. 12C shows mass spectrometry with Ace2-IgG4 standards to determine the concentration of decoy in NLF 1X samples (AAVrh91) from 2 animals in each doing group. FIG. 12D shows mass spectrometry with Ace2-IgG4 standards to determine the concentration of decoy in NLF 10X samples (AAVrh91) from 2 animals in each doing group.

### Example 6. Engineered Ace2 Decoy for Prevention Against SARS Infections and Post-Infection Therapy of Viruses

### A. AAV-Mediated Delivery of Ace2 - Fc Decoy for Prevention of SARS-CoV-2 Infection

| | |
|---|---|
| Therapeutic Goal/Indication | Prevention of SARS-CoV-2 infection. |
| | Prioritize individuals at high risk for SARS-CoV-2 infection and where available prophylactic measures are suboptimal. |
| Ph 1 Study Design | Double-blind, placebo controlled, ascending dose study to assess Safety, Tolerability and Proof of Mechanism |
| Study Treatment | Single Intranasal administration using MAD Device |
| Population | • Male and females with known immunodeficiency (including but not limited to): |
| | • Patients on immunosuppressive treatment |
| | • Solid organ transplant |
| | • Solid tumor |
| | • Hematological malignancy |
| | • Primary immune deficiency |
| | • Immunocompromise due to infection (for example, HIV) |
| Sample Size | 21 subjects (3 cohort dose levels with 7 subjects each) |
| Endpoints | • Safety and tolerability |
| | • Transgene ACE2-Fc decoy protein expression in nasal lavage and serum |
| | • Immune response to capsid and transgene |
| | • SARS-CoV-2/COVID-19 clinical events (exploratory endpoint) |

| | |
|---|---|
| Therapeutic Goal/Indication | Treatment of SARS-CoV-2 infection. Prioritize individuals at high risk for SARS-CoV-2 infection and where available prophylactic measures are suboptimal. |
| Ph 1 Study Design | Double-blind, placebo controlled, ascending dose study to assess Safety, Tolerability and Proof of Mechanism |
| Study Treatment | Systemic Intravenous administration of ACE2R-Fc protein |
| Population | • Male and females with active SARS-CoV-2 infection |
| Sample Size | 21 subjects (3 cohort dose levels with 7 subjects each) |
| Endpoints | • Safety and tolerability |
| | • ACE2-Fc decoy protein pharmacokinetics and pharmacodynamics |
| | • SARS-CoV-2 viral load/COVID-19 disease progression |
| | • Immune response to ACE2-Fc |

AAVrh91.CB7.CI.hAce2GTP14HL-IgG.rBG (AAVrh91.CDY14HL-Fc4; comprising hAce2-Variant2-IgG4 (Variant2))) is a final drug product (FDP) that consists of a non-replicating recombinant adeno-associated viral (rAAV) vector active ingredient and a formulation buffer.
Inverted Terminal Repeat (ITR): The ITRs are identical, reverse complementary sequences derived from AAV2 (130 base pairs [bp], GenBank: NC-001401) that flank all components of the vector genome. The ITRs function as both the origin of vector DNA replication and the packaging signal for the vector genome when AAV and adenovirus helper functions are provided in trans. As such, the ITR sequences represent the only cis sequences required for vector genome replication and packaging.
CB7 promoter: This promoter is composed of a hybrid between a CMV IE enhancer and a chicken β-actin promoter (SEQ ID NO: 122).
Human Cytomegalovirus Immediate-Early Enhancer (CMV IE): This enhancer sequence obtained from human-derived CMV (382 bp, GenBank: K03104.1) increases expression of downstream transgenes (SEQ ID NO: 120).
Chicken β-Actin Promoter (CB): This ubiquitous promoter (281 bp, GenBank: X00182.1) was selected to drive transgene expression in any cell type (SEQ ID NO: 121).
Chimeric Intron (CI): The hybrid intron consists of a chicken β-actin splice donor (973 bp, GenBank: X00182.1) and a rabbit β-globin splice acceptor element. The intron is transcribed, but removed from the mature messenger ribonucleic acid (mRNA) by splicing, bringing together the sequences on either side of it. The presence of an intron in an expression cassette has been shown to facilitate the transport of mRNA from the nucleus to the cytoplasm, thus enhancing the accumulation of a steady level of mRNA for translation. This is a common feature in gene vectors intended for increased levels of gene expression (SEQ ID NO: 123).
Coding sequence: The coding sequence consists of a portion of the complementary deoxyribonucleic acid (cDNA) of the human ACE2 gene encoding a secreted version of the human ACE2 protein (1845 bp; 615 amino acids [aa], GenBank: AB193259.1), which is the receptor protein used by SARS-CoV-2 for cell entry. The secreted version of human ACE2 protein has been mutated to eliminate peptidase activity and improve antiviral potency. A consensus sequence for the human IgG4 Fc region cDNA sequence (687 bp, 229 aa), including the entire hinge region that connects the Fc to the variable domains in the full length antibody, is included immediately 3′ of the ACE2 sequence to generate a fusion protein that allows the protein to be secreted as a dimer, potentially increasing avidity for the spike protein on the virus surface. The Fc from IgG4 was chosen to have reduced or no effector function.
Rabbit β-Globin Polyadenylation Signal (rBG PolyA): The rBG polyadenylation (PolyA) signal (127 bp, GenBank: V00882.1) facilitates efficient polyadenylation of the transgene mRNA in cis. This element functions as a signal for transcriptional termination, a specific cleavage event at the 3′ end of the nascent transcript and the addition of a long polyadenylation tail (SEQ ID NO: 124).

FDP is delivered via IN administration using a MAD Nasal™ Device into each nostril (referred to as nare hereafter). Two aliquots of 0.2 mL each are administered into each nare for a total administered volume of 0.4 mL per nare, leading to a total administered volume of 0.8 mL per subject. The three dose cohorts include: Cohort 1 (low dose - 5.0 x10¹⁰ GC or 3.00 x10¹¹ GC), Cohort 2 (mid dose - 5.0 x 10¹¹ GC or 1.00 x 10¹² GC), and Cohort 3 (high dose - 5.0 x 10¹² GC or 3.00 x10¹² GC).

The product for the Phase 1 FIH clinical trial is manufactured by transient transfection of HEK293 cells using plasmid DNA followed by downstream purification.

### Example 7. Extended Data Figures Legends

FIGS. 22A to 22E show design and characterization of initial ACE2 decoy construct. Schematic representation of the initial ACE2-Fc4 decoy constructs. ACE2 decoy constructs contained the extracellular domain of ACE2 (amino acids 18-615) with one of three candidate signal peptides (IL-2, native, or thrombin). In some constructs two catalytic histidine residues were mutated to asparagine to abrogate enzymatic activity (designated NN). Constructs were designed with no Fc, or the Fc domain of IgG1 or IgG4. Constructs were expressed in HEK293 cells and detected in supernatant using a sandwich ELISA to ACE2 (for constructs without an Fc domain) or an ELISA with SARS-CoV-2 spike protein as a capture antigen and an anti-human IgG polyclonal antibody for detection (for Fc fusion proteins). The candidate ACE2-NN-Fc4 fusion protein was expressed in HEK293 cells, purified by protein A chromatography, and analyzed by SDS PAGE under reducing and nonreducing conditions. The affinity of the purified ACE2-NN-Fc4 decoy protein for monomeric spike protein in solution was quantified by Biacore SPR. kon = 2.6 × 10⁵ M⁻¹ s⁻¹, k_{off} = 0.00093 s⁻¹, t_{½} = 745 s, K_{D} = 3.5 nM, Rₘₐₓ = 67 RU. The purified ACE2-NN-Fc4 protein was titrated against Wuhan CoV2 pseudotyped lentivirus bearing a luciferase reporter. The IC₅₀ was obtained from a fit of these data (15 ug/ml). The candidate construct (ACE2-NN-Fc4) was packaged in an AAV vector (hu68 capsid) and administered IN to WT mice. Seven days after administration, BAL was collected for measurement of transgene expression using an ELISA with SARS-CoV-2 spike protein as a capture antigen to confirm that the decoy receptor expressed in vivo was functional. BAL from similar experiments was 6-fold diluted from the ASF as determined by comparison of BAL and serum urea. Thus, we determined that ASF concentrations of the decoy were likely below 2 ug/ml. Two NHPs (IDs 258 and 396) received 9 × 10¹² GC of an AAVhu68 vector expressing a soluble ACE2-NN-Fc fusion protein via the MAD. Nasal lavage samples were collected weekly after vector administration and concentrated 10-fold for analysis. The concentration of the decoy receptor in NLF was measured by MS. Urea measurements in similar experiments indicate that 10X nasal lavage is ~8-fold diluted from ASF. We therefore determined that ASF concentrations of the decoy were less than 100 ng/ml.

FIGS. 23A to 23D show design and selection of primary and secondary yeast display libraries. Structure of CoV-2 RBD (blue spheres) bound to human ACE2 (green ribbons, red and yellow spheres) (6M17.pdb [Yan, R. et al. Structural basis for the recognition of SARS-CoV-2 by full length human ACE2. Science 367, 1444-1448, doi:10.1126/science.abb2762 (2020)]). Most ACE2 contacts with RBD are limited to the amino acids 18-88 (red spheres) and a patch of amino acids that are more C terminal (yellow spheres). The gene sequence for ACE2 is shown below in the same coloring. We designed two primary yeast display libraries: 1) the whole ACE2 gene fragment was mutagenized (Whole) and 2) the mutagenesis was limited to only the first 96 amino acids (NC) to concentrate the mutagenesis on the region most likely to impact RBD binding. The regions shaded gray were subjected to error- prone PCR to introduce mutations. Deep sequencing of yeast display plasmids extracted from the final round of sorting for the Whole and NC libraries. The fractional rate of mutation at each position in 18-615 of ACE2 is plotted. Improving mutations occurred mostly in the first 96 amino acids regardless of the input library. These include RBD contact residues, second-shell residues, and the distal consensus N-glycan site at position 90, an apparent negative regulator of RBD binding. Though C-terminal mutation load was generally associated with poor ACE2 expression (data not shown), several consensus C-terminal mutations emerged from the whole ACE2 primary sorts. These include a substitution to Y at position 330, which we identified in a clone with improved binding. A detailed plot of the mutational frequencies in Whole and NC library final round sorts for residues 18-100. The libraries yielded many of the same mutants in this region with improved binding activity. Schematic representation of secondary library design. We isolated 300 yeast colonies from the sorted primary (Whole and NC) libraries, analyzed them individually for RBD binding and ACE2 expression by flow cytometry, and selected 90 isolates with validated binding improvements. Next, we generated a secondary library by shuffling selected ACE2 genes using the staggered extension process (StEP) method [Aguinaldo, A. M. & Arnold, F. H. Staggered extension process (StEP) in vitro recombination. Methods Mol Biol 231, 105-110, doi:10.1385/1-59259-395-X:105 (2003).]. Given that most improving mutations were N-terminal, we shuffled only residues 18-103 of the input templates (orange shaded region in the schematic), matching these with a mixture of unmutated and N330Y C-terminal DNAs in a multi-fragment assembly yeast transformation.

FIGS. 24A and 24C show a schematic of parallel paths to the generation of affinity matured ACE2 decoy. After generating improved RBD binding sequences from a primary round of sorting, we undertook a parallel path to digitally recombine the most frequent mutations in addition to continued diversification and sorting. Primary library hits, digital recombinants of those hits and isolated clones from the second, more stringent round of yeast display sorting were all cloned as Fc4-fusion proteins and screened in a CoV2 pseudotype neutralization assay. We selected 300 clones from primary yeast display library sorts for clonal RBD binding analysis using flow cytometry in the yeast display format and selected 90 clones with validated binding improvements. A pool of plasmid DNA from those 90 isolates was subjected to deep sequencing for mutational analysis, and the rates of all possible amino acid substitutions are presented in this heat map by amino acid position. Black squares represent the wt amino acid at each position. The goal was to generate a collection of synthetic (digital) recombinants of the observed mutations in this data set. We grouped subsets of the validated mutations into 7 regions (p1 -p7) and assigned frequencies to the mutations based loosely on observed frequencies in the data set, allowing for the wt residue at 10% or 50% depending on the position. We biased the mutation selection towards second-shell positions to avoid directly remodeling the ACE2:RBD contact positions where possible. In order to maximize the chance of mutations working together productively, we chose the groupings in based on 3D structure (6M17.pdb) such that direct contacts between groups would be minimized. We randomly selected primary screen mutation combinations based on the frequencies in, and had these digital recombinants synthesized for cloning as secreted IgG Fc4 fusion proteins. We screened primary yeast display hits, digital recombinants, and secondary yeast display hits in a pseudotyped lentivirus reporter assay for CoV-2 neutralization at one or two dilutions from the expression supernatant, noting the expression titer relative to ACE2-wt-Fc4 control. Mutations associated with the 5 best digital recombinant hits and the 5 best hits from a secondary round of yeast display sorting.

We performed RBD binding assay using Surface Plasmon Resonance (SPR) assay. Briefly, raw data (colored lines) and fits (black lines) for immobilized ACE2-wt-Fc4 on the SPR chip surface binding injected RBDs (concentrations listed). Parameters of the fits, including the dissociation equilibrium constant (KD) are listed below each panel. These data contributed to FIGS. 19B and 19C. B. Raw data (colored lines) and fits (black lines) for immobilized hAce2-Variant2-Fc4 (CDY14HL-Fc4) on the SPR chip surface binding injected RBDs (concentrations listed).

FIGS. 25A and 25B show challenge study in mice. Average weight loss (percentage) in males and females hACE2-TG mice that received Challenge Placebo and Challenge Decoy.

FIGS. 26A to 26I show AAV capsid selection for NHP IN delivery. mRNA barcode enrichment in airway tissues for a mixture of 9 barcoded serotypes delivered IN at 2.7×10¹¹ GC each. We assigned four uniquely barcoded transgenes to each capsid at manufacture. Data show the enrichment score (tissue abundance in RT-PCR-NGS/ injection mixture abundance in PCR- NGS) for all 4 barcodes per capsid with mean and SD. Four NHP were IN dosed with rh91 or hu68 vectors encoding decoy transgenes at 5×10¹² GC. Data show the biodistribution of vector genomes in airway tissues 28 days after dosing. AAVrh91 achieved higher gene transfer in upper airway tissues, particularly in the maxillary sinuses and cavity septum. Gene transfer in lower airway tissues was more variable.

### Example 8. Toxicology Study of Intranasal Administration of Final Drug Product (AAV) to Adult Cynomolgus Macaque NHPs

In this study, the toxicology effect of AAVrh91.CB7.CI.hAce2GTP14HL-IgG4.rBG (AAVrh91.CDY14HL-Fc4 or AAVrh91. hAce2GTP14HL-IgG4; comprising hAce2-Variant2-IgG4 (Variant2)) post administration is examined. The purpose of the 120-day study is to evaluate the safety, tolerability, biodistribution, and excretion of AAVrh91.CB7.CI.hAce2GTP14HL-IgG.rBG following intranasal (IN) administration using the MAD110 Nasal Device in an anatomically relevant model of adult cynomolgus macaque NHPs. Safety pharmacology (CNS, cardio/respiratory and renal), pharmacokinetics (PK), immunological readouts, expression data, neutralization (BAL, nasal lavages), and angiotensin related assessments at 30 days and 120 days post administration is examined.

Table below shows a summarized layout of the IND-enabling NHP Pharm/Tox study.

| | | | | |
|---|---|---|---|---|
| | Group 1 (Control) | Group 2 (Low Dose) | Group 2 (Mid-Dose) | Group 3 (High Dose) |
| Number of Macaques | 4 | 6 | 6 | 6 |
| Sex | M+F | M+F | M+F | M+F |
| Age | Adult (3-8 years) | Adult (3-8 years) | Adult (3-8 years) | Adult (3-8 years) |
| Test Article | Vehicle (FFB) | FDP | FDP | FDP |
| ROA^{a} | IN | IN | IN | IN |
| Vector Dose | N/A | 1.02 × 10¹¹ GC | 3.40 × 10¹¹ GC | 1.02 × 10¹² GC |
| Necropsy Day | Day 30 (N=2) Day 120 (N=2) | Day 30 (N=3) Day 120 (N=3) | Day 30 (N=3) Day 120 (N=3) | Day 30 (N=3) Day 120 (N=3) |

Intranasal (IN) administration is performed using the MAD Nasal device. A total volume of 0.28 mL will be administered, which will be delivered in four aliquots of 0.070 mL (two aliquots per nare). Abbreviation: F, female; FFB, final formulation buffer; GC, genome copies; FDP (final drug product), AAVrh91.CB7.CI.hAce2GTP14HL-IgG4.rBG; IN, intranasal; M, male; N, number of animals; N/A, not applicable, ROA, route of administration.

Cage-side clinical observations and evaluation of vital signs, body weights, and clinical pathology of the blood (CBC with differentials, clinical chemistries, and a coagulation panel) are considered safety readouts for IN administration of AAV vectors and are therefore be obtained at frequent intervals throughout the study. The potential of AAVrh91.CB7.CI.hAce2GTP14HL-IgG4.rBG to interact with the renin angiotensin system, angiotensin II, angiotensin-(1-7), and angiotensin-(1-9) is assessed as part of the clinical pathology panel.

The expression of the transgene product (i.e., hAce2GTP14HL-IgG4) is measured in serum, NLF, and BALF using a SARS-CoV-2 spike binding assay and MS analysis.

NAb responses against the AAVrh91 capsid are measured at baseline to assess the impact on transduction (biodistribution) and throughout the study to assess the kinetics of the NAb response. PBMCs are collected at baseline and every 30 days thereafter to evaluate T cell responses to the capsid and/or transgene product using an interferon gamma (IFN-γ) enzyme-linked immunospot (ELISpot) assay. The time points for PBMC collection were selected because T cell and B cell immune responses typically occur within 30 days in NHPs. At necropsy, tissue-resident lymphocytes from the spleen and liver are also collected for evaluation of T cell responses to the capsid and/or transgene product.

Blood is collected to assess vector distribution, and urine and feces are collected to assess vector excretion (shedding). These samples are collected at frequent time points and quantified by qPCR to enable assessment of the kinetics of vector distribution and excretion post treatment.

A comprehensive list of tissues is collected at necropsy for histopathology and tissue biodistribution. These tissues were selected based on the biodistribution data from above mentioned studies, and include possible target tissues of the airways, along with additional tissues of the nervous system (brain, spinal cord, peripheral nerves, DRG) and highly perfused peripheral organs (such as the liver and kidneys).

The dose examined are: low dose - 5.0 × 10¹⁰ GC, mid-dose - 5.0 × 10¹¹ GC, high dose - 5.0 × 10¹² GC. These examined doses are used for dose scaling from animal subject to human subject, which is based on the surface area of nasal cavity. More specifically, the surface area of the human nasal cavity (0.0181 m²) is approximately 2.94 times greater than the surface area of the NHP nasal cavity (0.00616 m²) [Hoffman G.M., Cracknell S., Damiano J.M., Macri N.P., & Moore S. (2014)]. “Inhalation Toxicology.” Handbook of Toxicology: CRC Press: 233-300). The administration volume in humans for the MAD Nasal device is 0.80 mL based on the manufacturer’s procedure guide. Therefore, the volume delivered into the NHP nasal cavity was calculated as follows: 0.80 mL ÷ 2.94 = 0.272 mL. To allow for improved accuracy when delivering small volumes in the NHP nasal cavity, we increased the volume to 0.28 mL to allow for accuracy of delivery of four aliquots of 0.070 mL instead of 0.068 mL. The vector doses are similarly scaled between NHPs and humans based on the surface area of the nasal cavity such that the low dose, mid-dose, and high dose for the planned Phase 1 FIH clinical trial are equivalent to the low dose, mid-dose, and high dose evaluated in the NHP toxicology study, respectively, and summarized in the table below.

Dose Scaling

| NHP (Adult) | Human (Adult) | Revised Doses | Revised Doses^{∗c} | |
|---|---|---|---|---|
| Low Dose^{a} | 5.0 × 10¹⁰ GC | 1.5 × 10¹¹ GC | 3.00 × 10¹¹ GC | 1.02 × 10¹¹ GC |
| Mid-Dose^{a} | 5.0 × 10¹¹ GC | 1.5 × 10¹² GC | 1.00 × 10¹² GC | 3.40 × 10¹² GC |
| High Dose^{a} | 5.0 × 10¹² GC | 1.5 × 10¹³ GC | 3.00 × 10¹² GC | 1.02 × 10¹² GC |
| Administration Volume^{b} | 0.28 mL | 0.8 mL | | |

^{a}Clinical doses have been selected to be equivalent to the doses in the planned NHP toxicology study. The clinical doses were extrapolated from NHPs to humans by scaling based on the surface area of the nasal cavity for adult humans (0.0181 m²) versus adult NHPs (0.00616 m²) (Hoffman et al., 2014). Since the human nasal cavity is 2.94 times greater than the surface area of the NHP nasal cavity, the doses administered to NHPs were multiplied by a factor of 2.94 to arrive at equivalent human doses.
^{b}The administration volume selected for use in humans is the recommended administration volume for the MAD Nasal™ device based on the manufacturer’s procedure guide. The administration volume selected for NHPs was therefore extrapolated from humans to NHPs by scaling based on the surface area of the nasal cavity for adult humans (0.0181 m²) versus adult NHPs (0.00616 m²) (Hoffman et al., 2014). Since the human nasal cavity is 2.94 times greater than the surface area of the NHP nasal cavity, the human administration volume was divided a factor of 2.94 to arrive at an equivalent NHP administration volume.
^{∗c}Revised dose is reflective of the 2.94-fold change in surface area of the nose between humans and rhesus macaques. The volume of vector delivered to the rhesus macaques was extrapolated based on the surface area of the nose as follows. The surface area of the human nose (0.0181 m2) is approximately 2.94 times greater than the surface area of the rhesus macaque nose (0.00616 m2) (Hoffman et al. 2014). As such, the volume delivered into the NHP nose was calculated as follows: 0.80 mL (the volume in which vector will be delivered in the human nose) ÷ 2.94 = 0.272 mL. To allow for improved accuracy when delivering small volumes in the macaque nose, we increased the volume to 0.28 mL to allow for accuracy of delivery of four aliquots of 0.070 mL instead of 0.068 mL.

Table immediately below shows a brief overview of the study set-up.

| | | | | | |
|---|---|---|---|---|---|
| Group Designati on | AAVrh91.CB7.CI.hAce2GTP1 4HL-IgG4.rBG / Vehicle (FFB) | AAVrh91.CB7.CI.hAce2G TP14HL-IgG4.rBG Dose (GC) | NHP ID | Sex | RO A |
| 1 | FFB | N/A | AT344G | Femal e | IN |
| | | | AF629LG | Male | |
| 2 | AAVrh91. hAce2GTP14HL-IgG4 | 1.02 × 10¹¹ | FR111A | Femal e | IN |
| | | | BM559B | Male | |
| | | | AY19O | Male | |
| 3 | AAVrh91. hAce2GTP14HL-IgG4 | 3.40 × 10¹¹ | AM685DB | Femal e | IN |
| | | | AT268I | Male | |
| | | | AC904KB | Male | |
| 4 | AAVrh91. hAce2GTP14HL-IgG4 | 1.02 × 10¹² | BM737C | Femal e | IN |
| | | | R701KD | Male | |
| | | | AT32J | Male | |
| 5 | FFB | N/A | SA83L | Femal e | IN |
| | | | V59ID | Male | |
| 6 | AAVrh91. hAce2GTP14HL-IgG4 | 1.02 × 10¹¹ | AV412A | Femal e | IN |
| | | | AG64KC | Male | |
| | | | GR691LF | Male | |
| 7 | AAVrh91. hAce2GTP14HL-IgG4 | 3.40 × 10¹¹ | AM778F | Femal e | IN |
| | | | AL211K | Male | |
| | | | VL63K | Male | |
| 8 | AAVrh91. hAce2GTP14HL-IgG4 | 1.02 × 10¹² | T410JA | Femal e | IN |
| | | | AM169EB | Male | |
| | | | AG184O | Male | |

Table immediately below shows decoy protein levels in collected nasal lavage fluid (NLF) as measured in ng/mL.

| Group Designation | Necropsy Day | NLF (ng/mL) | | | Assuming 55-fold dilution^{∗∗} (ng/mL in ASF) |
|---|---|---|---|---|---|
| | | Study day 7 | | | |
| | | Left | Right | Average | |
| 1 | Day 30±3 | ND | ND | ND | |
| | | ND | ND | | |
| 2 | Day 30±3 | 0.124 | 0.151 | 0.142 | 7.58 |
| | | ND | 0.151 | | |
| | | ND | ND | | |
| 3 | Day 30±3 | 0.192 | ND | 0.241 | 13.3 |
| | | 0.426 | 0.300 | | |
| | | 0.135 | 0.154 | | |
| 4 | Day 30±3 | 0.763 | 0.872 | 0.987 | 54.3 |
| | | 1.133 | 0.944 | | |
| | | 1.051 | 1.156 | | |
| 5 | Day 120±4 | ND | ND | ND | |
| | | ND | ND | | |
| 6 | Day 120±4 | 0.096 | 0.123 | 0.126 | 7.0 |
| | | 0.118 | 0.122 | | |
| | | 0.151 | 0.149 | | |
| 7 | Day 120±4 | 0.393 | 0.273 | 0.420 | 23.1 |
| | | 0.420 | 0.578 | | |
| | | 0.393 | 0.464 | | |
| 8 | Day 120±4 | 1.007 | 0.851 | 0.946 | 52.0 |
| | | 0.871 | 0.929 | | |
| | | 1.226 | 0.792 | | |

^{∗∗}Dilution factor could range up to 80-fold (55-fold was average in Examples 1-4, and 7).

FIG. 34 shows correlation of protein decoy expression in collected NLF following AAVrh91.hAce2GTP14HL-IgG4 administration at various doses (1.02 × 10¹¹, 3.40 × 10¹², and 1.02 × 10¹² GC).

FIG. 35 shows hAce2 decoy protein levels in NLF, plotted as ng/mL, measured in NHPs at day 30 and day 120 following AAVrh91.hAce2GTP14HL-IgG4 administration at various doses (1.02 × 10¹¹, 3.40 × 1012, and 1.02 × 10¹² GC) in NHPs.

FIG. 21D shows a comparison of hAce2 decoy protein level expression in NHPs, plotted as ng/mL, following administration with either AAVrh91.CDY14HL-Fc4, AAVrh91.CDY14HL-Fc4, AAVhu68.CDY14HL-Fc4, or AAVhu68.CDY14-Fc4 administered at doses of 5 x 10¹² GC or 5 × 10¹¹ GC.

### EXAMPLE 9. Pharmacology and Transmission Study in Ferrets

Ferret AAV-transduction and COVID-transmission study was performed to evaluate the protective efficacy of AAVrh91.CB7.CI.hAce2GTP14HL-IgG4.rBG (AAVrh91.GTP14HL; comprising hAce2-Variant2-IgG4 (Variant2)) or AAVhuu68.CB7.CI.hAce2GTP14HL-IgG4.rBG (AAVhu68.GTP14HL; comprising hAce2-Variant2-IgG4 (Variant2)) when administered intranasally (IN) to ferrets prior to challenge with SARS-CoV2 (USA-WA1/2020 variant).

In this pilot study, we examined efficacy of the AAV.hAce2 decoy fusion when administered at a dose 2.5 x 10¹² GC (n=6), throughout duration of the study, an overview of which is summarized in table below.

| Group | N | Clinical Observations | Treatment (IN)^{a} SD: 0 | | Nasal Wash^{b} | Nasal Swab | Termination |
|---|---|---|---|---|---|---|---|
| 1 | 2 | Daily Observations for study duration and body weight collections during anesthetic events | TA1-AAVhu68.GTP14HL Dose: 2.5 X 10¹² GC/animal | 400 µL (200 µL each nare) | SD: 2, 7 | SD: 2, 7 | SD 7: Terminal Bronchoalveolar Lavage (BAL), necropsy with tissue collection for histopathology and biodistribution |
| 2 | 2 | | TA2 -AAVrh91.GTP14HL Dose: 2.5 X 10¹² GC/animal | | | | |
| 3 | 3 | | TA1 -AAVhu68.GTP14HL Dose: 2.5 X 10¹² GC/animal | | SD: 2, 7, 14, 21, 28 | SD: 2, 7, 14, 21, 28 | SD 28: Terminal Bronchoalveolar Lavage (BAL), necropsy with tissue collection for histopathology and biodistribution |
| 4 | 3 | | TA2 -AAVrh91.GTP14HL Dose: 2.5 X 10¹² GC/animal | | | | |

^{a}Dose to be used is same concentration of max. dose in the clinical trial (5×10¹² GC in 0.8 ml) in 400 µl for ferrets
SD - study day

Briefly, ferrets were intranasally administered with 2.5 x 10¹² GC/animal of research grade vector preps. The nasal lavage fluid (NLF) was collected on days 7, 14, 21, and 28 for determination of protein level by mass spectrometry. Necropsy was performed on day 7, in some animals with BALF collection for mass spectrometry, to examine for blood contamination.

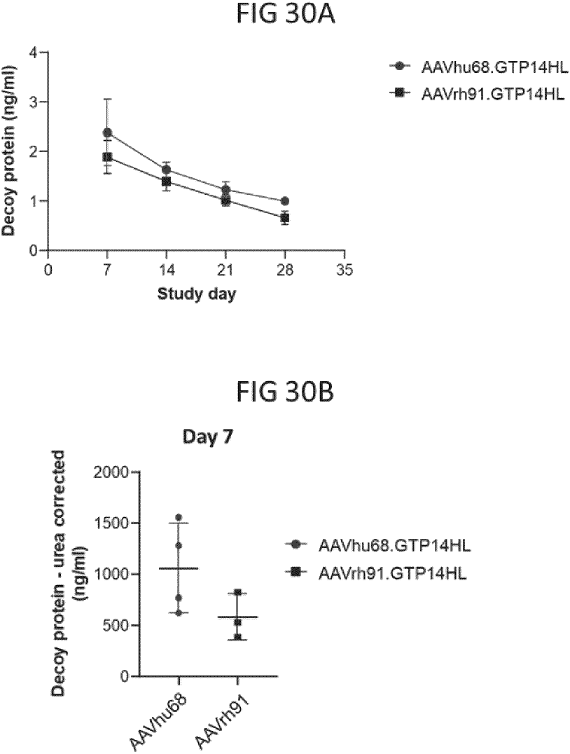

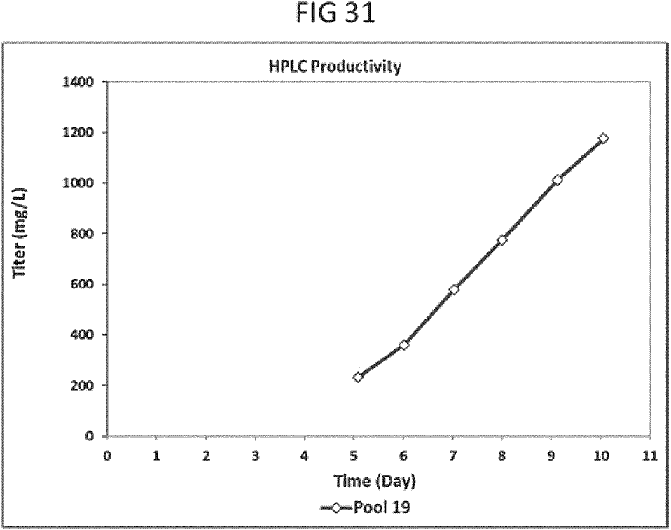

FIG. 30A shows decoy protein levels as determined by mass spectrometry analysis (ng/mL) post administration of either AAVhu68.GTP14HL or AAVrh91.GTP14HL.

FIG. 30B shows decoy protein levels as determined by mass spectrometry analysis (ng/mL) post administration of either AAVhu68.GTP14HL or AAVrh91.GTP14HL, and plotted as urea-corrected decoy protein concentration (ng/mL).

Furthermore, we performed an additional ferret transduction study to confirm the previously obtained results of the pilot study at a dose of a dose of 2.5×10¹² GC. The layout of the study is summarized in the table below.

| Group | N | Weights and Clinical Observations | Treatment (IN) - SD: 0 | | Nasal Wash | Nasal Swab | Termination |
|---|---|---|---|---|---|---|---|
| 1 | 3 | Daily weights and observations during challenge period | TA1 -AAVhu68.GTP14HL | 400 µL (200 µL each nare) | SD 1, 3, 5, 7 | SD 0-7 | SD 7: Euth. |
| 2 | 3 | | TA2 -AAVrh91.GTP14HL | | | | |

Next, a study of the transmission of CoV2 in ferrets is performed, the layout of the study of which is summarized in a table below.

| Grou p | N | Treatment (IN) | Dose | T x D a y s | Challeng e (IN) | Weights and clinical observation | Nasal Swabs | Nasal Wash | Termination |
|---|---|---|---|---|---|---|---|---|---|
| 1 | 6 | Donor (no treatment) | N/A | N / A | SARS-CoV-2 on SD -2 (5.4 × 10⁵ TCID₅₀) | Weights on SD -7, SD -2 through SD 7, SD 14 Daily observations from SD -6 through SD 14 | SD: 2 through SD 7 SD 14 | SD: 1, 3, 5, 7, 14 | SD 14: Euth. |
| 2 | 6 | Mock (Placebo) | 400 µL (200 µL each nare) | S D : 7 | Not challenge d. Animals will be co-housed with Group 1 animals on SD 0 | | | | |
| 3 | 6 | Clinical Candidate | | | | | | | |

The outcomes of the following study are clinical observations, nasal swabs and washes for viral load, nasal washes for clinical candidate concentration.

### Example 10. In Vitro Neutralization of CoV2 Variants by Engineered hAce2 Protein Decoy Fusion.

In this study, we further examined the potency (neutralization activity) of the engineered hAce2 decoy in the in vitro neutralization assay against different CoV2 variants. The neutralization assay (pseudotyped lentivirus neutralization assay) was performed as described above. The engineered hAce2 decoy protein, which were used, were purified by affinity chromatography followed by size-exclusion chromatography. It is noted that hAce2-MR27HL-Variant-IgG1 showed increased expression in vitro while maintaining similar or same potency against SARS-CoV2 as in comparison to hAce2-Variant2-IgG (CDY14HL-Fc).

The neutralization activity efficacy was examined for the AAV Decoy GTP14HL-Fc4 (hACe2-Variant2-IgG4Fc) and for the Protein Decoy MR27HL-Fc1 (hAce2-MR27HL-Variant-IgG1 Fc), against various SARS-CoV2 (CoV2 variants) and presented in the table immediately below as a IC₅₀ (IC50) values in ng/mL.

| RBD Mutation | CoV Variant | IC50 (ng/mL) | |
|---|---|---|---|
| | | AAV Decoy GTP14HL-Fc4 (hACe2-Variant2-IgG1Fc) IC50 (ng/mL) | Protein Decoy MR27HL-Fc1 IC50 (ng/mL) |
| - | Wuhan | 37 | 33 |
| N501Y | alpha | 29 | nd |
| K417M, E484K, N501Y | beta | 28 | 17 |
| L452R, E484Q | kappa | 16 | 45 |
| K417T, E484K, N501Y | gamma | 16 | nd |
| L452R | epsilon | 23 | nd |
| S477N, E484K | iota | 53 | nd |
| E484K | zeta | 43 | 110 |
| L452Q, F490S | lambda | 6 | 10 |
| L452R, T478K | delta | 11 | 13 |
| K417N, L452R. T478K | delta plus | 8 | 12 |
| R346K, E484K, N501Y | mu | 18 | 24 |
| N/A | CoV1 | 10 | 11 |
| S371L, S373P, S375F, K417N, N440K, G446S, S447N, T478K, E484A, Q493K, G496S, Q498R, N501Y, Y505H | omicron | 20 | 71 |

These results show that engineered hACE2 decoys display broad tolerance to continued CoV2 evolution.

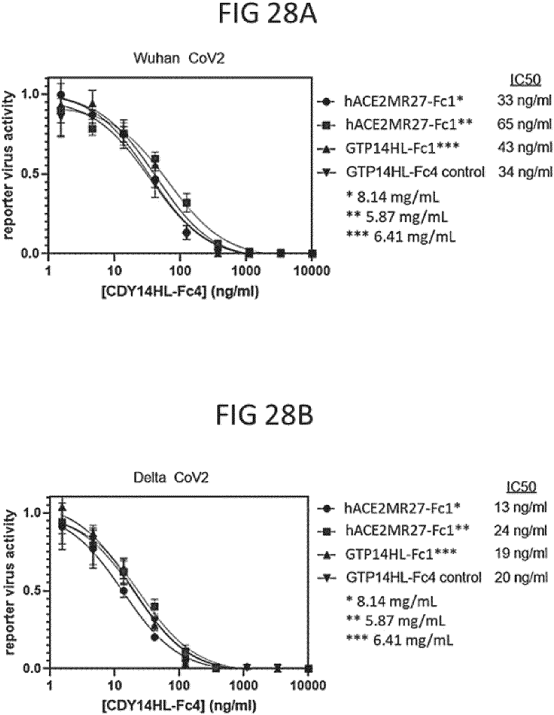

FIG. 27 shows a plot of neutralization data as measured across different sampled pools of the purified engineered hAce2 decoy Fc1 (IgG1 Fc) fusion proteins, and compared with the engineered hAce2 decoy Fc4 (IgG4 Fc) fusion protein. FIG. 28A shows a plot of neutralization data against Wuhan CoV2, as measured across different sampled pools of the purified engineered hAce2 decoy Fc1 (IgG1 Fc) fusion proteins, and compared with the engineered hAce2 decoy Fc4 (IgG4 Fc) fusion protein. FIG. 28B shows a plot of neutralization data against Delta CoV2 (variant), as measured across different sampled pools of the purified engineered hAce2 decoy Fc1 (IgG1 Fc) fusion proteins, and compared with the engineered hAce2 decoy Fc4 (IgG4 Fc) fusion protein. These results show that engineered hAce2 decoy fusion proteins display broad tolerance to continued CoV2 evolution.

Furthermore, we compared efficacy in neutralization of GTP14HL-IgG1 Fc (hAce2(variant 2)-IgG1Fc) in comparison to GTP14HL-IgG4 FC (hAce2(variant 2)-IgG4Fc), as summarized in table below.

IC50(ng/mL)

| Decoy | Wuhan | Delta |
|---|---|---|
| GTP14HL-Fc4 | 37 | 11 |
| GTP14-HL-Fc1 | 19 | 19 |
| MR27HL_Fc1 | 33 | 13 |

Additionally, we examined the correlation between the increase in transmissibility in CoV2 viruses and the engineered hAce2 neutralization efficacy, which is summarized in the table below.

| | | Variants of Interest/Variants of Concern (VOI/VOC) | | | | |
|---|---|---|---|---|---|---|
| | Wuhan | alpha | beta | gamma | kappa | delta |
| Transmissibility increase vs. VOI/VOC | - | 29% | 25% | 38% | 48% | 97% |
| Decoy IC50 (ng/mL) | 37 | 29 | 28 | 16 | 16 | 11 |

These results show that engineered hAce2 decoys gain potency as CoV2 evolves toward higher transmissibility.

### EXAMPLE 11. High Activity of an Affinity Matured ACE2 Decoy Against Omicron SARS-CoV-2 and Pre-emergent Coronaviruses

We previously generated an affinity-matured decoy inhibitor based on the receptor target of the SARS-CoV-2 spike protein, angiotensin converting enzyme 2 (ACE2), and deployed it in an adeno-associated viral vector (rAAV) for intranasal delivery and passive prophylaxis against COVID-19. Here we demonstrate exceptional binding or neutralizing potency of this ACE2 decoy against SARS-CoV-2 variants including omicron, as well as diverse ACE2-dependent coronaviruses. Here we discuss a strategy of decoy-based treatment and passive protection to mitigate the ongoing COVID-19 pandemic and future airway virus threats.

### A. Summary

Viral sequences can change dramatically during pandemics lasting multiple years. Likewise, evolution over centuries has generated genetically diverse virus families posing similar threats to humans. This variation presents a challenge to drug development, in both the breadth of achievable protection against related groups of viruses and the durability of therapeutic agents or vaccines during extended outbreaks. This phenomenon has played out dramatically during the coronavirus disease 2019 (COVID-19) pandemic. The highly divergent Omicron variant of severe acute respiratory syndrome coronavirus 2 (SARS-CoV-2) has upended previous gains won by vaccine and monoclonal antibody development. Moreover, ecological surveys have increasingly revealed a broad class of SARS-CoV-2-like viruses in animals, each poised to cause a future human pandemic. In this study, we evaluate an alternative to antibody-based protection and prevention-a decoy molecule based on the SARS-CoV-2 receptor. Our engineered decoy has proven resistant to SARS-CoV-2 evolution during the ongoing COVID-19 pandemic and can neutralize all variants of concern, including Omicron BA.1 and Omicron BA.2. We also demonstrate the exceptional binding and neutralizing potency of this ACE2 decoy against SARS-CoV-2 variants including Omicron BA.1 and Omicron BA.2. Tight decoy binding tracks with human ACE2 binding of viral spike receptor-binding domains across diverse clades of coronaviruses. Furthermore, in a coronavirus that cannot bind human ACE2, a variant that acquired human ACE2 binding was bound by the decoy with nanomolar affinity. Furthermore, the decoy binds tightly to a broad class of sarbecoviruses related to pandemic SARS-CoV-2 and SARS-CoV-1, indicating that receptor decoys offer advantages over monoclonal antibodies and may be deployed during the COVID-19 pandemic and future coronavirus outbreaks to prevent and treat severe illness.

### B. Introduction

Monoclonal antibody therapeutics with the ability to bind spike protein of severe acute respiratory syndrome coronavirus 2 (SARS-CoV-2) and prevent cell entry have been critical tools in managing the coronavirus disease 2019 (COVID-19) pandemic [Wec AZ, Wrapp D, Herbert AS, Maurer DP, Haslwanter D, Sakharkar M, et al. Broad neutralization of SARS-related viruses by human monoclonal antibodies. Science. 2020;369(6504):731-6; Weinreich DM, Sivapalasingam S, Norton T, Ali S, Gao H, Bhore R, et al. REGN-COV2, a Neutralizing Antibody Cocktail, in Outpatients with Covid-19. N Engl J Med. 2021;384(3):238-51]. These drugs prevent hospitalizations when applied early in the course of infection [Weinreich DM, Sivapalasingam S, Norton T, Ali S, Gao H, Bhore R, et al. REGEN-COV Antibody Combination and Outcomes in Outpatients with Covid-19. N Engl J Med. 2021;385(23):e81] and can provide critical passive protection for vulnerable populations of immunocompromised patients who cannot mount a protective response to vaccines [O′Brien MP, Hou P, Weinreich DM. Subcutaneous REGEN-COV Antibody Combination to Prevent Covid-19. Reply. N Engl J Med. 2021;385(20):e70]. However, monoclonals have proven susceptible to SARS-CoV-2 evolution [Starr TN, Greaney AJ, Addetia A, Hannon WW, Choudhary MC, Dingens AS, et al. Prospective mapping of viral mutations that escape antibodies used to treat COVID-19. Science. 2021;371(6531):850-4]. This susceptibility may arise because the spike epitopes most sensitive to neutralization have been under intense selection as the virus has made gains in transmissibility and its ability to evade human immunity [Barton MI, MacGowan SA, Kutuzov MA, Dushek O, Barton GJ, van der Merwe PA. Effects of common mutations in the SARS-CoV-2 Spike RBD and its ligand, the human ACE2 receptor on binding affinity and kinetics. Elife. 2021;10]. Detailed structural analyses have highlighted that the SARS-CoV-2 receptor-binding domain (RBD) is obscured from antibodies by a thick glycan coat that it is particularly evident when the RBD is in the downward conformation, enabling it to evade neutralization. Additionally, the mobility of the RBD between up and down conformations may itself present an issue for RBD:antibody interactions [Turonova B, Sikora M, Schurmann C, Hagen WJH, Welsch S, Blanc FEC, et al. In situ structural analysis of SARS-CoV-2 spike reveals flexibility mediated by three hinges. Science. 2020;370(6513):203-8]. Thus, monoclonal antibodies that target the RBD in the upward conformation may show reduced susceptibility [Turonova B, Sikora M, Schurmann C, Hagen WJH, Welsch S, Blanc FEC, et al. In situ structural analysis of SARS-CoV-2 spike reveals flexibility mediated by three hinges. Science. 2020;370(6513):203-8; Pierri CL. SARS-CoV-2 spike protein: flexibility as a new target for fighting infection. Signal Transduct Target Ther. 2020;5(1):254].

Several groups have identified several sites present in the functional binding epitope of the RBD spike protein that have undergone mutations to evade all currently available classes of monoclonal antibodies [Ding C, He J, Zhang X, Jiang C, Sun Y, Zhang Y, et al. Crucial Mutations of Spike Protein on SARS-CoV-2 Evolved to Variant Strains Escaping Neutralization of Convalescent Plasmas and RBD-Specific Monoclonal Antibodies. Front Immunol. 2021;12:693775; Greaney AJ, Loes AN, Crawford KHD, Starr TN, Malone KD, Chu HY, et al. Comprehensive mapping of mutations in the SARS-CoV-2 receptor-binding domain that affect recognition by polyclonal human plasma antibodies. Cell Host Microbe. 2021;29(3):463-76 e6; Greaney AJ, Starr TN, Barnes CO, Weisblum Y, Schmidt F, Caskey M, et al. Mapping mutations to the SARS-CoV-2 RBD that escape binding by different classes of antibodies. Nat Commun. 2021;12(1):4196.]. The most significant escape mutants identified are K417 for class 1 antibodies, L452R and E484 for class 2 antibodies, and R346, K444, and G446-450 sites for class 3 antibodies. It is concerning that many of these mutations are present in emergent CoV-2 variants, which also show reduced susceptibility to monoclonal antibodies [Ding C, He J, Zhang X, Jiang C, Sun Y, Zhang Y, et al. Crucial Mutations of Spike Protein on SARS-CoV-2 Evolved to Variant Strains Escaping Neutralization of Convalescent Plasmas and RBD-Specific Monoclonal Antibodies. Front Immunol. 2021;12:693775; Zhou H, Tada T, Dcosta BM, Landau NR. Neutralization of SARS-CoV-2 Omicron BA.2 by Therapeutic Monoclonal Antibodies. bioRxiv. 2022; Campbell F, Archer B, Laurenson-Schafer H, Jinnai Y, Konings F, Batra N, et al. Increased transmissibility and global spread of SARS-CoV-2 variants of concern as at June 2021. Euro Surveill. 2021;26(24)]. While comprehensive structural analysis suggests potential improvements for targeting monoclonal antibodies and other biologics to the spike protein for inhibition [Mercurio I, Tragni V, Busto F, De Grassi A, Pierri CL. Protein structure analysis of the interactions between SARS-CoV-2 spike protein and the human ACE2 receptor: from conformational changes to novel neutralizing antibodies. Cell Mol Life Sci. 2021;78(4):1501-22; Starr TN, Czudnochowski N, Liu Z, Zatta F, Park YJ, Addetia A, et al. SARS-CoV-2 RBD antibodies that maximize breadth and resistance to escape. Nature. 2021;597(7874):97-102; Walls AC, Park YJ, Tortorici MA, Wall A, McGuire AT, Veesler D. Structure, Function, and Antigenicity of the SARS-CoV-2 Spike Glycoprotein. Cell. 2020;181(2):281-92 e6; Wrapp D, Wang N, Corbett KS, Goldsmith JA, Hsieh CL, Abiona O, et al. Cryo-EM structure of the 2019-nCoV spike in the prefusion conformation. Science. 2020;367(6483):1260-3], the fact remains that SARS-CoV-2 evolution during the course of the pandemic has generated variants which have evaded essentially the whole spectrum of clinical monoclonal candidates to date [Starr TN, Czudnochowski N, Liu Z, Zatta F, Park YJ, Addetia A, et al. SARS-CoV-2 RBD antibodies that maximize breadth and resistance to escape. Nature. 2021;597(7874):97-102; Zhang L, Narayanan KK, Cooper L, Chan KK, Devlin CA, Aguhob A, et al. An engineered ACE2 decoy receptor can be administered by inhalation and potently targets the BA.1 and BA.2 omicron variants of SARS-CoV-2. bioRxiv. 2022]. Furthermore, evidence suggests that single monoclonals applied in a therapeutic setting can rapidly give rise to escape mutants [Van Egeren D, Novokhodko A, Stoddard M, Tran U, Zetter B, Rogers M, et al. Risk of rapid evolutionary escape from biomedical interventions targeting SARS-CoV-2 spike protein. PLoS One. 2021;16(4):e0250780; Fenaux H, Gueneau R, Chaghouri A, Henry B, Mouna L, Roque-Afonso AM, et al. Emergence of SARS-CoV-2 resistance mutations in a patient who received anti-SARS-COV2 spike protein monoclonal antibodies: a case report. BMC Infect Dis. 2021;21(1):1223; Jensen B, Luebke N, Feldt T, Keitel V, Brandenburger T, Kindgen-Milles D, et al. Emergence of the E484K mutation in SARS-COV-2-infected immunocompromised patients treated with bamlanivimab in Germany. Lancet Reg Health Eur. 2021;8:100164; Rockett RJ, Basile K, Maddocks S, Fong W, Agius JE, Mackinnon JJ, et al. RESISTANCE CONFERRING MUTATIONS IN SARS-CoV-2 DELTA FOLLOWING SOTROVIMAB INFUSION. medRxiv. 2021:2021.12.18.21267628]. Together, these findings call into question the ability of the antibody platform to keep pace with the course of the COVID-19 pandemic or to be of use in future pandemics caused by other coronaviruses.

Receptor decoys may provide a mode of viral neutralization that is more resistant to continued viral evolution and escape-mutant generation [Higuchi Y, Suzuki T, Arimori T, Ikemura N, Mihara E, Kirita Y, et al. Engineered ACE2 receptor therapy overcomes mutational escape of SARS-CoV-2. Nat Commun. 2021;12(1):3802]. SARS-CoV-2 evolution has occurred in a way that retains tight binding to its primary cell entry receptor, angiotensin-converting enzyme 2 [Letko M, Marzi A, Munster V. Functional assessment of cell entry and receptor usage for SARS-CoV-2 and other lineage B betacoronaviruses. Nat Microbiol. 2020;5(4):562-9]. We and others have developed affinity-matured, soluble ACE2 decoy molecules that potently neutralize SARS-CoV-2 [Higuchi et al., 2021, cited above; [Examples 1-4, and 7]; Chan KK, Tan TJC, Narayanan KK, Procko E. An engineered decoy receptor for SARS-CoV-2 broadly binds protein S sequence variants. Sci Adv. 2021;7(8); Glasgow A, Glasgow J, Limonta D, Solomon P, Lui I, Zhang Y, et al. Engineered ACE2 receptor traps potently neutralize SARS-CoV-2. Proc Natl Acad Sci U S A. 2020;117(45):28046-55; Havranek B, Chan KK, Wu A, Procko E, Islam SM. Computationally Designed ACE2 Decoy Receptor Binds SARS-CoV-2 Spike (S) Protein with Tight Nanomolar Affinity. J Chem Inf Model. 2021;61(9):4656-69; Jing W, Procko E. ACE2-based decoy receptors for SARS coronavirus 2. Proteins. 2021;89(9):1065-78; Chan KK, Dorosky D, Sharma P, Abbasi SA, Dye JM, Kranz DM, et al. Engineering human ACE2 to optimize binding to the spike protein of SARS coronavirus 2. Science. 2020;369(6508):1261-5]. Our soluble, Fc-fused decoy, CDY14HL-Fc4 (hAce2-Variant2-IgG), contains six (6) amino acid substitutions that improve neutralization of CoV2 variants by 300-fold versus un-engineered ACE2 and contain an active site mutation that ablate its endogenous angiotensin-cleaving activity (as described in Examples 1-4, and 7 above). Furthermore, CDY14HL-Fc4 maintains tight binding or neutralizing activity for the distantly-related sarbecoviruses WIV1-CoV and SARS-CoV-1 despite being engineered for improved activity against SARS-CoV-2. This suggests that this decoy may be a useful tool to combat future pandemics from currently pre-emergent ACE2-dependent coronaviruses.

Here we evaluate the binding and neutralization activity of CDY14HL-Fc4 against a wide range of emerging SARS-CoV-2 variants, including Omicron BA.1 and Omicron BA.2, and a wide range of pre-emergent coronaviruses with similarity to SARS-CoV-1 and SARS-CoV-2. These studies suggest the broad utility of decoy-based viral entry inhibitors in combating current and future coronavirus pandemics.

### C. Results

### C.1. CDY14HL-Fc4 Maintains Tight Binding to Diverse SARS-CoV-2 Variants

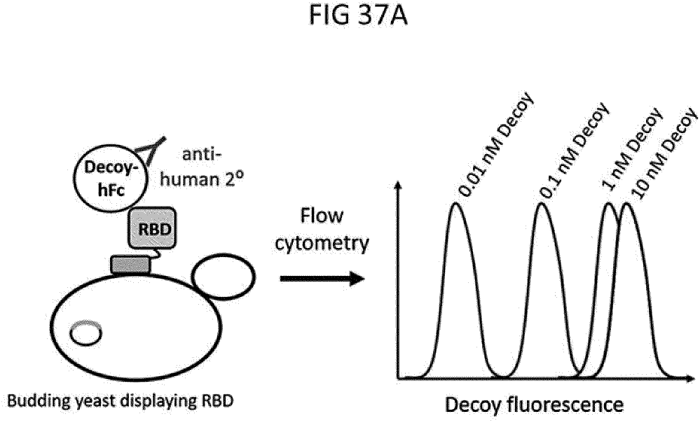

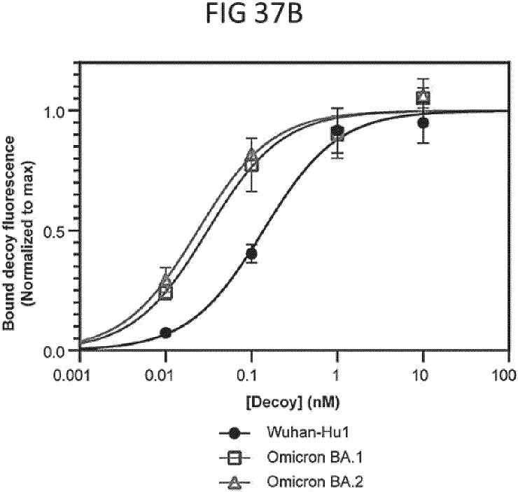

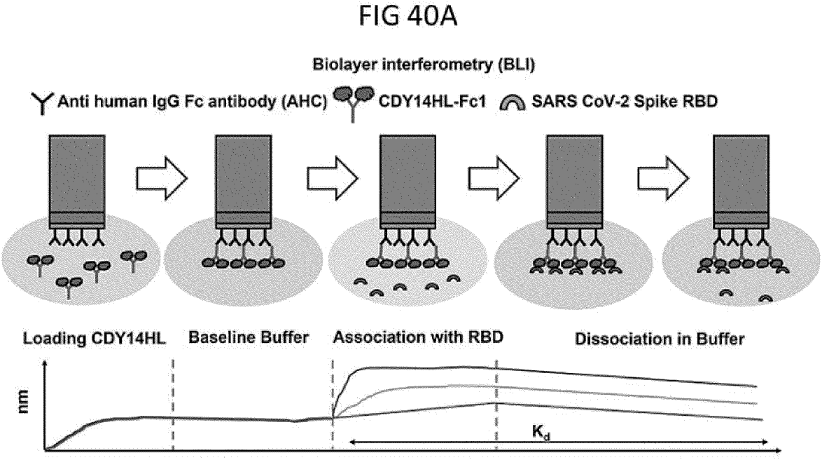

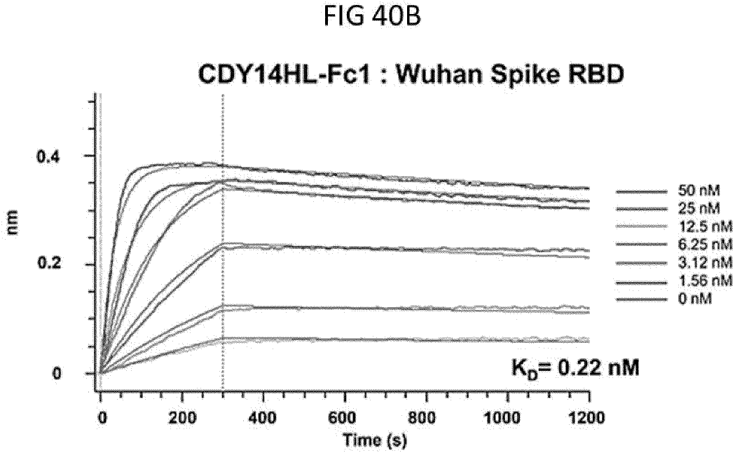

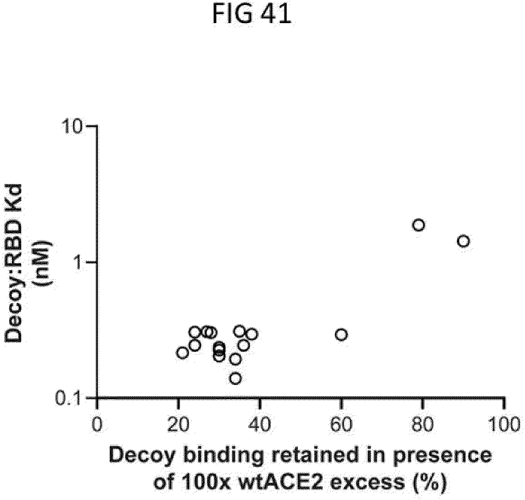

We set out to evaluate the ability of our engineered ACE2 decoy to neutralize emerging SARS-CoV-2 strains. As a first step we assessed binding to variant RBDs using a yeast display system [Angelini A, Chen TF, de Picciotto S, Yang NJ, Tzeng A, Santos MS, et al. Protein Engineering and Selection Using Yeast Surface Display. Methods Mol Biol. 2015;1319:3-36] (FIG. 37A). Briefly, RBDs from SARS-CoV-2 variants are individually expressed in budding yeast as fusion proteins and are trafficked to the external cell wall. Decoy or un-engineered ACE2 are incubated with the yeast, stained with antibodies, and the relative level of decoy binding is assessed by flow cytometry. We generated budding yeast displaying viral RBDs as fusion proteins to the cell-surface-tethered yeast protein Aga2. We then incubated the RBD yeast with CDY14HL-Fc1 fusion protein and assessed decoy binding via flow cytometry by staining bound decoy with a fluorescent secondary antibody. CDY14HL-Fc1 bound the ancestral (Wuhan-Hu-1) RBD with an apparent affinity of 0.16 nM (FIG. 37B). FIG. 37B shows representative decoy binding data for the RBD from the ancestral (Wuhan-Hu1) SARS-CoV-2 strain. FIG. 36 shows Engineered ACE2 decoys bind diverse ACE2-dependent CoVs, plotted as decoy fluorescence for each specified CoV strain. This result is in good agreement with 0.21 nM affinity obtained for the interaction using an orthogonal technique, biolayer interferometry (BLI, FIGS. 40A and 40B), as well as with the picomolar binding affinity we previously measured for the engineered decoy:RBD interaction using surface plasmon resonance [Examples 1-4, and 7]. FIGS. 40A and 40B show Biolayer interferometry (BLI) kinetics for hAce2GTP14HL-Fc1 and SARS-CoV-2 Variants. FIG. 40A shows depiction of Biolayer interferometry assay format. hAce2GTP14HL-Fc1 was immobilized as ligand and SARS-CoV-2 variant RBD was used as analyte. FIG. 40B shows a representative fitted sensogram for hAce2GTP14HL-Fc1 and CoV-2 Wuhan-Hu1 spike RBD.

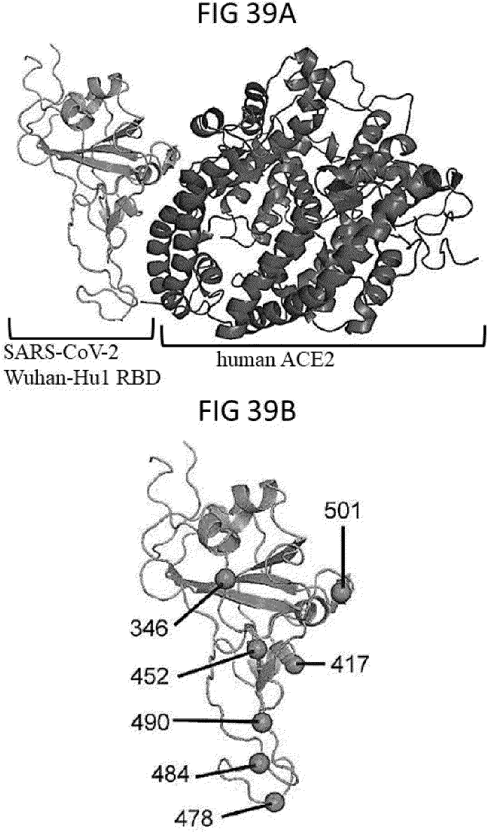

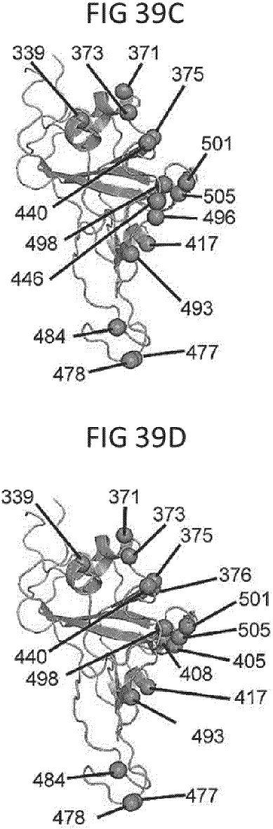

Since we first described CDY14HL-Fc [Examples 1-4, and 7], several SARS-CoV-2 Variants of Concern (VoC) have emerged with far greater transmissibility and clinical sequalae than the original Wuhan strain with most of this evolution occurring at the RBD:ACE2 interface (image not shown) [Campbell F, Archer B, Laurenson-Schafer H, Jinnai Y, Konings F, Batra N, et al. Increased transmissibility and global spread of SARS-CoV-2 variants of concern as at June 2021. Euro Surveill. 2021;26(24)]. FIG. 39A shows the complex [Xu C, Wang Y, Liu C, Zhang C, Han W, Hong X, et al. Conformational dynamics of SARS-CoV-2 trimeric spike glycoprotein in complex with receptor ACE2 revealed by cryo-EM. Sci Adv. 2021;7(1)] between the SARS-CoV-2 Wuhan-Hu1 RBD (yellow ribbons) and human ACE2 (blue ribbons). FIG. 39B shows the Wuhan-Hu1 RBD from the ACE2 complex structure alone. Red spheres indicate the α-carbon of amino acid residues mutated in several pre-Omicron SARS-CoV-2 variant strains. Amino acid position numbers are show for each mutated residue. FIG. 39C shows the Wuhan-Hu1 RBD from the ACE2 complex structure alone. Red spheres indicate the α-carbon of amino acid residues mutated around the interface with ACE2 in Omicron BA.1. FIG. 39D shows the Wuhan-Hu1 RBD from the ACE2 complex structure alone. Red spheres indicate the α-carbon of amino acid residues mutated around the interface with ACE2 in Omicron.BA.2

We used the yeast display system to evaluate decoy binding to 5 of these VoCs (Iota, Delta, Delta Plus, Lambda, and Mu). We included an additional RBD mutant not observed in natural SARS-CoV-2 isolates. This RBD sequence, “Delta 4+” contains 4 additional substitutions (K417N, N439K, E484K and N501Y) derived from systematic analysis of RBD mAb epitopes. These substitutions are hypothesized to hold maximum potential for antibody escape [Liu Y, Arase N, Kishikawa J-i, Hirose M, Li S, Tada A, et al. The SARS-CoV-2 Delta variant is poised to acquire complete resistance to wild-type spike vaccines. bioRxiv. 2021:2021.08.22.457114]. Remarkably, CDY14HL-Fc4 maintains sub-nanomolar binding affinity for all VoC RBDs, including Iota, Delta +, Lambda and Mu, and the “Delta 4+” RBD (FIG. 37B and table immediately below). This is consistent with the broad resistance of CDY14HL-Fc4 to SARS-CoV-2 variant evolution observed previously in binding and pseudotype neutralization studies [Examples 1-4, and 7].

| | | |
|---|---|---|
| Strain | RBD mutations relative to Wuhan-Hu1 | Yeast assay RBD binding K_{D} (nM) |
| CoV-2 (Wuhan-Hu-1) | none | 0.14 |
| Delta | L452R T478K | 0.21 |
| Delta + | K417N L452R T478K | 0.21 |
| Delta with 439 K/484K501Y | K417N N439K L452R T478K E484K N501Y | 0.07 |
| Iota | E484K | 0.10 |
| Kappa | L452R E484Q | NC |
| Lambda | L452Q F490S | 0.20 |
| Mu | R346K E484K N501Y | 0.10 |
| Omicron BA.1 | G339D S371L S373P S375F K417N N440K G446S S477N T478K E484A Q493K G496S Q498R N501Y Y505H | 0.031 |
| Omicron BA.2 | G339D S371F S373P S375F T376A D405N R408S K417N N440K S477N T478K E484A Q493R Q498R N501Y Y505H | 0.024 |
| Zeta | E484K | NC |

Table immediately above shows, fitted values of the dissociation equilibrium constant (KD) for SARS-CoV-2 variants (relative levels of decoy binding to variant RBDs under several conditions as assessed by the yeast-display system). NC: not collected.

### C.2. CDY14HL Maintains Potent Neutralization for Diverse SARS-CoV-2 Variants

Next, we investigated whether broad decoy affinity for SARS-CoV-2 variants we observed using the yeast display binding assay would translate to potent viral neutralization. We first examined the omicron VoC. Unlike previous variants, which contain one, two, or three RBD mutations, the Omicron BA.1 RBD differs from the ancestral strain by 15 amino acids (FIG. 39C), while the Omicron BA.2 variant RBD differs by 16 amino acids from the ancestral strain (FIG. 39D). [Miller NL, Clark T, Raman R, Sasisekharan R. Insights on the mutational landscape of the SARS-CoV-2 Omicron variant. bioRxiv. 2021]. This level of mutation has caused a reduction in efficacy of first-generation vaccines and most of the monoclonal antibodies developed for therapeutic and passive prophylaxis applications [Wilhelm A, Widera M, Grikscheit K, Toptan T, Schenk B, Pallas C, et al. Reduced Neutralization of SARS-CoV-2 Omicron Variant by Vaccine Sera and Monoclonal Antibodies. medRxiv. 2021:2021.12.07.21267432; Cao Y, Wang J, Jian F, Xiao T, Song W, Yisimayi A, et al. Omicron escapes the majority of existing SARS-CoV-2 neutralizing antibodies. Nature. 2021; Cameroni E, Bowen JE, Rosen LE, Saliba C, Zepeda SK, Culap K, et al. Broadly neutralizing antibodies overcome SARS-CoV-2 Omicron antigenic shift. Nature. 2021; Dejnirattisai W, Huo J, Zhou D, Zahradnik J, Supasa P, Liu C, et al. Omicron-B.1.1.529 leads to widespread escape from neutralizing antibody responses. bioRxiv. 2021; VanBlargan L, Errico J, Halfmann P, Zost S, Crowe J, Purcell L, et al. An infectious SARS-CoV-2 B.1.1.529 Omicron virus escapes neutralization by therapeutic monoclonal antibodies. Res Sq. 2021; Dejnirattisai W, Shaw RH, Supasa P, Liu C, Stuart AS, Pollard AJ, et al. Reduced neutralisation of SARS-CoV-2 omicron B.1.1.529 variant by post-immunisation serum. Lancet. 2022;399(10321):234-6]. Since our decoy was engineered for optimal binding to the ancestral strain, we sought to determine if this degree of antigenic drift would impact binding to the Omicron strains. Remarkably, the CDY14HL-Fc1 bound both Omicron strain RBDs with sub-nanomolar affinity as measured by the RBD yeast display assay (FIG. 37B and Table immediately above). The decoy bound yeast-displayed Omicron RBDs at least four-fold more tightly than the ancestral RBD (0.04 nM and 0.03 nM for BA.1 and BA.2 strains, respectively, Table immediately above). We have also confirmed sub-nanomolar decoy affinity for the Omicron strain RBDs using BLI, though the two distinct assay formats produced different rank-orders of variant affinities (FIGS. 40A and 40B).

We next investigated whether the broad decoy affinity for SARS-CoV-2 variants observed using the yeast display binding assay would translate to potent viral neutralization. We used a lentivirus harboring a luciferase reporter gene and pseudotyped with the SARS-CoV-2 spike protein from the omicron strain to measure the neutralization potency (IC50) of purified CDY14HL-Fc4 decoy (FIG. 33). FIG. 33 shows viral neutralization assay using lentiviruses pseudotyped with the ancestral (Wuhan Hu1) or Omicron variant spike protein. CDY14HL-Fc4 neutralizes Omicron BA.1 and BA.2 more potently than the ancestral strain (18 ng/ml for BA.1 and 30 ng/ml for BA.2 vs. 35 ng/ml for Wuhan-Hu-1, FIG. 33 and Table immediately below). We extended this approach to include all VoCs not previously evaluated. CDY14HL-Fc4 neutralizes all SARS-CoV-2 strain pseudotypes tested, including lambda, kappa, delta, delta +, mu and zeta, with IC50 values near or below the potency of the ancestral strain, Wuhan, against which it was engineered (summarized in table immediately below).

| Strain | RBD mutations relative to Wuhan-Hu1 | Pseudotype neutralization assay IC50 (ng/ml) |
|---|---|---|
| CoV-2 (Wuhan-Hu-1) | none | 35 |
| Delta | L452R T478K | 14 |
| Delta + | K417N L452R T478K | 10 |
| Delta with 439K/484K501Y | K417N N439K L452R T478K E484K N501Y | NC |
| Iota | E484K | NC |
| Kappa | L452R E484Q | 18 |
| Lambda | L452Q F490S | 11 |
| Mu | R346K E484K N501Y | 21 |
| Omicron BA.1 | G339D S371L S373P S375F K417N N440K G446S S477N T478K E484A Q493K G496S Q498R N501Y Y505H | 18 |
| Omicron BA.2 | G339D S371F S373P S375F T376A D405N R408S K417N N440K S477N T478K E484A Q493R Q498R N501Y Y505H | 30 |
| Zeta | E484K | 37 |

Table of CDY14HL neutralization IC50 values collected for SARS-CoV-2 variant pseudotypes along with the RBD mutations of each variant.

Together with these binding data, these variant neutralization data indicate that the CDY14HL decoy has a broad tolerance for CoV-2 strain evolution, losing virtually no potency in the face of extensive viral spike remodeling under the evolutionary pressures of a several-year-long global pandemic.

### C.3. CDY14HL Binds Diverse ACE2-dependent CoVs

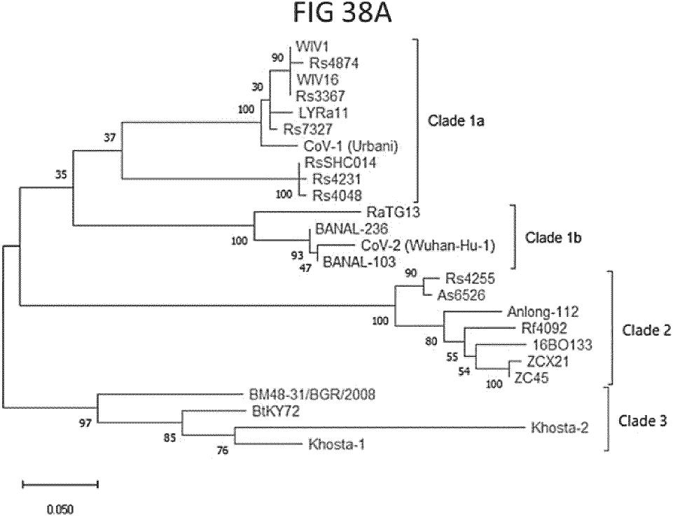

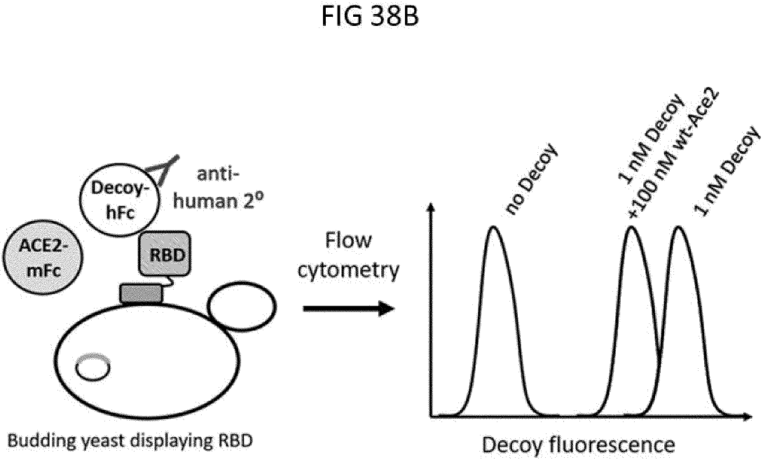

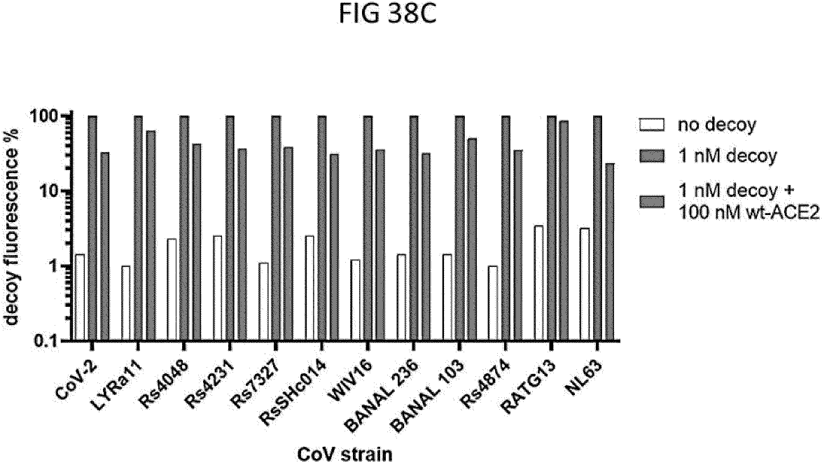

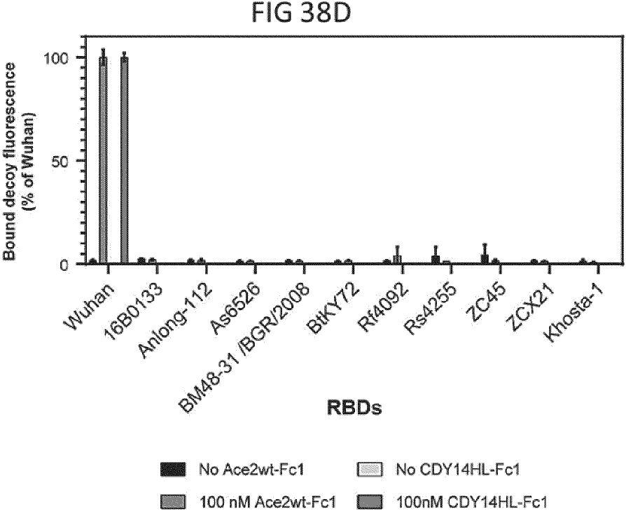

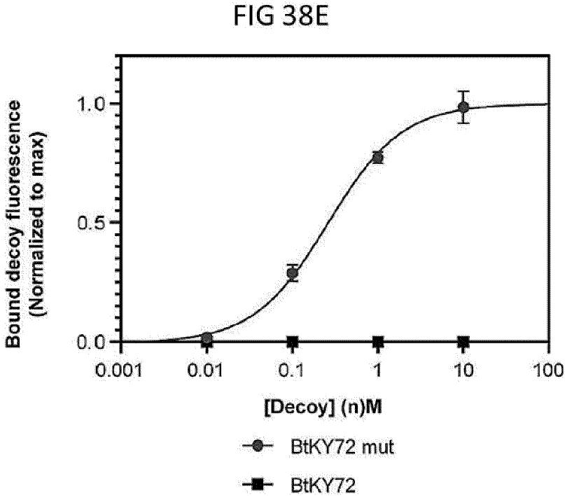

Given the broad activity towards SARS-CoV-2 variants with diverse spike sequences, we next evaluated the ability of CDY14HL-Fc4 to bind diverse RBDs from coronaviruses with pandemic potential. Aside from SARS-CoV-1 and SARS-CoV-2, we identified 23 sarbecoviruses isolated from bats across Asia, Africa, and Europe, many of which are thought to use ACE2 as a receptor (FIG. 38A) [Letko, M., et al., 2020, cited above; Islam A, Ferdous J, Sayeed MA, Islam S, Kaisar Rahman M, Abedin J, et al. Spatial epidemiology and genetic diversity of SARS-CoV-2 and related coronaviruses in domestic and wild animals. PLoS One. 2021;16(12):e0260635] We cloned synthetic RBD genes into the yeast display format for binding analysis (Table immediately below).

| | Virus | Genome | Spike protein | RBD aa | K_{D} (nM) |
|---|---|---|---|---|---|
| | NL63 | AY567487.2 | AAS58177.1 | 481-614 | 0.22 |
| Clade 1a | CoV-1 (Urbani) | AY278741.1 | AAP13441.1 | 317-569 | 0.25 |
| | LYRa11 | KF569996.1 | AHX37558.1 | 321-520 | 0.29 |
| | Rs3367 | KC881006.1 | AGZ48818.1 | 318-570 | 0.19 |
| | Rs4048 | KY417144.1 | ATO98132.1 | 318-517 | 0.31 |
| | Rs4231 | KY417146.1 | ATO98157.1 | 317-516 | 0.30 |
| | Rs4874 | KY417150.1 | ATO98205.1 | 317-516 | 0.23 |
| | Rs7327 | KY417151.1 | ATO98218.1 | 318-517 | 0.31 |
| | RsSHC014 | KC881005.1 | AGZ48806.1 | 318-517 | 0.31 |
| | WIV1 | KF367457.1 | AGZ48831.1 | 318-570 | 0.20 |
| | WIV16 | KT444582.1 | ALK02457.1 | 318-510 | 0.24 |
| Clade 1b | BANAL-103 | MZ937001.1 | UAY13229.1 | 326-526 | 0.30 |
| | BANAL-236 | MZ937003.2 | UAY13253.1 | 326-526 | 0.24 |
| | CoV-2 (Wuhan-Hu-1) | NC_045512.2 | YP_009724390.1 | 330-530 | 0.14 |
| | RaTG13 | MN996532.2 | QHR63300.2 | 319-542 | 1.40 |
| Clade 2 | 16BO133 | KY938558.1 | ASO66810.1 | 317-549 | N/A |
| | Anlong-112 | KY770859.1 | ARI44804.1 | 314-547 | N/A |
| | As6526 | KY417142.1 | ATO98108.1 | 322-554 | N/A |
| | Rf4092 | KY417145.1 | ATO98145.1 | 315-548 | N/A |
| | Rs4255 | KY417149.1 | ATO98193.1 | 322-554 | N/A |
| | ZC45 | MG772933.1 | AVP78031.1 | 327-560 | N/A |
| | ZCX21 | MG772934.1 | AVP78042.1 | 326-559 | N/A |
| Clade 3 | BM48-31 | GU190215.1 | ADK66841.1 | 321-570 | N/A |
| | BtKY72 | KY352407.1 | APO40579.1 | 320-519 | N/A |
| | BtKY72 K493Y/T498W | ^{∗} | ^{∗} | ^{∗} | 0.26 |
| | Khosta-1 | MZ190137.1 | QVN46559.1 | 309-528 | N/A |
| | Khosta-2 | MZ190138.1 | QVN46569.1 | 307-526 | 1.90 |

[Islam A, Ferdous J, Sayeed MA, Islam S, Kaisar Rahman M, Abedin J, et al. Spatial epidemiology and genetic diversity of SARS-CoV-2 and related coronaviruses in domestic and wild animals. PLoS One. 2021;16(12):e0260635; Temmam S, Vongphayloth K, Baquero E, Munier S, Bonomi M, Regnault B, et al. Bat coronaviruses related to SARS-CoV-2 and infectious for human cells. Nature. 2022; He B, Zhang Y, Xu L, Yang W, Yang F, Feng Y, et al. Identification of diverse alphacoronaviruses and genomic characterization of a novel severe acute respiratory syndrome-like coronavirus from bats in China. J Virol. 2014;88(12):7070-82; Hu B, Zeng LP, Yang XL, Ge XY, Zhang W, Li B, et al. Discovery of a rich gene pool of bat SARS-related coronaviruses provides new insights into the origin of SARS coronavirus. PLoS Pathog. 2017;13(11):e1006698; Ge XY, Li JL, Yang XL, Chmura AA, Zhu G, Epstein JH, et al. Isolation and characterization of a bat SARS-like coronavirus that uses the ACE2 receptor. Nature. 2013;503(7477):535-8; Yang XL, Hu B, Wang B, Wang MN, Zhang Q, Zhang W, et al. Isolation and Characterization of a Novel Bat Coronavirus Closely Related to the Direct Progenitor of Severe Acute Respiratory Syndrome Coronavirus. J Virol. 2015;90(6):3253-6; Zhou P, Yang XL, Wang XG, Hu B, Zhang L, Zhang W, et al. Addendum: A pneumonia outbreak associated with a new coronavirus of probable bat origin. Nature. 2020;588(7836):E6; Tao Y, Tong S. Complete Genome Sequence of a Severe Acute Respiratory Syndrome-Related Coronavirus from Kenyan Bats. Microbiol Resour Announc. 2019;8(28); Drexler JF, Gloza-Rausch F, Glende J, Corman VM, Muth D, Goettsche M, et al. Genomic characterization of severe acute respiratory syndrome-related coronavirus in European bats and classification of coronaviruses based on partial RNA-dependent RNA polymerase gene sequences. J Virol. 2010;84(21):11336-49; Hu D, Zhu C, Ai L, He T, Wang Y, Ye F, et al. Genomic characterization and infectivity of a novel SARS-like coronavirus in Chinese bats. Emerg Microbes Infect. 2018;7(1):154; Kim Y, Son K, Kim YS, Lee SY, Jheong W, Oem JK. Complete genome analysis of a SARS-like bat coronavirus identified in the Republic of Korea. Virus Genes. 2019;55(4):545-9; Lin XD, Wang W, Hao ZY, Wang ZX, Guo WP, Guan XQ, et al. Extensive diversity of coronaviruses in bats from China. Virology. 2017;507:1-10; Alkhovsky S, Lenshin S, Romashin A, Vishnevskaya T, Vyshemirsky O, Bulycheva Y, et al. SARS-like Coronaviruses in Horseshoe Bats (Rhinolophus spp.) in Russia, 2020. Viruses. 2022;14(1)].

Additionally, we included the RBD from the human coronavirus NL63, an alpha-CoV with a genetically distinct RBD that has been shown to use ACE2 for cell entry [Hofmann H, Pyrc K, van der Hoek L, Geier M, Berkhout B, Pohlmann S. Human coronavirus NL63 employs the severe acute respiratory syndrome coronavirus receptor for cellular entry. Proc Natl Acad Sci U S A. 2005;102(22):7988-93]. We determined the binding affinities of yeast-displayed RBD to CDY14HL-Fc4 by flow cytometry (FIG. 38B). Several patterns in decoy binding emerged. Beta-CoVs from clades 1a and 1b (CoV-1-like and CoV-2-like CoVs, respectively, isolated from southern China and Laos) bound CDY14HL with sub-nanomolar affinities in the yeast display assay. The exception to this trend was RaTG13, which had a dissociation equilibrium constant of 1.97 nM (table immediately above). Research has recently shown that RaTG13 also binds human ACE2 more weakly than other members of the clade [Wrobel AG, Benton DJ, Xu P, Roustan C, Martin SR, Rosenthal PB, et al. Author Correction: SARS-CoV-2 and bat RaTG13 spike glycoprotein structures inform on virus evolution and furin-cleavage effects. Nat Struct Mol Biol. 2020;27(10):1001]. This behavior suggests that affinity for the decoy and the endogenous human ACE2 receptor are closely linked.

Except for Khosta-2(56), beta-CoVs from clades 2 and 3 (isolated in Asia and Europe/Africa, respectively) did not bind CDY14HL-Fc1. Based on a recent survey of RBD:ACE2 usage (Starr TN, Zepeda SK, Walls AC, Greaney AJ, Alkhovsky S, Veesler D, et al. ACE2 binding is an ancestral and evolvable trait of sarbecoviruses. Nature. 2022;603(7903):913-8), we suspected these clade 2 and 3 RBDs were not capable of human ACE2 binding, which we confirmed in the yeast display system using a soluble wild-type human ACE2-Fc1 fusion protein at 100 nM (FIG. 38D). FIG. 38D shows yeast displayed RBDs used to test for decoy or wt-ACE2 binding at 100 nM. Interestingly, Bloom and colleagues recently discovered that a K493Y/T498W double mutation to the RBD of the clade 3 CoV BtKY72 confers human ACE2 binding [Starr, TN., Et al., 2022]. We found that our decoy bound BtKY72 K493Y/T498W RBD with sub-nanomolar affinity, though it could not bind wt BtKY72 (Table immediately above, FIG. 38E). This suggests that if distant CoVs acquire mutations that confer the potential for zoonotic spread, CDY14HL would retain potent inhibitory potential.

These binding data are in broad agreement with our previous work that demonstrated tight decoy binding to RBDs from SARS-CoV1 and WIV1-CoV [Examples 1-4, and 7]; indeed, we confirm SARS-CoV-1 and WIV1-CoV binding here using the yeast display assay (Table immediately above). However, we reasoned that viral neutralization comes about not only due to the decoy affinity for the viral target, but also from the ability of the decoy to compete for viral binding with the endogenous ACE2 receptor. To assess this possibility, we employed a competitive binding assay between the decoy and ACE2 receptor in the yeast system. We incubated RBD yeast with a low concentration of decoy (1 nM of CDY14HL-hFc; 95 ng/ml) along with a 100-fold molar excess of wt-ACE2 (100 nM of wt-ACE2-mFc, with a mouse Fc fusion to distinguish it from the decoy). We assessed the level of decoy binding retained in the presence of receptor competition by flow cytometry and compared these values across the set of RBDs (FIG. 38B). FIG. 38B shows a schematic representation measuring the competition between decoy and endogenous ACE2 receptor using yeast-displayed RBDs.

The positive control, the RBD from the well-neutralized ancestral SARS-CoV-2 strain, retained 34% of decoy binding in the presence of 100-fold molar excess of wt-ACE2-mFc (FIG. 38C). Similar to SARS-CoV-2, all clade 1a and 1b sarbecovirus RBDs tested retained at least 24% binding in the competition assay. Khosta-2, a clade 3 RBD with weak decoy binding (table immediately above), retained 79% percent decoy binding in the presence of 100-fold excess wt-ACE2. Similarly, the relatively weak-binding RBD from RaTG13 also retained almost complete decoy binding under competition. These examples highlight that decoy affinity alone may be a poor determinant of the ability to compete with endogenous ACE2 as an inhibitor (FIG. 41). FIG. 41 shows plotted graph of evaluation of the relationship between decoy affinity and the amount of decoy retained in a competitive binding assay. The lone alpha-CoV in our study, NL63, retained the lowest fraction of decoy binding in the competition assay (21%, FIG. 38C). Further study is needed to determine whether this result indicates a lower neutralizing potency of the decoy for the genetically distinct ACE2-dependent alpha-CoVs. Together with the observed sub-nanomolar decoy binding affinity, these competitive binding data predict broad and potent neutralization of beta-CoVs that can bind human ACE2. As many factors including subtle shifts in co-receptor usage and cell entry mechanism can define the potency of an inhibitor [Singh M, Bansal V, Feschotte C. A Single-Cell RNA Expression Map of Human Coronavirus Entry Factors. Cell Rep. 2020;32(12):108175], further studies with diverse CoVs in pseudotyped and live-virus systems will be required to confirm this finding.

### D. Discussion

Currently available COVID-19 treatments such as monoclonal antibodies are fast becoming less effective as new variants emerge [Zhou H, Tada T, Dcosta BM, Landau NR. Neutralization of SARS-CoV-2 Omicron BA.2 by Therapeutic Monoclonal Antibodies. bioRxiv. 2022; VanBlargan LA, Errico JM, Halfmann PJ, Zost SJ, Crowe JE, Jr., Purcell LA, et al. An infectious SARS-CoV-2 B.1.1.529 Omicron virus escapes neutralization by therapeutic monoclonal antibodies. Nat Med. 2022;28(3):490-5]. Alternative protein-based viral neutralizers exist, including camelid-derived single chain antibodies. While these have several advantages over conventional antibodies with respect to small size, delivery modalities, and manufacturability [Sasisekharan R. Preparing for the Future -Nanobodies for Covid-19? N Engl J Med. 2021;384(16):1568-71; Huo J, Mikolajek H, Le Bas A, Clark JJ, Sharma P, Kipar A, et al. A potent SARS-CoV-2 neutralising nanobody shows therapeutic efficacy in the Syrian golden hamster model of COVID-19. Nat Commun. 2021;12(1):5469; Koenig PA, Das H, Liu H, Kummerer BM, Gohr FN, Jenster LM, et al. Structure-guided multivalent nanobodies block SARS-CoV-2 infection and suppress mutational escape, Science. 2021;371(6530).], nanobodies still recognize a specific epitope on the SARS-CoV-2 RBD spike surface and thus are still susceptible to mutations that could eliminate this binding epitope.

The affinity-matured, soluble ACE2 decoy termed CDY14HL-Fc4 binds and neutralizes SARS-CoV2 strains from the early pandemic as well as the related pandemic sarbecovirus, SARS-CoV1 [Examples 1-4, and 7]. In more recent studies provided herein, we show that the affinity-matured decoy retains broad neutralizing activity against every SARS-CoV2 variant tested including Delta, Delta Plus, Omicron BA.1, and Omicron BA.2. These results highlight a major advantage of receptor-based decoy molecules over other viral spike inhibitor biologics; unlike monoclonal antibodies, decoy binding of a variant is tightly linked to receptor binding and thus viral fitness through evolution.

Our original strategy for deploying the decoy was in the context of prevention of SARS-CoV2 infection. This was accomplished through the creation of an AAV vector expressing the decoy that is administered via a nasal administration to engineer proximal airway cells to express neutralizing levels of the decoy at the airway surface where the virus enters. This approach is expected to be particularly useful in immune compromised patients who do not generate protective immunity following active vaccination. The decoy is also useful as a therapeutic protein for treatment and prevention in high-risk groups, following parenteral administration.

The relentless emergence of new highly-transmissible SARS-CoV2 variants in the current pandemic reminds us how vulnerable we are to the power of zoonosis and intense pressure that pandemic viruses are under to evolve into more pathogenic and/or transmissible variants. This experience suggests the importance of proactively developing countermeasures against future pandemics, which will likely be caused by a coronavirus based on the recent history of SARS, Middle East respiratory syndrome, and COVID-19. Indeed, CoVs constitute a major fraction of pre-zoonotic viruses ranked by multiple genetic and environmental factors for pandemic potential [Grange ZL, Goldstein T, Johnson CK, Anthony S, Gilardi K, Daszak P, et al. Ranking the risk of animal-to-human spillover for newly discovered viruses. Proc Natl Acad Sci U S A. 2021;118(15)]. This threat compelled us to evaluate the competitive binding of our ACE2 decoy to spike proteins from a variety of animal coronaviruses with zoonotic potential, particularly sarbecoviruses that can bind human ACE2, or that whose viral spike proteins only need a small number of mutations to acquire human ACE2 binding. We were delighted to find that the decoy retained very high binding activity against spike proteins from every pre-emergent strain studied that also had the ability to bind human ACE2, suggesting the broad utility of the decoy in the current and future coronavirus pandemics.

COVID-19 has illustrated how powerful the drive for viral fitness can be in circumventing immunity generated from previous infection, vaccines or antibody therapeutics. This rapid evolution is substantially amplified in the setting of a world-wide pandemic caused by a highly transmissible virus. The use of a decoy protein that is based on a soluble version of a viral receptor significantly restricts virus escape since any mutation that diminished decoy binding will diminish viral fitness.

### E. Materials and Methods

### CoV Pseudotyped Lentiviral Neutralization Assay

Replication-incompetent lentiviruses pseudotyped with CoV spike proteins and packaging for a Renilla luciferase reporter gene were purchased from Integral Molecular: RVP-701L Wuhan (lot CL-114B), RVP-763L Delta (lot CL-267A), RVP-736L Zeta (lot CL-255A), RVP-730L Kappa (lot CL-247A), RVP-768L Omicron (lot CL-297A), RVP-767L Mu (lot CL-274A), RVP-766L Lambda (lot CL-259A), and RVP-765L Delta + (lot CL-258A). We performed neutralization assays using human embryonic kidney 293T cells overexpressing ACE2 (Integral Molecular) as previously described [Examples 1-4, and 7].

### Recombinant Protein Production

To generate wt-ACE2-Fc for competitive binding assays, we cloned human ACE2 (1-615) fused to a C-terminal mouse IgG2a Fc into pcDNA3.1. We transfected the plasmid into Expi293 cells for expression. The supernatant was collected and exchanged to 0.1 M sodium phosphate, pH 7.2 and 150 mM NaCl buffer for purification on Protein A Sepharose 4B (ThermoFisher). The protein was eluted in 0.1 M citric acid, pH 3.0 and neutralized in 1 M Tris, pH 9.0 before a final buffer exchange to 25 mM HEPES pH 7.2 and 150 mM NaCl by size-exclusion chromatography with Superose 6 resin (Cytiva). For these studies, we cloned the engineered CDY14HL 1-615 fragment in front of the human IgG1 Fc domain for expression and purification. We previously characterized a decoy fusion to human IgG4 Fc [Examples 1-4, and 7], but found that Fc1 and Fc4 decoy fusions behave similarly with respect to binding and neutralization (e.g., the IC50 values against Wuhan-Hu1 pseudotypes were 37 ng/ml and 35 ng/ml for CDY14HL-Fc4 and CDY14HL-Fc1, respectively).

### Phylogenic Tree Construction

The RBD sequences of the CoVs were taken from spike protein coding sequences downloaded from the National Center for Biotechnology Information (NCBI). Using MEGA X [Kumar S, Stecher G, Li M, Knyaz C, Tamura K. MEGA X: Molecular Evolutionary Genetics Analysis across Computing Platforms. Mol Biol Evol. 2018;35(6):1547-9], we aligned the amino acid sequences in ClustalW and constructed a phylogenic tree using maximum likelihood analysis.

### Yeast Display Binding Assays

The nucleic acid sequences of the CoV RBDs were taken from NCBI: CoV-2 (NC_045512.2), NL63 (AY567487), LYRa11 (KF569996), Rs4048 (KY417144), Rs4231 (KY417146), Rs7327 (KY417151), RsSHC014 (KC881005), WIV16 (KT444582.1), BANAL-236 (MZ937003), BANAL-103 (MZ937001), Rs4874 (KY417150), and RaTG13 (MN996532.2). We cloned the RBDs into a plasmid between an upstream Aga2 gene and a downstream hemagglutinin (HA) epitope tag with flexible GSG linkers. The plasmid has a low-copy centromeric origin similar to that of pTCON2 [Chao G, Lau WL, Hackel BJ, Sazinsky SL, Lippow SM, Wittrup KD. Isolating and engineering human antibodies using yeast surface display. Nat Protoc. 2006;1(2):755-68.]. Plasmids were transformed into EBY100 using the Frozen-EZ Yeast Transformation II Kit (Zymo). We grew colonies in SD-trp media before induction in log phase for 24 hr at 30° C. in SG-CAA [Chao G, et al., 2006, cited above]. For competition or direct binding assays, we incubated the yeast with CDY14HL-Fc1 with or without wt-ACE-mFc for 30 min at 25° C. before staining the sample with goat anti-human fluorescein isothiocyanate (FITC; ThermoFisher A18812) and rabbit anti-HA-PE (Cell Signaling Technology 14904S). We used phosphate-buffered saline with 0.1% bovine serum albumin for all staining and washes. For the titration of CDY14HL-Fc1, we incubated the yeast with 1:10 dilution series of CDY14HL-Fc1 at 25° C. for 6 hr. The yeast were analyzed on an ACEA NovoCyte flow cytometer. We determined the level of CDY14HL-Fc1 binding by taking the mean FITC signal for 500 RBD+ yeast cells collected for each condition. We fitted the decoy concentration versus the decoy binding signal in GraphPadPrism using a three-parameter fit to the binding isotherm.

All animal procedures and protocols were approved by the Institutional Animal Care and Use Committee of the University of Pennsylvania.

### EXAMPLE 12. Examination of the Engineered hAce2 Decoy Protein Efficacy in Various In Vivo Models: Generating an Injectable Ace2 Decoy Protein Drug for Treatment of Severe CoV Infection

The mutant hAce2 soluble decoy protein therapeutic constructs are: GTP14HL-Fc1 (CDY14HL-hIgG1-Fc; hAce2-GTP14-HL-IgG1; original engineered Ace2 with 7 mutations), MR27HL-Fc1 (MR27HL-hIgG1-Fc; 5 mutations (same as GTP14HL decoy plus reversion at aa 49 and 79)), GTP14HL-Fc1* (CDY14HL-hIgG1-Fc; hAce2-GTP14-HL-IgG1), and MR27HL-Fc1* (MR27HL-hIgG1-Fc) (*shortened hinge/linker between decoy and Fc region). The injectable hAce2 decoy protein drugs were generated in CHO Cell line (CHO stable cell lines were created for stable production of hAce2 decoy proteins).

In the production process, various aspects have been varied for optimization, such as varying leader peptides (native vs. heterologous), the promoters (CMV, EF1, EEF2), the selection marker stringency (high and low), addition of co-transfection of additional glycosylation enzymes to promote sialyation.

For the production process, CHO pools were created in which 500 ml fed-batch cultures were generated, tittered, purified and assessed for activity across a variety of metrics.

Briefly, for the GMP-suitable manufacturing process a stable cell line created. Horizon Discovery Limited HD-BIOP3 GS KO CHO-K1 Cells were used for stable expression of constructs which were co-transfected with Leap-In Transposase mRNA into CHO cells. Chemically defined, animal component free cell culture media components were used. Some low pH instability were identified but process controls are implemented to manage for this. Fusion protein has been undergone tangential flow filtration concentration and buffer exchange to maintain monomer form (i.e., aggregation is a commonly reported problem with fusion proteins). Additional downstream operations steps are added for orthogonal adventitious viral inactivation and/or removal. The manufacturing process development allows for a high stability formulation for ease of standard temperature storage.

Test runs for hAce2 decoy protein production was performed in a “10 L” suspension bioreactor, in which we observed good yield for a fusion protein. FIG. 35 shows exemplary pool hAce2 decoy protein expression levels (mg/L) in a 10-day process in a 10 L Stirred Tank Controlled Bioreactor. These results show about 1.2 g/L of protein purified using a one or two step process with higher percent monomer purity and good recovery (~90%).

### Examination of the Engineered hAce2 Decoy Protein Efficacy in Mice.

In this study, we intravenously administered (IV) purified engineered hAce2 decoy protein as sampled from various purified pools. Serum was collected on days 1 and day 7 of the study for determination of the protein levels by mass spectrometry (as described herein). Necropsy was performed on day 7 of the study with BALF collection performed for mass spectrometry analysis. The study layout and obtained data is summarized in the table below.

| Pool | Dose (mg/kg) | Day 1 | Day 7 | |
|---|---|---|---|---|
| | | Average Serum Ace-2 (µg/mL) | Average Serum Ace-2 (ng/mL) | Average BALF Ace-2 (µg/mL) |
| N/A | 0 (Vehicle) | ND | ND | ND |
| hAce2MR27-Fc1 (1.6 mg/mL) | 0.2 | 3.746 | 0.905 | 0.043 |
| | 1.1 | 12.222 | 2.708 | 0.116 |
| hAce2MR27-Fc1 (8.4 mg/mL) | 1.1 | 7.708 | 1.536 | 0.047 |
| | 3.0 | 17.111 | 3.258 | 0.100 |
| GTP14HL-Fc1 (3.9 mg/mL) | 0.2 | 1.337 | ND | ND |
| | 1.1 | 4.997 | 1.052 | 0.033 |
| GTP14HL-Fc1 (2.3 mg/mL) | 0.2 | 2.439 | 0.608 | 0.020 |
| | 1.1 | 5.454 | 1.024 | 0.035 |
| hAce2MR27-Fc1 (2.2 mg/mL) | 0.2 | 1.318 | ND | ND |
| | 1.1 | 6.044 | 1.195 | 0.036 |
| GTP14HLFc1 (P36) | 0.2 | 1.128 | 0.284 | ND |
| | 1.1 | 4.036 | 0.921 | 0.077 |
| MR27HL-Fc1 (5.87 mg/mL) | 0.2 | 0.998 | 0.204 | ND |
| MR27HL-Fc1 (23.12 mg/mL) | 1.1 | 4.038 | 0.834 | 0.067 |

### Examination of the Engineered hAce2 Decoy Protein Efficacy in Hamsters.

In this pilot study, we examined pharmacokinetics of the engineered hAce2 decoy fusion protein in hamsters, following intraperitoneal (IP) administration. Serum was collected on days 1 and 8 for determination of protein levels by mass spectrometry (as described herein). Additionally, nasal lavage fluid (NLF) samples were collected for mass spectrometry analysis to determine protein levels. The study layout and obtained data is summarized in the table below.

| Pool | Dose (mg/kg) | Day 1 | | Day 8 | |
|---|---|---|---|---|---|
| | | Average Serum Ace-2 (ug/mL) | Average NLF Ace-2 (ng/mL) | Average Serum Ace-2 (ug/mL) | Average NLF Ace-2 (ng/mL) |
| hAce2MR27-Fc1 (8.4 mg/mL) | 3 | 34.11 | 0.33 | 30.04 | 4.16 |
| hAce2MR27-Fc1 (8.4 mg/mL) | 10 | 114.41 | 1.09 | 97.56 | 19.37 |
| hAce2MR27-Fc1 (8.4 mg/mL) | 30 | 312.40 | 3.11 | 274.86 | 50.13 |
| GTP14HL-Fc1 (3.9 mg/mL) | 3 | 36.12 | 0.35 | 29.35 | 5.59 |
| GTP14HL-Fc1 (2.3 mg/mL) | 3 | 33.54 | 0.36 | 28.83 | 13.77 |
| hAce2MR27-Fc1 (2.2 mg/mL) | 3 | 33.72 | 0.32 | 31.63 | 6.10 |

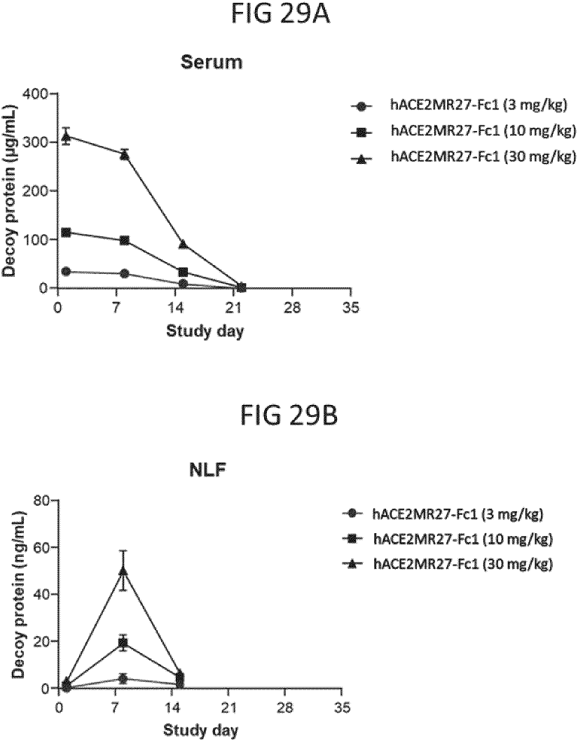

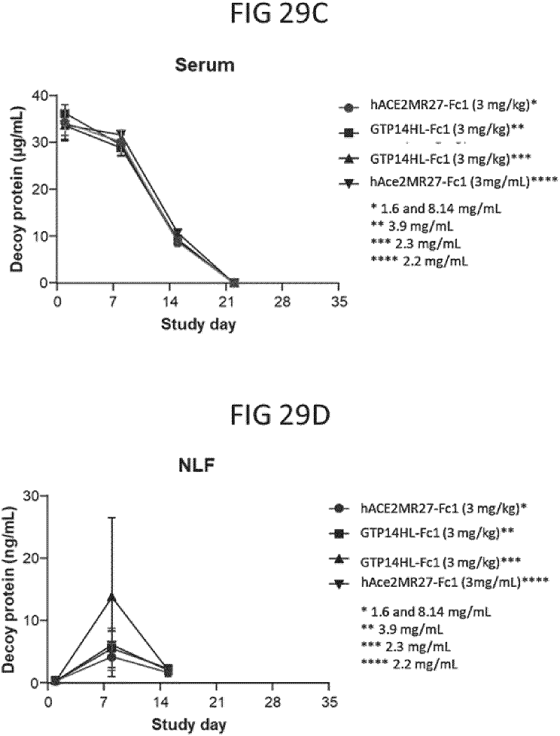

FIGS. 29A to 29D shows results of the pharmacokinetic examination of the engineered hAce2 decoy IgG1 Fc fusion protein in vivo in hamsters. FIG. 29A shows concentration of the engineered hAce2 decoy Fc1 fusion protein, as measured with mass spectrometry in collected serum samples, post intraperitoneal administration of hAceMR27HL-Variant-IgG1 Fc decoy fusion at doses of 3 mg/kg, 10 mg/kg, and 30 mg/kg. FIG. 29B shows concentration of the engineered hAce2 decoy Fc1 fusion protein, as measured with mass spectrometry in collected NJF samples, post intraperitoneal administration of hAceMR27HL-Variant-IgG1 Fc decoy fusion at doses of 3 mg/kg, 10 mg/kg, and 30 mg/kg. FIG. 29C shows concentration of the engineered hAce2 decoy Fc1 fusion protein, as measured with mass spectrometry in collected serum samples, post intraperitoneal administration of hAceMR27HL-Variant-IgG1 Fc or GTP14HL-Fc1 decoy fusion from various pools of protein samples, when administered at a dose of 3 mg/kg. FIG. 29D shows concentration of the engineered hAce2 decoy Fc1 fusion protein, as measured with mass spectrometry in collected NLF samples, post intraperitoneal administration of hAceMR27HL-Variant-IgG1 Fc or GTP14HL-Fc1 decoy fusion from various pools of protein samples, when administered at a dose of 3 mg/kg.

Furthermore, we examined potency of the hAce2 decoy protein from serum in hamsters following administration with hAceMR27HL-Variant-IgG1 Fc decoy.

From these data, the values for half-life of the engineered hAce2 decoy IgG1 Fc fusion protein were calculated, and summarized in table below.

| Pool (protein) | Half-life (days) |
|---|---|
| hAce2MR27HL-Fc1 (8.4 mg/mL) | 7.15 |
| GTP14HL-Fc1 (3.9 mg/mL) | 7.15 |
| GTP14HL-Fc1 (2.3 mg/mL) | 7.41 |
| hAce2MR27-Fc1 (2.2 mg/mL) | 8.53 |

Next, a further pharmacokinetics study in hamsters is performed to confirm the results of the pilot study and to evaluate engineered hAce2 decoy efficacy when administered intravenously or intraperitoneally at doses of 0.3 mg/kg, 3 mg/kg, 10 mg/kg, and 30 mg/kg (n=9/sex/group, 90 total, males and females). Safety endpoints are clinical signs, body weight, clinical pathology (hematology, coagulation, and clinical chemistry), anti-decoy antibodies, full histopathology. Pharmacokinetic endpoints are serum decoy protein levels (peak and half-life), decoy point levels in BALF / NLF as measured by mass spectrometry. Additional endpoints are neutralization assay on BALF / NLF and serum, spike binding assay on BALF / NLF and serum.

Additionally, pilot prophylaxis study (n=5/sex/group) is performed to evaluate the use of engineered hAce2 decoy Fc1 fusion protein in preventing the SARS CoV2 infection.

| | | | | |
|---|---|---|---|---|
| Group Designation | 1 | 2 | 3 | 4 |
| Number of Hamsters | 10 | 10 | 10 | 10 |
| Sex/Age | M+F/6-10 weeks | M+F/6-10 weeks | M+F/6-10 weeks | M+F/6-10 weeks |
| TA/CA Dosing (D-3) | Vehicle | hAce2MR27HL-Fc1 (8.4 mg/mL) | hAce2MR27HL-Fc1 (8.4 mg/mL) | hAce2MR27HL-Fc1 (8.4 mg/mL) |
| ROA | IV ROV | IV ROV | IV ROV | IV ROV |
| Number of Hamsters | 10 | 10 | 10 | 10 |
| Volume | 300 µl | 300 µl | 300 µl | 300 µl |
| Dose | NA | 3 (Low) | 10 (Med) | 30 (High) |
| SARS-COV-2 Challenge Dose (D0) (IN) | Delta (1:10 dilⁿ) | Delta (1:10 dilⁿ) | Delta (1:10 dilⁿ) | Delta (1:10 dilⁿ) |
| Necropsy Day | 7 | 7 | 7 | 7 |

Levels of genomic viral RNA, subgenomic viral RNA, and TCID50 are determined on the following samples: lung, nasal turbinates, and trachea. Additionally, oral swabs are performed, from samples of which both viral load and TCID50 assay are completed (i.e., on the same swab). To minimize the unwanted side effects in the lung pathology read out, BALF samples are not collected. Serum is collected on day 2 post dosing and at termination of the study to confirm levels of the engineered decoy protein. Weights are measured to give insight into the effectiveness of the decoy protein.

Similar to the above outlined prophylaxis study, a pilot treatment challenge study evaluating protein decoy efficacy in hamsters is performed, wherein the CoV2 challenge is performed on D0, and the protein decoy is administered on Day 1.

An expanded challenge study using protein decoy (IV administered) is performed in hamsters to further examine prophylactic effect when challenged with CoV2 variants (Wuhan, UK, RSA, Delta, Lamda).

### Examination of the Engineered hAce2 Decoy Protein Efficacy in NHPs.

A pilot pharmacokinetics study is performed in non-human primates (NHPs) to evaluate engineered hAce2 decoy IgG1 fusion protein efficacy in NHP (n=2). Mid (30 mg/kg) and high doses are administered via intravenous (IV) infusion (60 minutes). A DSI transmitter is surgically implanted to allow for remote collection of data (telemetry; blood pressure, heart rate, temperature). Safety endpoints are clinical signs, body weight, clinical pathology (hematology, coagulation, and clinical chemistry), urinalysis, cytokines, blood pressure, heart rate, ECG, angiotensin system (Ang II, Ang 1-7, Ang 1-9), anti-decoy antibodies, ELISPOTs, full histopathology. Pharmacokinetic endpoints are serum decoy protein levels (peak and half-life), decoy point levels in BALF/nasal swabs as measured by mass spectrometry. Additional endpoints are neutralization assay on BALF/nasal swabs, spike binding assay on BALF/nasal swabs and serum. The samples are collected throughout the duration of the study, i.e., day 30 and day 180. A further safety pharmacokinetic study is performed in NHPs (n=32) to confirm the result of the pilot study.

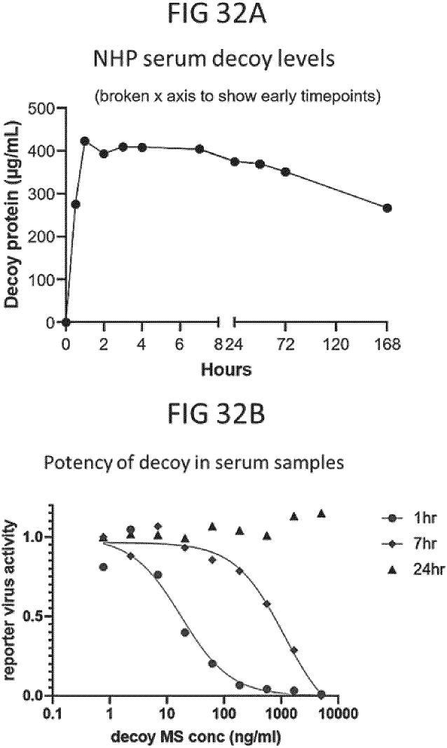

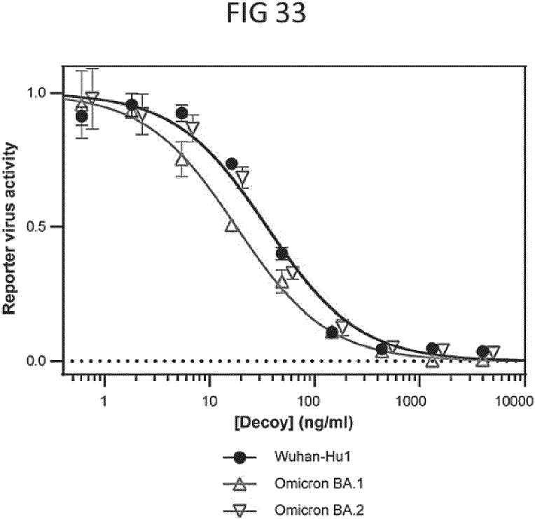

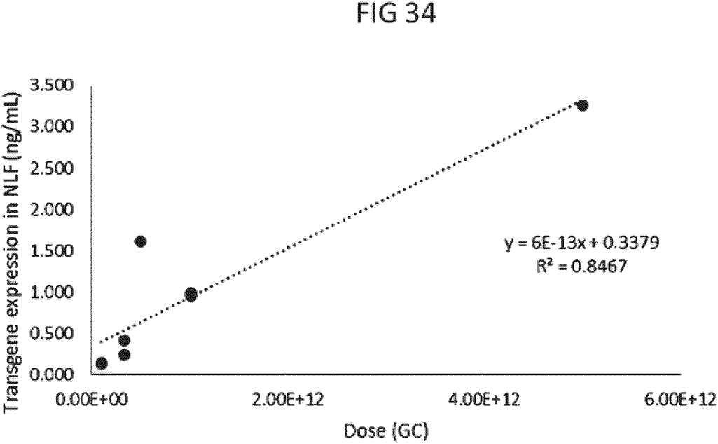

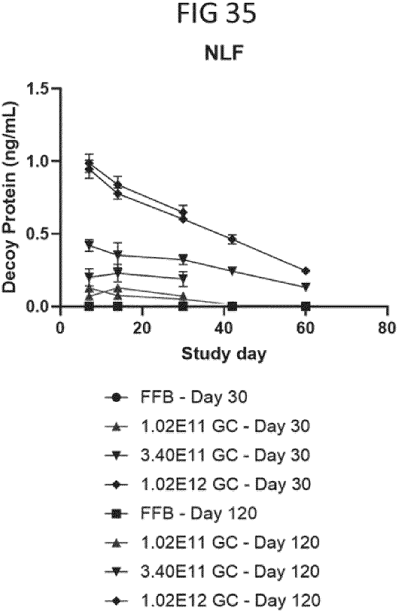

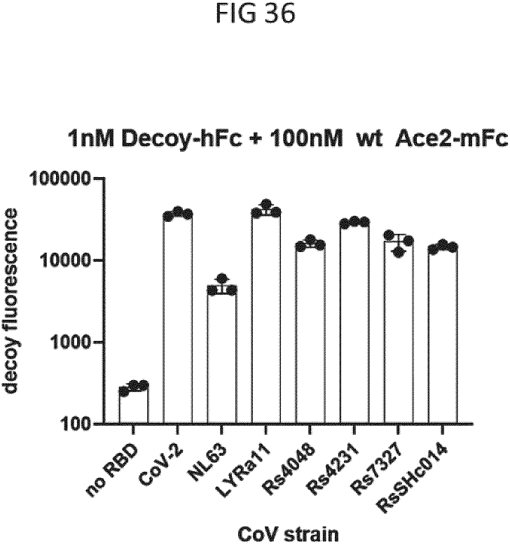

FIG. 32A shows serum decoy levels in NHP following administration by IV infusion of hAce2 decoy protein (MR27HL-Fc1) at a dose of 30 mg/kg (broken x axis for showing early timepoints (Hours)). FIG. 32B shows potency of decoy in serum samples of NHP following administration by IV infusion of hAce2 decoy protein (MR27HL-Fc1) at a dose of 30 mg/kg, plotted as a reporter virus activity over decoy MS concentration (ng/ml) at 1 hour, 7 hours, and 24 hours post administration. Potency of decoy was measured by CoV2 pseudotype neutralization assay (described herein).

1 Chan, K. K., Tan, T. J. C., Narayanan, K. K. & Procko, E. An engineered decoy receptor for SARS-CoV-2 broadly binds protein S sequence variants. Sci Adv 7, doi:10.1126/sciadv.abf1738 (2021).

2 Glasgow, A. et al. Engineered ACE2 receptor traps potently neutralize SARS-CoV-2. Proc Natl Acad Sci U S A 117, 28046-28055, doi:10.1073/pnas.2016093117 (2020).

3 Adam, V. S. et al. Adeno-associated virus 9-mediated airway expression of antibody protects old and immunodeficient mice against influenza virus. Clin Vaccine Immunol 21, 1528-1533, doi:10.1128/CVI.00572-14 (2014).

4 Laursen, N. S. et al. Universal protection against influenza infection by a multidomain antibody to influenza hemagglutinin. Science 362, 598-602, doi:10.1126/science.aaq0620 (2018).

5 Limberis, M. P. et al. Intranasal antibody gene transfer in mice and ferrets elicits broad protection against pandemic influenza. Sci Transl Med 5, 187ra172, doi:10.1126/scitranslmed.3006299 (2013).

6 Limberis, M. P. et al. Vectored expression of the broadly neutralizing antibody FI6 in mouse airway provides partial protection against a new avian influenza A virus, H7N9. Clin Vaccine Immunol 20, 1836-1837, doi:10.1128/CVI.00545-13 (2013).

7 Angelini, A. et al. Protein Engineering and Selection Using Yeast Surface Display. Methods Mol Biol 1319, 3-36, doi:10.1007/978-1-4939-2748-7_1 (2015).

8 Chan, K. K. et al. Engineering human ACE2 to optimize binding to the spike protein of SARS coronavirus 2. Science 369, 1261-1265, doi:10.1126/science.abc0870 (2020).

9 Aguinaldo, A. M. & Arnold, F. H. Staggered extension process (StEP) in vitro recombination. Methods Mol Biol 231, 105-110, doi:10.1385/1-59259-395-X:105 (2003).

10 Eckert-Boulet, N., Pedersen, M. L., Krogh, B. O. & Lisby, M. Optimization of ordered plasmid assembly by gap repair in Saccharomyces cerevisiae. Yeast 29, 323-334, doi:10.1002/yea.2912 (2012).

11 Boder, E. T., Midelfort, K. S. & Wittrup, K. D. Directed evolution of antibody fragments with monovalent femtomolar antigen-binding affinity. Proc Natl Acad Sci U S A 97, 10701-10705, 290 doi:10.1073/pnas.170297297 (2000).

12 Guy, J. L., Jackson, R. M., Jensen, H. A., Hooper, N. M. & Turner, A. J. Identification of critical active-site residues in angiotensin-converting enzyme-2 (ACE2) by site-directed mutagenesis. FEBS J 272, 3512-3520, doi:10.1111/j.1742-4658.2005.04756.x (2005).

13 Starr, T. N. et al. Prospective mapping of viral mutations that escape antibodies used to treat COVID-19. Science 371, 850-854, doi:10.1126/science.abf9302 (2021).

14 Thomson, E. C. et al. Circulating SARS-CoV-2 spike N439K variants maintain fitness while evading antibody-mediated immunity. Cell 184, 1171-1187 e1120, doi:10.1016/j.cell.2021.01.037 (2021).

15 Wang, P. et al. Antibody Resistance of SARS-CoV-2 Variants B.1.351 and B.1.1.7. Nature, doi:10.1038/s41586-021-03398-2 (2021).

16 Oude Munnink, B. B. et al. Transmission of SARS-CoV-2 on mink farms between humans and mink and back to humans. Science 371, 172-177, doi:10.1126/science.abe5901 (2021).

17 Menachery, V. D. et al. SARS-like WIV1-CoV poised for human emergence. Proc Natl Acad Sci U S A 113, 3048-3053, doi:10.1073/pnas.1517719113 (2016).

18 Weissman, D. et al. D614G Spike Mutation Increases SARS CoV-2 Susceptibility to Neutralization. Cell Host Microbe 29, 23-31 e24, doi:10.1016/j.chom.2020.11.012 (2021).

19 Yurkovetskiy, L. et al. Structural and Functional Analysis of the D614G SARS-CoV-2 Spike Protein Variant. Cell 183, 739-751 e738, doi:10.1016/j.cell.2020.09.032 (2020).

20 Winkler, E. S. et al. SARS-CoV-2 infection of human ACE2-transgenic mice causes severe lung inflammation and impaired function. Nature Immunology 21, 1327-1335, doi:10.1038/s41590- 020-0778-2 (2020).

21 Zheng, J. et al. COVID-19 treatments and pathogenesis including anosmia in K18-hACE2 mice. Nature 589, 603-607, doi:10.1038/s41586-020-2943-z (2021).

22 Yinda, C. K. et al. K18-hACE2 mice develop respiratory disease resembling severe COVID-19. PLOS Pathogens 17, e1009195, doi:10.1371/journal.ppat.1009195 (2021).

23 Adachi, K., Enoki, T., Kawano, Y., Veraz, M. & Nakai, H. Drawing a high-resolution functional map of adeno-associated virus capsid by massively parallel sequencing. Nat Commun 5, 3075, doi:10.1038/ncomms4075 (2014).

24 Kemp, S. A. et al. SARS-CoV-2 evolution during treatment of chronic infection. Nature, doi:10.1038/s41586-021-03291-y (2021).

25 Ozono, S. et al. SARS-CoV-2 D614G spike mutation increases entry efficiency with enhanced ACE2-binding affinity. Nat Commun 12, 848, doi:10.1038/s41467-021-21118-2 (2021).

26 Zhou, D. et al. Evidence of escape of SARS-CoV-2 variant B.1.351 from natural and vaccine-induced sera. Cell, doi:10.1016/j.cell.2021.02.037 (2021).

27 Zhang, H. et al. Expression of the SARS-CoV-2 ACE2 Receptor in the Human Airway Epithelium. Am J Respir Crit Care Med 202, 219-229, doi:10.1164/rccm.202003-0541OC (2020).

28 Bunyavanich, S., Do, A. & Vicencio, A. Nasal Gene Expression of Angiotensin-Converting Enzyme 2 in Children and Adults. JAMA 323, 2427-2429, doi:10.1001/jama.2020.8707 (2020).

29 Ramanathan, M., Ferguson, I. D., Miao, W. & Khavari, P. A. SARS-CoV-2 B.1.1.7 and B.1.351 Spikevariants bind human ACE2 with increased affinity. bioRxiv, doi:10.1101/2021.02.22.432359 (2021).

30 Joaquín Cáceres, C. et al. Efficacy of GC-376 against SARS-CoV-2 virus infection in the K18 hACE2 transgenic mouse model. bioRxiv, 2021.2001.2027.428428, doi:10.1101/2021.01.27.428428 (2021).

31 Rosenfeld, R. et al. Post-exposure protection of SARS-CoV-2 lethal infected K18-hACE2 transgenic mice by neutralizing human monoclonal antibody. Nature Communications 12, 944, doi:10.1038/s41467-021-21239-8 (2021).

32 Limberis, M. P. & Wilson, J. M. Adeno-associated virus serotype 9 vectors transduce murine alveolar and nasal epithelia and can be readministered. Proc Natl Acad Sci U S A 103, 12993-12998, doi:10.1073/pnas.0601433103 (2006).

33 Latinne, A. et al. Origin and cross-species transmission of bat coronaviruses in China. Nat Commun 11, 4235, doi:10.1038/s41467-020-17687-3 (2020).

34 Xu, C. et al. Conformational dynamics of SARS-CoV-2 trimeric spike glycoprotein in complex with receptor ACE2 revealed by cryo-EM. Sci Adv 7, doi:10.1126/sciadv.abe5575 (2021).

35 Gietz, R. D. & Schiestl, R. H. Large-scale high-efficiency yeast transformation using the LiAc/SS carrier DNA/PEG method. Nat Protoc 2, 38-41, doi:10.1038/nprot.2007.15 (2007).

36 Chao, G. et al. Isolating and engineering human antibodies using yeast surface display. Nat Protoc 1, 755-768, doi:10.1038/nprot.2006.94 (2006).

37 Lock, M. et al. Rapid, simple, and versatile manufacturing of recombinant adeno-associated viral vectors at scale. Hum Gene Ther 21, 1259-1271, doi:10.1089/hum.2010.055 (2010).

38 Lock, M., Alvira, M. R., Chen, S. J. & Wilson, J. M. Absolute determination of single-stranded and self-complementary adeno-associated viral vector genome titers by droplet digital PCR. Hum Gene Ther Methods 25, 115-125, doi:10.1089/hgtb.2013.131 (2014).

39 Kaulbach, H. C., White, M. V., Igarashi, Y., Hahn, B. K. & Kaliner, M. A. Estimation of nasal epithelial lining fluid using urea as a marker. J Allergy Clin Immunol 92, 457-465, doi:10.1016/0091-6749(93)90125-y (1993).

40 Baum, A. et al. REGN-COV2 antibodies prevent and treat SARS-CoV-2 infection in rhesus macaques and hamsters. Science 370, 1110-1115, doi:10.1126/science.abe2402 (2020).

41 Yan, R. et al. Structural basis for the recognition of SARS-CoV-2 by full-length human ACE2. Science 367, 1444-1448, doi:10.1126/science.abb2762 (2020).

42. Barton MI, MacGowan SA, Kutuzov MA, Dushek O, Barton GJ, van der Merwe PA. Effects of common mutations in the SARS-CoV-2 Spike RBD and its ligand, the human ACE2 receptor on binding affinity and kinetics. Elife. 2021;10.

43. Van Egeren D, Novokhodko A, Stoddard M, Tran U, Zetter B, Rogers M, et al. Risk of rapid evolutionary escape from biomedical interventions targeting SARS-CoV-2 spike protein. PLoS One. 2021;16(4):e0250780.

44. Corey L, Beyrer C, Cohen MS, Michael NL, Bedford T, Rolland M. SARS-CoV-2 Variants in Patients with Immunosuppression. N Engl J Med. 2021;385(6):562-6.

45. Truong TT, Ryutov A, Pandey U, Yee R, Goldberg L, Bhojwani D, et al. Increased viral variants in children and young adults with impaired humoral immunity and persistent SARS-CoV-2 infection: A consecutive case series. EBioMedicine. 2021;67:103355.

46. Weigang S, Fuchs J, Zimmer G, Schnepf D, Kern L, Beer J, et al. Within-host evolution of SARS-CoV-2 in an immunosuppressed COVID-19 patient as a source of immune escape variants. Nat Commun. 2021;12(1):6405.

47. Sims JJ, Greig JA, Michalson KT, Lian S, Martino RA, Meggersee R, et al. Intranasal gene therapy to prevent infection by SARS-CoV-2 variants. PLoS Pathog. 2021;17(7):e1009544.

48. Planas D, Veyer D, Baidaliuk A, Staropoli I, Guivel-Benhassine F, Rajah MM, et al. Reduced sensitivity of SARS-CoV-2 variant Delta to antibody neutralization. Nature. 2021;596(7871):276-80.

49. Liu Y, Arase N, Kishikawa J-i, Hirose M, Li S, Tada A, et al. The SARS-CoV-2 Delta variant is poised to acquire complete resistance to wild-type spike vaccines. bioRxiv. 2021:2021.08.22.457114.

50. Wilhelm A, Widera M, Grikscheit K, Toptan T, Schenk B, Pallas C, et al. Reduced Neutralization of SARS-CoV-2 Omicron Variant by Vaccine Sera and Monoclonal Antibodies. medRxiv. 2021:2021.12.07.21267432.

51. Wec AZ, Wrapp D, Herbert AS, Maurer DP, Haslwanter D, Sakharkar M, et al. Broad neutralization of SARS-related viruses by human monoclonal antibodies. Science. 2020;369(6504):731-6.

52. Weinreich DM, Sivapalasingam S, Norton T, Ali S, Gao H, Bhore R, et al. REGN-COV2, a Neutralizing Antibody Cocktail, in Outpatients with Covid-19. N Engl J Med. 2021;384(3):238-51.

53. Weinreich DM, Sivapalasingam S, Norton T, Ali S, Gao H, Bhore R, et al. REGEN-COV Antibody Combination and Outcomes in Outpatients with Covid-19. N Engl J Med. 2021;385(23):e81.

54. O’Brien MP, Hou P, Weinreich DM. Subcutaneous REGEN-COV Antibody Combination to Prevent Covid-19. Reply. N Engl J Med. 2021;385(20):e70.

55. Starr TN, Greaney AJ, Addetia A, Hannon WW, Choudhary MC, Dingens AS, et al. Prospective mapping of viral mutations that escape antibodies used to treat COVID-19. Science. 2021;371(6531):850-4.

56. Fenaux H, Gueneau R, Chaghouri A, Henry B, Mouna L, Roque-Afonso AM, et al. Emergence of SARS-CoV-2 resistance mutations in a patient who received anti-SARS-COV2 spike protein monoclonal antibodies: a case report. BMC Infect Dis. 2021;21(1):1223.

57. Jensen B, Luebke N, Feldt T, Keitel V, Brandenburger T, Kindgen-Milles D, et al. Emergence of the E484K mutation in SARS-COV-2-infected immunocompromised patients treated with bamlanivimab in Germany. Lancet Reg Health Eur. 2021;8:100164.

58. Rockett RJ, Basile K, Maddocks S, Fong W, Agius JE, Mackinnon JJ, et al. RESISTANCE CONFERRING MUTATIONS IN SARS-CoV-2 DELTA FOLLOWING SOTROVIMAB INFUSION. medRxiv. 2021:2021.12.18.21267628.

59. Higuchi Y, Suzuki T, Arimori T, Ikemura N, Mihara E, Kirita Y, et al. Engineered ACE2 receptor therapy overcomes mutational escape of SARS-CoV-2. Nat Commun. 2021;12(1):3802.

60. Letko M, Marzi A, Munster V. Functional assessment of cell entry and receptor usage for SARS-CoV-2 and other lineage B betacoronaviruses. Nat Microbiol. 2020;5(4):562-9.

61. Chan KK, Tan TJC, Narayanan KK, Procko E. An engineered decoy receptor for SARS-CoV-2 broadly binds protein S sequence variants. Sci Adv. 2021;7(8).

62. Glasgow A, Glasgow J, Limonta D, Solomon P, Lui I, Zhang Y, et al. Engineered ACE2 receptor traps potently neutralize SARS-CoV-2. Proc Natl Acad Sci U S A. 2020;117(45):28046-55.

63. Havranek B, Chan KK, Wu A, Procko E, Islam SM. Computationally Designed ACE2 Decoy Receptor Binds SARS-CoV-2 Spike (S) Protein with Tight Nanomolar Affinity. J Chem Inf Model. 2021;61(9):4656-69.

64. Jing W, Procko E. ACE2-based decoy receptors for SARS coronavirus 2. Proteins. 2021;89(9):1065-78.

65. Chan KK, Dorosky D, Sharma P, Abbasi SA, Dye JM, Kranz DM, et al. Engineering human ACE2 to optimize binding to the spike protein of SARS coronavirus 2. Science. 2020;369(6508):1261-5.

66. Angelini A, Chen TF, de Picciotto S, Yang NJ, Tzeng A, Santos MS, et al. Protein Engineering and Selection Using Yeast Surface Display. Methods Mol Biol. 2015;1319:3-36.

67. Campbell F, Archer B, Laurenson-Schafer H, Jinnai Y, Konings F, Batra N, et al. Increased transmissibility and global spread of SARS-CoV-2 variants of concern as at June 2021. Euro Surveill. 2021;26(24).

68. Miller NL, Clark T, Raman R, Sasisekharan R. Insights on the mutational landscape of the SARS-CoV-2 Omicron variant. bioRxiv. 2021.

69. Cao Y, Wang J, Jian F, Xiao T, Song W, Yisimayi A, et al. Omicron escapes the majority of existing SARS-CoV-2 neutralizing antibodies. Nature. 2021.

70. Cameroni E, Bowen JE, Rosen LE, Saliba C, Zepeda SK, Culap K, et al. Broadly neutralizing antibodies overcome SARS-CoV-2 Omicron antigenic shift. Nature. 2021.

71. Dejnirattisai W, Huo J, Zhou D, Zahradnik J, Supasa P, Liu C, et al. Omicron-B.1.1.529 leads to widespread escape from neutralizing antibody responses. bioRxiv. 2021.

72. VanBlargan L, Errico J, Halfmann P, Zost S, Crowe J, Purcell L, et al. An infectious SARS-CoV-2 B.1.1.529 Omicron virus escapes neutralization by therapeutic monoclonal antibodies. Res Sq. 2021.

73. Dejnirattisai W, Shaw RH, Supasa P, Liu C, Stuart AS, Pollard AJ, et al. Reduced neutralisation of SARS-CoV-2 omicron B.1.1.529 variant by post-immunisation serum. Lancet. 2022;399(10321):234-6.

74. Islam A, Ferdous J, Sayeed MA, Islam S, Kaisar Rahman M, Abedin J, et al. Spatial epidemiology and genetic diversity of SARS-CoV-2 and related coronaviruses in domestic and wild animals. PLoS One. 2021;16(12):e0260635.

75. Hofmann H, Pyrc K, van der Hoek L, Geier M, Berkhout B, Pohlmann S. Human coronavirus NL63 employs the severe acute respiratory syndrome coronavirus receptor for cellular entry. Proc Natl Acad Sci U S A. 2005;102(22):7988-93.

76. Wrobel AG, Benton DJ, Xu P, Roustan C, Martin SR, Rosenthal PB, et al. Author Correction: SARS-CoV-2 and bat RaTG13 spike glycoprotein structures inform on virus evolution and furin-cleavage effects. Nat Struct Mol Biol. 2020;27(10):1001.

77. Walls AC, Park YJ, Tortorici MA, Wall A, McGuire AT, Veesler D. Structure, Function, and Antigenicity of the SARS-CoV-2 Spike Glycoprotein. Cell. 2020;181(2):281-92 e6.

78. Grange ZL, Goldstein T, Johnson CK, Anthony S, Gilardi K, Daszak P, et al. Ranking the risk of animal-to-human spillover for newly discovered viruses. Proc Natl Acad Sci U S A. 2021;118(15).

79. Kumar S, Stecher G, Li M, Knyaz C, Tamura K. MEGA X: Molecular Evolutionary Genetics Analysis across Computing Platforms. Mol Biol Evol. 2018;35(6):1547-9.

80. Xu C, Wang Y, Liu C, Zhang C, Han W, Hong X, et al. Conformational dynamics of SARS-CoV-2 trimeric spike glycoprotein in complex with receptor ACE2 revealed by cryo-EM. Sci Adv. 2021;7(1).

All documents cited in this specification are incorporated herein by reference. U.S. Provisional Application No. 63/143,614, filed Jan. 29, 2021, U.S. Provisional Application No. 63/160,511, filed Mar. 12, 2021, and U.S. Provisional Pat. Application No. 63/166,686, filed Mar. 26, 2021, U.S. Provisional Pat. Application No. 63/215,159, filed Jun. 25, 2021, U.S. Provisional Pat. Application No. 63/253,654, filed Oct. 8, 2021, U.S. Provisional Pat. Application No. 63/300,154, filed Jan. 17, 2022, International Patent Application No. PCT/US22/14406, filed Jan. 28, 2022, and International Patent Application No. PCT/US22/14407, filed Jan. 28, 2022 which are incorporated herein by reference. While the invention has been described with reference to particular embodiments, it will be appreciated that modifications can be made without departing from the spirit of the invention. Such modifications are intended to fall within the scope of the appended claims.

1. A recombinant AAV (rAAV) comprising an AAV capsid and a vector genome packaged therein, wherein the vector genome comprises a 5′ inverted terminal repeat (ITR), a nucleic acid sequence encoding at least one mutant hAce2 soluble decoy protein under the control of regulatory control sequences which direct expression of the hAce2 soluble decoy protein, and a 3′ ITR, wherein the mutant hAce2 soluble decoy protein comprises an amino acid sequence of:
(a) SEQ ID NO: 12 (hAce2-Variant2) or an amino acid sequence at least 95% identical thereto, optionally fused to an immunoglobulin Fc region;
(b) SEQ ID NO: 10 (hAce2-Variantl) or an amino acid sequence at least 95% identical thereto, optionally fused to an immunoglobulin Fc region;
(c) SEQ ID NO: 14 (hAce2-Variant3) or an amino acid sequence at least 95% identical thereto, optionally fused to an immunoglobulin Fc region;
(d) SEQ ID NO: 16 (hAce2-Variant4) or an amino acid sequence at least 95% identical thereto, optionally fused to an immunoglobulin Fc region;
(e) SEQ ID NO: 72 (hAce2-Variant5) or an amino acid sequence at least 95% identical thereto, optionally fused to an immunoglobulin Fc region;
(f) SEQ ID NO: 73 (hAce2-Variant6) or an amino acid sequence at least 95% identical thereto, optionally fused to an immunoglobulin Fc region; or
(g) SEQ ID NO: 105 (hAce2-MR27) or an amino acid sequence at least 95% identical thereto, optionally fused to an immunoglobulin Fc region.

2. The rAAV according to claim 1, wherein the nucleic acid sequence encoding the mutant hAce2 soluble decoy protein is selected from:
(a) SEQ ID NO: 11 or a sequence at least 90% identical thereto encoding SEQ ID NO: 12 (hAce2-Variant2);
(b) SEQ ID NO: 9 or a sequence at least 90% identical thereto encoding SEQ ID NO: 10 (hAce2-Variantl);
(c) SEQ ID NO: 13 or a sequence at least 90% identical thereto encoding SEQ ID NO: 14 (hAce2-Variant3);
(d) SEQ ID NO: 15 or a sequence at least 90% identical thereto encoding SEQ ID NO: 16 (hAce2-Variant4); or
(e) SEQ ID NO: 104 or a sequence at least 90% identical thereto encoding SEQ ID NO: 105 (hAce2-MR27).

3. The rAAV according to claim 1, wherein the mutant hAce2 soluble decoy is a hAce2 soluble decoy fusion protein further comprising an immunoglobulin Fc region.

4. The rAAV according to claim 1, wherein the immunoglobulin region is a human IgG Fc.

5. The rAAV according to claim 1, wherein the mutant hAce soluble decoy protein is:
(a) a protein comprising SEQ ID NO: 4 (a hAce2-Variant2-IgG4 fusion) or an amino acid sequence at least 95% identical thereto;
(b) a protein comprising SEQ ID NO: 2 (hAce2-Variant1-IgG4 fusion) or an amino acid sequence at least 95% identical thereto;
(c) a protein comprising SEQ ID NO: 6 (hAce2-Variant3-IgG4 fusion) or an amino acid sequence at least 95% identical thereto;
(d) a protein comprising SEQ ID NO: 8 (hAce2-Variant4-IgG4 fusion) or an amino acid sequence at least 95% identical thereto;
(e) a protein comprising SEQ ID NO: 94 (hAce2-Variant2-IgG1 fusion) or an amino acid sequence at least 95% identical thereto;
(f) a protein comprising SEQ ID NO: 98 (hAce2-Variant2-IgG1 fusion with “GS” linker) or an amino acid sequence at least 95% identical thereto;
(g) a protein comprising SEQ ID NO: 96 (hAce2-MR27-IgG1 fusion) or an amino acid sequence at least 95% identical thereto;
(h) a protein comprising SEQ ID NO: 100 (hAce2-MR27-IgG1 fusion with GS linker) or an amino acid sequence at least 95% identical thereto;
(i) a protein comprising SEQ ID NO: 72 and SEQ ID NO: 77 (hAce2-Variant5-IgG4 fusion) or an amino acid sequence at least 95% identical thereto; or
(j) a protein comprising SEQ ID NO: 73 and SEQ ID NO: 77 (hAce2-Variant6-IgG4 fusion), or an amino acid sequence at least 95% identical thereto.

6. The rAAV according to claim 1, wherein the mutant hAce2 soluble decoy protein is a fusion protein, wherein the nucleic acid sequence encoding the hAce2 decoy fusion protein is:
(a) SEQ ID NO: 3 or a sequence at least 90% identical thereto encoding SEQ ID NO: 4 (hAce2-Variant2-IgG4 fusion);
(b) SEQ ID NO: 1 or a sequence at least 90% identical thereto encoding SEQ ID NO: 2 (hAce2-Variant1-IgG4 fusion);
(c) SEQ ID NO: 5 or a sequence at least 90% identical thereto encoding SEQ ID NO: 6 (hAce2-Variant3-IgG4 fusion);
(d) SEQ ID NO: 7 or a sequence at least 90% identical thereto encoding SEQ ID NO: 8 (hAce2-Variant4-IgG4 fusion);
(e) SEQ ID NO: 93 (hAce2-Variant2-IgG1 fusion) or a sequence at least 90% identical thereto encoding SEQ ID NO: 94;
(f) SEQ ID NO: 97 (hAce2-Variant2-IgGl fusion with “GS” linker) or a sequence at least 90% identical thereto encoding SEQ ID NO: 98;
(g) SEQ ID NO: 95 (hAce2-MR27-IgG1 fusion) or a sequence at least 90% identical thereto encoding SEQ ID NO: 96; or
(h) SEQ ID NO: 99 (hAce2-MR27-IgG1 fusion with GS linker) or a sequence at least 90% identical thereto encoding SEQ ID NO: 100.

7. The rAAV according to claim 1, wherein the mutant hAce2 is fused to the Fc domain via a flexible GSG linker, wherein the GSG linker is selected from about 3 to about 123 amino acids in total.

8. The rAAV according to claim 1, wherein the mutant hAce2 is directly fused to the Fc domain.

9. The rAAV according to claim 1, wherein the protein comprises an Fc domain at the C-terminus of the soluble Ace2 protein.

10. The rAAV according to claim 1, wherein the regulatory control sequences comprise one or more of: a promoter, at least one enhancer, at least one intron, and a polyadenylation A signal.

11. The rAAV according to claim 1, wherein the regulatory sequences comprise a CB7 hybrid promoter, a chicken beta actin intron, and a rabbit beta globin polyA.

12. The rAAV according to claim 1, wherein the vector genome comprises an expression cassette having a nucleotide sequence of: SEQ ID NO: 117 (hAce2-Variant2-IgG4), SEQ ID NO: 116 (hAce2-Variant1-IgG4), SEQ ID NO: 118 (hAce2-Variant3-IgG4), SEQ ID NO: 119 (hAce2-Variant4-IgG4), SEQ ID NO: 86 (hAce2-Variant2-IgG1); SEQ ID NO: 88 (hAce2-MR27HL-IgG1); SEQ ID NO: 90 (hAce2-Variant2-IgG1-revised linker), or SEQ ID NO: 92 (hAce2-MR27HL-IgG1-revised linker).

13. The rAAV according to claim 1, wherein the vector genome has the nucleic acid sequence of: SEQ ID NO: 19 (hAce2-Variant2-IgG4), SEQ ID NO: 17 (hAce2-GTP14-IgG4), SEQ ID NO: 21 (hAce2-Variant3-IgG4), SEQ ID NO: 23 (hAce2-Variant4-IgG4), SEQ ID NO: 85 (hAce2-Variant2-IgG1), SEQ ID NO: 87 (hAce2-MR27HL-IgG1), SEQ ID NO: 89 (hAce2-Variant2-IgG1-revised linker), or SEQ ID NO: 91 (hAce2-MR27HL-IgG1-revised linker).

14. A recombinant AAV (rAAV) comprising an AAV capsid and a vector genome packaged therein, wherein the vector genome comprises a 5′ inverted terminal repeat (ITR), a nucleic acid sequence encoding a fusion protein comprising a signal peptide, a mutant hAce2 soluble decoy protein, and an optional immunoglobulin Fc region, wherein the coding sequences for the fusion protein are under the control of regulatory control sequences which direct expression of the fusion protein, and a 3′ ITR, wherein the mutant hAce2 soluble decoy protein comprises a mutant amino acid in (a) R or M at residue 14 (K changed to R or M) and (b) V or K at residue (18) (E changed to V or K), and at least one further residue:
(c) P at residue (22) (L changed to P);
(d) R at residue (25) (Q changes to R);
(e) A at residue (30) (S changed to A);
(f) A at residue (42) (V changed to A);
(g) I or F at residue (62) (L changes to I or F);
(h) D at residue (73) (N changed to D);
(i) P at residue (74) (L changed to P);
(j) Y at residue (313) (N changed to Y); and/or
(k) H at residue (328) (H changed to L), wherein the mutant hAce2 soluble decoy protein amino acid position is based on SEQ ID NO: 81 or 83, or a hAce2 decoy protein at least 95% identical to SEQ ID NO: 81 or 83.

15. The rAAV according to claim 14 comprising an amino acid sequence selected from (i) the substitutions of (a), (b), and (j) or (ii) the substitutions of (a), (b), (g), and (j).

16. The rAAV according to claim 14, wherein the signal peptide is a human signal peptide.

17. The rAAV according to claim 14, wherein the signal peptide is the native human Ace2 signal.

18. An rAAV according to claim 1, wherein the capsid is an AAVrh91 capsid, an AAV9 capsid, an AAVhu68 capsid, an AAV5 capsid, an AAV6 capsid, or an AAV6.2 capsid.

19. A pharmaceutical composition comprising at least one recombinant AAV according to claim 1 and one or more of any of: a pharmaceutically acceptable diluent, a suspending agent, a preservative, and/or a surfactant.

20. The pharmaceutical composition according to claim 19, wherein the composition is formulated for intranasal administration, intravenous administration, intrapulmonary administration, or intraperitoneal administration.

21. A method of treating and/or preventing one or more symptoms of a betacoronavirus or another virus mediated by an Ace2 receptor by administering or co-administering a pharmaceutically effective amount of at least one rAAV according to claim 1 or combinations thereof.

22. The method according to claim 21, wherein the betacoronavirus is SARS-CoV2 and the symptoms are selected from one or more of fever, cough, gastrointestinal distress, nausea, vomiting, diarrhea, eye pain, breathing difficulty, loss of taste, and/or loss of smell.

23. The method according to claim 21, wherein the method comprises administering or co-administering the rAAV or a pharmaceutical composition intranasally, optionally in combination regimen with a hAce2 soluble decoy protein.

24. The method according to claim 21, wherein the method comprises administering or co-administering the rAAV or a pharmaceutical composition via inhalation, optionally in a combination regimen with a hAce2 soluble decoy protein.

25. The method according to claim 21, wherein the method comprises administering or co-administering the rAAV or a pharmaceutical composition intravenously, optionally in a combination regimen with a hAce2 soluble decoy protein.

26. A packaging host cell in culture or suspension comprising:
(a) a nucleic acid molecule encoding a vector genome comprising a 5′ inverted terminal repeat, an expression cassette comprising a mutant soluble hAce2 fusion protein as defined in claim 1, and a 3′ inverted terminal repeat;
(b) nucleic acid sequences encoding an rAAV capsid protein under control of sequences which regulate expression of the capsid protein in the packaging host cell; and
(c) helper sequences for replication and packaging of the vector genome into the rAAV capsid.

27. An rAAV stock produced from the packaging host cell according to claim 26.
